SEQUENCE LISTING (177 sequences)

SEQ ID NO: 1 - protein, 345 aa, Mariniphaga sediminis
MKALVLDEYNQLNYRDFPEPEIHSGEVLIEIKACGICGSDVHGMDGSTGRRKPPLVMGHEASGVIIETGSDVKNWKTGDRVTFDSTVYPLNDWFTLKGHYNLSNNRQVLGVSPGEYRRHGAFAERLAVPQHILHRIPENVSFVEAAMVEPIAVAAHAVNVSGLKPGNSAMVAGAGMVGVFVVQLLKLFGANPVVVIDIDHKKLKLAHEFGADFTFNPKEKDIDERILQLTKSRGFDLTFEVVGITETVQTCITNTRKGGTVVLVGNLSPEIQFPLQKVVTSEITVQGSCAIKGEYELALDLLAAKKVDVSKLVSAVAPLSEGAEWFKRLYEKEPGLNKVILIPDK

SEQ ID NO: 2 - protein, 325 aa, unidentified
  source 1..325: note = Candidatus Dormibacteraeota bacterium
MKAMVWLGPERMEVRDLPAPRPGAGEVLVQTEAAGICGSEVEAYLGRMANRTPPLVMGHEYAGLVTEVGEGVDRVWSRRRVAVNPIVGCGRCRNCAAGDRNLCPDRHLLGIAAPGGFAAFSVVPERCLFSLPEGMDARLGALTKPLANGVHLIRKSGAGRGRVVVIGAGTIGLGCLQAALLHGAEQVDVVERHPGRRRHALALGARQAVAELADVEPGADLVVDAAGSEATRRAAVELVGPAGTASFVGLHSDETVLPWHRIIRANIRVQGTFGYADRDFQQALDWLAAGKAAIPLSELRPLDDGPAAFERLARGPIDEIKVFLA

SEQ ID NO: 3 - protein, 337 aa, unidentified
  source 1..337: note = Candidatus Falkowbacteria bacterium
MKSLIYYGPHDLRYGDSPKPKIRSGEVLLRIKSVGICGTDLHIYNGGMKNLPLPLIMGHEFSGVIEELARDVKNFKVGDRVIAEHVINCGKCSYCLSGKPNLCANSRIIGVHRPGALAEYLAVPADLVYKIPAKLSFEEGAMIEPLSIAYYAVKQARSILGKKVAVIGQGPIGLLLDQILRDAGAEVIGIDIADWRLKLAKKKKWADHTINSSQKTWLKKIQKIAPEGVDTVFEVVGREETAEMAIEIARRDGDVFILGVFESAASLNMMNIVKKELNVFGSWTCAFSFPPSIALAVAKKVDLKSLITNRYDVKDGALAFKESASYKGKRIKTVINF

SEQ ID NO: 4 - protein, 347 aa, unidentified
  source 1..347: note = Acidobacteria bacterium
MKAMLLSQYRRLEIAELPTPIPGRGELLIHVAACGICGSDVHGYDGSSGRRIPPIVMGHEAAGRVAALGPQVTGWSEGDRVTFDSTISCGTCPYCSRGEINLCDRRQVLGVSCGDYRCAGAFAEYVIVPQRIAYKLPDALSFSEAATLEAVAVAVHAVSLSQVSPGTQALVIGAGMIGLLTLQALRAAGCSSVVIADVDASRLRLAKEVGATETLNATGDEFVAEIFKRTNGAGTDLAVEAVGIDATVRTAINSVRKGGTVTLVGNITPEVTLPLQKVVTRQIRIQGSCASAGEYPRAIQLLTSGAIKVKPLITAVAPLEDGPQWFERLYSREPNLMKVVLTPGSVS

SEQ ID NO: 5 - protein, 344 aa, unidentified
  source 1..344: note = Spirochaetaceae bacterium
MKAMVLKEYKYLEIEDIPIPHIEAADELLVRVKATAICGSDIHGFDGSTGRRKPPLVMGHEASGVVESVGSEVKSLKIGDRVTFDSTVWCGECYFCRQGQVNLCDKRRVLGVSCDEYRKDGTFAEYVIVPERIVYKLPDNVAFEEAALTEPVSVAMHAFDITGMKPGENAAVIGSGLIGLLLIQIIRAYSPHLIIAFDTDEKRRKAAIEAGADIALDPQDPESHKTVLSMTNNRGVDRAFEAVGASAAIGTAIESVRKGSSVTLIGNISNKAEIPLQSVVTRQIALLGSCAISGEYPVSLALMANRTIDVRQVISAIAPLEEGPLWFERLYKREPGLLKVVLTP

SEQ ID NO: 6 - protein, 348 aa, unidentified
  source 1..348: note = Candidatus Hydrogenedentes bacterium
MKALRLYAPGDLRLEEIPIPLPRPDEVLVRVLACGICGSDIPRVFEHGTYKYPLVPGHEFSGIVEKVGSETGKDWVGRHVVVYPLIPCRQCSSCYSGLYTQCLNYDYLGSRRDGGFAEYVTVPVNNIIPVPNGISGEISAITEPCAVALHAVRKAQACVGDTVLITGAGTIGILIALWARAAGAEVFMTDIVVGKLKLARSLGFQHLLDAKQTNVSEWVKQKCKHGVDIVFETTGHPAGLETSIDSCSITGKIILVGNPSFEEMKVPAKQYGMILRKEIQITGIWNSLINEYPKNEWQVVLQEFKKESSLYSKIITHKVPIEGLKELFVTIYNKQVFPVKAMFINKEI

SEQ ID NO: 7 - protein, 337 aa, Rhizobium sp.
MKAVVYTQPDAVAVLEREMPALVPGEVLLRIDASGICGSDLHAYHGHDPRRKPGLVLGHEFAGTVVASSDESRLPVGRRVTANPLVTCGHCDYCLQGRDNLCANRGMVGMSRPGALSEYLAIPMRCAIAIPEDLPSVSAALTEPAATALHAVNLTMRAMSRPLPEARVLIIGGGAIGMLTGALVGAYGCRSLAVAETNALRRASVRRHLACEVLDPTANAPAESSFDVVIDAVGAAPTRHLALAATRPGGVIMHIGLQAWSSEIDMRKLTLAEITLLGTYTYTMADMHATVRALASGSFGSLEWVETRALTDAPSAFADLAAGRVAAGKVVLLPQQD

SEQ ID NO: 8 - protein, 347 aa, Citrobacter freundii
MKSVVIHAEGDVRVEERPIPHLQTDEDVLVKVVSSGLCGSDIPRIFNRGAHYYPITLGHEFSGYVESYGAGVTDLQPGDAVACVPLLPCFHCPQCERGYFSLCKQYQFVGSRSEGGNAEYVVMKRANLFRLPSDMPIEDGAFIEPITVGLHAFHLAQGCEGKNVIIVGAGTIGLLALQCARELGAKSITAIDINPQKLALAKELGATYVFNSREMSATEIHSDLENVQFDQLILETAGTPQTVTLSIEIAGPRAQLALVGTLHHDLALASGVFGQILRKELTILGSWMNYSSPWPGEEWVTAARLLTEKRLQLEPLIAHSGDAESFASAVKSLNGAPMQGKILLKLS

SEQ ID NO: 9 - protein, 346 aa, Escherichia coli
MKSVVNDTDGIVRIAESVIPEIKHQDQVRVKIASSGLCGSDLPRIFKNGAHYYPITLGHEFSGYIDAVGSGVDDLHPGDAVACVPLLPCFTCPECLKGFYSQCAKYDFIGSRRDGGFAEYIVVKRKNVFALPTDMSIEDGAFIEPITVGLHAFHLAQGCENKNVIIIGAGTIGLLAIQCAVALGAKSVTAIDISSEKLALAKSFGAMLTFNSLEMSAPQMQGVLRELRFNQLILETAGVPKTVELAVEIAGPHAQLALVGTLHQDLHLTSTTFGKILRKELTVIGSWMNYSSPWPGQEWETASRLLTERKLSLEPLIAHRGSFESFAQAVRDIARNAMPGKVLLIP

SEQ ID NO: 10 - protein, 339 aa, Escherichia coli
  source 1..339: note = Escherichia coli (strain K12)
MKSILIEKPNQLAIVEREIPTPSAGEVRVKVKLAGICGSDSHIYRGHNPFAKYPRVIGHEFFGVIDAVGEGVESARVGERVAVDPVVSCGHCYPCSIGKPNVCTTLAVLGVHADGGFSEYAVVPAKNAWKIPEAVADQYAVMIEPFTIAANVTGHGQPTENDTVLVYGAGPIGLTIVQVLKGVYNVKNVIVADRIDERLEKAKESGADWAINNSQTPLGEIFTEKGIKPTLIIDAACHPSILKEAVTLASPAARIVLMGFSSEPSEVIQQGITGKELSIFSSRLNANKFPIVIDWLSKGLIKPEKLITHTFDFQHVADAISLFEQDQKHCCKVLLTFSE

SEQ ID NO: 11 - protein, 357 aa, Lactobacillus salivarius
  source 1..357: note = Lactobacillus salivarius cp400
MNIPTKMKAVVKSEAGYDNMSLQEIPVPEVKGKQVLMKVAYTGICGTDIHTFKGEYANAVTPLVLGHEFSGKVVAVGDEVTKVKPGDRVTSETTFDTCGECVYCKNKEYNLCENRSGIGTKANGSMANYVLTREESVHVLPDNVSYKLAAMSEPLASCVHAMYQKTPFTLHDTVLIIGPGPMGLLSLQIAKEIGAFTIVSGITKDSDRLEIAKELGADIVVDTQKEDLAKIVMEATNGVGVTKVYDCSGATPAVNAALPLLKKGGTFQQVGLFAKPLNELDERTIIQHEITYVGSRSQNPYDWPIAIHLEAKGAINEDKMVTKVFDLDHWREAFEAMMAGKELKVLIASNPDDETLN

SEQ ID NO: 12 - protein, 350 aa, Clostridium difficile
MKAAVLHGTNDMRFEDIEIKPCESDEVKIKVMAAGICGSDPPRVLKHWKYPVPAIPGHEFSGVIAEVGKDVKNVKVGDRVVAIPFIPCNECEYCKRGLFSLCDDHGMLGAKSFGAFAEYVNIKATNVLPIGDMDFEDAAMIEPLAVAMHGVLNIGVQVGDTVAVMGSGTMGQLVIQGLKIAGAGTIIAVDISDNKLRESKELGADIIINAKDINPVEKIKELTGGKGVDIALECAGSKITQEQCLLITKKKSKIGFLGIAYSDITLSEEAFENIFRKELELKGFWNSYSAPFPGQEWTKGINLVNEGKIKLKEMVSHRFSLEDTYKAFEMIRDRKEEFNKILILPQGVEK

SEQ ID NO: 13 - protein, 350 aa, Clostridium difficile
MKSVRFYGIRDTRVEDVDVPKILEKDDVIIKVKVAGICGSDISKYSKTGPHMVGEILGHEFSGEVAQVGKEVRSFKIGDRVAVCPAMPCFECDECKKGLYSRCNNVAIIGNKELGGCFAEYTKVKERNLIKIPDEISYETAAALEPVCIAGHGLFRSEAKVGDTVVVLGTGPIGLFSIQWAKIFGSTKIIAVDVFDEKLDLAKELGADICINAKEKNIVEEIKRLTDGDGADIVIESAGTPLTCGQVLLLAKKGGTVLYAGVPYGDVALTREQFEKIVRSELTVKGTWFGNSFPFPGKEWSAGLYHMQKGDMNVEKLVTHRINLEEAPAYFEKVYKRDIFFGKIMINIDN

SEQ ID NO: 14 - protein, 349 aa, Lactobacillus rhamnosus
MKASMLEDLNKFSVKEIDIPSPKKDEVVVKVMAAGTCGSDSHKMISGWKYGYPAVMGHEFSGIVTQLGENVSNVSVGQHVAVAPFIPCFKCHYCQIGLFQMCENYSMLGQQKFGGFEQYVSVPARNVLDIGKMSFEEGALIEPMAVAAHAVMGIKPELGDTVAVFGLGTVGDLVVRLLISSGATNVIGIDIDDQKLEKGLDEGCTHVINSAKESLEEKIMEYTDGLGVDISMECAGSKITEEQTLLVTKRRGKVGFVGIAYSDVLLHQKAFENIFRHELTVTGFWNSYSAPFPGREWTNSIQLVNRGRIKIKDLITHRFELEDMQKAFNMITTRSESFNKVMFFPNGIN

SEQ ID NO: 15 - protein, 354 aa, Bacillus halodurans
MKALNLYGIQDLRFEETPAPSIEHDDDIIIKVKAVGICGSDLSRYKKLGPYVPGMTFGHEFAGEVVKIGRSVTGFSIGDRVAACPTYTCGQCRYCQLGEPTRCERLSVIGARHPGAYAEYVKLPAKHVIPLPNVVNYDEAALIEPASVVAHGFYRTNIKPGASVAIMGVGSIGLLAVQWAKIFGATTVFAIDIDEQKLNVANQLGADVLISSLQRPAHKQILEYTNGIGVDVAVESAGTPSTSAQVFALPKKGGEVVFLGIPYADVQIERFYFEKIVRNELHVYGSWNALSSPFPGKEWATTIHYMSSGQLNVAPMISYRLPLAKGPETFQQIAKGELKPTKVLFYPEKLSERK

SEQ ID NO: 16 - protein, 351 aa, unidentified
  source 1..351: note = Lachnospiraceae bacterium
MKVLSLHKPGDIRFEDAPMPEPGEGEVLLKVMACGVCSSDEARIFKDGAHIMPIVPGHEFCGQVVKTGAGVDESLIGRKASVFPMLPCFECPSCKRGDYATCSNYKYYGSRNDGGMGEYRAIKAWNLNLLDDSVDYCVGALSEPSAVGHHATEVGGVKAGDKVFVIGSGTIGLLIGFFAQAKGAEVWIGARRKESNDFAASLGFKTIDTNNTEDEVAKATDGNGMDVVFEAVGSNKCLIEAIGSVTAGGTLVTVGNPVGDMNLPRDVYWRILRKQLTVRGTWNSSYSDKANDWHEVAALMKEKKIPFEKLITRKYKLSQCDEAFDFFRDHSIQKSRVMFVMHEKESEAYNG

SEQ ID NO: 17 - protein, 336 aa, Faecalibacterium sp.
  source 1..336: note = Faecalibacterium sp. OF03-6AC
MVNMMKALVFTAPHTFEYRDVPRPEPAPDEVLVEVKACAICGSDVHGSSGENGRRIPPLVMGHEASGTIAQLGEAVQSWAVGDRVTFDSTEYCGYCWFCTHGMHNLCAQRRIVGVACTEYRKNGAMAQYIAVKARTLYALPPAVSFEQGCLVEPFAVGMHAVLRSGLAAGQTAAVIGDGTIGLMTLIAAKSQGAQVCISGHHTRPMQLAAQLGALDCAQLPQLTAERGADVVYDAVGSQSSFTQAMELVRKGGRIMVIGNHARTIDFPLQRCITEQIDVQFSYSSAGEYPVCLQAIAEGRADLTGLWQSFPLAEGAQVFDRLVHRDESLTKAVLIP

SEQ ID NO: 18 - protein, 357 aa, Agathobacter ruminis
MKAYVLDGIGQLNQHEYVLRDLQPGEALVQVAAAGICGSDLPRIFETGTYHFPTIPGHEFAGVVKELYADADEEMLRQWMGKRVAVFPLIPCRTCRSCANHQYEMCSNYNYLGSRTDGGFAEYCIVPVWNLLQLPDEVSFAQAAMMEPAAVALHAIRRGGLHPDHPETGIGKRVLVMGLGTIGGLIAQWLSQCGVTELTATGHHAPQEELMHRYANENYRFINTNQTAWTDDENTYDLIFDCINDSESVQQALRYVKPGGQIVAVGNPKKDLTLQKDIYWKLLRKQVCMTGTWNSSFSHDSNDDWQILLRYLQEQKLHPQALITHRLPFAQLPKGLEIMRLREEFYNKIMIDLTVCS

SEQ ID NO: 19 - protein, 342 aa, Alicyclobacillus acidoterrestris
MKAVTYTPDQGFALVEVETPQLGPDEVLLQVEACGVCGSDRQVVAGESVPTGTAFPLVMGHEIAGRIAALGETVTDWHVGDSVIVHPFVACGTCAPCVHGQPNLCVRQVCIGYQRPGGFAEQVAVPARQLVRRPAELAPAAAALVVDAFATPYHAMRAAGVESGQTVLIIGTGGLGLAALMLAMGFHVGRLGAVTRRQEGIAIAEAHGAQMVVVTDQDERTIARQLRRWSGASGIDVVIDTVATADTITLAAESVRPGGTIAIVGMSADTALYPVAKTVRRGLTFVASYGSVREDVEQLVSWVAEGRLDPVRLVAGALPLHEAARAFAPDRPAGRWVIDPSE

SEQ ID NO: 20 - protein, 357 aa, Ruminococcus sp.
  source 1..357: note = Ruminococcus sp. KGMB03662
MKSLINIPEKMKALVLYGVGDLRVTEVDVPKLEKGTVLLKIKACGICSSDMPRCFVTGTYHFPTIPGHEFSGQIVAVGDDVDESLLGKRSCVFPMLPCKHCKACKMEEYAQCSGYSYFGSRQDGAWAEYLVVPVWNLVPFDDTLDYKTAALCEPAAVSMHAVNIGDIKEGQNVVIIGTGTIGFMIALFAKERTKTGKVVIAGRSKNKLEYAKKLGFDVVDTSVDDLPAQVKKIIGIDGADVTFEAVGSNEAIASAIESTGALGSIVLVGNPSTDLILPKNIYWSILRKQITVKGSWNSSYNSRVNDWKKALEVFEHSDVNFADLITHTFTIEENEKAFSAIRNTGEFTLKVMFTFDD

SEQ ID NO: 21 - protein, 348 aa, unidentified
  source 1..348: note = Clostridiaceae bacterium
MKAVVLHGKNDICYEEWPEPILKTGTVKIHVKACGICGSDIPRVLGDSAHYYPIILGHEFSGCVTEVADDVDKIQVGDHVAGVPLLPCMKCDDCQQGNYSQCKHYSFIGSREQGGFADYVVIPADNVVKVDPTLPYEQVALFEPCTVALHALKVSSYKGGKRVAILGGGTIGLFALQWAKIYGASQVTVFGRSKEHLKIAAALGADTTISTLDDNFMTEAMRVTEKKGFDYIYESAGSTITMKLAFELAANHAKICFIGTPTENLTFSPKLWENMNRKEFQLMGSWMSCSAPFPGDEWKETAHFFNTGQLRYVPEMFFAQYPMDDVKKAFEQFHNKNKVKGRILLKNK

SEQ ID NO: 22 - protein, 337 aa, unidentified
  source 1..337: note = Thermoplasmata archaeon
MKVAVWYGDKNIVLEDRPIPSIGEKDVLVRVKSSSICGADLHAYKGLSKRRKPPLVMGHEFSGMVERLGDETKGLKVGDRVVVEPIISCGKCKACKKGKTNVCENRKLIGLNLDGAFAEYVSVPYEKCHELPRDLSFDEGAIIEPLAVAVHAVNITPISSNDNVLIIGSGVIGLLITQLLRSRTSGDILVTGRRDYLLEKAREFGATDAINVIDCDAVKTLLKKHGRVDVVFEAVGSNETVNQSISLVESGGSITLVGMLDREMTVDMMEVVAREITLRGDYGYTTDDFKEAIELASSGKVDLKGLITHVLPLDEIKKGFDILAERRENVIKVVINP

SEQ ID NO: 23 - protein, 343 aa, unidentified
  source 1..343: note = Lachnospiraceae bacterium
MKALVLEEIGKLTLKEVETPVPGRGEVLLRIHACGICSSDKARLFKNGTHTMPLIPGHEISGEIVAGGPGIREELIGRRAAVFPLIPCRQCDSCRIGKYAQCEHYDYFGSRRDGGFAEYLAVPVFNINLLPDDMDYHTAALCEPATVASHALKRAKMKSGDIVTIIGTGTVGLLAAGIARIRRASKIIIVGRSKEKIMNAAHFADHVINAEGWDVNEAVASLTEGHMSDVVLETGGNSDACTYALMAAGKGTTVMFIGNPEEPIVFNRDIYWKILRNELTLKGTWNSGYAPEDNDWNDVIRYMADGQIPADKIITDSFPLEDYNKAFELLLSDRFYTKIMFDI

SEQ ID NO: 24 - protein, 348 aa, Prevotella marshii
  source 1..348: note = Prevotella marshii DSM 16973 = JCM 13450
MKANVLYHIGDLRYTDVALPELHDGEALVRVRACGICGSDVARVFTTGTYHFPTIIGHEFSGEVVETYSAADAEWVGRRVSVFPLIPCRHCDACHHGQYEMCTCYNYLGSRTDGGFAEYVAVPVWNLFRLPDSVSYEEGAMMEPAAVAMHALKIARVGMGDTVAITGPGTIGMILTQLIQRIGGTKAVLIGRTQAKLDYARRIGIRHVCNSGTEHVADRVGQLTGGRGADIAIEGTGASEPMNLCLNLVRSEGRIVAMGNPQGDMLFRKDAYWKLLRKQLTLRGTWNSTYGLGRDDWQDISALLAEGALNLRGLITHRLPLAELQQGLSLMRDAAVYTNKVMIVDEQG

SEQ ID NO: 25 - protein, 337 aa, Blautia sp.
  source 1..337: note = Blautia sp. OM05-6
MRAYELHGVNDLRREDIEKPEIPSGWVLVQVKASGICSSDIPRIFTNGTYHFPTIPGHEFSGVVAAYGEGVPEERVGKRVGIFPLIPCRTCPQCRQKKYEMCEHYDYLGSRRDGGFAEYVAVPDWNLLELPENVSFREAAMLEPLSVALHAVKRSGVKPGDTAAVIGTGMIGFAAAAWAKALGAETVFVIGRGEAKRAIAEQIPGISYVTEEESKAIQADVVVEAVGTPQAVSSAVLLARPGGSIVLMGNPSGDLAMEKNVYWRILRKQLNLMGTWNSSYEKDEACDWTEALDALSKKKIPAQALITHCFPSEKLMDGLELMKNHKEPYCKVMVSWE

SEQ ID NO: 26 - protein, 354 aa, unidentified
  source 1..354: note = Candidatus Bathyarchaeota archaeon
MKRMRAAILYGPKDIRIENIEMPKVKPEWVLIKVRATGICGSDLHFYKEKSFIPIISDVGKGRYVPGHEFSGEVYELGSKVKGLKKGDRVGVEPIIGCGKCQWCKVGCYNLCEHYKLIGFHYIGGMAEYCVAPAENCFKLPNNVSFESAAMLDCIAVAIHAVNKAEIRGGDSVLIVGAGAIGLFALQAALIQGASDVYVVGKYEFQLEKARKLGATAVINAKNESFSEKIMEITSGKGVDKVIEAVGGASNVISDVLDVTRKKGTIVTTGIFTRPLSVDFFKILTKELTITSSWGYCYFDRRKEFGTALDFLAKEKINTQELITHKYPLEKVSEAFETALNKNKEKSIKVEIIF

SEQ ID NO: 27 - protein, 350 aa, unidentified
  source 1..350: note = Lachnospiraceae bacterium
MKAWVLHGIGDIQYEEVKEPQSAGDEVIMKVMSVGICGSDIPRIYKTGTYHFPTIPGHEFSGIVADVGAGVNPKWKNKRVGVFPLIPCHVCASCKKGQYEMCRNYGYLGSRQNGGFAEYAAVPVKNLIELPDSVSMQEAAMLEPMAVAVHAMRRARPMTGDRIVVCGLGTIGMLLVSFLRETGIRDIFVIGNKAFQRITILKTGLQENCYCDSRVQDVEKWLMERTEGKGADVFFECVGRNETISQAVNLTAPGGSVCLVGNPCSDMTLEREVYWKILRNQLTITGTWNSSFTHEIEDDWHYVLDRLEQGKISPGDLISHKFPLKNLESGLHIMRDKTQDYIKIMAEINV

SEQ ID NO: 28 - protein, 348 aa, Alkalihalobacillus ligniniphilus
MKAAVLHSENLFTYEEVEIDPCKDDEVKIKVMAAGICGSDTHKMTSKWKYPLPTIMGHEFSGFIVEIGEKVTNASIGDRVVAIPFLPCNDCEYCKRGQFSLCEDHGMVGSKSFGGFAEYVNIKSTNVLPIGDMDFEEAAMIEPLAVALHGVLNIHPRIGDIAAVFGIGTIGILTIQWLKIAGVKEVIAIDISDNKLADAHAMGCERTINPFKENLLEKIDEYTNGRGVDIAFECAGSKVTQEQCLLVTAKRGKVGYLGIAYSDVHLSEQAFENIFRRELTLKGFWNSYSAPFPGEEWTKSIDFINKGRIKLKELISHRFKLQDTQKAFDMMINREETYGKVLILPNKE

SEQ ID NO: 29 - protein, 348 aa, Jeotgalibacillus soli
MKAAVLHSENHFIYEEIDIAPCQDDEVKIKVIATGICGSDTHKMTGKWKYPLPAVMGHEFSGYIVEKGKNVTNAAIGDRVVAIPFLPCNDCEYCKRGQFSLCEDHGMVGSKSFGGFAEYVNIESSNVLPIGDMEFEEAAMIEPLAVALHGVLNIHPRVGDVAAVFGVGTIGMLTIQWLKIAGAKEIIAIDLSDEKLADARAMGCERTINPLNEDLLEKINEYTNGMGVDIAFECAGSKITQEQCLLITRKQGKIGYLGIAYSDVHLSEKAFENIFRRELTLKGFWNSYSAPFPGEEWTKSIEFIKNGRIKLKELISHRFKLEDTQKAFDMMINREEKYGKVLILPNEE

SEQ ID NO: 30 - protein, 350 aa, Heyndrickxia sporothermodurans
MKAAVLHSENEIIYQDIEISECQPNEVKVKVMAAGICGSDTHKMVSKWKYPLPAIMGHEFAGYVVEKGKDVTNVEIGDRVAAIPFIPCNECEYCLRGQFSLCENHGMIGSKSFGAFAEFVNIPAKNVLPIGEMSFEDAAMIEPSAVSLHGVLNIHPQLGDVVAVFGVGTIGILAIQWLRVAGVKEIIAIDISDKKLQDAMDLGCEKSINPLKEDLIKKIYEYTNGLGVDIAFECAGSKITQEQCLLITRKQGKVGYLGIAYSDVTLSEKAFENIFRRELTLKGFWNSYSAPFPGKEWMNTIEFVQQGKIKLKELVSHRYKLEDTQKAFNMMINREEEYNKVMIIPNEELE

SEQ ID NO: 31 - protein, 346 aa, Clostridium fungisolvens
MKAAVLHSVGDIRYENIEIKPYGDDEVKIKVKAAGICGSDPPRALKKWKYPVPAILGHEFSGYIVEVGKNVKGFKVGDRVVAVPLEPCHQCEYCKKGQFSLCESYDMLGATSFGGFAEYANVKATNVLKIDDIDYEEAAMIEPLAVALHGVLNINPQLGDTVAVLGAGTIGQLTIQWLKVSGVEKIIAVDISDKKISEAMSLGADIGINALKENPVEKIMELTNGFGVDICIECAGSKITQEQCLLVTKKKGKIGYQGIGHAGIELSEVAFEGIFRKELNIQGFWNSYSAPFPGQEWFKSIEYVKQKKIKLKQLISHRFSLEDTAKAFEMIGERKEEYNKIMIVQD

SEQ ID NO: 32 - protein, 348 aa, Alkalihalobacillus nanhaiisediminis
MKAAVLHSENHFIYQDMESFECQNDEVKIKVMAAGICGSDTHKMVSKWKYPLPAIMGHEFSGYVVEKGSDVANVEIGDRVVAIPFLPCHTCEYCKSGVYSLCEDHGMIGSKSYGGFAEYVNLKATNVLPIGNMDFEEAAMIEPLAVALHGVLHIQPRLGDVAAVFGVGTIGMLAIQWLKIAGVKEIIAIDISDEKLAEAKAMGCERTINPLQDNLIEKVHEYTNGLGVDVALECAGSKITQEQCLLITRKKGEIGYLGIAYSNVVLSEQAFENIFRKELTLKGFWNCYSAPFPGEEWTKSIDFILKGRIKLKELISHRFPLEETQKAFDLMINRKETYGKVMIIPNKE

SEQ ID NO: 33 - protein, 348 aa, Neobacillus cucumis
MKAAVLHSENQIVFQEFEIGELKPDEVKVKVMAAGICGSDTHKMVSKWKYPLPAIMGHEFAGYIVEKGKDVVHVEVGDRVAGIPFVPCNDCEYCFRGQFSLCENHGMVGSKSFGAFAEYVNIPAKNVLPIGDMDFEDAAMIEPSAVALHGVLNIHPQIGDVVAVFGVGTIGILALQWLKVAGVKEIIAIDISDKKLQDAIALGCERTINPLKENLLEKIHEYTNGLGVDIAIECAGSKITQEQCLLITRKQGKVGYLGIAYSDINLSEKAFENIFRRELKINGFWNSYSAPFPGKEWTNTVEYVKQGKIKLKELISHRFGLEDTQQAFNMIINREEEYNKVMIVPNGE

SEQ ID NO: 34 - protein, 341 aa, Staphylococcus aureus
MINQVYQLVAPRQFDVTYNNVDIYGNHVIVRPLYLSICAADQRYYTGRRDENVLRKKLPMSLVHEAVGEVVFDSKGVFEKGTKVVMVPNTPTEQHHIIAENYLASSYFRSSGYDGFMQDYVVMAHDRIVPLPNDIDLSTISYTELVSVSYHAIQRFERKSIPLKNSFGIWGDGNLGYITAILLRKLYPEAKIYVFGKTDYKLSHFSFVDDIFTVNQIPDDLKIDHAFECVGGKGSQVALQQIVEHISPEGSIALLGVSELPVEVNTRLVLEKGLTLIGSSRSGSKDFEQVVDLYRKYPDIVEKLALLKGHEINVCTLQDIVQAFEMDLSTSWGKTVLKWTI

SEQ ID NO: 35 - protein, 350 aa, Staphylococcus aureus
  source 1..350: note = Staphylococcus aureus subsp. aureus 71193
MLLFEVVLPMINQVYQLVAPRQFEVTYNNVDIYSDYVIVRPLYMSICAADQRYYTGSRDENVLSQKLPMSLIHEGVGEVVFDSKGVFNKGTKVVMVPNTPTEKDDVIAENYLKSSYFRSSGHDGFMQDFVLLNHDRAVPLPDDIDLSIISYTELVTVSLHAIRRFEKKSISNKNTFGIWGDGNLGYITAILLRKLYPESKIYVFGKTDYKLSHFSFVDDVFFINKIPEGLTFDHAFECVGGRGSQSAINQMIDYISPEGSIALLGVSEFPVEVNTRLVLEKGLTLIGSSRSGSKDFQDVVDLYIQYPDIVDKLALLKGQEFEIATINDLTEAFEADLSTSWGKTVLKWIM

SEQ ID NO: 36 - protein, 341 aa, Eubacterium ventriosum
MINTVYQLVAPRRFEIKYSDIDLNNNKVIVRPTYLSICNADQRYYQGLRDAEILRKKLPMALIHEAIGEVVRDPSGNFKTGELVVMVPNTPVEEDDIIAENYLRSSKFRASGFDGFMQEYVEITPDRLVRLPQNIDRTVAAFTEIVSVSVHAIGRFDKIAHKRRNVVGIWGDGNLGYITAVFFKTMFPETKLYIFGVNPDKLQDFTFADATFTVEHIPEDVKIDHAFECVGGAGSGKAINQIIDYINPEGTISILGVSEYPVPINTRMVLEKGLRIFGSSRSGVADFEKTVALYKSNPEIINYLSNIVGAVVPVRTIEDMKKAFEMDIQKAFGKTIMMWDK

SEQ ID NO: 37 - protein, 343 aa, Pradoshia sp.
  source 1..343: note = Pradoshia sp. D12
MINQVYRLVSPRQFEVAYNDRSLQYHEDFVVVRPTHLSICAADQRYYTGSRGKEAMAKKLPMALIHEAIGKVVYDPKGEIKNGTMVVMIPNTPTEKDEVIGENYLRSSKFRSSGYDGFMQDYVFMGRDRIVELPEGIDPYVVAFTELMTITTHVLSRFESRSHKRRNVFGVWGDGNLGFITTLFLKKMYKDCKVIIFGKTGYKLDNFSFVDEVYTIDEIPEDLRIDHAFECVGGKGSQYAIDQMIDHLHPEGSIALMGVSEFPVEVNTRMILEKGITLIGSSRSGREDFVRTVNFLKENPDVVEYLGTLVGEVQEVKTISDILSAFEKDLTSSWGKTVMEWKI

SEQ ID NO: 38 - protein, 341 aa, Lactobacillus plantarum
  source 1..341: note = Lactobacillus plantarum EGD-AQ4
MLNQVYRLVDPRQIEVQTVAEEITDNDIVVRPKYLSVCHADTRYFTGQRPQATLRQKLPMALIHEGVGEVVKDPQNKFKPGTLVAMVPNTPFETDPIIKENYLPTSKFRSSGYDGFMQEYVTLHRDRAIVVPDNFDHQMSAFIEMVSVGVHALTQLEAVMDADRKIIGIWGDGNLGFITATLVKQIFPDSQLMIFGRHRSKLDYFSFADKTYLVDDIPSDLQVSQALECTGGRGSESAIAQIIQHIRPMGTAILMGVSEDPVGIDTRSVLAEGLTLRGVSRSGRADFQRAIDILTKSPVTRERLQNLVGFTRKVSTIQDITDFFEGALTNYWGKAVMEWDV

SEQ ID NO: 39 - protein, 337 aa, Ruminococcus sp.
  source 1..337: note = uncultured Ruminococcus sp.
MINYVYQLVSPHVFSLKYEEVDFKKEVIIRPKCMAVCHADQRYFLGRRDAKILRQKLPMALIHECCGQVVWDGTGSFQRGQKVVLIPNVPGRDDGVTYENYAEGSRFLSSGCDGFMREYVNLSPDRVVPFEQVPLPVAAITEFVSVAVHAAGRFMRAAHTRREAIGVWGDGSLAYVVCCVLRRLLPESRIFVVGKHERKLLQFSFADRTFLFDSLPEDFHIDHAFECTGGEGSYYAIDDIITAVRPQGAVMLMGVSENRVPVRTRDILEKGLTFTGCSRSGREDFVKAVELMEQPQFQKRLSAIIYEDRPVHTVADLSRVFETDLNTPFKTVFRWEI

SEQ ID NO: 40 - protein, 338 aa, Kandleria vitulina
MINTVYRLIAPKSIRADFVDLKLDEERIIIRPTYLSICAADQRYYTGSRGRVVMKQKLPMALIHEAVGEVVYDPKGELPVGTHVVMIPNTPNEKDDIIKENYLRSSQFRASGFDGFMQNVVSMRRDLVIPFDYEPEVGVLLELMSVGMNAVSTFKKHAHDRKDAIGIWGNGNVGFVTALILRKEFPDAKIYVFGVEEHKNRYFSFADDTFIINDIPEDLQLDHAFECVGGRGSEAAISQIIDYIKPQGTINLMGVSENPVPINTRMVLEKGLQLLGNSRSNYDDFKKSVDMMANNKDVREYLSTIVSEMVVVNTIDDMIRAFEDDTYNDFKTVMKWEI

SEQ ID NO: 41 - protein, 341 aa, Lactiplantibacillus plantarum
MLNQVYRLVSPRQFEVETVAEEVDADDLIVRPKFLSICHADTRYFFGQRSQAALRAKLPMALIHEAVGEVVYDPRNEYKPGTLVAMVPNTPVKKDSIIQENYLPSSRFRSSGYDGFMQEYVCLRRDRAITVPSDFDPHMAAFIEMVSVGVQALTKLESTMDEDQRVIGIWGDGNLGYITATLVKQIFPDSQLLVFGRHQSKLDYFSFADKTYLVDDIPADVKVSQALECTGGQGSEAAIQQIIQHIQPMGTAILMGVSENPVGIDTRSVLAEGLTLRGVSRSSREDFKRAVDILSSSPVTRERLQNLVGYVQKVSSIQDITNFFDSALTNYWGKEVMEWDV

SEQ ID NO: 42 - protein, 342 aa, unidentified
  source 1..342: note = Lachnospiraceae bacterium
MINTLYKLTAPLIVESFYENIDCHSDNIIVRPEKLSICKADIRYYFGMRNAKVLKEKLPLALIHEACGSVVYDITGKFKAGDQVVMLPNIPGINMQYHENYRRDSLFRSSGADGFMQELVSIPQSQVIKFEKMDQNIAAFTEFISVAIHAVSSYLDRSAGKRNGIKRKIGVWGSGAMGYAVSCVLKYFIPSAEITVIGGSKAKLDMFSFVNHTCSLSELPDDAEFDDCFECVGGAQSGNAINQIIDHILPEGLLLLLGVSEEAVPVNTRMVLEKGITLLGRSRSERSDFIKTIDILDTNENIRRRISNLISGEVEINEVNDIHKAFNQSKIADYKIILNWRI

SEQ ID NO: 43 - DNA, 1599 bp, synthetic construct
  source 1..1599: mol_type = other DNA
cttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactcgggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcgataacccagacatgaacaagctccagttccatttgatgctggatgaatttttctaatggtgttcaccactgttgcttcccctttgttggagcgtccgtcgttctgatggcgctctgagcccaaaatatcccttcttcgcccgagagcaagcctcggagtgtgcttgcaatctacttttcctactaaagttatcttcttgtggcttctgaatttgaatagaacgtgttcagacttgttagctaaatttgttgatgcgagatacttgacagaatcccgttgatgaactcatctgtgtgaccaccgcctggtgatggaatctcgcgcggaagccgtggtgttactgactctcaagcaattcagtagataaatttgtataacatcattgcaaggggtgctagcaatcaattgtccaataccaaacggcggcaagtgcaaaggggcgatagagagtttctgcgaggaagggaaggaatccgtatgcctgaaaacttttgtacaagtgtctttgtaagggcgaggtctgctctgacatctctctgagatctgagatctgatacctgcaggataacttcgtataatgtatgctatacgaagttataaccagatcgaagctcaccccttgttgcaacaagacgagttggttgctcaccataagagcaagaacattgtcatcactgcttacagtcccttgggcaacaatgtcgctggtaaaccacctctgactgagaaccccggtattgtggatgctgctaagcgtttgaaccatactcctgctgctgtgctcattgcttggggtattcaacgcgggtacagcgtcttggtcaagtcagttacaccctcacggatcaagagtaactttgaacagatcactctgtctgatgaggaattccaacgggttaccaacctcatcaaggagtacggtgagagccgtaacaacgttcctttcaattacaagccttcgtggtccattgacgtctttggtacccaggacgaggcta

SEQ ID NO: 44 - DNA, 4618 bp, synthetic construct
  source 1..4618: mol_type = other DNA
tgcagagccttgcaaatgatgaaagaccaatggaattgatcattgattattgatgatgcatgatgaaagagatagaaacagatgcaagatgggatggcgcgatgatcaaatgataatgaacgttcaacccttcaaaattgaaactctcaggctgagcggctgagcaagctctgagaatgtgggtcttcgaagactttcgcttgagcatggtgcatgtgcaaagagatggaatgtggaattgttgtaggtaggaacacagcccggtagagcggcgcagtggctcagcgaagctgggagaacgactgaaccggtcttgagattcatcatctcagcgtgcgtcagcgcgtcagcttcagcgtttgagaatggcacggactgacctgtcgtacatcccaaggaggcccgtgcgctatactcgccttgtggcctaatctgcgatcaacccatcctcaacgaaagcgcaagtagcgactaagcacactatctttcgggtcggtgcggtcacagtatcggcatctctgtctttagctacgatggtgtgcgtgtaactgatcagtacacccgctgtatcaaaactctatcttcaatccgctaagaatcgtcaccatcagtacatcagtacatcagtgggctaagatctaatggtgtcgttcgacggccgcgggcactagccacgagccaagagcggtacagttgctgcgaaatggtgggcagcctgacaggttcagtttgaataggatgaaaacgccatgaagaacagagaagctggcaattgacgatagtggggcgagcgcacgctgcttgctggtccgagcgtaggacgaacgacaaaccatgggcggggccaccaggccccagggagggcgtcactggttggatgtcgtctgttaggctagagctcaggcgctcagggcctctggcccaagcctcggacgcaagcctctgacgagcgagcgcgcgctggggcgggtctgcgcaggaacggcaggactgccgagttggttgaatattgaatggttggatgattggtttgctgggtttggttttgacacgcacggcacaggacaagtgagtttggaggttggatttgcgcaggtagaaagggcgctcggcctcagggcctcagcgtcagcagacgagctcgccagtccaggtcagtacccagtgttcgttacttcccaccgtccagagtttgctgctgcctctcagcttgattcgtgtcggagaagtagaaatggtcacagcgtaccacacactgtccccatgcctgcgtcctgctccacgagtgtttagccacaaaaatttatggacaaccttactgagcctgtgctggggggtgctttagcaccagcactggcgccatggcgtcaaggttaattccactgtcattcgacaaccttcaactgatgggggcaactgggcacaaaaccttggatatataaccaactccaactcccacggttttttaccttttgctcttctccccaggactcactacttctactcctctattcatcttcaccaaaatgtgagacctaatatcactgctgtacagacaagactgtgtatattaggtctcaatgaaagccctcgttttggacgaatacaatcaattgaattaccgtgatttccccgagcccgaaatccactccggagaggtgctgatcgagatcaaggcgtgcggtatttgtggctctgatgtgcacggtatggacggatcgaccggacgtcgaaaaccaccgctggttatgggtcacgaagcctctggcgtcattatcgaaaccggtagcgatgttaagaactggaaaacaggtgaccgagtcacgtttgatagcaccgtgtacccacttaatgactggttcactctgaaaggccactacaacctcagcaacaatcgacaagtgttgggagtgtctcctggcgagtatcgccgtcatggtgcgtttgccgaacgacttgcggttccgcaacatatcctgcaccgaatcccagagaacgttagcttcgtggaggctgcgatggttgagcccatcgctgtggcagctcacgcggtgaatgtttctggattgaagccgggcaattcggctatggttgcgggcgcgggcatggtcggagtgtttgttgtccaactgctgaagctttttggagctaaccctgttgtggttattgacatcgatcacaagaaactgaagcttgctcacgaattcggtgctgactttacattcaatcctaaagaaaaggatattgacgaacgaatcttgcaactcacaaaatcgcgcggttttgaccttacatttgaagtcgtcggcatcaccgagacggtccagacctgcattaccaacacacgtaagggtggcacggtggttcttgttggaaacttgtcccccgaaatccaatttcccctgcaaaaagtcgttacttctgaaatcactgtgcagggctcttgtgccatcaagggtgaatacgagctggcactggacctcttggccgcaaagaaggttgacgtctcgaaactcgtttcggctgttgcaccgctgtctgagggagcggaatggtttaagcgactttacgagaaggaaccgggattgaataaagtgatcctcattccagataagtaacacggatcaacaaaaatgtgcgccagctttgttgtgtgcccttttcctctagcggatcctatccacatttcgtgaatgaaccgattaagctctgacgagatttccttgttttctgtagatacctcttgtagaggtgtcaaagttatgttgtgatcgagagagttacctgcaaggtattatgcaagcggcttgatgaggacactcaactacgtgtgagtaggagtgcgttgctcagcagttgacgcgtgagccgcttgagcagcttgataaaggtcctaatcgtcttccagctgttggcagacgacaatgtgctgtagtagtaggataggcccgtaaaagtgtagaagcaataggttgagactgactgtaatgggtacttgcgttcaaagactgaaagtccaaaggattgctcaaaggcccctgaggtctgagcctctgagcgcgctcttcgtcaaaattcgccggggccatcggtcatcctctcgagggcagcatcttcctgcttgaagactgagtccgtctcagataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatgcatcatgcatcatgcatcatgcgtcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatgggtaaggaaaagacacacgtatcgcgaccccgactcaactccaacatggacgcagacctgtacggatacaaatgggctcgagataatgtcggacagtcgggtgcgacgatctacagactctatggaaaaccggacgcccccgagctctttcttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactcgggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcg

SEQ ID NO: 45 - DNA, 4558 bp, synthetic construct
  source 1..4558: mol_type = other DNA
tgcagagccttgcaaatgatgaaagaccaatggaattgatcattgattattgatgatgcatgatgaaagagatagaaacagatgcaagatgggatggcgcgatgatcaaatgataatgaacgttcaacccttcaaaattgaaactctcaggctgagcggctgagcaagctctgagaatgtgggtcttcgaagactttcgcttgagcatggtgcatgtgcaaagagatggaatgtggaattgttgtaggtaggaacacagcccggtagagcggcgcagtggctcagcgaagctgggagaacgactgaaccggtcttgagattcatcatctcagcgtgcgtcagcgcgtcagcttcagcgtttgagaatggcacggactgacctgtcgtacatcccaaggaggcccgtgcgctatactcgccttgtggcctaatctgcgatcaacccatcctcaacgaaagcgcaagtagcgactaagcacactatctttcgggtcggtgcggtcacagtatcggcatctctgtctttagctacgatggtgtgcgtgtaactgatcagtacacccgctgtatcaaaactctatcttcaatccgctaagaatcgtcaccatcagtacatcagtacatcagtgggctaagatctaatggtgtcgttcgacggccgcgggcactagccacgagccaagagcggtacagttgctgcgaaatggtgggcagcctgacaggttcagtttgaataggatgaaaacgccatgaagaacagagaagctggcaattgacgatagtggggcgagcgcacgctgcttgctggtccgagcgtaggacgaacgacaaaccatgggcggggccaccaggccccagggagggcgtcactggttggatgtcgtctgttaggctagagctcaggcgctcagggcctctggcccaagcctcggacgcaagcctctgacgagcgagcgcgcgctggggcgggtctgcgcaggaacggcaggactgccgagttggttgaatattgaatggttggatgattggtttgctgggtttggttttgacacgcacggcacaggacaagtgagtttggaggttggatttgcgcaggtagaaagggcgctcggcctcagggcctcagcgtcagcagacgagctcgccagtccaggtcagtacccagtgttcgttacttcccaccgtccagagtttgctgctgcctctcagcttgattcgtgtcggagaagtagaaatggtcacagcgtaccacacactgtccccatgcctgcgtcctgctccacgagtgtttagccacaaaaatttatggacaaccttactgagcctgtgctggggggtgctttagcaccagcactggcgccatggcgtcaaggttaattccactgtcattcgacaaccttcaactgatgggggcaactgggcacaaaaccttggatatataaccaactccaactcccacggttttttaccttttgctcttctccccaggactcactacttctactcctctattcatcttcaccaaaatgtgagacctaatatcactgctgtacagacaagactgtgtatattaggtctcaatgaaagctatggtgtggcttggacccgaacgaatggaagtgcgagatcttcctgcgccgcgaccgggtgcaggtgaagtgctggttcagacggaagctgctggaatttgtggctctgaagtggaggcctatttgggtcgcatggctaatcgaacaccgccgttggtgatgggtcacgaatatgctggacttgtcaccgaggtcggagagggagtcgaccgcgtctggtcccgtcgacgcgttgcggttaacccgattgttggttgcggtcgttgtcgaaactgcgccgccggtgatcgaaacttgtgtcctgatcgtcaccttcttggcattgcagcgccaggaggattcgcagcgttctcggtggttcccgaacgatgcctcttttctctgcctgaaggaatggatgcgcgactgggcgcgctcaccaagccattggccaacggtgtgcatcttattcgcaaaagcggcgcgggacgaggccgagttgtggttatcggcgcgggaactattggactcggatgtttgcaagctgctttgttgcacggtgctgagcaagttgacgtcgttgaacgccatccgggacgacgccgtcacgcacttgccctgggagcacgccaggcggtggcggaattggcagatgtcgaacctggtgctgatcttgtggttgatgccgcaggatccgaagcgacacgccgcgcggccgtggaattggttggcccggcgggaacggcctcttttgttggactccactccgacgaaactgtcctgccttggcatcgcattatccgagccaatattcgagttcaaggaacatttggctacgcagatcgtgatttccagcaagctcttgactggttggcagcgggaaaggcagccattccattgtctgaactccgccccttggacgatggcccagcggccttcgagcgccttgctcgtggaccgatcgacgaaatcaaagtctttttggcttaacacggatcaacaaaaatgtgcgccagctttgttgtgtgcccttttcctctagcggatcctatccacatttcgtgaatgaaccgattaagctctgacgagatttccttgttttctgtagatacctcttgtagaggtgtcaaagttatgttgtgatcgagagagttacctgcaaggtattatgcaagcggcttgatgaggacactcaactacgtgtgagtaggagtgcgttgctcagcagttgacgcgtgagccgcttgagcagcttgataaaggtcctaatcgtcttccagctgttggcagacgacaatgtgctgtagtagtaggataggcccgtaaaagtgtagaagcaataggttgagactgactgtaatgggtacttgcgttcaaagactgaaagtccaaaggattgctcaaaggcccctgaggtctgagcctctgagcgcgctcttcgtcaaaattcgccggggccatcggtcatcctctcgagggcagcatcttcctgcttgaagactgagtccgtctcagataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatgcatcatgcatcatgcatcatgcgtcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatgggtaaggaaaagacacacgtatcgcgaccccgactcaactccaacatggacgcagacctgtacggatacaaatgggctcgagataatgtcggacagtcgggtgcgacgatctacagactctatggaaaaccggacgcccccgagctctttcttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactcgggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcg

SEQ ID NO: 46 - DNA, 4594 bp, synthetic construct
  source 1..4594: mol_type = other DNA
tgcagagccttgcaaatgatgaaagaccaatggaattgatcattgattattgatgatgcatgatgaaagagatagaaacagatgcaagatgggatggcgcgatgatcaaatgataatgaacgttcaacccttcaaaattgaaactctcaggctgagcggctgagcaagctctgagaatgtgggtcttcgaagactttcgcttgagcatggtgcatgtgcaaagagatggaatgtggaattgttgtaggtaggaacacagcccggtagagcggcgcagtggctcagcgaagctgggagaacgactgaaccggtcttgagattcatcatctcagcgtgcgtcagcgcgtcagcttcagcgtttgagaatggcacggactgacctgtcgtacatcccaaggaggcccgtgcgctatactcgccttgtggcctaatctgcgatcaacccatcctcaacgaaagcgcaagtagcgactaagcacactatctttcgggtcggtgcggtcacagtatcggcatctctgtctttagctacgatggtgtgcgtgtaactgatcagtacacccgctgtatcaaaactctatcttcaatccgctaagaatcgtcaccatcagtacatcagtacatcagtgggctaagatctaatggtgtcgttcgacggccgcgggcactagccacgagccaagagcggtacagttgctgcgaaatggtgggcagcctgacaggttcagtttgaataggatgaaaacgccatgaagaacagagaagctggcaattgacgatagtggggcgagcgcacgctgcttgctggtccgagcgtaggacgaacgacaaaccatgggcggggccaccaggccccagggagggcgtcactggttggatgtcgtctgttaggctagagctcaggcgctcagggcctctggcccaagcctcggacgcaagcctctgacgagcgagcgcgcgctggggcgggtctgcgcaggaacggcaggactgccgagttggttgaatattgaatggttggatgattggtttgctgggtttggttttgacacgcacggcacaggacaagtgagtttggaggttggatttgcgcaggtagaaagggcgctcggcctcagggcctcagcgtcagcagacgagctcgccagtccaggtcagtacccagtgttcgttacttcccaccgtccagagtttgctgctgcctctcagcttgattcgtgtcggagaagtagaaatggtcacagcgtaccacacactgtccccatgcctgcgtcctgctccacgagtgtttagccacaaaaatttatggacaaccttactgagcctgtgctggggggtgctttagcaccagcactggcgccatggcgtcaaggttaattccactgtcattcgacaaccttcaactgatgggggcaactgggcacaaaaccttggatatataaccaactccaactcccacggttttttaccttttgctcttctccccaggactcactacttctactcctctattcatcttcaccaaaatgtgagacctaatatcactgctgtacagacaagactgtgtatattaggtctcaatgaagtcgttgatttactatggcccgcacgatctgcgatatggagattctcctaagccaaagatccgatctggagaagtcctcctccgaatcaaaagcgtcggtatctgcggcactgacctccacatctataacggaggcatgaaaaatctgccgttgcctctgattatgggtcacgagttcagcggagttattgaagagcttgcccgagacgtgaagaactttaaggttggagatcgagtgatcgctgaacacgttatcaattgtggaaaatgttcgtattgtcttagcggcaagccgaatctgtgtgctaactctcgcattattggagttcaccgccccggagcgctcgccgaatacttggctgttcccgcggacctggtgtacaagatcccggcgaaactgtcttttgaagagggcgcaatgatcgaacccctttctatcgcctattatgccgtgaagcaagctcgtagcatcctgggtaaaaaggtggcggtcatcggccaaggacccatcggcctgctgcttgaccaaatcctccgcgacgcaggcgctgaagtcattggcattgacatcgcagattggcgattgaaactcgcgaagaaaaagaagtgggcagatcatacaatcaactccagccagaaaacctggctcaagaagatccagaagatcgcaccagaaggcgtggacacagtgtttgaggttgttggccgtgaggaaactgccgagatggctattgaaatcgcgcgtcgagatggtgacgtttttatcctcggtgtctttgaaagcgccgcatctttgaacatgatgaatattgttaagaaggaactcaacgtgttcggaagctggacctgtgcgttttctttccccccttcgattgctctcgccgttgccaagaaagttgatttgaaatccttgatcacgaatcgatatgacgtgaaggacggtgccctggcatttaaggagtctgcctcttataaaggtaagcgaatcaagactgttatcaacttttaacacggatcaacaaaaatgtgcgccagctttgttgtgtgcccttttcctctagcggatcctatccacatttcgtgaatgaaccgattaagctctgacgagatttccttgttttctgtagatacctcttgtagaggtgtcaaagttatgttgtgatcgagagagttacctgcaaggtattatgcaagcggcttgatgaggacactcaactacgtgtgagtaggagtgcgttgctcagcagttgacgcgtgagccgcttgagcagcttgataaaggtcctaatcgtcttccagctgttggcagacgacaatgtgctgtagtagtaggataggcccgtaaaagtgtagaagcaataggttgagactgactgtaatgggtacttgcgttcaaagactgaaagtccaaaggattgctcaaaggcccctgaggtctgagcctctgagcgcgctcttcgtcaaaattcgccggggccatcggtcatcctctcgagggcagcatcttcctgcttgaagactgagtccgtctcagataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatgcatcatgcatcatgcatcatgcgtcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatgggtaaggaaaagacacacgtatcgcgaccccgactcaactccaacatggacgcagacctgtacggatacaaatgggctcgagataatgtcggacagtcgggtgcgacgatctacagactctatggaaaaccggacgcccccgagctctttcttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactcgggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcg

SEQ ID NO: 47 - DNA, 4624 bp, synthetic construct
  source 1..4624: mol_type = other DNA
tgcagagccttgcaaatgatgaaagaccaatggaattgatcattgattattgatgatgcatgatgaaagagatagaaacagatgcaagatgggatggcgcgatgatcaaatgataatgaacgttcaacccttcaaaattgaaactctcaggctgagcggctgagcaagctctgagaatgtgggtcttcgaagactttcgcttgagcatggtgcatgtgcaaagagatggaatgtggaattgttgtaggtaggaacacagcccggtagagcggcgcagtggctcagcgaagctgggagaacgactgaaccggtcttgagattcatcatctcagcgtgcgtcagcgcgtcagcttcagcgtttgagaatggcacggactgacctgtcgtacatcccaaggaggcccgtgcgctatactcgccttgtggcctaatctgcgatcaacccatcctcaacgaaagcgcaagtagcgactaagcacactatctttcgggtcggtgcggtcacagtatcggcatctctgtctttagctacgatggtgtgcgtgtaactgatcagtacacccgctgtatcaaaactctatcttcaatccgctaagaatcgtcaccatcagtacatcagtacatcagtgggctaagatctaatggtgtcgttcgacggccgcgggcactagccacgagccaagagcggtacagttgctgcgaaatggtgggcagcctgacaggttcagtttgaataggatgaaaacgccatgaagaacagagaagctggcaattgacgatagtggggcgagcgcacgctgcttgctggtccgagcgtaggacgaacgacaaaccatgggcggggccaccaggccccagggagggcgtcactggttggatgtcgtctgttaggctagagctcaggcgctcagggcctctggcccaagcctcggacgcaagcctctgacgagcgagcgcgcgctggggcgggtctgcgcaggaacggcaggactgccgagttggttgaatattgaatggttggatgattggtttgctgggtttggttttgacacgcacggcacaggacaagtgagtttggaggttggatttgcgcaggtagaaagggcgctcggcctcagggcctcagcgtcagcagacgagctcgccagtccaggtcagtacccagtgttcgttacttcccaccgtccagagtttgctgctgcctctcagcttgattcgtgtcggagaagtagaaatggtcacagcgtaccacacactgtccccatgcctgcgtcctgctccacgagtgtttagccacaaaaatttatggacaaccttactgagcctgtgctggggggtgctttagcaccagcactggcgccatggcgtcaaggttaattccactgtcattcgacaaccttcaactgatgggggcaactgggcacaaaaccttggatatataaccaactccaactcccacggttttttaccttttgctcttctccccaggactcactacttctactcctctattcatcttcaccaaaatgtgagacctaatatcactgctgtacagacaagactgtgtatattaggtctcaatgaaggcaatgttgcttagccagtatcgacgactggaaattgccgagttgccgacaccgatccctggtcgtggcgaactcttgatccatgtcgcggcatgtggtatttgcggaagcgatgtgcacggatacgatggcagcagcggacgacgtatccctccaatcgtgatgggccatgaggctgcaggacgtgttgcggcactcggtccgcaggtcacgggctggtcggaaggtgatcgcgtcacattcgatagcaccatctcctgcggcacttgcccttactgctcccgcggcgagattaacctctgcgaccgtcgtcaagtgcttggagtttcgtgcggtgactatcgttgcgctggagcttttgcggagtatgttattgtgccgcaacgcattgcttataaattgccggatgccttgagcttctcggaggccgcaactttggaagcggtcgcggttgctgtgcacgctgttagcctttcccaggtttcgccaggtactcaggcactggttattggcgctggtatgattggcctcctcaccttgcaagcgctgcgcgcagccggttgcagctccgtggtgatcgcggacgttgatgcgtcccgtctgcgtttggctaaagaggttggcgcaaccgaaacgcttaatgcgacaggagatgaatttgttgccgaaatcttcaaacgcacaaacggagcgggcacagaccttgccgtcgaagctgtcggtatcgatgctaccgtccgaacggccattaactctgtccgaaaaggcggtacagttaccctggtcggcaatatcacgccggaggtcactcttccgcttcagaaggtggtgacacgccagattcgaatccaaggttcttgcgcatccgctggtgaatacccccgagctatccagctccttacgtctggcgctattaaagtcaaacctctcattaccgctgtggccccgcttgaagacggtcctcagtggtttgagcgcctttacagccgcgagcctaatcttatgaaggtggtccttaccccgggcagcgtgtcttaacacggatcaacaaaaatgtgcgccagctttgttgtgtgcccttttcctctagcggatcctatccacatttcgtgaatgaaccgattaagctctgacgagatttccttgttttctgtagatacctcttgtagaggtgtcaaagttatgttgtgatcgagagagttacctgcaaggtattatgcaagcggcttgatgaggacactcaactacgtgtgagtaggagtgcgttgctcagcagttgacgcgtgagccgcttgagcagcttgataaaggtcctaatcgtcttccagctgttggcagacgacaatgtgctgtagtagtaggataggcccgtaaaagtgtagaagcaataggttgagactgactgtaatgggtacttgcgttcaaagactgaaagtccaaaggattgctcaaaggcccctgaggtctgagcctctgagcgcgctcttcgtcaaaattcgccggggccatcggtcatcctctcgagggcagcatcttcctgcttgaagactgagtccgtctcagataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatgcatcatgcatcatgcatcatgcgtcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatgggtaaggaaaagacacacgtatcgcgaccccgactcaactccaacatggacgcagacctgtacggatacaaatgggctcgagataatgtcggacagtcgggtgcgacgatctacagactctatggaaaaccggacgcccccgagctctttcttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactcgggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcg

SEQ ID NO: 48 - DNA, 4615 bp, synthetic construct
  source 1..4615: mol_type = other DNA
tgcagagccttgcaaatgatgaaagaccaatggaattgatcattgattattgatgatgcatgatgaaagagatagaaacagatgcaagatgggatggcgcgatgatcaaatgataatgaacgttcaacccttcaaaattgaaactctcaggctgagcggctgagcaagctctgagaatgtgggtcttcgaagactttcgcttgagcatggtgcatgtgcaaagagatggaatgtggaattgttgtaggtaggaacacagcccggtagagcggcgcagtggctcagcgaagctgggagaacgactgaaccggtcttgagattcatcatctcagcgtgcgtcagcgcgtcagcttcagcgtttgagaatggcacggactgacctgtcgtacatcccaaggaggcccgtgcgctatactcgccttgtggcctaatctgcgatcaacccatcctcaacgaaagcgcaagtagcgactaagcacactatctttcgggtcggtgcggtcacagtatcggcatctctgtctttagctacgatggtgtgcgtgtaactgatcagtacacccgctgtatcaaaactctatcttcaatccgctaagaatcgtcaccatcagtacatcagtacatcagtgggctaagatctaatggtgtcgttcgacggccgcgggcactagccacgagccaagagcggtacagttgctgcgaaatggtgggcagcctgacaggttcagtttgaataggatgaaaacgccatgaagaacagagaagctggcaattgacgatagtggggcgagcgcacgctgcttgctggtccgagcgtaggacgaacgacaaaccatgggcggggccaccaggccccagggagggcgtcactggttggatgtcgtctgttaggctagagctcaggcgctcagggcctctggcccaagcctcggacgcaagcctctgacgagcgagcgcgcgctggggcgggtctgcgcaggaacggcaggactgccgagttggttgaatattgaatggttggatgattggtttgctgggtttggttttgacacgcacggcacaggacaagtgagtttggaggttggatttgcgcaggtagaaagggcgctcggcctcagggcctcagcgtcagcagacgagctcgccagtccaggtcagtacccagtgttcgttacttcccaccgtccagagtttgctgctgcctctcagcttgattcgtgtcggagaagtagaaatggtcacagcgtaccacacactgtccccatgcctgcgtcctgctccacgagtgtttagccacaaaaatttatggacaaccttactgagcctgtgctggggggtgctttagcaccagcactggcgccatggcgtcaaggttaattccactgtcattcgacaaccttcaactgatgggggcaactgggcacaaaaccttggatatataaccaactccaactcccacggttttttaccttttgctcttctccccaggactcactacttctactcctctattcatcttcaccaaaatgtgagacctaatatcactgctgtacagacaagactgtgtatattaggtctcaatgaaagcgatggtgttgaaagaatataagtatctggaaatcgaagacattcctattccgcacatcgaagcagcggacgaactgttggtccgcgtgaaggctaccgccatctgcggttcggatatccatggttttgatggttccaccggacgccgtaaaccgccattggtcatgggacacgaggcatcgggtgttgtcgaaagcgtcggaagcgaagttaaatcgctgaaaatcggtgaccgtgtcaccttcgactccacagtttggtgcggcgaatgctacttttgccgacagggtcaggttaacctgtgcgacaaacgacgtgtgcttggtgtgtcgtgcgacgaataccgcaaggacggtacctttgcggaatatgtcattgtgcccgagcgtattgtgtataagttgccggataatgttgccttcgaagaagcagcattgactgagccggtttctgtcgcgatgcacgcatttgatatcaccggaatgaagccaggagaaaatgcggccgttatcggtagcggtcttatcggactcctcctgattcaaattatccgtgcttattcgccccacttgatcatcgcgttcgatacagacgaaaaacgccgaaaagcagctatcgaagctggagccgatattgcattggacccccaagatcctgagtcgcataaaactgtcctgagcatgactaataaccgaggtgtcgaccgagcatttgaggctgtgggcgcctcggcagcgatcggtacagctattgaatccgtccgaaaaggctcgtccgtcactctgatcggcaatattagcaataaagctgagatcccgctccaatctgtggttacgcgccaaattgcccttttgggttcgtgcgctatttctggagagtatcctgtctctttggctttgatggcgaatcgcactattgacgtgcgtcaagttatctccgcaatcgcccctttggaggagggtcctctgtggttcgaacgcttgtataaacgcgaacctggtcttttgaaggttgttctcacgccgtaacacggatcaacaaaaatgtgcgccagctttgttgtgtgcccttttcctctagcggatcctatccacatttcgtgaatgaaccgattaagctctgacgagatttccttgttttctgtagatacctcttgtagaggtgtcaaagttatgttgtgatcgagagagttacctgcaaggtattatgcaagcggcttgatgaggacactcaactacgtgtgagtaggagtgcgttgctcagcagttgacgcgtgagccgcttgagcagcttgataaaggtcctaatcgtcttccagctgttggcagacgacaatgtgctgtagtagtaggataggcccgtaaaagtgtagaagcaataggttgagactgactgtaatgggtacttgcgttcaaagactgaaagtccaaaggattgctcaaaggcccctgaggtctgagcctctgagcgcgctcttcgtcaaaattcgccggggccatcggtcatcctctcgagggcagcatcttcctgcttgaagactgagtccgtctcagataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatgcatcatgcatcatgcatcatgcgtcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatgggtaaggaaaagacacacgtatcgcgaccccgactcaactccaacatggacgcagacctgtacggatacaaatgggctcgagataatgtcggacagtcgggtgcgacgatctacagactctatggaaaaccggacgcccccgagctctttcttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactcgggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcg

SEQ ID NO: 49 - DNA, 4627 bp, synthetic construct
  source 1..4627: mol_type = other DNA
tgcagagccttgcaaatgatgaaagaccaatggaattgatcattgattattgatgatgcatgatgaaagagatagaaacagatgcaagatgggatggcgcgatgatcaaatgataatgaacgttcaacccttcaaaattgaaactctcaggctgagcggctgagcaagctctgagaatgtgggtcttcgaagactttcgcttgagcatggtgcatgtgcaaagagatggaatgtggaattgttgtaggtaggaacacagcccggtagagcggcgcagtggctcagcgaagctgggagaacgactgaaccggtcttgagattcatcatctcagcgtgcgtcagcgcgtcagcttcagcgtttgagaatggcacggactgacctgtcgtacatcccaaggaggcccgtgcgctatactcgccttgtggcctaatctgcgatcaacccatcctcaacgaaagcgcaagtagcgactaagcacactatctttcgggtcggtgcggtcacagtatcggcatctctgtctttagctacgatggtgtgcgtgtaactgatcagtacacccgctgtatcaaaactctatcttcaatccgctaagaatcgtcaccatcagtacatcagtacatcagtgggctaagatctaatggtgtcgttcgacggccgcgggcactagccacgagccaagagcggtacagttgctgcgaaatggtgggcagcctgacaggttcagtttgaataggatgaaaacgccatgaagaacagagaagctggcaattgacgatagtggggcgagcgcacgctgcttgctggtccgagcgtaggacgaacgacaaaccatgggcggggccaccaggccccagggagggcgtcactggttggatgtcgtctgttaggctagagctcaggcgctcagggcctctggcccaagcctcggacgcaagcctctgacgagcgagcgcgcgctggggcgggtctgcgcaggaacggcaggactgccgagttggttgaatattgaatggttggatgattggtttgctgggtttggttttgacacgcacggcacaggacaagtgagtttggaggttggatttgcgcaggtagaaagggcgctcggcctcagggcctcagcgtcagcagacgagctcgccagtccaggtcagtacccagtgttcgttacttcccaccgtccagagtttgctgctgcctctcagcttgattcgtgtcggagaagtagaaatggtcacagcgtaccacacactgtccccatgcctgcgtcctgctccacgagtgtttagccacaaaaatttatggacaaccttactgagcctgtgctggggggtgctttagcaccagcactggcgccatggcgtcaaggttaattccactgtcattcgacaaccttcaactgatgggggcaactgggcacaaaaccttggatatataaccaactccaactcccacggttttttaccttttgctcttctccccaggactcactacttctactcctctattcatcttcaccaaaatgtgagacctaatatcactgctgtacagacaagactgtgtatattaggtctcaatgaaggcactccgcctgtacgctccaggtgacttgcgtttggaggaaatcccaattcctttgccacgtcccgatgaggtgttggttcgcgtgcttgcgtgcggtatctgcggttcggatatcccccgtgtttttgaacacggtacgtataaatacccattggtgccgggccatgagttttccggtattgttgaaaaagtcggctccgaaacgggaaaagattgggttggtcgtcacgttgtcgtctatcccctcatcccgtgtcgacaatgctcctcttgctactctggtctttatacccaatgcctcaactacgactacttgggatctcgccgtgacggaggatttgcagagtatgtgactgtccctgtgaataatattatcccggtccctaatggaatctctggagagatctcggcgatcactgagccatgtgcggtcgcccttcatgccgttcgcaaggcccaagcctgcgtgggtgacacggtccttattacgggagctggaaccattggtatcttgatcgccctctgggcgcgagcagcaggtgccgaagtctttatgactgacattgtggtgggaaagttgaaactggctcgttcgttgggttttcagcatctgctggacgccaaacaaacaaatgtttctgaatgggttaagcaaaagtgtaaacacggagttgatatcgtgttcgaaaccacgggacatcccgcgggactcgaaacgtcgattgactcttgttctattacaggcaaaattattctcgtgggcaatcccagcttcgaagagatgaaagttccggctaaacaatatggaatgattctgcgcaaggagatccagattactggaatttggaattcgctcatcaacgaatatccaaaaaacgagtggcaagttgtgctgcaagagttcaagaaggagtcgtccttgtactcgaagattatcacgcataaggttcccattgagggccttaaggaattgtttgtgacgatttacaacaaacaggtctttccggtgaaggctatgtttatcaataaggagatctaacacggatcaacaaaaatgtgcgccagctttgttgtgtgcccttttcctctagcggatcctatccacatttcgtgaatgaaccgattaagctctgacgagatttccttgttttctgtagatacctcttgtagaggtgtcaaagttatgttgtgatcgagagagttacctgcaaggtattatgcaagcggcttgatgaggacactcaactacgtgtgagtaggagtgcgttgctcagcagttgacgcgtgagccgcttgagcagcttgataaaggtcctaatcgtcttccagctgttggcagacgacaatgtgctgtagtagtaggataggcccgtaaaagtgtagaagcaataggttgagactgactgtaatgggtacttgcgttcaaagactgaaagtccaaaggattgctcaaaggcccctgaggtctgagcctctgagcgcgctcttcgtcaaaattcgccggggccatcggtcatcctctcgagggcagcatcttcctgcttgaagactgagtccgtctcagataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatgcatcatgcatcatgcatcatgcgtcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatgggtaaggaaaagacacacgtatcgcgaccccgactcaactccaacatggacgcagacctgtacggatacaaatgggctcgagataatgtcggacagtcgggtgcgacgatctacagactctatggaaaaccggacgcccccgagctctttcttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactcgggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcg

SEQ ID NO: 50 - DNA, 4594 bp, synthetic construct
  source 1..4594: mol_type = other DNA
tgcagagccttgcaaatgatgaaagaccaatggaattgatcattgattattgatgatgcatgatgaaagagatagaaacagatgcaagatgggatggcgcgatgatcaaatgataatgaacgttcaacccttcaaaattgaaactctcaggctgagcggctgagcaagctctgagaatgtgggtcttcgaagactttcgcttgagcatggtgcatgtgcaaagagatggaatgtggaattgttgtaggtaggaacacagcccggtagagcggcgcagtggctcagcgaagctgggagaacgactgaaccggtcttgagattcatcatctcagcgtgcgtcagcgcgtcagcttcagcgtttgagaatggcacggactgacctgtcgtacatcccaaggaggcccgtgcgctatactcgccttgtggcctaatctgcgatcaacccatcctcaacgaaagcgcaagtagcgactaagcacactatctttcgggtcggtgcggtcacagtatcggcatctctgtctttagctacgatggtgtgcgtgtaactgatcagtacacccgctgtatcaaaactctatcttcaatccgctaagaatcgtcaccatcagtacatcagtacatcagtgggctaagatctaatggtgtcgttcgacggccgcgggcactagccacgagccaagagcggtacagttgctgcgaaatggtgggcagcctgacaggttcagtttgaataggatgaaaacgccatgaagaacagagaagctggcaattgacgatagtggggcgagcgcacgctgcttgctggtccgagcgtaggacgaacgacaaaccatgggcggggccaccaggccccagggagggcgtcactggttggatgtcgtctgttaggctagagctcaggcgctcagggcctctggcccaagcctcggacgcaagcctctgacgagcgagcgcgcgctggggcgggtctgcgcaggaacggcaggactgccgagttggttgaatattgaatggttggatgattggtttgctgggtttggttttgacacgcacggcacaggacaagtgagtttggaggttggatttgcgcaggtagaaagggcgctcggcctcagggcctcagcgtcagcagacgagctcgccagtccaggtcagtacccagtgttcgttacttcccaccgtccagagtttgctgctgcctctcagcttgattcgtgtcggagaagtagaaatggtcacagcgtaccacacactgtccccatgcctgcgtcctgctccacgagtgtttagccacaaaaatttatggacaaccttactgagcctgtgctggggggtgctttagcaccagcactggcgccatggcgtcaaggttaattccactgtcattcgacaaccttcaactgatgggggcaactgggcacaaaaccttggatatataaccaactccaactcccacggttttttaccttttgctcttctccccaggactcactacttctactcctctattcatcttcaccaaaatgtgagacctaatatcactgctgtacagacaagactgtgtatattaggtctcaatgaaggcagtcgtgtacacgcaaccagacgcagtggccgtccttgagcgtgaaatgcctgcactggtgccaggagaggtgcttcttcgaattgatgccagcggaatttgcggttccgatttgcacgcatatcatggacatgacccacgtcgtaaacctggacttgtgttgggtcacgagtttgcaggcaccgtggttgcttctagcgatgaaagccgcctccctgtgggtcgacgcgtcacggctaatcccctcgttacatgcggacactgtgattactgccttcagggccgtgacaatctgtgtgcgaatcgtggcatggtgggtatgtctcgccctggtgccctttctgagtatcttgccattcccatgcgatgcgctattgctattcccgaagatctgccgagcgtgtctgctgcgcttaccgaacctgctgcgacggcactgcacgcagtcaatcttacaatgcgagcgatgagccgacctctcccggaggcgcgtgttttgattatcggaggtggagcgatcggcatgttgaccggagcacttgtcggtgcgtacggctgtcgctcccttgcggtcgcggaaaccaacgccctgcgccgtgcgtccgtccgacgtcatcttgcatgcgaggttttggaccctacagcgaacgccccggctgagtcttcgttcgacgtggtcattgacgcagtgggtgcggcaccaacccgccatctggcactggcggcaacgcgaccaggaggtgttatcatgcacattggactccaggcatggtccagcgaaatcgatatgcgtaagctcactcttgctgaaatcactctcctcggtacgtatacatacacgatggcggatatgcatgcgaccgtgcgagcacttgcctctggaagcttcggatctctggaatgggtcgagacgcgagccctgacagatgcgccatctgcttttgctgaccttgctgcgggtcgagtcgcggcgggcaaggttgtgcttctcccgcaacaggactaacacggatcaacaaaaatgtgcgccagctttgttgtgtgcccttttcctctagcggatcctatccacatttcgtgaatgaaccgattaagctctgacgagatttccttgttttctgtagatacctcttgtagaggtgtcaaagttatgttgtgatcgagagagttacctgcaaggtattatgcaagcggcttgatgaggacactcaactacgtgtgagtaggagtgcgttgctcagcagttgacgcgtgagccgcttgagcagcttgataaaggtcctaatcgtcttccagctgttggcagacgacaatgtgctgtagtagtaggataggcccgtaaaagtgtagaagcaataggttgagactgactgtaatgggtacttgcgttcaaagactgaaagtccaaaggattgctcaaaggcccctgaggtctgagcctctgagcgcgctcttcgtcaaaattcgccggggccatcggtcatcctctcgagggcagcatcttcctgcttgaagactgagtccgtctcagataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatgcatcatgcatcatgcatcatgcgtcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatgggtaaggaaaagacacacgtatcgcgaccccgactcaactccaacatggacgcagacctgtacggatacaaatgggctcgagataatgtcggacagtcgggtgcgacgatctacagactctatggaaaaccggacgcccccgagctctttcttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactcgggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcg

SEQ ID NO: 51 - DNA, 4624 bp, synthetic construct
  source 1..4624: mol_type = other DNA
tgcagagccttgcaaatgatgaaagaccaatggaattgatcattgattattgatgatgcatgatgaaagagatagaaacagatgcaagatgggatggcgcgatgatcaaatgataatgaacgttcaacccttcaaaattgaaactctcaggctgagcggctgagcaagctctgagaatgtgggtcttcgaagactttcgcttgagcatggtgcatgtgcaaagagatggaatgtggaattgttgtaggtaggaacacagcccggtagagcggcgcagtggctcagcgaagctgggagaacgactgaaccggtcttgagattcatcatctcagcgtgcgtcagcgcgtcagcttcagcgtttgagaatggcacggactgacctgtcgtacatcccaaggaggcccgtgcgctatactcgccttgtggcctaatctgcgatcaacccatcctcaacgaaagcgcaagtagcgactaagcacactatctttcgggtcggtgcggtcacagtatcggcatctctgtctttagctacgatggtgtgcgtgtaactgatcagtacacccgctgtatcaaaactctatcttcaatccgctaagaatcgtcaccatcagtacatcagtacatcagtgggctaagatctaatggtgtcgttcgacggccgcgggcactagccacgagccaagagcggtacagttgctgcgaaatggtgggcagcctgacaggttcagtttgaataggatgaaaacgccatgaagaacagagaagctggcaattgacgatagtggggcgagcgcacgctgcttgctggtccgagcgtaggacgaacgacaaaccatgggcggggccaccaggccccagggagggcgtcactggttggatgtcgtctgttaggctagagctcaggcgctcagggcctctggcccaagcctcggacgcaagcctctgacgagcgagcgcgcgctggggcgggtctgcgcaggaacggcaggactgccgagttggttgaatattgaatggttggatgattggtttgctgggtttggttttgacacgcacggcacaggacaagtgagtttggaggttggatttgcgcaggtagaaagggcgctcggcctcagggcctcagcgtcagcagacgagctcgccagtccaggtcagtacccagtgttcgttacttcccaccgtccagagtttgctgctgcctctcagcttgattcgtgtcggagaagtagaaatggtcacagcgtaccacacactgtccccatgcctgcgtcctgctccacgagtgtttagccacaaaaatttatggacaaccttactgagcctgtgctggggggtgctttagcaccagcactggcgccatggcgtcaaggttaattccactgtcattcgacaaccttcaactgatgggggcaactgggcacaaaaccttggatatataaccaactccaactcccacggttttttaccttttgctcttctccccaggactcactacttctactcctctattcatcttcaccaaaatgtgagacctaatatcactgctgtacagacaagactgtgtatattaggtctcaatgaagtccgtcgttattcacgccgaaggagatgtccgtgtggaagagcgacccattccccatttgcaaacggatgaagatgttttggtgaaagtcgtgtcttccggactttgtggcagcgacattccccgcatcttcaatcgtggcgctcattattacccaatcacacttggccatgaattctcgggctatgtcgagtcctatggagcgggtgtcactgacttgcaaccaggtgatgctgtcgcttgtgtgccgctcctcccgtgttttcactgcccccagtgcgagcgaggttacttctctctgtgtaagcagtatcaatttgtcggttctcgcagcgagggaggaaacgcagagtatgtcgttatgaaacgagcgaatcttttccgactcccgtccgatatgccaattgaagacggagctttcattgagcccatcaccgtcggtctgcatgccttccaccttgcgcagggttgcgagggaaagaatgtcattatcgtgggtgcaggcaccattggcctccttgccctgcaatgtgcacgagaactcggagccaaatcgattaccgcaatcgacatcaacccacagaagcttgcattggcaaaggagctgggcgcgacttacgtgtttaattcccgagaaatgtcggcgaccgaaattcactctgatttggaaaatgtgcagttcgaccaacttattctcgaaacggctggaacgcctcagactgtcacgctctccatcgaaattgcgggtccccgagcccaattggcgcttgttggtacgttgcaccacgatttggcgcttgcgtcgggtgtctttggtcaaatcttgcgcaaagagcttactatcctcggatcttggatgaactacagctctccctggccaggcgaagaatgggtcactgcggcacgtctgctgacggagaaacgtttgcaattggaaccacttattgcgcactccggagatgccgaatcctttgcgtctgcagtgaagtctctcaatggcgcacctatgcaaggtaaaattctgctcaagctttcgtaacacggatcaacaaaaatgtgcgccagctttgttgtgtgcccttttcctctagcggatcctatccacatttcgtgaatgaaccgattaagctctgacgagatttccttgttttctgtagatacctcttgtagaggtgtcaaagttatgttgtgatcgagagagttacctgcaaggtattatgcaagcggcttgatgaggacactcaactacgtgtgagtaggagtgcgttgctcagcagttgacgcgtgagccgcttgagcagcttgataaaggtcctaatcgtcttccagctgttggcagacgacaatgtgctgtagtagtaggataggcccgtaaaagtgtagaagcaataggttgagactgactgtaatgggtacttgcgttcaaagactgaaagtccaaaggattgctcaaaggcccctgaggtctgagcctctgagcgcgctcttcgtcaaaattcgccggggccatcggtcatcctctcgagggcagcatcttcctgcttgaagactgagtccgtctcagataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatgcatcatgcatcatgcatcatgcgtcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatgggtaaggaaaagacacacgtatcgcgaccccgactcaactccaacatggacgcagacctgtacggatacaaatgggctcgagataatgtcggacagtcgggtgcgacgatctacagactctatggaaaaccggacgcccccgagctctttcttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactcgggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcg

SEQ ID NO: 52 - DNA, 4621 bp, synthetic construct
  source 1..4621: mol_type = other DNA
tgcagagccttgcaaatgatgaaagaccaatggaattgatcattgattattgatgatgcatgatgaaagagatagaaacagatgcaagatgggatggcgcgatgatcaaatgataatgaacgttcaacccttcaaaattgaaactctcaggctgagcggctgagcaagctctgagaatgtgggtcttcgaagactttcgcttgagcatggtgcatgtgcaaagagatggaatgtggaattgttgtaggtaggaacacagcccggtagagcggcgcagtggctcagcgaagctgggagaacgactgaaccggtcttgagattcatcatctcagcgtgcgtcagcgcgtcagcttcagcgtttgagaatggcacggactgacctgtcgtacatcccaaggaggcccgtgcgctatactcgccttgtggcctaatctgcgatcaacccatcctcaacgaaagcgcaagtagcgactaagcacactatctttcgggtcggtgcggtcacagtatcggcatctctgtctttagctacgatggtgtgcgtgtaactgatcagtacacccgctgtatcaaaactctatcttcaatccgctaagaatcgtcaccatcagtacatcagtacatcagtgggctaagatctaatggtgtcgttcgacggccgcgggcactagccacgagccaagagcggtacagttgctgcgaaatggtgggcagcctgacaggttcagtttgaataggatgaaaacgccatgaagaacagagaagctggcaattgacgatagtggggcgagcgcacgctgcttgctggtccgagcgtaggacgaacgacaaaccatgggcggggccaccaggccccagggagggcgtcactggttggatgtcgtctgttaggctagagctcaggcgctcagggcctctggcccaagcctcggacgcaagcctctgacgagcgagcgcgcgctggggcgggtctgcgcaggaacggcaggactgccgagttggttgaatattgaatggttggatgattggtttgctgggtttggttttgacacgcacggcacaggacaagtgagtttggaggttggatttgcgcaggtagaaagggcgctcggcctcagggcctcagcgtcagcagacgagctcgccagtccaggtcagtacccagtgttcgttacttcccaccgtccagagtttgctgctgcctctcagcttgattcgtgtcggagaagtagaaatggtcacagcgtaccacacactgtccccatgcctgcgtcctgctccacgagtgtttagccacaaaaatttatggacaaccttactgagcctgtgctggggggtgctttagcaccagcactggcgccatggcgtcaaggttaattccactgtcattcgacaaccttcaactgatgggggcaactgggcacaaaaccttggatatataaccaactccaactcccacggttttttaccttttgctcttctccccaggactcactacttctactcctctattcatcttcaccaaaatgtgagacctaatatcactgctgtacagacaagactgtgtatattaggtctcaatgaaatctgttgtgaacgatacggacggtattgttcgtattgcagagtcggtgattcccgaaattaaacaccaagatcaagtgcgtgttaagatcgcctcttccggcctttgtggttctgacctgccgcgcatttttaagaacggagcacactattatcccatcactctcggacacgaattttcgggatacatcgatgcagttggctccggtgtcgatgatcttcatcctggtgacgccgtggcatgtgttccccttctgccctgttttacctgtccagaatgtctgaaaggcttttacagccaatgtgccaagtacgattttattggatcccgtcgtgatggtggtttcgcggagtatatcgtggttaagcgaaaaaacgtgtttgctttgcccactgacatgtctattgaagatggagcctttatcgaaccaattacggttggccttcacgcgttccacctcgcgcaaggttgcgaaaataagaatgtcattattattggcgccggtactatcggcctgcttgcaatccagtgtgccgtcgctctcggtgccaagtctgttaccgccatcgacatctcgtctgaaaaactcgccctggccaagtcttttggagcgatgttgacgttcaattccttggagatgtccgcccctcagatgcaaggagtcctccgagaattgcgattcaatcagttgattctggaaaccgctggcgtgcctaagaccgtggaactggcggtcgagattgctggaccccacgctcaactggccttggttggcactttgcaccaagacttgcatcttacgagcaccacttttggaaaaatcctgcgcaaagagcttactgtcatcggatcttggatgaattattcgtccccttggcctggtcaagaatgggagactgcatctcgtctcctcaccgagcgaaagttgtctctcgaaccgttgattgctcatcgtggcagctttgaatcgttcgctcaggctgtccgagacattgctcgcaatgccatgccaggaaaagtgcttctgattccttaacacggatcaacaaaaatgtgcgccagctttgttgtgtgcccttttcctctagcggatcctatccacatttcgtgaatgaaccgattaagctctgacgagatttccttgttttctgtagatacctcttgtagaggtgtcaaagttatgttgtgatcgagagagttacctgcaaggtattatgcaagcggcttgatgaggacactcaactacgtgtgagtaggagtgcgttgctcagcagttgacgcgtgagccgcttgagcagcttgataaaggtcctaatcgtcttccagctgttggcagacgacaatgtgctgtagtagtaggataggcccgtaaaagtgtagaagcaataggttgagactgactgtaatgggtacttgcgttcaaagactgaaagtccaaaggattgctcaaaggcccctgaggtctgagcctctgagcgcgctcttcgtcaaaattcgccggggccatcggtcatcctctcgagggcagcatcttcctgcttgaagactgagtccgtctcagataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatgcatcatgcatcatgcatcatgcgtcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatgggtaaggaaaagacacacgtatcgcgaccccgactcaactccaacatggacgcagacctgtacggatacaaatgggctcgagataatgtcggacagtcgggtgcgacgatctacagactctatggaaaaccggacgcccccgagctctttcttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactcgggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcg

SEQ ID NO: 53 - DNA, 4600 bp, synthetic construct
  source 1..4600: mol_type = other DNA
tgcagagccttgcaaatgatgaaagaccaatggaattgatcattgattattgatgatgcatgatgaaagagatagaaacagatgcaagatgggatggcgcgatgatcaaatgataatgaacgttcaacccttcaaaattgaaactctcaggctgagcggctgagcaagctctgagaatgtgggtcttcgaagactttcgcttgagcatggtgcatgtgcaaagagatggaatgtggaattgttgtaggtaggaacacagcccggtagagcggcgcagtggctcagcgaagctgggagaacgactgaaccggtcttgagattcatcatctcagcgtgcgtcagcgcgtcagcttcagcgtttgagaatggcacggactgacctgtcgtacatcccaaggaggcccgtgcgctatactcgccttgtggcctaatctgcgatcaacccatcctcaacgaaagcgcaagtagcgactaagcacactatctttcgggtcggtgcggtcacagtatcggcatctctgtctttagctacgatggtgtgcgtgtaactgatcagtacacccgctgtatcaaaactctatcttcaatccgctaagaatcgtcaccatcagtacatcagtacatcagtgggctaagatctaatggtgtcgttcgacggccgcgggcactagccacgagccaagagcggtacagttgctgcgaaatggtgggcagcctgacaggttcagtttgaataggatgaaaacgccatgaagaacagagaagctggcaattgacgatagtggggcgagcgcacgctgcttgctggtccgagcgtaggacgaacgacaaaccatgggcggggccaccaggccccagggagggcgtcactggttggatgtcgtctgttaggctagagctcaggcgctcagggcctctggcccaagcctcggacgcaagcctctgacgagcgagcgcgcgctggggcgggtctgcgcaggaacggcaggactgccgagttggttgaatattgaatggttggatgattggtttgctgggtttggttttgacacgcacggcacaggacaagtgagtttggaggttggatttgcgcaggtagaaagggcgctcggcctcagggcctcagcgtcagcagacgagctcgccagtccaggtcagtacccagtgttcgttacttcccaccgtccagagtttgctgctgcctctcagcttgattcgtgtcggagaagtagaaatggtcacagcgtaccacacactgtccccatgcctgcgtcctgctccacgagtgtttagccacaaaaatttatggacaaccttactgagcctgtgctggggggtgctttagcaccagcactggcgccatggcgtcaaggttaattccactgtcattcgacaaccttcaactgatgggggcaactgggcacaaaaccttggatatataaccaactccaactcccacggttttttaccttttgctcttctccccaggactcactacttctactcctctattcatcttcaccaaaatgtgagacctaatatcactgctgtacagacaagactgtgtatattaggtctcaatgaaatctattctgattgagaaaccaaaccaattggccattgttgaacgtgaaatcccaactccatccgccggtgaagttcgtgtgaaagtgaagctggcgggaatctgcggatccgattcccatatttaccgtggtcataacccatttgcaaaatatcctcgtgtgatcggacacgaattttttggtgtcattgacgcagtcggagaaggtgttgaaagcgcccgcgttggtgaacgagttgcagtggatcccgtggtgtcgtgcggccattgctatccctgctctatcggaaagcccaatgtttgtactaccttggcggtgctgggcgtgcatgcggacggaggcttctctgagtacgcggtggttccggccaagaacgcgtggaagattcccgaggcagttgccgatcaatatgccgtgatgattgagccgtttacaatcgccgcgaacgtgacgggacacggtcagccaactgaaaatgacactgttctggtgtatggcgctggacccattggcctcacaattgttcaagtcctgaaaggagtgtataatgtcaaaaacgtgattgtcgccgatcgaattgacgagcgacttgagaaggccaaggaatccggagctgactgggctattaataatagccagacaccgctgggcgagattttcactgaaaaaggcattaagccgacgctgattattgatgccgcttgtcacccctcgattctgaaggaggcggtgacgctggcttctccagcggcacgtattgttctgatgggattctcgtctgaaccatctgaagttattcagcaaggaattacgggcaaggagctctctattttctccagccgccttaacgcaaacaagttccccatcgtgatcgattggctctccaagggcctgattaaaccagaaaagctcattacgcacacgtttgattttcaacatgttgcggacgctatcagcctttttgagcaagaccagaaacactgctgtaaggtgctgctcactttctccgagtaacacggatcaacaaaaatgtgcgccagctttgttgtgtgcccttttcctctagcggatcctatccacatttcgtgaatgaaccgattaagctctgacgagatttccttgttttctgtagatacctcttgtagaggtgtcaaagttatgttgtgatcgagagagttacctgcaaggtattatgcaagcggcttgatgaggacactcaactacgtgtgagtaggagtgcgttgctcagcagttgacgcgtgagccgcttgagcagcttgataaaggtcctaatcgtcttccagctgttggcagacgacaatgtgctgtagtagtaggataggcccgtaaaagtgtagaagcaataggttgagactgactgtaatgggtacttgcgttcaaagactgaaagtccaaaggattgctcaaaggcccctgaggtctgagcctctgagcgcgctcttcgtcaaaattcgccggggccatcggtcatcctctcgagggcagcatcttcctgcttgaagactgagtccgtctcagataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatgcatcatgcatcatgcatcatgcgtcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatgggtaaggaaaagacacacgtatcgcgaccccgactcaactccaacatggacgcagacctgtacggatacaaatgggctcgagataatgtcggacagtcgggtgcgacgatctacagactctatggaaaaccggacgcccccgagctctttcttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactcgggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcg

SEQ ID NO: 54 - DNA, 4654 bp, synthetic construct
  source 1..4654: mol_type = other DNA
tgcagagccttgcaaatgatgaaagaccaatggaattgatcattgattattgatgatgcatgatgaaagagatagaaacagatgcaagatgggatggcgcgatgatcaaatgataatgaacgttcaacccttcaaaattgaaactctcaggctgagcggctgagcaagctctgagaatgtgggtcttcgaagactttcgcttgagcatggtgcatgtgcaaagagatggaatgtggaattgttgtaggtaggaacacagcccggtagagcggcgcagtggctcagcgaagctgggagaacgactgaaccggtcttgagattcatcatctcagcgtgcgtcagcgcgtcagcttcagcgtttgagaatggcacggactgacctgtcgtacatcccaaggaggcccgtgcgctatactcgccttgtggcctaatctgcgatcaacccatcctcaacgaaagcgcaagtagcgactaagcacactatctttcgggtcggtgcggtcacagtatcggcatctctgtctttagctacgatggtgtgcgtgtaactgatcagtacacccgctgtatcaaaactctatcttcaatccgctaagaatcgtcaccatcagtacatcagtacatcagtgggctaagatctaatggtgtcgttcgacggccgcgggcactagccacgagccaagagcggtacagttgctgcgaaatggtgggcagcctgacaggttcagtttgaataggatgaaaacgccatgaagaacagagaagctggcaattgacgatagtggggcgagcgcacgctgcttgctggtccgagcgtaggacgaacgacaaaccatgggcggggccaccaggccccagggagggcgtcactggttggatgtcgtctgttaggctagagctcaggcgctcagggcctctggcccaagcctcggacgcaagcctctgacgagcgagcgcgcgctggggcgggtctgcgcaggaacggcaggactgccgagttggttgaatattgaatggttggatgattggtttgctgggtttggttttgacacgcacggcacaggacaagtgagtttggaggttggatttgcgcaggtagaaagggcgctcggcctcagggcctcagcgtcagcagacgagctcgccagtccaggtcagtacccagtgttcgttacttcccaccgtccagagtttgctgctgcctctcagcttgattcgtgtcggagaagtagaaatggtcacagcgtaccacacactgtccccatgcctgcgtcctgctccacgagtgtttagccacaaaaatttatggacaaccttactgagcctgtgctggggggtgctttagcaccagcactggcgccatggcgtcaaggttaattccactgtcattcgacaaccttcaactgatgggggcaactgggcacaaaaccttggatatataaccaactccaactcccacggttttttaccttttgctcttctccccaggactcactacttctactcctctattcatcttcaccaaaatgtgagacctaatatcactgctgtacagacaagactgtgtatattaggtctcaatgaacattccgaccaagatgaaggcagtcgttaaatcggaggcaggttatgacaatatgtccctgcaagagattcctgtcccagaagttaagggaaaacaggtgttgatgaaagtcgcctatacgggtatctgcggtacggacattcatacctttaaaggcgagtacgccaatgccgtgaccccacttgtcctcggccatgaattttctggcaaggtcgttgccgtcggagatgaagttaccaaggtcaaaccgggtgaccgagttacctccgaaacaacatttgatacatgcggagagtgtgtgtactgtaaaaataaggaatataacctgtgcgaaaaccgttcgggtattggtacgaaagccaatggatcgatggctaattatgtgcttacgcgagaggagtctgtgcatgtccttcctgacaatgtttcctacaaacttgccgcgatgtctgagccgctggcttcctgtgtgcacgcaatgtaccagaaaaccccgttcacccttcatgatacagttctcattatcggccccggacctatgggtttgctctctctgcagattgcaaaggaaattggagcctttaccatcgtctccggtattacgaaagacagcgaccgcctggaaatcgcaaaagaacttggagccgacatcgttgttgacacgcagaaggaagacttggcgaagatcgtcatggaggctactaacggagtgggagttaccaaagtctatgattgtagcggcgctacaccagcggtgaatgctgccctcccccttctgaagaagggaggtacttttcagcaggttggcctgtttgcaaagcctctgaacgaattggatgaacgtacaattattcagcacgaaattacatacgtgggatcccgttctcagaatccgtatgattggcctatcgccattcacctggaggccaaaggtgctattaatgaagataaaatggttacgaaagtcttcgatttggatcattggcgtgaggcattcgaggctatgatggctggaaaagaactcaaggttcttattgccagcaatccagacgacgaaactcttaattaacacggatcaacaaaaatgtgcgccagctttgttgtgtgcccttttcctctagcggatcctatccacatttcgtgaatgaaccgattaagctctgacgagatttccttgttttctgtagatacctcttgtagaggtgtcaaagttatgttgtgatcgagagagttacctgcaaggtattatgcaagcggcttgatgaggacactcaactacgtgtgagtaggagtgcgttgctcagcagttgacgcgtgagccgcttgagcagcttgataaaggtcctaatcgtcttccagctgttggcagacgacaatgtgctgtagtagtaggataggcccgtaaaagtgtagaagcaataggttgagactgactgtaatgggtacttgcgttcaaagactgaaagtccaaaggattgctcaaaggcccctgaggtctgagcctctgagcgcgctcttcgtcaaaattcgccggggccatcggtcatcctctcgagggcagcatcttcctgcttgaagactgagtccgtctcagataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatgcatcatgcatcatgcatcatgcgtcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatgggtaaggaaaagacacacgtatcgcgaccccgactcaactccaacatggacgcagacctgtacggatacaaatgggctcgagataatgtcggacagtcgggtgcgacgatctacagactctatggaaaaccggacgcccccgagctctttcttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactcgggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcg

SEQ ID NO: 55 - DNA, 4579 bp, synthetic construct
  source 1..4579: mol_type = other DNA
tgcagagccttgcaaatgatgaaagaccaatggaattgatcattgattattgatgatgcatgatgaaagagatagaaacagatgcaagatgggatggcgcgatgatcaaatgataatgaacgttcaacccttcaaaattgaaactctcaggctgagcggctgagcaagctctgagaatgtgggtcttcgaagactttcgcttgagcatggtgcatgtgcaaagagatggaatgtggaattgttgtaggtaggaacacagcccggtagagcggcgcagtggctcagcgaagctgggagaacgactgaaccggtcttgagattcatcatctcagcgtgcgtcagcgcgtcagcttcagcgtttgagaatggcacggactgacctgtcgtacatcccaaggaggcccgtgcgctatactcgccttgtggcctaatctgcgatcaacccatcctcaacgaaagcgcaagtagcgactaagcacactatctttcgggtcggtgcggtcacagtatcggcatctctgtctttagctacgatggtgtgcgtgtaactgatcagtacacccgctgtatcaaaactctatcttcaatccgctaagaatcgtcaccatcagtacatcagtacatcagtgggctaagatctaatggtgtcgttcgacggccgcgggcactagccacgagccaagagcggtacagttgctgcgaaatggtgggcagcctgacaggttcagtttgaataggatgaaaacgccatgaagaacagagaagctggcaattgacgatagtggggcgagcgcacgctgcttgctggtccgagcgtaggacgaacgacaaaccatgggcggggccaccaggccccagggagggcgtcactggttggatgtcgtctgttaggctagagctcaggcgctcagggcctctggcccaagcctcggacgcaagcctctgacgagcgagcgcgcgctggggcgggtctgcgcaggaacggcaggactgccgagttggttgaatattgaatggttggatgattggtttgctgggtttggttttgacacgcacggcacaggacaagtgagtttggaggttggatttgcgcaggtagaaagggcgctcggcctcagggcctcagcgtcagcagacgagctcgccagtccaggtcagtacccagtgttcgttacttcccaccgtccagagtttgctgctgcctctcagcttgattcgtgtcggagaagtagaaatggtcacagcgtaccacacactgtccccatgcctgcgtcctgctccacgagtgtttagccacaaaaatttatggacaaccttactgagcctgtgctggggggtgctttagcaccagcactggcgccatggcgtcaaggttaattccactgtcattcgacaaccttcaactgatgggggcaactgggcacaaaaccttggatatataaccaactccaactcccacggttttttaccttttgctcttctccccaggactcactacttctactcctctattcatcttcaccaaaatgaaagccgcggtcctccatggaacgaacgacatgcgcttcgaggatattgaaatcaagccgtgcgaaagcgacgaggtcaagatcaaggtcatggccgctggcatttgtggatctgatcccccccgagtgctgaaacattggaagtatcctgttccggctattcctggacatgagttctctggtgttatcgcggaggtcggaaaagatgtgaaaaatgtcaaggtgggtgaccgagttgtggcgatcccctttattccgtgtaatgaatgtgagtactgtaaacgaggtctttttagcctttgtgacgatcacggaatgctgggtgccaaatcctttggagcatttgccgagtatgtgaacattaaggcgactaatgtccttcccatcggcgatatggatttcgaagacgcagcgatgattgagccccttgccgtcgccatgcacggagtgttgaacatcggagttcaagtgggagatacggtcgccgtcatgggctcgggcacaatgggtcaacttgtgattcagggtcttaaaattgctggtgctggcactatcattgctgttgacatttctgataacaaactgcgagagtccaaggaattgggcgctgacattatcattaacgcaaaggacattaaccctgttgaaaaaatcaaagagcttacaggcggaaaaggagtggacatcgctctggaatgtgcgggttccaaaattacccaagagcaatgcctcttgatcactaaaaaaaagtcgaagatcggtttcctgggaattgcgtactctgacattaccttgtcggaggaggcgttcgagaacatctttcgcaaagagctcgaacttaaaggattctggaacagctattcggcaccctttcctggccaagagtggaccaagggcattaatcttgttaacgagggaaaaattaagctgaaggaaatggtttcgcaccgcttttctcttgaggatacgtataaagcgttcgagatgatccgagatcgcaaggaagagtttaacaaaatcctgatccttccccagggcgtggaaaaataacacggatcaacaaaaatgtgcgccagctttgttgtgtgcccttttcctctagcggatcctatccacatttcgtgaatgaaccgattaagctctgacgagatttccttgttttctgtagatacctcttgtagaggtgtcaaagttatgttgtgatcgagagagttacctgcaaggtattatgcaagcggcttgatgaggacactcaactacgtgtgagtaggagtgcgttgctcagcagttgacgcgtgagccgcttgagcagcttgataaaggtcctaatcgtcttccagctgttggcagacgacaatgtgctgtagtagtaggataggcccgtaaaagtgtagaagcaataggttgagactgactgtaatgggtacttgcgttcaaagactgaaagtccaaaggattgctcaaaggcccctgaggtctgagcctctgagcgcgctcttcgtcaaaattcgccggggccatcggtcatcctctcgagggcagcatcttcctgcttgaagactgagtccgtctcagataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatgcatcatgcatcatgcatcatgcgtcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatgggtaaggaaaagacacacgtatcgcgaccccgactcaactccaacatggacgcagacctgtacggatacaaatgggctcgagataatgtcggacagtcgggtgcgacgatctacagactctatggaaaaccggacgcccccgagctctttcttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactcgggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcg

SEQ ID NO: 56 - DNA, 4633 bp, synthetic construct
  source 1..4633: mol_type = other DNA
tgcagagccttgcaaatgatgaaagaccaatggaattgatcattgattattgatgatgcatgatgaaagagatagaaacagatgcaagatgggatggcgcgatgatcaaatgataatgaacgttcaacccttcaaaattgaaactctcaggctgagcggctgagcaagctctgagaatgtgggtcttcgaagactttcgcttgagcatggtgcatgtgcaaagagatggaatgtggaattgttgtaggtaggaacacagcccggtagagcggcgcagtggctcagcgaagctgggagaacgactgaaccggtcttgagattcatcatctcagcgtgcgtcagcgcgtcagcttcagcgtttgagaatggcacggactgacctgtcgtacatcccaaggaggcccgtgcgctatactcgccttgtggcctaatctgcgatcaacccatcctcaacgaaagcgcaagtagcgactaagcacactatctttcgggtcggtgcggtcacagtatcggcatctctgtctttagctacgatggtgtgcgtgtaactgatcagtacacccgctgtatcaaaactctatcttcaatccgctaagaatcgtcaccatcagtacatcagtacatcagtgggctaagatctaatggtgtcgttcgacggccgcgggcactagccacgagccaagagcggtacagttgctgcgaaatggtgggcagcctgacaggttcagtttgaataggatgaaaacgccatgaagaacagagaagctggcaattgacgatagtggggcgagcgcacgctgcttgctggtccgagcgtaggacgaacgacaaaccatgggcggggccaccaggccccagggagggcgtcactggttggatgtcgtctgttaggctagagctcaggcgctcagggcctctggcccaagcctcggacgcaagcctctgacgagcgagcgcgcgctggggcgggtctgcgcaggaacggcaggactgccgagttggttgaatattgaatggttggatgattggtttgctgggtttggttttgacacgcacggcacaggacaagtgagtttggaggttggatttgcgcaggtagaaagggcgctcggcctcagggcctcagcgtcagcagacgagctcgccagtccaggtcagtacccagtgttcgttacttcccaccgtccagagtttgctgctgcctctcagcttgattcgtgtcggagaagtagaaatggtcacagcgtaccacacactgtccccatgcctgcgtcctgctccacgagtgtttagccacaaaaatttatggacaaccttactgagcctgtgctggggggtgctttagcaccagcactggcgccatggcgtcaaggttaattccactgtcattcgacaaccttcaactgatgggggcaactgggcacaaaaccttggatatataaccaactccaactcccacggttttttaccttttgctcttctccccaggactcactacttctactcctctattcatcttcaccaaaatgtgagacctaatatcactgctgtacagacaagactgtgtatattaggtctcaatgaagtcggtccgattttatggaattcgtgatacacgagtcgaagacgttgacgtgcccaagattctggaaaaggatgacgtcattatcaaggtcaaggtggcgggcatctgtggttctgacattagcaaatactcgaaaacaggcccgcacatggttggcgagattttgggacacgagttctccggcgaggtcgctcaggtcggcaaggaagttcgctccttcaagatcggagatcgagtcgcagtttgccctgcgatgccctgcttcgagtgtgatgagtgcaaaaaaggtttgtattcccgttgcaataatgtcgctatcatcggaaataaggagctcggaggatgttttgcggagtacacgaaagttaaagagcgcaatctgattaaaattccagatgagattagctacgaaaccgctgcggcccttgaacccgtctgtatcgcaggacatggtcttttccgctctgaagccaaggtcggagatacggtcgttgtgctgggaacgggtcctatcggattgttcagcattcagtgggccaaaatctttggttccacgaagattatcgcggttgacgtgtttgacgaaaaactggacttggcgaaagagctcggtgcggatatttgtattaatgctaaggagaaaaatatcgttgaagagatcaaacgactgacagacggagatggtgcagatattgtcattgagtctgccggtaccccgcttacatgtggccaggtgcttctgctggcgaaaaaaggtggtactgtcttgtatgcaggcgtcccatacggcgacgtcgcccttactcgagaacaatttgagaaaatcgttcgaagcgagcttacggtgaaaggaacttggtttggcaattctttcccatttccgggaaaggaatggtctgctggcctgtaccacatgcagaaaggagacatgaacgtggaaaagcttgtcactcaccgtatcaatctggaagaagctccagcatacttcgagaaggtttataaacgtgatatcttctttggtaagattatgattaatattgacaattaacacggatcaacaaaaatgtgcgccagctttgttgtgtgcccttttcctctagcggatcctatccacatttcgtgaatgaaccgattaagctctgacgagatttccttgttttctgtagatacctcttgtagaggtgtcaaagttatgttgtgatcgagagagttacctgcaaggtattatgcaagcggcttgatgaggacactcaactacgtgtgagtaggagtgcgttgctcagcagttgacgcgtgagccgcttgagcagcttgataaaggtcctaatcgtcttccagctgttggcagacgacaatgtgctgtagtagtaggataggcccgtaaaagtgtagaagcaataggttgagactgactgtaatgggtacttgcgttcaaagactgaaagtccaaaggattgctcaaaggcccctgaggtctgagcctctgagcgcgctcttcgtcaaaattcgccggggccatcggtcatcctctcgagggcagcatcttcctgcttgaagactgagtccgtctcagataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatgcatcatgcatcatgcatcatgcgtcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatgggtaaggaaaagacacacgtatcgcgaccccgactcaactccaacatggacgcagacctgtacggatacaaatgggctcgagataatgtcggacagtcgggtgcgacgatctacagactctatggaaaaccggacgcccccgagctctttcttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactcgggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcg

SEQ ID NO: 57 - DNA, 4576 bp, synthetic construct
  source 1..4576: mol_type = other DNA
tgcagagccttgcaaatgatgaaagaccaatggaattgatcattgattattgatgatgcatgatgaaagagatagaaacagatgcaagatgggatggcgcgatgatcaaatgataatgaacgttcaacccttcaaaattgaaactctcaggctgagcggctgagcaagctctgagaatgtgggtcttcgaagactttcgcttgagcatggtgcatgtgcaaagagatggaatgtggaattgttgtaggtaggaacacagcccggtagagcggcgcagtggctcagcgaagctgggagaacgactgaaccggtcttgagattcatcatctcagcgtgcgtcagcgcgtcagcttcagcgtttgagaatggcacggactgacctgtcgtacatcccaaggaggcccgtgcgctatactcgccttgtggcctaatctgcgatcaacccatcctcaacgaaagcgcaagtagcgactaagcacactatctttcgggtcggtgcggtcacagtatcggcatctctgtctttagctacgatggtgtgcgtgtaactgatcagtacacccgctgtatcaaaactctatcttcaatccgctaagaatcgtcaccatcagtacatcagtacatcagtgggctaagatctaatggtgtcgttcgacggccgcgggcactagccacgagccaagagcggtacagttgctgcgaaatggtgggcagcctgacaggttcagtttgaataggatgaaaacgccatgaagaacagagaagctggcaattgacgatagtggggcgagcgcacgctgcttgctggtccgagcgtaggacgaacgacaaaccatgggcggggccaccaggccccagggagggcgtcactggttggatgtcgtctgttaggctagagctcaggcgctcagggcctctggcccaagcctcggacgcaagcctctgacgagcgagcgcgcgctggggcgggtctgcgcaggaacggcaggactgccgagttggttgaatattgaatggttggatgattggtttgctgggtttggttttgacacgcacggcacaggacaagtgagtttggaggttggatttgcgcaggtagaaagggcgctcggcctcagggcctcagcgtcagcagacgagctcgccagtccaggtcagtacccagtgttcgttacttcccaccgtccagagtttgctgctgcctctcagcttgattcgtgtcggagaagtagaaatggtcacagcgtaccacacactgtccccatgcctgcgtcctgctccacgagtgtttagccacaaaaatttatggacaaccttactgagcctgtgctggggggtgctttagcaccagcactggcgccatggcgtcaaggttaattccactgtcattcgacaaccttcaactgatgggggcaactgggcacaaaaccttggatatataaccaactccaactcccacggttttttaccttttgctcttctccccaggactcactacttctactcctctattcatcttcaccaaaatgaaagcttccatgctggaggacttgaacaaattcagcgtcaaagagatcgacatcccgtcccccaaaaaggatgaggtcgtcgtgaaggtgatggcggcaggcacgtgtggatccgattcgcataaaatgattagcggatggaaatatggctatcccgcggtgatgggacacgaattttctggaattgtgacacaactgggagaaaacgtgtccaacgtcagcgtcggtcaacacgtcgccgtggctcccttcattccctgcttcaagtgtcattattgtcaaatcggactcttccagatgtgtgaaaattattccatgctcggtcagcaaaaatttggaggtttcgagcaatacgtttccgtcccggcacgtaacgttctggacatcggcaagatgtcgttcgaggaaggtgcccttattgaaccgatggcagttgcggcccacgctgttatgggaattaaaccggagctgggtgatacagttgcggtcttcggcctcggtacagtcggagatcttgttgtccgtcttcttatttcctcgggagcgacaaacgtcattggcattgacattgacgaccagaagcttgaaaagggactggatgagggttgcacacacgtgatcaactctgccaaagagtctcttgaagaaaagatcatggaatacaccgacggtctgggtgtcgatatttccatggaatgcgccggcagcaaaatcactgaggagcaaacactgctcgttacgaaacgccgtggaaaggttggatttgtcggtattgcttatagcgacgttctgctccatcagaaagcatttgagaacatcttccgccacgagcttacggtgactggattttggaattcctattccgcaccatttcctggacgcgagtggaccaactcgatccaattggttaatcgtggtcgtatcaagatcaaagatctcattactcatcgatttgagcttgaagacatgcaaaaagcattcaatatgatcaccactcgttcggaatcgtttaataaggtcatgtttttccccaacggcatcaattaacacggatcaacaaaaatgtgcgccagctttgttgtgtgcccttttcctctagcggatcctatccacatttcgtgaatgaaccgattaagctctgacgagatttccttgttttctgtagatacctcttgtagaggtgtcaaagttatgttgtgatcgagagagttacctgcaaggtattatgcaagcggcttgatgaggacactcaactacgtgtgagtaggagtgcgttgctcagcagttgacgcgtgagccgcttgagcagcttgataaaggtcctaatcgtcttccagctgttggcagacgacaatgtgctgtagtagtaggataggcccgtaaaagtgtagaagcaataggttgagactgactgtaatgggtacttgcgttcaaagactgaaagtccaaaggattgctcaaaggcccctgaggtctgagcctctgagcgcgctcttcgtcaaaattcgccggggccatcggtcatcctctcgagggcagcatcttcctgcttgaagactgagtccgtctcagataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatgcatcatgcatcatgcatcatgcgtcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatgggtaaggaaaagacacacgtatcgcgaccccgactcaactccaacatggacgcagacctgtacggatacaaatgggctcgagataatgtcggacagtcgggtgcgacgatctacagactctatggaaaaccggacgcccccgagctctttcttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactcgggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcg

SEQ ID NO: 58 - DNA, 4645 bp, synthetic construct
  source 1..4645: mol_type = other DNA
tgcagagccttgcaaatgatgaaagaccaatggaattgatcattgattattgatgatgcatgatgaaagagatagaaacagatgcaagatgggatggcgcgatgatcaaatgataatgaacgttcaacccttcaaaattgaaactctcaggctgagcggctgagcaagctctgagaatgtgggtcttcgaagactttcgcttgagcatggtgcatgtgcaaagagatggaatgtggaattgttgtaggtaggaacacagcccggtagagcggcgcagtggctcagcgaagctgggagaacgactgaaccggtcttgagattcatcatctcagcgtgcgtcagcgcgtcagcttcagcgtttgagaatggcacggactgacctgtcgtacatcccaaggaggcccgtgcgctatactcgccttgtggcctaatctgcgatcaacccatcctcaacgaaagcgcaagtagcgactaagcacactatctttcgggtcggtgcggtcacagtatcggcatctctgtctttagctacgatggtgtgcgtgtaactgatcagtacacccgctgtatcaaaactctatcttcaatccgctaagaatcgtcaccatcagtacatcagtacatcagtgggctaagatctaatggtgtcgttcgacggccgcgggcactagccacgagccaagagcggtacagttgctgcgaaatggtgggcagcctgacaggttcagtttgaataggatgaaaacgccatgaagaacagagaagctggcaattgacgatagtggggcgagcgcacgctgcttgctggtccgagcgtaggacgaacgacaaaccatgggcggggccaccaggccccagggagggcgtcactggttggatgtcgtctgttaggctagagctcaggcgctcagggcctctggcccaagcctcggacgcaagcctctgacgagcgagcgcgcgctggggcgggtctgcgcaggaacggcaggactgccgagttggttgaatattgaatggttggatgattggtttgctgggtttggttttgacacgcacggcacaggacaagtgagtttggaggttggatttgcgcaggtagaaagggcgctcggcctcagggcctcagcgtcagcagacgagctcgccagtccaggtcagtacccagtgttcgttacttcccaccgtccagagtttgctgctgcctctcagcttgattcgtgtcggagaagtagaaatggtcacagcgtaccacacactgtccccatgcctgcgtcctgctccacgagtgtttagccacaaaaatttatggacaaccttactgagcctgtgctggggggtgctttagcaccagcactggcgccatggcgtcaaggttaattccactgtcattcgacaaccttcaactgatgggggcaactgggcacaaaaccttggatatataaccaactccaactcccacggttttttaccttttgctcttctccccaggactcactacttctactcctctattcatcttcaccaaaatgtgagacctaatatcactgctgtacagacaagactgtgtatattaggtctcaatgaaggctctcaatttgtacggtatccaggacctccgctttgaggaaacacccgctccctctatcgaacatgatgacgatatcattatcaaagtcaaggctgtcggaatctgcggatccgatttgtcgcgttataagaaactgggaccttatgtccctggaatgacttttggacacgaattcgcgggagaggtcgtcaaaattggtcgttccgtgacgggattttctatcggtgatcgtgtcgctgcttgccctacttatacttgcggccagtgccgttactgtcaactcggcgagccgacacgatgtgagcgactgtctgtgatcggcgcacgccacccaggtgcatacgcagaatacgtcaaacttcccgcgaagcatgtcatccccttgcccaacgttgttaactatgacgaggccgccctcattgaaccggcttcggttgttgcgcacggcttttatcgtactaacattaagccaggtgcaagcgtcgctattatgggcgttggctcgatcggtctgctcgccgtccaatgggcaaaaatcttcggtgcgacaacggtctttgccattgatattgacgaacagaaacttaacgtcgctaatcaacttggagcagatgtcttgatctccagcctccaacgacccgcacacaagcaaatccttgaatataccaacggaatcggtgttgacgtggctgtggagtctgctggtacaccttctacctccgcccaggtgtttgcgctgccgaaaaaaggtggtgaagttgtctttcttggtattccttatgccgatgtccagattgaacgattttacttcgaaaaaattgttcgtaatgaactgcacgtttacggatcttggaatgcactctcctccccattcccgggtaaagagtgggccacgactatccattatatgtcgtccggacaattgaatgtcgcgcccatgatttcctaccgactccctctcgctaagggccccgagacgttccagcagatcgcaaagggtgaactcaagcccacaaaagtccttttctatcctgagaagttgtctgagcgcaagtaacacggatcaacaaaaatgtgcgccagctttgttgtgtgcccttttcctctagcggatcctatccacatttcgtgaatgaaccgattaagctctgacgagatttccttgttttctgtagatacctcttgtagaggtgtcaaagttatgttgtgatcgagagagttacctgcaaggtattatgcaagcggcttgatgaggacactcaactacgtgtgagtaggagtgcgttgctcagcagttgacgcgtgagccgcttgagcagcttgataaaggtcctaatcgtcttccagctgttggcagacgacaatgtgctgtagtagtaggataggcccgtaaaagtgtagaagcaataggttgagactgactgtaatgggtacttgcgttcaaagactgaaagtccaaaggattgctcaaaggcccctgaggtctgagcctctgagcgcgctcttcgtcaaaattcgccggggccatcggtcatcctctcgagggcagcatcttcctgcttgaagactgagtccgtctcagataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatgcatcatgcatcatgcatcatgcgtcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatgggtaaggaaaagacacacgtatcgcgaccccgactcaactccaacatggacgcagacctgtacggatacaaatgggctcgagataatgtcggacagtcgggtgcgacgatctacagactctatggaaaaccggacgcccccgagctctttcttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactcgggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcg

SEQ ID NO: 59 - DNA, 4636 bp, synthetic construct
  source 1..4636: mol_type = other DNA
tgcagagccttgcaaatgatgaaagaccaatggaattgatcattgattattgatgatgcatgatgaaagagatagaaacagatgcaagatgggatggcgcgatgatcaaatgataatgaacgttcaacccttcaaaattgaaactctcaggctgagcggctgagcaagctctgagaatgtgggtcttcgaagactttcgcttgagcatggtgcatgtgcaaagagatggaatgtggaattgttgtaggtaggaacacagcccggtagagcggcgcagtggctcagcgaagctgggagaacgactgaaccggtcttgagattcatcatctcagcgtgcgtcagcgcgtcagcttcagcgtttgagaatggcacggactgacctgtcgtacatcccaaggaggcccgtgcgctatactcgccttgtggcctaatctgcgatcaacccatcctcaacgaaagcgcaagtagcgactaagcacactatctttcgggtcggtgcggtcacagtatcggcatctctgtctttagctacgatggtgtgcgtgtaactgatcagtacacccgctgtatcaaaactctatcttcaatccgctaagaatcgtcaccatcagtacatcagtacatcagtgggctaagatctaatggtgtcgttcgacggccgcgggcactagccacgagccaagagcggtacagttgctgcgaaatggtgggcagcctgacaggttcagtttgaataggatgaaaacgccatgaagaacagagaagctggcaattgacgatagtggggcgagcgcacgctgcttgctggtccgagcgtaggacgaacgacaaaccatgggcggggccaccaggccccagggagggcgtcactggttggatgtcgtctgttaggctagagctcaggcgctcagggcctctggcccaagcctcggacgcaagcctctgacgagcgagcgcgcgctggggcgggtctgcgcaggaacggcaggactgccgagttggttgaatattgaatggttggatgattggtttgctgggtttggttttgacacgcacggcacaggacaagtgagtttggaggttggatttgcgcaggtagaaagggcgctcggcctcagggcctcagcgtcagcagacgagctcgccagtccaggtcagtacccagtgttcgttacttcccaccgtccagagtttgctgctgcctctcagcttgattcgtgtcggagaagtagaaatggtcacagcgtaccacacactgtccccatgcctgcgtcctgctccacgagtgtttagccacaaaaatttatggacaaccttactgagcctgtgctggggggtgctttagcaccagcactggcgccatggcgtcaaggttaattccactgtcattcgacaaccttcaactgatgggggcaactgggcacaaaaccttggatatataaccaactccaactcccacggttttttaccttttgctcttctccccaggactcactacttctactcctctattcatcttcaccaaaatgtgagacctaatatcactgctgtacagacaagactgtgtatattaggtctcaatgaaagtccttagcctgcataagcctggcgatattcgatttgaggacgctccaatgcctgagccaggagagggcgaagtgctgctgaaagttatggcatgcggcgtctgttcgtcggacgaggctcgaattttcaaggacggtgcccatattatgccaattgttcctggccacgagttctgtggccaagtggtgaaaacgggagcaggagtggacgaaagcctcatcggccgaaaagcgagcgtcttcccaatgcttccctgtttcgaatgcccttcttgtaaacgaggtgactacgcaacctgttctaactataaatactatggttcccgcaacgatggtggaatgggagaataccgagcaatcaaggcgtggaacctgaaccttctggatgattccgtcgattattgcgtcggcgcactgtccgagccatccgcggttggccatcacgccactgaagtgggaggagtgaaagccggagacaaggttttcgtcattggtagcggaaccatcggacttttgatcggcttcttcgctcaggctaagggtgcagaggtttggatcggtgcgcgtcgtaaagagagcaacgatttcgcggcgagcctgggttttaaaacgatcgacaccaataataccgaagacgaggttgcaaaggccacggatggtaacggcatggatgttgtcttcgaagcagttggtagcaataagtgtcttattgaagctattggatctgtcacggcgggaggcacacttgtcaccgtgggcaatcctgtgggagacatgaatcttccccgagatgtgtactggcgcatcctccgaaagcaactcacggtccgaggtacttggaactctagctactctgacaaggccaatgactggcacgaagtggccgcgttgatgaaagaaaaaaaaatcccgttcgaaaaactcatcacccgtaagtacaaactttctcaatgcgatgaggccttcgattttttccgtgaccactctattcagaagtcccgcgtgatgtttgttatgcacgaaaaagaatccgaagcttacaacggataacacggatcaacaaaaatgtgcgccagctttgttgtgtgcccttttcctctagcggatcctatccacatttcgtgaatgaaccgattaagctctgacgagatttccttgttttctgtagatacctcttgtagaggtgtcaaagttatgttgtgatcgagagagttacctgcaaggtattatgcaagcggcttgatgaggacactcaactacgtgtgagtaggagtgcgttgctcagcagttgacgcgtgagccgcttgagcagcttgataaaggtcctaatcgtcttccagctgttggcagacgacaatgtgctgtagtagtaggataggcccgtaaaagtgtagaagcaataggttgagactgactgtaatgggtacttgcgttcaaagactgaaagtccaaaggattgctcaaaggcccctgaggtctgagcctctgagcgcgctcttcgtcaaaattcgccggggccatcggtcatcctctcgagggcagcatcttcctgcttgaagactgagtccgtctcagataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatgcatcatgcatcatgcatcatgcgtcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatgggtaaggaaaagacacacgtatcgcgaccccgactcaactccaacatggacgcagacctgtacggatacaaatgggctcgagataatgtcggacagtcgggtgcgacgatctacagactctatggaaaaccggacgcccccgagctctttcttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactcgggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcg

SEQ ID NO: 60 - DNA, 4591 bp, synthetic construct
  source 1..4591: mol_type = other DNA
tgcagagccttgcaaatgatgaaagaccaatggaattgatcattgattattgatgatgcatgatgaaagagatagaaacagatgcaagatgggatggcgcgatgatcaaatgataatgaacgttcaacccttcaaaattgaaactctcaggctgagcggctgagcaagctctgagaatgtgggtcttcgaagactttcgcttgagcatggtgcatgtgcaaagagatggaatgtggaattgttgtaggtaggaacacagcccggtagagcggcgcagtggctcagcgaagctgggagaacgactgaaccggtcttgagattcatcatctcagcgtgcgtcagcgcgtcagcttcagcgtttgagaatggcacggactgacctgtcgtacatcccaaggaggcccgtgcgctatactcgccttgtggcctaatctgcgatcaacccatcctcaacgaaagcgcaagtagcgactaagcacactatctttcgggtcggtgcggtcacagtatcggcatctctgtctttagctacgatggtgtgcgtgtaactgatcagtacacccgctgtatcaaaactctatcttcaatccgctaagaatcgtcaccatcagtacatcagtacatcagtgggctaagatctaatggtgtcgttcgacggccgcgggcactagccacgagccaagagcggtacagttgctgcgaaatggtgggcagcctgacaggttcagtttgaataggatgaaaacgccatgaagaacagagaagctggcaattgacgatagtggggcgagcgcacgctgcttgctggtccgagcgtaggacgaacgacaaaccatgggcggggccaccaggccccagggagggcgtcactggttggatgtcgtctgttaggctagagctcaggcgctcagggcctctggcccaagcctcggacgcaagcctctgacgagcgagcgcgcgctggggcgggtctgcgcaggaacggcaggactgccgagttggttgaatattgaatggttggatgattggtttgctgggtttggttttgacacgcacggcacaggacaagtgagtttggaggttggatttgcgcaggtagaaagggcgctcggcctcagggcctcagcgtcagcagacgagctcgccagtccaggtcagtacccagtgttcgttacttcccaccgtccagagtttgctgctgcctctcagcttgattcgtgtcggagaagtagaaatggtcacagcgtaccacacactgtccccatgcctgcgtcctgctccacgagtgtttagccacaaaaatttatggacaaccttactgagcctgtgctggggggtgctttagcaccagcactggcgccatggcgtcaaggttaattccactgtcattcgacaaccttcaactgatgggggcaactgggcacaaaaccttggatatataaccaactccaactcccacggttttttaccttttgctcttctccccaggactcactacttctactcctctattcatcttcaccaaaatgtgagacctaatatcactgctgtacagacaagactgtgtatattaggtctcaatggtcaacatgatgaaagccctcgtgtttactgcgccccatacgttcgagtatcgtgacgttccccgacccgaacccgcccccgacgaggtcctggttgaggtgaaagcatgcgcaatttgtggttctgatgttcacggtagctcgggcgaaaacggtcgacgcattcctcccttggtgatgggccatgaggcaagcggcactatcgcacaattgggcgaagctgtgcagtcgtgggcagtgggagatcgcgttacatttgactctactgagtactgcggctactgttggttttgcacgcacggtatgcacaatttgtgcgcccagcgacgtatcgtgggcgttgcgtgcacagagtaccgtaaaaatggtgcaatggctcaatacattgcagtgaaagcccgtaccctttatgctttgccgccggcggtctcctttgaacagggttgccttgtcgaaccctttgctgtgggtatgcatgctgtcttgcgatctggactggcagcgggacagacggccgctgtgattggagatggaacaatcggtctgatgactcttattgcggccaagagccagggagcacaagtctgtatcagcggacaccacacccgtcccatgcagctcgcagcccagctcggagccctggattgtgcccaactgccccagctgacagctgagcgaggtgcggacgttgtttacgacgctgttggctctcaatccagctttacgcaagcgatggagcttgtccgtaagggaggccgaattatggttatcggcaatcatgcacgcacgatcgattttcctttgcaacgttgtatcacggaacagatcgacgttcagttttcgtacagctctgccggtgagtatcctgtttgtcttcaagccattgctgagggacgcgcagatttgacaggactgtggcaatctttcccactggctgagggcgctcaagttttcgaccgcctggttcaccgagatgagtccctgacgaaagccgttttgatcccataacacggatcaacaaaaatgtgcgccagctttgttgtgtgcccttttcctctagcggatcctatccacatttcgtgaatgaaccgattaagctctgacgagatttccttgttttctgtagatacctcttgtagaggtgtcaaagttatgttgtgatcgagagagttacctgcaaggtattatgcaagcggcttgatgaggacactcaactacgtgtgagtaggagtgcgttgctcagcagttgacgcgtgagccgcttgagcagcttgataaaggtcctaatcgtcttccagctgttggcagacgacaatgtgctgtagtagtaggataggcccgtaaaagtgtagaagcaataggttgagactgactgtaatgggtacttgcgttcaaagactgaaagtccaaaggattgctcaaaggcccctgaggtctgagcctctgagcgcgctcttcgtcaaaattcgccggggccatcggtcatcctctcgagggcagcatcttcctgcttgaagactgagtccgtctcagataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatgcatcatgcatcatgcatcatgcgtcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatgggtaaggaaaagacacacgtatcgcgaccccgactcaactccaacatggacgcagacctgtacggatacaaatgggctcgagataatgtcggacagtcgggtgcgacgatctacagactctatggaaaaccggacgcccccgagctctttcttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactcgggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcg

SEQ ID NO: 61 - DNA, 4654 bp, synthetic construct
  misc_feature 1..4654: note = synthetic
  source 1..4654: mol_type = other DNA
tgcagagccttgcaaatgatgaaagaccaatggaattgatcattgattattgatgatgcatgatgaaagagatagaaacagatgcaagatgggatggcgcgatgatcaaatgataatgaacgttcaacccttcaaaattgaaactctcaggctgagcggctgagcaagctctgagaatgtgggtcttcgaagactttcgcttgagcatggtgcatgtgcaaagagatggaatgtggaattgttgtaggtaggaacacagcccggtagagcggcgcagtggctcagcgaagctgggagaacgactgaaccggtcttgagattcatcatctcagcgtgcgtcagcgcgtcagcttcagcgtttgagaatggcacggactgacctgtcgtacatcccaaggaggcccgtgcgctatactcgccttgtggcctaatctgcgatcaacccatcctcaacgaaagcgcaagtagcgactaagcacactatctttcgggtcggtgcggtcacagtatcggcatctctgtctttagctacgatggtgtgcgtgtaactgatcagtacacccgctgtatcaaaactctatcttcaatccgctaagaatcgtcaccatcagtacatcagtacatcagtgggctaagatctaatggtgtcgttcgacggccgcgggcactagccacgagccaagagcggtacagttgctgcgaaatggtgggcagcctgacaggttcagtttgaataggatgaaaacgccatgaagaacagagaagctggcaattgacgatagtggggcgagcgcacgctgcttgctggtccgagcgtaggacgaacgacaaaccatgggcggggccaccaggccccagggagggcgtcactggttggatgtcgtctgttaggctagagctcaggcgctcagggcctctggcccaagcctcggacgcaagcctctgacgagcgagcgcgcgctggggcgggtctgcgcaggaacggcaggactgccgagttggttgaatattgaatggttggatgattggtttgctgggtttggttttgacacgcacggcacaggacaagtgagtttggaggttggatttgcgcaggtagaaagggcgctcggcctcagggcctcagcgtcagcagacgagctcgccagtccaggtcagtacccagtgttcgttacttcccaccgtccagagtttgctgctgcctctcagcttgattcgtgtcggagaagtagaaatggtcacagcgtaccacacactgtccccatgcctgcgtcctgctccacgagtgtttagccacaaaaatttatggacaaccttactgagcctgtgctggggggtgctttagcaccagcactggcgccatggcgtcaaggttaattccactgtcattcgacaaccttcaactgatgggggcaactgggcacaaaaccttggatatataaccaactccaactcccacggttttttaccttttgctcttctccccaggactcactacttctactcctctattcatcttcaccaaaatgtgagacctaatatcactgctgtacagacaagactgtgtatattaggtctcaatgaaggcctatgttttggatggtattggccagctgaatcaacatgagtatgtcctccgagacttgcagcctggagaagccctcgttcaagtggctgcggctggtatctgcggtagcgacctgccccgcatctttgagactggaacttatcattttccaactatcccaggccatgaattcgcaggtgttgtcaaagaactttatgctgatgccgacgaagaaatgctccgacaatggatgggtaagcgcgttgcggtgtttccactgattccctgtcgaacctgtcgaagctgcgcgaatcatcagtacgagatgtgttctaactataattacctcggctcgcgtacggacggaggcttcgctgaatactgtatcgtgcctgtttggaacttgttgcagttgccggacgaggttagcttcgcacaggccgctatgatggaaccggcagctgtggcattgcatgcaatccgacgtggcggcctgcaccccgaccaccccgagaccggtatcggcaagcgtgttctggttatgggacttggcactattggcggattgatcgcccagtggcttagccaatgtggcgttacggagctcaccgccactggtcaccacgccccccaggaagagcttatgcaccgatatgctaacgagaactaccgattcattaatactaatcaaactgcttggaccgatgacgaaaacacctacgatcttatttttgactgcattaatgacagcgaatccgttcagcaggcgttgcgctacgtcaaacccggtggtcagattgttgctgttggcaacccgaaaaaagatcttactcttcaaaaggacatctattggaaactcctgcgaaaacaagtgtgtatgaccggaacatggaactcgagcttcagccatgattcgaacgatgactggcagatcctccttcgataccttcaggagcaaaagctgcatccgcaggcacttatcacacatcgccttccgtttgcccaacttcccaagggcttggagatcatgcgtcttcgtgaagagttttacaacaaaattatgatcgacttgactgtctgttcttaacacggatcaacaaaaatgtgcgccagctttgttgtgtgcccttttcctctagcggatcctatccacatttcgtgaatgaaccgattaagctctgacgagatttccttgttttctgtagatacctcttgtagaggtgtcaaagttatgttgtgatcgagagagttacctgcaaggtattatgcaagcggcttgatgaggacactcaactacgtgtgagtaggagtgcgttgctcagcagttgacgcgtgagccgcttgagcagcttgataaaggtcctaatcgtcttccagctgttggcagacgacaatgtgctgtagtagtaggataggcccgtaaaagtgtagaagcaataggttgagactgactgtaatgggtacttgcgttcaaagactgaaagtccaaaggattgctcaaaggcccctgaggtctgagcctctgagcgcgctcttcgtcaaaattcgccggggccatcggtcatcctctcgagggcagcatcttcctgcttgaagactgagtccgtctcagataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatgcatcatgcatcatgcatcatgcgtcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatgggtaaggaaaagacacacgtatcgcgaccccgactcaactccaacatggacgcagacctgtacggatacaaatgggctcgagataatgtcggacagtcgggtgcgacgatctacagactctatggaaaaccggacgcccccgagctctttcttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactcgggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcg

SEQ ID NO: 62 - DNA, 4609 bp, synthetic construct
  source 1..4609: mol_type = other DNA
tgcagagccttgcaaatgatgaaagaccaatggaattgatcattgattattgatgatgcatgatgaaagagatagaaacagatgcaagatgggatggcgcgatgatcaaatgataatgaacgttcaacccttcaaaattgaaactctcaggctgagcggctgagcaagctctgagaatgtgggtcttcgaagactttcgcttgagcatggtgcatgtgcaaagagatggaatgtggaattgttgtaggtaggaacacagcccggtagagcggcgcagtggctcagcgaagctgggagaacgactgaaccggtcttgagattcatcatctcagcgtgcgtcagcgcgtcagcttcagcgtttgagaatggcacggactgacctgtcgtacatcccaaggaggcccgtgcgctatactcgccttgtggcctaatctgcgatcaacccatcctcaacgaaagcgcaagtagcgactaagcacactatctttcgggtcggtgcggtcacagtatcggcatctctgtctttagctacgatggtgtgcgtgtaactgatcagtacacccgctgtatcaaaactctatcttcaatccgctaagaatcgtcaccatcagtacatcagtacatcagtgggctaagatctaatggtgtcgttcgacggccgcgggcactagccacgagccaagagcggtacagttgctgcgaaatggtgggcagcctgacaggttcagtttgaataggatgaaaacgccatgaagaacagagaagctggcaattgacgatagtggggcgagcgcacgctgcttgctggtccgagcgtaggacgaacgacaaaccatgggcggggccaccaggccccagggagggcgtcactggttggatgtcgtctgttaggctagagctcaggcgctcagggcctctggcccaagcctcggacgcaagcctctgacgagcgagcgcgcgctggggcgggtctgcgcaggaacggcaggactgccgagttggttgaatattgaatggttggatgattggtttgctgggtttggttttgacacgcacggcacaggacaagtgagtttggaggttggatttgcgcaggtagaaagggcgctcggcctcagggcctcagcgtcagcagacgagctcgccagtccaggtcagtacccagtgttcgttacttcccaccgtccagagtttgctgctgcctctcagcttgattcgtgtcggagaagtagaaatggtcacagcgtaccacacactgtccccatgcctgcgtcctgctccacgagtgtttagccacaaaaatttatggacaaccttactgagcctgtgctggggggtgctttagcaccagcactggcgccatggcgtcaaggttaattccactgtcattcgacaaccttcaactgatgggggcaactgggcacaaaaccttggatatataaccaactccaactcccacggttttttaccttttgctcttctccccaggactcactacttctactcctctattcatcttcaccaaaatgtgagacctaatatcactgctgtacagacaagactgtgtatattaggtctcaatgaaggccgtgacttacactcccgaccagggttttgcattggtcgaggttgagacaccccagcttggacctgacgaagttcttctccaggtggaggcttgtggcgtctgcggaagcgaccgtcaagtcgttgcaggcgaatcggttcctaccggtaccgccttccctctggtgatgggccacgagattgccggacgtatcgccgcgttgggcgaaactgtgacagattggcatgttggagactctgtgattgtccatcccttcgtggcatgtggtacgtgcgcaccctgtgttcacggccagcctaacttgtgcgtccgacaggtgtgtattggttatcaacgacctggaggatttgcagagcaagttgctgtcccggctcgtcaactggttcgacgtccagcagaattggcaccggccgccgcagcattggttgtggatgcgtttgccacgccataccacgcgatgcgagcggcgggtgttgagagcggccaaaccgttttgattattggaaccggcggacttggtctcgcggcacttatgctggccatgggcttccatgtcggccgtctgggcgcggtcacgcgacgccaagagggtattgcaatcgcggaggctcatggcgcgcagatggttgtcgtcacggaccaggatgagcgtacaattgcgcgccagcttcgccgctggtctggcgcatccggcattgacgtcgtcatcgatacggtcgctactgctgacactattacattggcagctgaaagcgttcgcccaggaggcactatcgctattgtcggaatgtcggccgacaccgccctctatcctgtcgctaagaccgttcgccgaggtctgacattcgttgcgagctacggatccgtccgagaggatgtcgagcagttggtcagctgggtggcagaaggtcgactggacccggttcgactcgtcgcgggagccctcccactccatgaagccgctcgtgcattcgccccggaccgacccgccggacgttgggttattgatccatctgaataacacggatcaacaaaaatgtgcgccagctttgttgtgtgcccttttcctctagcggatcctatccacatttcgtgaatgaaccgattaagctctgacgagatttccttgttttctgtagatacctcttgtagaggtgtcaaagttatgttgtgatcgagagagttacctgcaaggtattatgcaagcggcttgatgaggacactcaactacgtgtgagtaggagtgcgttgctcagcagttgacgcgtgagccgcttgagcagcttgataaaggtcctaatcgtcttccagctgttggcagacgacaatgtgctgtagtagtaggataggcccgtaaaagtgtagaagcaataggttgagactgactgtaatgggtacttgcgttcaaagactgaaagtccaaaggattgctcaaaggcccctgaggtctgagcctctgagcgcgctcttcgtcaaaattcgccggggccatcggtcatcctctcgagggcagcatcttcctgcttgaagactgagtccgtctcagataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatgcatcatgcatcatgcatcatgcgtcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatgggtaaggaaaagacacacgtatcgcgaccccgactcaactccaacatggacgcagacctgtacggatacaaatgggctcgagataatgtcggacagtcgggtgcgacgatctacagactctatggaaaaccggacgcccccgagctctttcttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactcgggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcg

SEQ ID NO: 63 - DNA, 4654 bp, synthetic construct
  source 1..4654: mol_type = other DNA
tgcagagccttgcaaatgatgaaagaccaatggaattgatcattgattattgatgatgcatgatgaaagagatagaaacagatgcaagatgggatggcgcgatgatcaaatgataatgaacgttcaacccttcaaaattgaaactctcaggctgagcggctgagcaagctctgagaatgtgggtcttcgaagactttcgcttgagcatggtgcatgtgcaaagagatggaatgtggaattgttgtaggtaggaacacagcccggtagagcggcgcagtggctcagcgaagctgggagaacgactgaaccggtcttgagattcatcatctcagcgtgcgtcagcgcgtcagcttcagcgtttgagaatggcacggactgacctgtcgtacatcccaaggaggcccgtgcgctatactcgccttgtggcctaatctgcgatcaacccatcctcaacgaaagcgcaagtagcgactaagcacactatctttcgggtcggtgcggtcacagtatcggcatctctgtctttagctacgatggtgtgcgtgtaactgatcagtacacccgctgtatcaaaactctatcttcaatccgctaagaatcgtcaccatcagtacatcagtacatcagtgggctaagatctaatggtgtcgttcgacggccgcgggcactagccacgagccaagagcggtacagttgctgcgaaatggtgggcagcctgacaggttcagtttgaataggatgaaaacgccatgaagaacagagaagctggcaattgacgatagtggggcgagcgcacgctgcttgctggtccgagcgtaggacgaacgacaaaccatgggcggggccaccaggccccagggagggcgtcactggttggatgtcgtctgttaggctagagctcaggcgctcagggcctctggcccaagcctcggacgcaagcctctgacgagcgagcgcgcgctggggcgggtctgcgcaggaacggcaggactgccgagttggttgaatattgaatggttggatgattggtttgctgggtttggttttgacacgcacggcacaggacaagtgagtttggaggttggatttgcgcaggtagaaagggcgctcggcctcagggcctcagcgtcagcagacgagctcgccagtccaggtcagtacccagtgttcgttacttcccaccgtccagagtttgctgctgcctctcagcttgattcgtgtcggagaagtagaaatggtcacagcgtaccacacactgtccccatgcctgcgtcctgctccacgagtgtttagccacaaaaatttatggacaaccttactgagcctgtgctggggggtgctttagcaccagcactggcgccatggcgtcaaggttaattccactgtcattcgacaaccttcaactgatgggggcaactgggcacaaaaccttggatatataaccaactccaactcccacggttttttaccttttgctcttctccccaggactcactacttctactcctctattcatcttcaccaaaatgtgagacctaatatcactgctgtacagacaagactgtgtatattaggtctcaatgaaaagcttgattaatatcccggaaaaaatgaaagccctcgttttgtatggtgttggtgatttgcgtgtcactgaagtggacgttccgaagctcgaaaaaggaacggtcctccttaaaattaaagcttgcggcatctgttcctccgacatgcctcgttgtttcgttactggcacttaccattttccgaccattccaggacacgagttctctggacaaatcgttgcggtgggtgatgatgttgatgaatccttgttgggaaagcgttcgtgcgttttccctatgcttccctgcaaacactgcaaagcttgcaagatggaggaatatgctcagtgtagcggctattcttacttcggctctcgccaagatggtgcctgggcggaatacctcgtggtcccggtgtggaacctggtgccctttgacgatacactcgattataaaactgcagccctgtgcgaaccagctgcggtctctatgcacgccgttaatattggagacattaaagaaggtcagaatgttgtgatcatcggaacgggtaccatcggattcatgattgctctctttgccaaagaacgtacgaaaacaggaaaggttgtgatcgcgggacgctcgaaaaataagctggaatacgcgaaaaagttgggcttcgatgtcgttgacacctcggttgacgatttgcctgcccaagtgaagaaaatcatcggtattgatggagctgacgtcacattcgaagcggtcggctccaatgaggccattgcctctgctattgaatctacgggcgctttgggttcgattgttcttgtcggtaatccttccactgatctcatcttgccaaagaatatttattggtccatcctccgaaagcaaattacagttaaaggttcctggaactcgtcttataactctcgcgttaatgactggaagaaagcgttggaggtgtttgagcatagcgacgtgaattttgcagacttgatcacacacacattcactatcgaagaaaacgagaaagctttttctgccatccgtaacactggagaattcactctcaaagtcatgtttaccttcgatgattaacacggatcaacaaaaatgtgcgccagctttgttgtgtgcccttttcctctagcggatcctatccacatttcgtgaatgaaccgattaagctctgacgagatttccttgttttctgtagatacctcttgtagaggtgtcaaagttatgttgtgatcgagagagttacctgcaaggtattatgcaagcggcttgatgaggacactcaactacgtgtgagtaggagtgcgttgctcagcagttgacgcgtgagccgcttgagcagcttgataaaggtcctaatcgtcttccagctgttggcagacgacaatgtgctgtagtagtaggataggcccgtaaaagtgtagaagcaataggttgagactgactgtaatgggtacttgcgttcaaagactgaaagtccaaaggattgctcaaaggcccctgaggtctgagcctctgagcgcgctcttcgtcaaaattcgccggggccatcggtcatcctctcgagggcagcatcttcctgcttgaagactgagtccgtctcagataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatgcatcatgcatcatgcatcatgcgtcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatgggtaaggaaaagacacacgtatcgcgaccccgactcaactccaacatggacgcagacctgtacggatacaaatgggctcgagataatgtcggacagtcgggtgcgacgatctacagactctatggaaaaccggacgcccccgagctctttcttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactcgggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcg

SEQ ID NO: 64 - DNA, 4627 bp, synthetic construct
  source 1..4627: mol_type = other DNA
tgcagagccttgcaaatgatgaaagaccaatggaattgatcattgattattgatgatgcatgatgaaagagatagaaacagatgcaagatgggatggcgcgatgatcaaatgataatgaacgttcaacccttcaaaattgaaactctcaggctgagcggctgagcaagctctgagaatgtgggtcttcgaagactttcgcttgagcatggtgcatgtgcaaagagatggaatgtggaattgttgtaggtaggaacacagcccggtagagcggcgcagtggctcagcgaagctgggagaacgactgaaccggtcttgagattcatcatctcagcgtgcgtcagcgcgtcagcttcagcgtttgagaatggcacggactgacctgtcgtacatcccaaggaggcccgtgcgctatactcgccttgtggcctaatctgcgatcaacccatcctcaacgaaagcgcaagtagcgactaagcacactatctttcgggtcggtgcggtcacagtatcggcatctctgtctttagctacgatggtgtgcgtgtaactgatcagtacacccgctgtatcaaaactctatcttcaatccgctaagaatcgtcaccatcagtacatcagtacatcagtgggctaagatctaatggtgtcgttcgacggccgcgggcactagccacgagccaagagcggtacagttgctgcgaaatggtgggcagcctgacaggttcagtttgaataggatgaaaacgccatgaagaacagagaagctggcaattgacgatagtggggcgagcgcacgctgcttgctggtccgagcgtaggacgaacgacaaaccatgggcggggccaccaggccccagggagggcgtcactggttggatgtcgtctgttaggctagagctcaggcgctcagggcctctggcccaagcctcggacgcaagcctctgacgagcgagcgcgcgctggggcgggtctgcgcaggaacggcaggactgccgagttggttgaatattgaatggttggatgattggtttgctgggtttggttttgacacgcacggcacaggacaagtgagtttggaggttggatttgcgcaggtagaaagggcgctcggcctcagggcctcagcgtcagcagacgagctcgccagtccaggtcagtacccagtgttcgttacttcccaccgtccagagtttgctgctgcctctcagcttgattcgtgtcggagaagtagaaatggtcacagcgtaccacacactgtccccatgcctgcgtcctgctccacgagtgtttagccacaaaaatttatggacaaccttactgagcctgtgctggggggtgctttagcaccagcactggcgccatggcgtcaaggttaattccactgtcattcgacaaccttcaactgatgggggcaactgggcacaaaaccttggatatataaccaactccaactcccacggttttttaccttttgctcttctccccaggactcactacttctactcctctattcatcttcaccaaaatgtgagacctaatatcactgctgtacagacaagactgtgtatattaggtctcaatgaaagctgtcgtcctgcatggtaaaaacgacatttgctatgaagaatggcctgaacccatcctgaaaacaggaacagttaagattcatgtcaaggcttgtggcatttgcggctctgacattcctcgcgttctcggagattctgcccattactacccgatcatcctcggccatgagttctccggttgtgtcactgaggtggctgatgacgttgacaagatccaggttggcgatcatgttgcaggagtcccccttctcccgtgcatgaaatgcgatgattgtcaacagggaaactattctcagtgtaaacattactcttttatcggcagccgcgagcaaggtggatttgcagattatgttgtgatcccagccgataacgtggttaaagtcgacccaactcttccttacgaacaggtcgcactcttcgagccttgtactgttgctttgcatgcactcaaagtctcctcgtataaaggaggaaaacgagtggcgatcctgggtggcggcacgattggtctttttgcactccagtgggctaaaatctatggcgcgtcccaagtcacagtcttcggtcgcagcaaggaacacttgaagatcgctgctgctctgggtgccgacacaactatttccactctggacgataattttatgacagaggctatgcgagtcacagagaaaaagggttttgactacatttacgagtccgccggttccacaatcaccatgaagcttgcttttgaactggcagccaaccacgcgaaaatctgtttcattggcacgcccacggaaaacctgactttcagccctaagctgtgggagaacatgaatcgaaaggagttccagcttatgggttcctggatgtcgtgttcggcgccgttccccggcgatgaatggaaggagacagcccatttctttaacaccggacaacttcgatatgtgcctgagatgtttttcgcacagtatcccatggacgatgttaaaaaagcgttcgagcagttccataacaagaataaagttaagggacgtatcctgctcaaaaacaagtaacacggatcaacaaaaatgtgcgccagctttgttgtgtgcccttttcctctagcggatcctatccacatttcgtgaatgaaccgattaagctctgacgagatttccttgttttctgtagatacctcttgtagaggtgtcaaagttatgttgtgatcgagagagttacctgcaaggtattatgcaagcggcttgatgaggacactcaactacgtgtgagtaggagtgcgttgctcagcagttgacgcgtgagccgcttgagcagcttgataaaggtcctaatcgtcttccagctgttggcagacgacaatgtgctgtagtagtaggataggcccgtaaaagtgtagaagcaataggttgagactgactgtaatgggtacttgcgttcaaagactgaaagtccaaaggattgctcaaaggcccctgaggtctgagcctctgagcgcgctcttcgtcaaaattcgccggggccatcggtcatcctctcgagggcagcatcttcctgcttgaagactgagtccgtctcagataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatgcatcatgcatcatgcatcatgcgtcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatgggtaaggaaaagacacacgtatcgcgaccccgactcaactccaacatggacgcagacctgtacggatacaaatgggctcgagataatgtcggacagtcgggtgcgacgatctacagactctatggaaaaccggacgcccccgagctctttcttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactcgggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcg

SEQ ID NO: 65 - DNA, 4594 bp, synthetic construct
  source 1..4594: mol_type = other DNA
tgcagagccttgcaaatgatgaaagaccaatggaattgatcattgattattgatgatgcatgatgaaagagatagaaacagatgcaagatgggatggcgcgatgatcaaatgataatgaacgttcaacccttcaaaattgaaactctcaggctgagcggctgagcaagctctgagaatgtgggtcttcgaagactttcgcttgagcatggtgcatgtgcaaagagatggaatgtggaattgttgtaggtaggaacacagcccggtagagcggcgcagtggctcagcgaagctgggagaacgactgaaccggtcttgagattcatcatctcagcgtgcgtcagcgcgtcagcttcagcgtttgagaatggcacggactgacctgtcgtacatcccaaggaggcccgtgcgctatactcgccttgtggcctaatctgcgatcaacccatcctcaacgaaagcgcaagtagcgactaagcacactatctttcgggtcggtgcggtcacagtatcggcatctctgtctttagctacgatggtgtgcgtgtaactgatcagtacacccgctgtatcaaaactctatcttcaatccgctaagaatcgtcaccatcagtacatcagtacatcagtgggctaagatctaatggtgtcgttcgacggccgcgggcactagccacgagccaagagcggtacagttgctgcgaaatggtgggcagcctgacaggttcagtttgaataggatgaaaacgccatgaagaacagagaagctggcaattgacgatagtggggcgagcgcacgctgcttgctggtccgagcgtaggacgaacgacaaaccatgggcggggccaccaggccccagggagggcgtcactggttggatgtcgtctgttaggctagagctcaggcgctcagggcctctggcccaagcctcggacgcaagcctctgacgagcgagcgcgcgctggggcgggtctgcgcaggaacggcaggactgccgagttggttgaatattgaatggttggatgattggtttgctgggtttggttttgacacgcacggcacaggacaagtgagtttggaggttggatttgcgcaggtagaaagggcgctcggcctcagggcctcagcgtcagcagacgagctcgccagtccaggtcagtacccagtgttcgttacttcccaccgtccagagtttgctgctgcctctcagcttgattcgtgtcggagaagtagaaatggtcacagcgtaccacacactgtccccatgcctgcgtcctgctccacgagtgtttagccacaaaaatttatggacaaccttactgagcctgtgctggggggtgctttagcaccagcactggcgccatggcgtcaaggttaattccactgtcattcgacaaccttcaactgatgggggcaactgggcacaaaaccttggatatataaccaactccaactcccacggttttttaccttttgctcttctccccaggactcactacttctactcctctattcatcttcaccaaaatgtgagacctaatatcactgctgtacagacaagactgtgtatattaggtctcaatgaaagtggcggtttggtatggtgacaaaaatattgtcttggaggaccgtccgatcccttctatcggtgaaaaggacgtcttggtccgtgtcaagtcgtcttccatctgcggagctgaccttcatgcctacaaaggcctctctaagcgccgcaagcctccattggtgatgggacatgaattttccggtatggttgaacgcctcggcgatgagacaaagggccttaaggtcggcgatcgtgtcgtggtggagcccattattagctgcggaaagtgcaaggcatgcaaaaagggaaaaacgaatgtctgcgaaaatcgtaagctgatcggtctgaacctcgacggtgctttcgctgaatatgtttctgtcccttatgaaaagtgccatgaactcccccgtgacctttcgttcgatgagggtgcgattatcgagccgttggccgtcgccgttcatgcggtcaatattactcccatctcgtcgaacgataacgtgcttatcattggtagcggtgtgattggcctcctcatcacacaattgcttcgaagccgcacttcgggcgacattttggttaccggacgtcgcgattacctccttgaaaaggcccgtgaatttggtgccacagatgcaatcaacgtgatcgactgcgatgctgtgaaaacgctgctcaaaaaacacggtcgcgtcgatgttgtcttcgaagcagtgggctctaacgagactgtgaaccagtccatttccctggtcgaatctggaggttctatcactctcgttggcatgctcgatcgcgagatgaccgttgatatgatggaggttgttgcccgtgaaatcactctgcgaggagattatggttatacaactgacgactttaaggaggcgatcgagctcgcttcttcgggtaaagttgatctcaaaggcctcatcacgcatgttctcccgctcgatgagatcaaaaagggcttcgatatcctggcagaacgccgagaaaatgtgatcaaagttgttattaacccataacacggatcaacaaaaatgtgcgccagctttgttgtgtgcccttttcctctagcggatcctatccacatttcgtgaatgaaccgattaagctctgacgagatttccttgttttctgtagatacctcttgtagaggtgtcaaagttatgttgtgatcgagagagttacctgcaaggtattatgcaagcggcttgatgaggacactcaactacgtgtgagtaggagtgcgttgctcagcagttgacgcgtgagccgcttgagcagcttgataaaggtcctaatcgtcttccagctgttggcagacgacaatgtgctgtagtagtaggataggcccgtaaaagtgtagaagcaataggttgagactgactgtaatgggtacttgcgttcaaagactgaaagtccaaaggattgctcaaaggcccctgaggtctgagcctctgagcgcgctcttcgtcaaaattcgccggggccatcggtcatcctctcgagggcagcatcttcctgcttgaagactgagtccgtctcagataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatgcatcatgcatcatgcatcatgcgtcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatgggtaaggaaaagacacacgtatcgcgaccccgactcaactccaacatggacgcagacctgtacggatacaaatgggctcgagataatgtcggacagtcgggtgcgacgatctacagactctatggaaaaccggacgcccccgagctctttcttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactcgggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcg

SEQ ID NO: 66 - DNA, 4612 bp, synthetic construct
  source 1..4612: mol_type = other DNA
tgcagagccttgcaaatgatgaaagaccaatggaattgatcattgattattgatgatgcatgatgaaagagatagaaacagatgcaagatgggatggcgcgatgatcaaatgataatgaacgttcaacccttcaaaattgaaactctcaggctgagcggctgagcaagctctgagaatgtgggtcttcgaagactttcgcttgagcatggtgcatgtgcaaagagatggaatgtggaattgttgtaggtaggaacacagcccggtagagcggcgcagtggctcagcgaagctgggagaacgactgaaccggtcttgagattcatcatctcagcgtgcgtcagcgcgtcagcttcagcgtttgagaatggcacggactgacctgtcgtacatcccaaggaggcccgtgcgctatactcgccttgtggcctaatctgcgatcaacccatcctcaacgaaagcgcaagtagcgactaagcacactatctttcgggtcggtgcggtcacagtatcggcatctctgtctttagctacgatggtgtgcgtgtaactgatcagtacacccgctgtatcaaaactctatcttcaatccgctaagaatcgtcaccatcagtacatcagtacatcagtgggctaagatctaatggtgtcgttcgacggccgcgggcactagccacgagccaagagcggtacagttgctgcgaaatggtgggcagcctgacaggttcagtttgaataggatgaaaacgccatgaagaacagagaagctggcaattgacgatagtggggcgagcgcacgctgcttgctggtccgagcgtaggacgaacgacaaaccatgggcggggccaccaggccccagggagggcgtcactggttggatgtcgtctgttaggctagagctcaggcgctcagggcctctggcccaagcctcggacgcaagcctctgacgagcgagcgcgcgctggggcgggtctgcgcaggaacggcaggactgccgagttggttgaatattgaatggttggatgattggtttgctgggtttggttttgacacgcacggcacaggacaagtgagtttggaggttggatttgcgcaggtagaaagggcgctcggcctcagggcctcagcgtcagcagacgagctcgccagtccaggtcagtacccagtgttcgttacttcccaccgtccagagtttgctgctgcctctcagcttgattcgtgtcggagaagtagaaatggtcacagcgtaccacacactgtccccatgcctgcgtcctgctccacgagtgtttagccacaaaaatttatggacaaccttactgagcctgtgctggggggtgctttagcaccagcactggcgccatggcgtcaaggttaattccactgtcattcgacaaccttcaactgatgggggcaactgggcacaaaaccttggatatataaccaactccaactcccacggttttttaccttttgctcttctccccaggactcactacttctactcctctattcatcttcaccaaaatgtgagacctaatatcactgctgtacagacaagactgtgtatattaggtctcaatgaaagcactggtgttggaggaaatcggtaaattgacccttaaagaggttgagacgcccgtgccaggacgcggagaggttttgttgcgcatccatgcctgtggtatttgctcttcggacaaagcccgtctcttcaagaacggtactcacaccatgccgcttatcccaggtcacgagatctcgggtgagattgttgcaggaggtcccggcattcgagaagagcttatcggccgacgcgctgcggtcttcccgttgattccttgtcgccaatgcgactcctgccgaatcggcaagtacgctcaatgcgagcattacgactatttcggatcgcgtcgagacggaggctttgctgagtacctcgctgtccccgtgtttaatatcaatcttcttcccgatgatatggattatcatacggctgcattgtgcgaaccagccacggttgcatctcatgcacttaagcgcgccaaaatgaaatctggtgatatcgttacgatcattggcactggcacggtcggcctcctggctgctggcatcgctcgtattcgacgagcatctaaaatcattatcgtcggacgaagcaaagaaaaaatcatgaatgcagctcatttcgctgatcatgtgatcaatgcagagggatgggacgtgaacgaggccgtggcttcgctgacggagggtcacatgagcgatgtggtcttggagactggcggcaacagcgatgcttgcacgtacgcgcttatggctgccggcaaaggtacgactgtgatgtttattggcaatcccgaagaaccgattgtttttaaccgtgatatttattggaagatcctccgaaacgaactgacactcaagggtacttggaattctggatatgctcctgaggataatgactggaatgacgtcatccgttatatggcagatggacagattccggcagataaaattatcaccgatagctttccattggaagattataataaggcattcgagcttcttctctcggatcgcttttacaccaaaatcatgtttgacatctaacacggatcaacaaaaatgtgcgccagctttgttgtgtgcccttttcctctagcggatcctatccacatttcgtgaatgaaccgattaagctctgacgagatttccttgttttctgtagatacctcttgtagaggtgtcaaagttatgttgtgatcgagagagttacctgcaaggtattatgcaagcggcttgatgaggacactcaactacgtgtgagtaggagtgcgttgctcagcagttgacgcgtgagccgcttgagcagcttgataaaggtcctaatcgtcttccagctgttggcagacgacaatgtgctgtagtagtaggataggcccgtaaaagtgtagaagcaataggttgagactgactgtaatgggtacttgcgttcaaagactgaaagtccaaaggattgctcaaaggcccctgaggtctgagcctctgagcgcgctcttcgtcaaaattcgccggggccatcggtcatcctctcgagggcagcatcttcctgcttgaagactgagtccgtctcagataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatgcatcatgcatcatgcatcatgcgtcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatgggtaaggaaaagacacacgtatcgcgaccccgactcaactccaacatggacgcagacctgtacggatacaaatgggctcgagataatgtcggacagtcgggtgcgacgatctacagactctatggaaaaccggacgcccccgagctctttcttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactcgggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcg

SEQ ID NO: 67 - DNA, 4627 bp, synthetic construct
  source 1..4627: mol_type = other DNA
tgcagagccttgcaaatgatgaaagaccaatggaattgatcattgattattgatgatgcatgatgaaagagatagaaacagatgcaagatgggatggcgcgatgatcaaatgataatgaacgttcaacccttcaaaattgaaactctcaggctgagcggctgagcaagctctgagaatgtgggtcttcgaagactttcgcttgagcatggtgcatgtgcaaagagatggaatgtggaattgttgtaggtaggaacacagcccggtagagcggcgcagtggctcagcgaagctgggagaacgactgaaccggtcttgagattcatcatctcagcgtgcgtcagcgcgtcagcttcagcgtttgagaatggcacggactgacctgtcgtacatcccaaggaggcccgtgcgctatactcgccttgtggcctaatctgcgatcaacccatcctcaacgaaagcgcaagtagcgactaagcacactatctttcgggtcggtgcggtcacagtatcggcatctctgtctttagctacgatggtgtgcgtgtaactgatcagtacacccgctgtatcaaaactctatcttcaatccgctaagaatcgtcaccatcagtacatcagtacatcagtgggctaagatctaatggtgtcgttcgacggccgcgggcactagccacgagccaagagcggtacagttgctgcgaaatggtgggcagcctgacaggttcagtttgaataggatgaaaacgccatgaagaacagagaagctggcaattgacgatagtggggcgagcgcacgctgcttgctggtccgagcgtaggacgaacgacaaaccatgggcggggccaccaggccccagggagggcgtcactggttggatgtcgtctgttaggctagagctcaggcgctcagggcctctggcccaagcctcggacgcaagcctctgacgagcgagcgcgcgctggggcgggtctgcgcaggaacggcaggactgccgagttggttgaatattgaatggttggatgattggtttgctgggtttggttttgacacgcacggcacaggacaagtgagtttggaggttggatttgcgcaggtagaaagggcgctcggcctcagggcctcagcgtcagcagacgagctcgccagtccaggtcagtacccagtgttcgttacttcccaccgtccagagtttgctgctgcctctcagcttgattcgtgtcggagaagtagaaatggtcacagcgtaccacacactgtccccatgcctgcgtcctgctccacgagtgtttagccacaaaaatttatggacaaccttactgagcctgtgctggggggtgctttagcaccagcactggcgccatggcgtcaaggttaattccactgtcattcgacaaccttcaactgatgggggcaactgggcacaaaaccttggatatataaccaactccaactcccacggttttttaccttttgctcttctccccaggactcactacttctactcctctattcatcttcaccaaaatgtgagacctaatatcactgctgtacagacaagactgtgtatattaggtctcaatgaaagccaatgttctgtaccacattggtgacctgcgatacactgacgttgccttgccggaactccacgacggtgaggccctcgtgcgagttcgtgcatgcggtatttgtggtagcgatgttgcccgagtcttcaccactggtacgtatcatttccccaccattattggccatgagttctcgggcgaagtcgttgaaacatattctgcggctgacgcggagtgggtgggtcgccgcgtgagcgtttttcccctcatcccttgtcgccattgtgatgcctgtcatcatggtcagtacgaaatgtgcacatgttacaactatctcggcagccgtaccgatggaggcttcgcagagtacgtggctgtcccggtctggaatctctttcgtctgccagattccgtgtcttacgaggagggcgcaatgatggaaccggccgctgtcgctatgcacgcgctcaagattgcccgtgtgggtatgggtgacactgtggcaattacaggtcccggaactattggtatgattttgacccaattgatccaacgtattggcggtacaaaagcggttctgatcggacgcacccaagcaaaattggattacgcacgacgcatcggcatccgtcatgtgtgcaattcgggaaccgagcacgtcgcggaccgcgttggacaacttacaggcggacgtggagccgacatcgcaattgaaggaaccggtgcgtcggaacccatgaatctgtgcttgaatttggtgcgctctgaaggacgtattgtcgctatgggaaacccccaaggtgacatgctgttccgaaaggatgcgtattggaagcttctgcgcaagcagctgacccttcgtggaacctggaattcgacatatggattgggccgagatgattggcaggatatttccgcccttcttgctgagggagcattgaacttgcgcggccttatcacacaccgactcccactggccgagctgcagcagggactcagcctcatgcgagacgcagcagtctacactaacaaagttatgatcgtcgacgagcaaggttaacacggatcaacaaaaatgtgcgccagctttgttgtgtgcccttttcctctagcggatcctatccacatttcgtgaatgaaccgattaagctctgacgagatttccttgttttctgtagatacctcttgtagaggtgtcaaagttatgttgtgatcgagagagttacctgcaaggtattatgcaagcggcttgatgaggacactcaactacgtgtgagtaggagtgcgttgctcagcagttgacgcgtgagccgcttgagcagcttgataaaggtcctaatcgtcttccagctgttggcagacgacaatgtgctgtagtagtaggataggcccgtaaaagtgtagaagcaataggttgagactgactgtaatgggtacttgcgttcaaagactgaaagtccaaaggattgctcaaaggcccctgaggtctgagcctctgagcgcgctcttcgtcaaaattcgccggggccatcggtcatcctctcgagggcagcatcttcctgcttgaagactgagtccgtctcagataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatgcatcatgcatcatgcatcatgcgtcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatgggtaaggaaaagacacacgtatcgcgaccccgactcaactccaacatggacgcagacctgtacggatacaaatgggctcgagataatgtcggacagtcgggtgcgacgatctacagactctatggaaaaccggacgcccccgagctctttcttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactcgggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcg

SEQ ID NO: 68 - DNA, 4594 bp, synthetic construct
  source 1..4594: mol_type = other DNA
tgcagagccttgcaaatgatgaaagaccaatggaattgatcattgattattgatgatgcatgatgaaagagatagaaacagatgcaagatgggatggcgcgatgatcaaatgataatgaacgttcaacccttcaaaattgaaactctcaggctgagcggctgagcaagctctgagaatgtgggtcttcgaagactttcgcttgagcatggtgcatgtgcaaagagatggaatgtggaattgttgtaggtaggaacacagcccggtagagcggcgcagtggctcagcgaagctgggagaacgactgaaccggtcttgagattcatcatctcagcgtgcgtcagcgcgtcagcttcagcgtttgagaatggcacggactgacctgtcgtacatcccaaggaggcccgtgcgctatactcgccttgtggcctaatctgcgatcaacccatcctcaacgaaagcgcaagtagcgactaagcacactatctttcgggtcggtgcggtcacagtatcggcatctctgtctttagctacgatggtgtgcgtgtaactgatcagtacacccgctgtatcaaaactctatcttcaatccgctaagaatcgtcaccatcagtacatcagtacatcagtgggctaagatctaatggtgtcgttcgacggccgcgggcactagccacgagccaagagcggtacagttgctgcgaaatggtgggcagcctgacaggttcagtttgaataggatgaaaacgccatgaagaacagagaagctggcaattgacgatagtggggcgagcgcacgctgcttgctggtccgagcgtaggacgaacgacaaaccatgggcggggccaccaggccccagggagggcgtcactggttggatgtcgtctgttaggctagagctcaggcgctcagggcctctggcccaagcctcggacgcaagcctctgacgagcgagcgcgcgctggggcgggtctgcgcaggaacggcaggactgccgagttggttgaatattgaatggttggatgattggtttgctgggtttggttttgacacgcacggcacaggacaagtgagtttggaggttggatttgcgcaggtagaaagggcgctcggcctcagggcctcagcgtcagcagacgagctcgccagtccaggtcagtacccagtgttcgttacttcccaccgtccagagtttgctgctgcctctcagcttgattcgtgtcggagaagtagaaatggtcacagcgtaccacacactgtccccatgcctgcgtcctgctccacgagtgtttagccacaaaaatttatggacaaccttactgagcctgtgctggggggtgctttagcaccagcactggcgccatggcgtcaaggttaattccactgtcattcgacaaccttcaactgatgggggcaactgggcacaaaaccttggatatataaccaactccaactcccacggttttttaccttttgctcttctccccaggactcactacttctactcctctattcatcttcaccaaaatgtgagacctaatatcactgctgtacagacaagactgtgtatattaggtctcaatgcgcgcctatgagcttcacggagtcaacgacttgcgtcgcgaggacatcgaaaaacctgagattccctctggatgggttcttgtgcaagtcaaagcgagcggcatttgcagctccgacatccctcgtatcttcacgaacggcacttatcactttcctaccattccgggccacgaattctctggagtcgtggccgcctatggcgaaggtgtcccggaagaacgcgttggcaaacgagtgggtatttttccgctgatcccgtgtcgtacatgtccccaatgccgccagaagaaatacgaaatgtgcgagcactatgattaccttggctcgcgtcgtgacggcggattcgccgaatacgttgcagtcccggactggaacctcctggaattgccggagaacgtttcgttccgcgaggcggcgatgctcgaacctctgtcggtggcacttcatgccgtgaaacgcagcggcgtgaaacctggtgatacagccgctgtgatcggcacaggaatgatcggttttgccgcagctgcttgggccaaagcactgggagcagaaacggtgttcgtgattggacgcggcgaggccaaacgcgctattgctgaacaaattcccggaatttcctatgtgactgaagaggaatccaaggccattcaggcggacgtggtcgtcgaagcagttggtacaccacaggctgtttcctccgcggtgttgcttgcacgaccgggaggttccattgttctgatgggcaacccatccggtgatcttgcgatggaaaaaaacgtctactggcgtatcctccgtaagcaacttaacttgatgggaacgtggaatagctcgtatgaaaaagacgaagcctgtgactggaccgaagcgctcgacgcattgtccaagaaaaagattcctgcccaagcacttatcactcattgttttcccagcgaaaaacttatggatggactggaattgatgaagaatcataaggaaccatactgtaaagttatggtcagctgggagtaacacggatcaacaaaaatgtgcgccagctttgttgtgtgcccttttcctctagcggatcctatccacatttcgtgaatgaaccgattaagctctgacgagatttccttgttttctgtagatacctcttgtagaggtgtcaaagttatgttgtgatcgagagagttacctgcaaggtattatgcaagcggcttgatgaggacactcaactacgtgtgagtaggagtgcgttgctcagcagttgacgcgtgagccgcttgagcagcttgataaaggtcctaatcgtcttccagctgttggcagacgacaatgtgctgtagtagtaggataggcccgtaaaagtgtagaagcaataggttgagactgactgtaatgggtacttgcgttcaaagactgaaagtccaaaggattgctcaaaggcccctgaggtctgagcctctgagcgcgctcttcgtcaaaattcgccggggccatcggtcatcctctcgagggcagcatcttcctgcttgaagactgagtccgtctcagataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatgcatcatgcatcatgcatcatgcgtcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatgggtaaggaaaagacacacgtatcgcgaccccgactcaactccaacatggacgcagacctgtacggatacaaatgggctcgagataatgtcggacagtcgggtgcgacgatctacagactctatggaaaaccggacgcccccgagctctttcttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactcgggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcg

SEQ ID NO: 69 - DNA, 4645 bp, synthetic construct
  source 1..4645: mol_type = other DNA
tgcagagccttgcaaatgatgaaagaccaatggaattgatcattgattattgatgatgcatgatgaaagagatagaaacagatgcaagatgggatggcgcgatgatcaaatgataatgaacgttcaacccttcaaaattgaaactctcaggctgagcggctgagcaagctctgagaatgtgggtcttcgaagactttcgcttgagcatggtgcatgtgcaaagagatggaatgtggaattgttgtaggtaggaacacagcccggtagagcggcgcagtggctcagcgaagctgggagaacgactgaaccggtcttgagattcatcatctcagcgtgcgtcagcgcgtcagcttcagcgtttgagaatggcacggactgacctgtcgtacatcccaaggaggcccgtgcgctatactcgccttgtggcctaatctgcgatcaacccatcctcaacgaaagcgcaagtagcgactaagcacactatctttcgggtcggtgcggtcacagtatcggcatctctgtctttagctacgatggtgtgcgtgtaactgatcagtacacccgctgtatcaaaactctatcttcaatccgctaagaatcgtcaccatcagtacatcagtacatcagtgggctaagatctaatggtgtcgttcgacggccgcgggcactagccacgagccaagagcggtacagttgctgcgaaatggtgggcagcctgacaggttcagtttgaataggatgaaaacgccatgaagaacagagaagctggcaattgacgatagtggggcgagcgcacgctgcttgctggtccgagcgtaggacgaacgacaaaccatgggcggggccaccaggccccagggagggcgtcactggttggatgtcgtctgttaggctagagctcaggcgctcagggcctctggcccaagcctcggacgcaagcctctgacgagcgagcgcgcgctggggcgggtctgcgcaggaacggcaggactgccgagttggttgaatattgaatggttggatgattggtttgctgggtttggttttgacacgcacggcacaggacaagtgagtttggaggttggatttgcgcaggtagaaagggcgctcggcctcagggcctcagcgtcagcagacgagctcgccagtccaggtcagtacccagtgttcgttacttcccaccgtccagagtttgctgctgcctctcagcttgattcgtgtcggagaagtagaaatggtcacagcgtaccacacactgtccccatgcctgcgtcctgctccacgagtgtttagccacaaaaatttatggacaaccttactgagcctgtgctggggggtgctttagcaccagcactggcgccatggcgtcaaggttaattccactgtcattcgacaaccttcaactgatgggggcaactgggcacaaaaccttggatatataaccaactccaactcccacggttttttaccttttgctcttctccccaggactcactacttctactcctctattcatcttcaccaaaatgtgagacctaatatcactgctgtacagacaagactgtgtatattaggtctcaatgaagcgtatgcgagctgccatcttgtatggaccaaaagacatccgaattgaaaacattgagatgcccaaggttaagccggagtgggtgcttattaaggttcgcgcaacaggtatctgcggttctgatctgcacttttacaaagagaagagctttattcctattattagcgacgtgggcaaaggacgctacgtccccggtcatgaattttcgggcgaggtctacgagttgggttcgaaggtcaaaggcctgaagaaaggagatcgcgtcggcgtcgaacccatcattggttgcggcaaatgtcagtggtgcaaggtcggatgttacaacttgtgtgagcactacaaattgattggctttcactatattggaggtatggctgagtattgtgttgctcctgctgagaattgttttaagctcccaaacaatgtttcgttcgaaagcgcggcaatgcttgactgtatcgccgtggctattcacgcagtgaataaagcagaaattcgcggcggtgacagcgttctcattgtcggcgccggtgccatcggactcttcgccctccaggcagcgctcatccagggagcgagcgacgtgtacgtcgttggaaaatacgaattccagttggagaaagctcgcaagttgggagcgacagctgtgattaacgctaaaaacgaatctttctccgaaaaaatcatggaaattacttcgggtaaaggagtcgataaggtcattgaggccgtcggcggagcctccaatgttatttctgacgtccttgacgtgactcgaaaaaagggtactatcgtgaccactggtatctttacacgaccactctcggtcgattttttcaaaattctcacaaaagagttgacgatcacttcgtcgtggggatactgttacttcgaccgtcgcaaggagtttggcacggcgctcgactttctggcaaaagaaaagatcaacactcaggaacttattacacataagtatcctttggagaaggtttccgaagcctttgagacggcactcaacaaaaataaggagaagagcatcaaggtggagattatcttctaacacggatcaacaaaaatgtgcgccagctttgttgtgtgcccttttcctctagcggatcctatccacatttcgtgaatgaaccgattaagctctgacgagatttccttgttttctgtagatacctcttgtagaggtgtcaaagttatgttgtgatcgagagagttacctgcaaggtattatgcaagcggcttgatgaggacactcaactacgtgtgagtaggagtgcgttgctcagcagttgacgcgtgagccgcttgagcagcttgataaaggtcctaatcgtcttccagctgttggcagacgacaatgtgctgtagtagtaggataggcccgtaaaagtgtagaagcaataggttgagactgactgtaatgggtacttgcgttcaaagactgaaagtccaaaggattgctcaaaggcccctgaggtctgagcctctgagcgcgctcttcgtcaaaattcgccggggccatcggtcatcctctcgagggcagcatcttcctgcttgaagactgagtccgtctcagataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatgcatcatgcatcatgcatcatgcgtcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatgggtaaggaaaagacacacgtatcgcgaccccgactcaactccaacatggacgcagacctgtacggatacaaatgggctcgagataatgtcggacagtcgggtgcgacgatctacagactctatggaaaaccggacgcccccgagctctttcttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactcgggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcg

SEQ ID NO: 70 - DNA, 4633 bp, synthetic construct
  source 1..4633: mol_type = other DNA
tgcagagccttgcaaatgatgaaagaccaatggaattgatcattgattattgatgatgcatgatgaaagagatagaaacagatgcaagatgggatggcgcgatgatcaaatgataatgaacgttcaacccttcaaaattgaaactctcaggctgagcggctgagcaagctctgagaatgtgggtcttcgaagactttcgcttgagcatggtgcatgtgcaaagagatggaatgtggaattgttgtaggtaggaacacagcccggtagagcggcgcagtggctcagcgaagctgggagaacgactgaaccggtcttgagattcatcatctcagcgtgcgtcagcgcgtcagcttcagcgtttgagaatggcacggactgacctgtcgtacatcccaaggaggcccgtgcgctatactcgccttgtggcctaatctgcgatcaacccatcctcaacgaaagcgcaagtagcgactaagcacactatctttcgggtcggtgcggtcacagtatcggcatctctgtctttagctacgatggtgtgcgtgtaactgatcagtacacccgctgtatcaaaactctatcttcaatccgctaagaatcgtcaccatcagtacatcagtacatcagtgggctaagatctaatggtgtcgttcgacggccgcgggcactagccacgagccaagagcggtacagttgctgcgaaatggtgggcagcctgacaggttcagtttgaataggatgaaaacgccatgaagaacagagaagctggcaattgacgatagtggggcgagcgcacgctgcttgctggtccgagcgtaggacgaacgacaaaccatgggcggggccaccaggccccagggagggcgtcactggttggatgtcgtctgttaggctagagctcaggcgctcagggcctctggcccaagcctcggacgcaagcctctgacgagcgagcgcgcgctggggcgggtctgcgcaggaacggcaggactgccgagttggttgaatattgaatggttggatgattggtttgctgggtttggttttgacacgcacggcacaggacaagtgagtttggaggttggatttgcgcaggtagaaagggcgctcggcctcagggcctcagcgtcagcagacgagctcgccagtccaggtcagtacccagtgttcgttacttcccaccgtccagagtttgctgctgcctctcagcttgattcgtgtcggagaagtagaaatggtcacagcgtaccacacactgtccccatgcctgcgtcctgctccacgagtgtttagccacaaaaatttatggacaaccttactgagcctgtgctggggggtgctttagcaccagcactggcgccatggcgtcaaggttaattccactgtcattcgacaaccttcaactgatgggggcaactgggcacaaaaccttggatatataaccaactccaactcccacggttttttaccttttgctcttctccccaggactcactacttctactcctctattcatcttcaccaaaatgtgagacctaatatcactgctgtacagacaagactgtgtatattaggtctcaatgaaagcctgggtgctgcacggtatcggagatatccaatacgaggaagttaaggagccgcagtcggctggcgacgaggttattatgaaagtgatgagcgtgggaatctgtggatctgacatccctcgcatttataaaaccggcacctatcatttcccaacaatccccggccacgaattctccggtattgtggccgatgttggtgccggagtcaacccgaaatggaagaataagcgcgtcggcgtgtttccattgatcccatgtcacgtttgtgcttcgtgtaaaaaaggtcaatatgaaatgtgtcgcaactacggatatctgggtagccgccaaaatggtggttttgcagagtatgcggcggttcctgtcaagaatctcatcgaactcccggactctgtctctatgcaggaagctgccatgcttgagccgatggccgtggcagtccatgctatgcgacgagcccgtcccatgaccggagatcgtattgtcgtgtgcggacttggaactatcggcatgcttctcgtgtcctttttgcgagagacgggaattcgagacatctttgtcatcggcaataaggcattccagcgaatcaccatcctcaaaactggcctgcaggaaaactgttattgtgattctcgcgtgcaggatgtcgagaagtggctcatggagcgaactgaaggaaagggcgcagacgtgttctttgagtgtgttggccgaaacgaaaccatctcccaagcagtcaacctgacggcacccggaggttcggtctgtctggttggtaatccttgttcggatatgacgcttgagcgtgaggtctactggaaaattctccgtaaccagcttactattactggcacctggaattcctcgttcacccacgagatcgaggacgactggcattacgtgttggatcgcctggaacaaggtaagatctcgcctggtgatctcatttctcacaaattcccgctcaagaacttggaatctggtctgcatattatgcgtgataagactcaagattatatcaaaatcatggccgagattaatgtgtaacacggatcaacaaaaatgtgcgccagctttgttgtgtgcccttttcctctagcggatcctatccacatttcgtgaatgaaccgattaagctctgacgagatttccttgttttctgtagatacctcttgtagaggtgtcaaagttatgttgtgatcgagagagttacctgcaaggtattatgcaagcggcttgatgaggacactcaactacgtgtgagtaggagtgcgttgctcagcagttgacgcgtgagccgcttgagcagcttgataaaggtcctaatcgtcttccagctgttggcagacgacaatgtgctgtagtagtaggataggcccgtaaaagtgtagaagcaataggttgagactgactgtaatgggtacttgcgttcaaagactgaaagtccaaaggattgctcaaaggcccctgaggtctgagcctctgagcgcgctcttcgtcaaaattcgccggggccatcggtcatcctctcgagggcagcatcttcctgcttgaagactgagtccgtctcagataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatgcatcatgcatcatgcatcatgcgtcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatgggtaaggaaaagacacacgtatcgcgaccccgactcaactccaacatggacgcagacctgtacggatacaaatgggctcgagataatgtcggacagtcgggtgcgacgatctacagactctatggaaaaccggacgcccccgagctctttcttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactcgggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcg

SEQ ID NO: 71 - DNA, 4573 bp, synthetic construct
  source 1..4573: mol_type = other DNA
tgcagagccttgcaaatgatgaaagaccaatggaattgatcattgattattgatgatgcatgatgaaagagatagaaacagatgcaagatgggatggcgcgatgatcaaatgataatgaacgttcaacccttcaaaattgaaactctcaggctgagcggctgagcaagctctgagaatgtgggtcttcgaagactttcgcttgagcatggtgcatgtgcaaagagatggaatgtggaattgttgtaggtaggaacacagcccggtagagcggcgcagtggctcagcgaagctgggagaacgactgaaccggtcttgagattcatcatctcagcgtgcgtcagcgcgtcagcttcagcgtttgagaatggcacggactgacctgtcgtacatcccaaggaggcccgtgcgctatactcgccttgtggcctaatctgcgatcaacccatcctcaacgaaagcgcaagtagcgactaagcacactatctttcgggtcggtgcggtcacagtatcggcatctctgtctttagctacgatggtgtgcgtgtaactgatcagtacacccgctgtatcaaaactctatcttcaatccgctaagaatcgtcaccatcagtacatcagtacatcagtgggctaagatctaatggtgtcgttcgacggccgcgggcactagccacgagccaagagcggtacagttgctgcgaaatggtgggcagcctgacaggttcagtttgaataggatgaaaacgccatgaagaacagagaagctggcaattgacgatagtggggcgagcgcacgctgcttgctggtccgagcgtaggacgaacgacaaaccatgggcggggccaccaggccccagggagggcgtcactggttggatgtcgtctgttaggctagagctcaggcgctcagggcctctggcccaagcctcggacgcaagcctctgacgagcgagcgcgcgctggggcgggtctgcgcaggaacggcaggactgccgagttggttgaatattgaatggttggatgattggtttgctgggtttggttttgacacgcacggcacaggacaagtgagtttggaggttggatttgcgcaggtagaaagggcgctcggcctcagggcctcagcgtcagcagacgagctcgccagtccaggtcagtacccagtgttcgttacttcccaccgtccagagtttgctgctgcctctcagcttgattcgtgtcggagaagtagaaatggtcacagcgtaccacacactgtccccatgcctgcgtcctgctccacgagtgtttagccacaaaaatttatggacaaccttactgagcctgtgctggggggtgctttagcaccagcactggcgccatggcgtcaaggttaattccactgtcattcgacaaccttcaactgatgggggcaactgggcacaaaaccttggatatataaccaactccaactcccacggttttttaccttttgctcttctccccaggactcactacttctactcctctattcatcttcaccaaaatgaaggccgcagtgctccattccgaaaatctgtttacgtacgaagaagtggaaatcgatccctgcaaagacgacgaggttaagatcaaggtgatggctgcgggtatctgtggttccgatacacataagatgacgagcaaatggaaatatccccttccgaccatcatgggccatgagttttcgggctttatcgtcgagattggtgaaaaggtcactaacgcgtcgattggcgatcgagttgtggcgatccctttcttgccgtgcaatgactgcgaatactgtaagcgtggacaattctcgttgtgcgaggatcacggcatggttggatccaaaagctttggtggtttcgcggagtacgtcaatatcaaaagcactaatgttcttccgattggtgacatggattttgaggaagcagccatgattgaacctctcgcagttgcgcttcacggcgtgttgaatattcatccgcgtattggagatattgcagccgtctttggtattggcacaatcggaatcctcacaatccaatggcttaaaattgcaggagtgaaagaggttattgcgatcgacatttccgataacaagctcgcagacgcccatgctatgggctgtgagcgtactatcaatccatttaaagagaacctccttgagaaaatcgacgaatatactaacggtcgcggtgttgacatcgcctttgagtgtgctggatctaaagtcacacaagaacagtgcctgctcgtgacagccaagcgaggaaaagtcggctatttgggtatcgcttactctgatgtccatttgtcggagcaagcttttgaaaacattttccgccgcgagctcacacttaaaggattttggaactcttactccgcaccattcccgggagaggaatggaccaaaagcattgacttcattaataagggtcgtattaaactgaaagagctgattagccaccgatttaagctccaagatactcagaaagcgtttgacatgatgatcaaccgagaagagacatacggcaaggtgcttattttgcccaacaaagagtaacacggatcaacaaaaatgtgcgccagctttgttgtgtgcccttttcctctagcggatcctatccacatttcgtgaatgaaccgattaagctctgacgagatttccttgttttctgtagatacctcttgtagaggtgtcaaagttatgttgtgatcgagagagttacctgcaaggtattatgcaagcggcttgatgaggacactcaactacgtgtgagtaggagtgcgttgctcagcagttgacgcgtgagccgcttgagcagcttgataaaggtcctaatcgtcttccagctgttggcagacgacaatgtgctgtagtagtaggataggcccgtaaaagtgtagaagcaataggttgagactgactgtaatgggtacttgcgttcaaagactgaaagtccaaaggattgctcaaaggcccctgaggtctgagcctctgagcgcgctcttcgtcaaaattcgccggggccatcggtcatcctctcgagggcagcatcttcctgcttgaagactgagtccgtctcagataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatgcatcatgcatcatgcatcatgcgtcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatgggtaaggaaaagacacacgtatcgcgaccccgactcaactccaacatggacgcagacctgtacggatacaaatgggctcgagataatgtcggacagtcgggtgcgacgatctacagactctatggaaaaccggacgcccccgagctctttcttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactcgggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcg

SEQ ID NO: 72 - DNA, 4573 bp, synthetic construct
  source 1..4573: mol_type = other DNA
tgcagagccttgcaaatgatgaaagaccaatggaattgatcattgattattgatgatgcatgatgaaagagatagaaacagatgcaagatgggatggcgcgatgatcaaatgataatgaacgttcaacccttcaaaattgaaactctcaggctgagcggctgagcaagctctgagaatgtgggtcttcgaagactttcgcttgagcatggtgcatgtgcaaagagatggaatgtggaattgttgtaggtaggaacacagcccggtagagcggcgcagtggctcagcgaagctgggagaacgactgaaccggtcttgagattcatcatctcagcgtgcgtcagcgcgtcagcttcagcgtttgagaatggcacggactgacctgtcgtacatcccaaggaggcccgtgcgctatactcgccttgtggcctaatctgcgatcaacccatcctcaacgaaagcgcaagtagcgactaagcacactatctttcgggtcggtgcggtcacagtatcggcatctctgtctttagctacgatggtgtgcgtgtaactgatcagtacacccgctgtatcaaaactctatcttcaatccgctaagaatcgtcaccatcagtacatcagtacatcagtgggctaagatctaatggtgtcgttcgacggccgcgggcactagccacgagccaagagcggtacagttgctgcgaaatggtgggcagcctgacaggttcagtttgaataggatgaaaacgccatgaagaacagagaagctggcaattgacgatagtggggcgagcgcacgctgcttgctggtccgagcgtaggacgaacgacaaaccatgggcggggccaccaggccccagggagggcgtcactggttggatgtcgtctgttaggctagagctcaggcgctcagggcctctggcccaagcctcggacgcaagcctctgacgagcgagcgcgcgctggggcgggtctgcgcaggaacggcaggactgccgagttggttgaatattgaatggttggatgattggtttgctgggtttggttttgacacgcacggcacaggacaagtgagtttggaggttggatttgcgcaggtagaaagggcgctcggcctcagggcctcagcgtcagcagacgagctcgccagtccaggtcagtacccagtgttcgttacttcccaccgtccagagtttgctgctgcctctcagcttgattcgtgtcggagaagtagaaatggtcacagcgtaccacacactgtccccatgcctgcgtcctgctccacgagtgtttagccacaaaaatttatggacaaccttactgagcctgtgctggggggtgctttagcaccagcactggcgccatggcgtcaaggttaattccactgtcattcgacaaccttcaactgatgggggcaactgggcacaaaaccttggatatataaccaactccaactcccacggttttttaccttttgctcttctccccaggactcactacttctactcctctattcatcttcaccaaaatgaaggcggcggtgcttcattctgagaatcacttcatttatgaagaaattgatattgctccttgtcaagatgatgaggttaaaatcaaagtcattgcgactggaatttgtggatcggacacgcacaagatgacaggcaaatggaaatatcctctccccgccgtgatgggacacgagttctccggatatatcgttgagaaaggaaagaacgtcactaatgcagccatcggtgaccgcgtcgtggcgatccctttcctcccgtgtaacgactgtgagtattgtaaacgtggccaatttagcctttgcgaagaccacggtatggtcggctcgaagtccttcggaggttttgctgagtacgttaatattgagtcttcgaatgtccttcccatcggagatatggaatttgaagaggcggccatgattgagccgctcgcggtggcccttcatggagtcttgaatattcatccgcgtgttggcgacgtcgcggccgtgttcggtgtcggtaccattggtatgctgaccatccagtggctgaaaattgcgggtgccaaggagatcatcgcaatcgacttgtcggatgaaaaacttgctgacgcgcgtgctatgggttgtgaacgtacgatcaatcccctgaacgaagaccttcttgaaaaaatcaacgaatacaccaacggaatgggagttgacatcgccttcgagtgtgctggctcgaagattacacaagaacagtgccttcttatcacccgcaagcaaggcaaaattggatacctgggcattgcttattccgatgtccacctgtctgagaaggctttcgagaacatttttcgacgcgagcttacgttgaaaggtttctggaattcgtattccgctccatttccgggtgaagaatggacaaaatcgattgaattcattaaaaacggccgcattaagttgaaggaactgatctctcaccgatttaagctggaggatacacaaaaagcatttgatatgatgatcaaccgtgaagagaaatatggtaaggttttgattcttccgaacgaggagtaacacggatcaacaaaaatgtgcgccagctttgttgtgtgcccttttcctctagcggatcctatccacatttcgtgaatgaaccgattaagctctgacgagatttccttgttttctgtagatacctcttgtagaggtgtcaaagttatgttgtgatcgagagagttacctgcaaggtattatgcaagcggcttgatgaggacactcaactacgtgtgagtaggagtgcgttgctcagcagttgacgcgtgagccgcttgagcagcttgataaaggtcctaatcgtcttccagctgttggcagacgacaatgtgctgtagtagtaggataggcccgtaaaagtgtagaagcaataggttgagactgactgtaatgggtacttgcgttcaaagactgaaagtccaaaggattgctcaaaggcccctgaggtctgagcctctgagcgcgctcttcgtcaaaattcgccggggccatcggtcatcctctcgagggcagcatcttcctgcttgaagactgagtccgtctcagataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatgcatcatgcatcatgcatcatgcgtcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatgggtaaggaaaagacacacgtatcgcgaccccgactcaactccaacatggacgcagacctgtacggatacaaatgggctcgagataatgtcggacagtcgggtgcgacgatctacagactctatggaaaaccggacgcccccgagctctttcttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactcgggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcg

SEQ ID NO: 73 - DNA, 4579 bp, synthetic construct
  source 1..4579: mol_type = other DNA
tgcagagccttgcaaatgatgaaagaccaatggaattgatcattgattattgatgatgcatgatgaaagagatagaaacagatgcaagatgggatggcgcgatgatcaaatgataatgaacgttcaacccttcaaaattgaaactctcaggctgagcggctgagcaagctctgagaatgtgggtcttcgaagactttcgcttgagcatggtgcatgtgcaaagagatggaatgtggaattgttgtaggtaggaacacagcccggtagagcggcgcagtggctcagcgaagctgggagaacgactgaaccggtcttgagattcatcatctcagcgtgcgtcagcgcgtcagcttcagcgtttgagaatggcacggactgacctgtcgtacatcccaaggaggcccgtgcgctatactcgccttgtggcctaatctgcgatcaacccatcctcaacgaaagcgcaagtagcgactaagcacactatctttcgggtcggtgcggtcacagtatcggcatctctgtctttagctacgatggtgtgcgtgtaactgatcagtacacccgctgtatcaaaactctatcttcaatccgctaagaatcgtcaccatcagtacatcagtacatcagtgggctaagatctaatggtgtcgttcgacggccgcgggcactagccacgagccaagagcggtacagttgctgcgaaatggtgggcagcctgacaggttcagtttgaataggatgaaaacgccatgaagaacagagaagctggcaattgacgatagtggggcgagcgcacgctgcttgctggtccgagcgtaggacgaacgacaaaccatgggcggggccaccaggccccagggagggcgtcactggttggatgtcgtctgttaggctagagctcaggcgctcagggcctctggcccaagcctcggacgcaagcctctgacgagcgagcgcgcgctggggcgggtctgcgcaggaacggcaggactgccgagttggttgaatattgaatggttggatgattggtttgctgggtttggttttgacacgcacggcacaggacaagtgagtttggaggttggatttgcgcaggtagaaagggcgctcggcctcagggcctcagcgtcagcagacgagctcgccagtccaggtcagtacccagtgttcgttacttcccaccgtccagagtttgctgctgcctctcagcttgattcgtgtcggagaagtagaaatggtcacagcgtaccacacactgtccccatgcctgcgtcctgctccacgagtgtttagccacaaaaatttatggacaaccttactgagcctgtgctggggggtgctttagcaccagcactggcgccatggcgtcaaggttaattccactgtcattcgacaaccttcaactgatgggggcaactgggcacaaaaccttggatatataaccaactccaactcccacggttttttaccttttgctcttctccccaggactcactacttctactcctctattcatcttcaccaaaatgaaggccgcggtcctgcactccgaaaatgagattatttaccaggacattgaaatttccgagtgtcaaccaaacgaagtgaaagttaaggtcatggcggctggaatctgtggttccgatacccataaaatggtgagcaaatggaaataccccctccctgcgatcatgggtcacgaatttgccggctacgtcgttgaaaaaggcaaagacgtgacgaacgtcgaaattggcgatcgtgttgccgctatcccgttcatcccatgcaacgagtgtgagtattgcctgcgaggtcagttcagcctttgcgaaaatcacggaatgatcggcagcaagtcctttggcgcatttgccgagtttgttaatattccagctaagaacgttctcccgattggcgaaatgtctttcgaagatgcagctatgatcgaaccgtctgcggtcagcctgcatggagtgctcaacattcacccacagctcggcgatgtcgttgctgtcttcggtgtgggaactattggaatcctcgcaattcagtggttgcgtgtcgccggcgtgaaagagattattgctattgacatttccgacaagaagctccaggacgctatggatctgggttgtgagaagtcgattaaccctttgaaggaagacttgattaagaaaatttatgaatacacgaatggcctcggcgttgacatcgcatttgaatgcgctggatctaaaattacgcaagaacagtgcctgctcattacccgtaaacaaggaaaggttggttatttgggaattgcgtacagcgatgttactctctctgaaaaagcctttgaaaatattttccgccgagagctcactctcaagggtttctggaattcgtattccgcgccgtttccaggaaaggagtggatgaacacgatcgagttcgtccaacagggcaaaattaagcttaaagaattggtgtcgcatcgctacaagctcgaagacacccagaaggcgtttaacatgatgatcaaccgagaagaagaatacaataaggtgatgatcatccccaacgaggaactggagtaacacggatcaacaaaaatgtgcgccagctttgttgtgtgcccttttcctctagcggatcctatccacatttcgtgaatgaaccgattaagctctgacgagatttccttgttttctgtagatacctcttgtagaggtgtcaaagttatgttgtgatcgagagagttacctgcaaggtattatgcaagcggcttgatgaggacactcaactacgtgtgagtaggagtgcgttgctcagcagttgacgcgtgagccgcttgagcagcttgataaaggtcctaatcgtcttccagctgttggcagacgacaatgtgctgtagtagtaggataggcccgtaaaagtgtagaagcaataggttgagactgactgtaatgggtacttgcgttcaaagactgaaagtccaaaggattgctcaaaggcccctgaggtctgagcctctgagcgcgctcttcgtcaaaattcgccggggccatcggtcatcctctcgagggcagcatcttcctgcttgaagactgagtccgtctcagataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatgcatcatgcatcatgcatcatgcgtcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatgggtaaggaaaagacacacgtatcgcgaccccgactcaactccaacatggacgcagacctgtacggatacaaatgggctcgagataatgtcggacagtcgggtgcgacgatctacagactctatggaaaaccggacgcccccgagctctttcttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactcgggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcg

SEQ ID NO: 74 - DNA, 4567 bp, synthetic construct
  source 1..4567: mol_type = other DNA
tgcagagccttgcaaatgatgaaagaccaatggaattgatcattgattattgatgatgcatgatgaaagagatagaaacagatgcaagatgggatggcgcgatgatcaaatgataatgaacgttcaacccttcaaaattgaaactctcaggctgagcggctgagcaagctctgagaatgtgggtcttcgaagactttcgcttgagcatggtgcatgtgcaaagagatggaatgtggaattgttgtaggtaggaacacagcccggtagagcggcgcagtggctcagcgaagctgggagaacgactgaaccggtcttgagattcatcatctcagcgtgcgtcagcgcgtcagcttcagcgtttgagaatggcacggactgacctgtcgtacatcccaaggaggcccgtgcgctatactcgccttgtggcctaatctgcgatcaacccatcctcaacgaaagcgcaagtagcgactaagcacactatctttcgggtcggtgcggtcacagtatcggcatctctgtctttagctacgatggtgtgcgtgtaactgatcagtacacccgctgtatcaaaactctatcttcaatccgctaagaatcgtcaccatcagtacatcagtacatcagtgggctaagatctaatggtgtcgttcgacggccgcgggcactagccacgagccaagagcggtacagttgctgcgaaatggtgggcagcctgacaggttcagtttgaataggatgaaaacgccatgaagaacagagaagctggcaattgacgatagtggggcgagcgcacgctgcttgctggtccgagcgtaggacgaacgacaaaccatgggcggggccaccaggccccagggagggcgtcactggttggatgtcgtctgttaggctagagctcaggcgctcagggcctctggcccaagcctcggacgcaagcctctgacgagcgagcgcgcgctggggcgggtctgcgcaggaacggcaggactgccgagttggttgaatattgaatggttggatgattggtttgctgggtttggttttgacacgcacggcacaggacaagtgagtttggaggttggatttgcgcaggtagaaagggcgctcggcctcagggcctcagcgtcagcagacgagctcgccagtccaggtcagtacccagtgttcgttacttcccaccgtccagagtttgctgctgcctctcagcttgattcgtgtcggagaagtagaaatggtcacagcgtaccacacactgtccccatgcctgcgtcctgctccacgagtgtttagccacaaaaatttatggacaaccttactgagcctgtgctggggggtgctttagcaccagcactggcgccatggcgtcaaggttaattccactgtcattcgacaaccttcaactgatgggggcaactgggcacaaaaccttggatatataaccaactccaactcccacggttttttaccttttgctcttctccccaggactcactacttctactcctctattcatcttcaccaaaatgaaggcagcggtgcttcattcggtcggagacatccgctacgaaaacatcgagatcaagccttacggagacgatgaagtgaaaatcaaagttaaagccgccggtatctgtggatctgacccgccacgtgcactcaaaaagtggaaatatcccgtgccagccattttgggtcacgagttctcgggttatatcgttgaagtgggaaagaacgtgaagggttttaaagtgggagatcgtgtcgttgccgtccctctggaaccgtgtcaccaatgtgaatactgcaagaaaggccaattttcgctctgcgaaagctacgatatgttgggtgctacttcctttggcggttttgcagagtacgctaacgtgaaggcaacaaatgttttgaaaattgacgatatcgactatgaagaggcggccatgattgaacctctcgcagtcgcactgcatggtgtgcttaatatcaatccgcaactcggtgatactgttgctgttttgggagctggaacgatcggtcaactcacgattcaatggctcaaggtttcgggagtcgagaagattatcgcagtcgacatttcggacaaaaaaattagcgaagctatgtccttgggagcagatattggtattaatgctcttaaagagaatccggtcgaaaagatcatggaacttacaaacggtttcggcgtggatatctgcattgaatgcgccggctctaagatcactcaggaacaatgtctgttggtcacgaagaaaaaaggtaagattggatatcagggtatcggtcacgccggaattgagttgtctgaagttgctttcgagggtattttccgtaaagaattgaacatccaaggtttttggaatagctacagcgccccgttccccggacaggaatggttcaaaagcatcgagtatgttaagcagaaaaaaatcaagctcaaacagttgatcagccatcgcttcagccttgaagacacagcgaaagcttttgagatgattggtgaacgtaaggaggagtataacaagatcatgattgtccaggactaacacggatcaacaaaaatgtgcgccagctttgttgtgtgcccttttcctctagcggatcctatccacatttcgtgaatgaaccgattaagctctgacgagatttccttgttttctgtagatacctcttgtagaggtgtcaaagttatgttgtgatcgagagagttacctgcaaggtattatgcaagcggcttgatgaggacactcaactacgtgtgagtaggagtgcgttgctcagcagttgacgcgtgagccgcttgagcagcttgataaaggtcctaatcgtcttccagctgttggcagacgacaatgtgctgtagtagtaggataggcccgtaaaagtgtagaagcaataggttgagactgactgtaatgggtacttgcgttcaaagactgaaagtccaaaggattgctcaaaggcccctgaggtctgagcctctgagcgcgctcttcgtcaaaattcgccggggccatcggtcatcctctcgagggcagcatcttcctgcttgaagactgagtccgtctcagataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatgcatcatgcatcatgcatcatgcgtcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatgggtaaggaaaagacacacgtatcgcgaccccgactcaactccaacatggacgcagacctgtacggatacaaatgggctcgagataatgtcggacagtcgggtgcgacgatctacagactctatggaaaaccggacgcccccgagctctttcttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactcgggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcg

SEQ ID NO: 75 - DNA, 4573 bp, synthetic construct
  source 1..4573: mol_type = other DNA
tgcagagccttgcaaatgatgaaagaccaatggaattgatcattgattattgatgatgcatgatgaaagagatagaaacagatgcaagatgggatggcgcgatgatcaaatgataatgaacgttcaacccttcaaaattgaaactctcaggctgagcggctgagcaagctctgagaatgtgggtcttcgaagactttcgcttgagcatggtgcatgtgcaaagagatggaatgtggaattgttgtaggtaggaacacagcccggtagagcggcgcagtggctcagcgaagctgggagaacgactgaaccggtcttgagattcatcatctcagcgtgcgtcagcgcgtcagcttcagcgtttgagaatggcacggactgacctgtcgtacatcccaaggaggcccgtgcgctatactcgccttgtggcctaatctgcgatcaacccatcctcaacgaaagcgcaagtagcgactaagcacactatctttcgggtcggtgcggtcacagtatcggcatctctgtctttagctacgatggtgtgcgtgtaactgatcagtacacccgctgtatcaaaactctatcttcaatccgctaagaatcgtcaccatcagtacatcagtacatcagtgggctaagatctaatggtgtcgttcgacggccgcgggcactagccacgagccaagagcggtacagttgctgcgaaatggtgggcagcctgacaggttcagtttgaataggatgaaaacgccatgaagaacagagaagctggcaattgacgatagtggggcgagcgcacgctgcttgctggtccgagcgtaggacgaacgacaaaccatgggcggggccaccaggccccagggagggcgtcactggttggatgtcgtctgttaggctagagctcaggcgctcagggcctctggcccaagcctcggacgcaagcctctgacgagcgagcgcgcgctggggcgggtctgcgcaggaacggcaggactgccgagttggttgaatattgaatggttggatgattggtttgctgggtttggttttgacacgcacggcacaggacaagtgagtttggaggttggatttgcgcaggtagaaagggcgctcggcctcagggcctcagcgtcagcagacgagctcgccagtccaggtcagtacccagtgttcgttacttcccaccgtccagagtttgctgctgcctctcagcttgattcgtgtcggagaagtagaaatggtcacagcgtaccacacactgtccccatgcctgcgtcctgctccacgagtgtttagccacaaaaatttatggacaaccttactgagcctgtgctggggggtgctttagcaccagcactggcgccatggcgtcaaggttaattccactgtcattcgacaaccttcaactgatgggggcaactgggcacaaaaccttggatatataaccaactccaactcccacggttttttaccttttgctcttctccccaggactcactacttctactcctctattcatcttcaccaaaatgaaagcagcagttttgcattccgagaatcacttcatttatcaggatatggagagcttcgagtgccagaatgacgaagtcaagatcaaggttatggctgccggcatctgcggaagcgatacgcataaaatggtttcgaaatggaaatatcccttgcctgcaattatgggacatgagttctccggttacgtcgtcgaaaagggtagcgatgtggcaaacgtcgagatcggcgaccgagtggttgccattccattcttgccctgtcacacttgtgagtattgcaagtccggtgtttattctttgtgtgaagatcacggtatgatcggttccaagagctacggtggcttcgcggagtatgtcaatctcaaggcaaccaacgtccttccaattggaaacatggattttgaggaggccgctatgatcgaaccgcttgcagtcgccctccacggcgttctccatattcagccgcgtctcggtgacgtggccgccgtttttggagtcggcaccattggcatgttggccatccaatggctgaagattgcgggagtgaaagaaattatcgctattgatatttcggatgagaagttggcggaagcgaaagccatgggatgcgaacgaacgattaatcccctccaagacaatctcatcgaaaaggtccacgagtataccaacggcttgggcgtggatgtcgccctcgagtgcgcgggatctaagattacacaggaacaatgtcttttgatcactcgcaaaaagggtgaaattggttacttgggaatcgcgtattctaatgtggttttgagcgaacaagcttttgagaacatctttcgtaaagagcttacgttgaagggtttttggaattgttatagcgctcccttccctggcgaggagtggacgaaatctatcgacttcattctgaagggccgcatcaagctgaaggagcttatcagccaccgctttcccctggaagaaacgcagaaggcattcgacctgatgattaatcgaaaagaaacctatggtaaggtgatgatcatccccaataaggaataacacggatcaacaaaaatgtgcgccagctttgttgtgtgcccttttcctctagcggatcctatccacatttcgtgaatgaaccgattaagctctgacgagatttccttgttttctgtagatacctcttgtagaggtgtcaaagttatgttgtgatcgagagagttacctgcaaggtattatgcaagcggcttgatgaggacactcaactacgtgtgagtaggagtgcgttgctcagcagttgacgcgtgagccgcttgagcagcttgataaaggtcctaatcgtcttccagctgttggcagacgacaatgtgctgtagtagtaggataggcccgtaaaagtgtagaagcaataggttgagactgactgtaatgggtacttgcgttcaaagactgaaagtccaaaggattgctcaaaggcccctgaggtctgagcctctgagcgcgctcttcgtcaaaattcgccggggccatcggtcatcctctcgagggcagcatcttcctgcttgaagactgagtccgtctcagataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatgcatcatgcatcatgcatcatgcgtcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatgggtaaggaaaagacacacgtatcgcgaccccgactcaactccaacatggacgcagacctgtacggatacaaatgggctcgagataatgtcggacagtcgggtgcgacgatctacagactctatggaaaaccggacgcccccgagctctttcttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactcgggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcg

SEQ ID NO: 76 - DNA, 4573 bp, synthetic construct
  source 1..4573: mol_type = other DNA
tgcagagccttgcaaatgatgaaagaccaatggaattgatcattgattattgatgatgcatgatgaaagagatagaaacagatgcaagatgggatggcgcgatgatcaaatgataatgaacgttcaacccttcaaaattgaaactctcaggctgagcggctgagcaagctctgagaatgtgggtcttcgaagactttcgcttgagcatggtgcatgtgcaaagagatggaatgtggaattgttgtaggtaggaacacagcccggtagagcggcgcagtggctcagcgaagctgggagaacgactgaaccggtcttgagattcatcatctcagcgtgcgtcagcgcgtcagcttcagcgtttgagaatggcacggactgacctgtcgtacatcccaaggaggcccgtgcgctatactcgccttgtggcctaatctgcgatcaacccatcctcaacgaaagcgcaagtagcgactaagcacactatctttcgggtcggtgcggtcacagtatcggcatctctgtctttagctacgatggtgtgcgtgtaactgatcagtacacccgctgtatcaaaactctatcttcaatccgctaagaatcgtcaccatcagtacatcagtacatcagtgggctaagatctaatggtgtcgttcgacggccgcgggcactagccacgagccaagagcggtacagttgctgcgaaatggtgggcagcctgacaggttcagtttgaataggatgaaaacgccatgaagaacagagaagctggcaattgacgatagtggggcgagcgcacgctgcttgctggtccgagcgtaggacgaacgacaaaccatgggcggggccaccaggccccagggagggcgtcactggttggatgtcgtctgttaggctagagctcaggcgctcagggcctctggcccaagcctcggacgcaagcctctgacgagcgagcgcgcgctggggcgggtctgcgcaggaacggcaggactgccgagttggttgaatattgaatggttggatgattggtttgctgggtttggttttgacacgcacggcacaggacaagtgagtttggaggttggatttgcgcaggtagaaagggcgctcggcctcagggcctcagcgtcagcagacgagctcgccagtccaggtcagtacccagtgttcgttacttcccaccgtccagagtttgctgctgcctctcagcttgattcgtgtcggagaagtagaaatggtcacagcgtaccacacactgtccccatgcctgcgtcctgctccacgagtgtttagccacaaaaatttatggacaaccttactgagcctgtgctggggggtgctttagcaccagcactggcgccatggcgtcaaggttaattccactgtcattcgacaaccttcaactgatgggggcaactgggcacaaaaccttggatatataaccaactccaactcccacggttttttaccttttgctcttctccccaggactcactacttctactcctctattcatcttcaccaaaatgaaagcagcagtgctccattctgagaatcagatcgtgtttcaagaatttgagatcggcgaacttaagccagacgaagttaaggtcaaagtgatggcagcgggcatctgtggctcggatacccacaagatggtgtcgaagtggaagtatcccttgccagcaatcatgggtcatgagtttgccggctatattgtcgaaaagggaaaagatgtggtgcatgtggaagtcggtgatcgtgtggcaggcatcccttttgtgccttgtaatgactgtgagtactgttttcgcggtcagttttccctttgcgagaatcacggtatggtgggttctaagtctttcggagcctttgcggaatatgttaacattccagccaaaaacgtgctgccgattggagacatggattttgaggacgcggcgatgattgagccttcggccgttgccctccatggagtcttgaacattcacccccaaatcggtgatgttgtggcggtttttggagttggcaccattggaatccttgcactccagtggttgaaagttgccggtgttaaagaaattattgccattgacatctcggacaagaagctccaggatgccatcgccctcggatgtgagcgcacgattaatccattgaaagagaatttgctggaaaaaattcatgagtatacgaatggtcttggagtggacatcgccattgaatgtgcgggcagcaagattacacaagaacagtgtcttcttattacgcgcaagcagggcaaagtcggctatcttggcatcgcttactccgatattaatctgtcggaaaaagcatttgaaaacatcttccgccgcgaactcaaaattaatggtttctggaattcttattcggctccgttcccaggtaaagagtggacaaacaccgtggagtatgtgaagcaaggcaagattaagctgaaggagcttatttcccaccgattcggactggaagacacgcagcaagcatttaatatgattatcaaccgagaggaggaatataataaagttatgattgtcccaaacggcgagtaacacggatcaacaaaaatgtgcgccagctttgttgtgtgcccttttcctctagcggatcctatccacatttcgtgaatgaaccgattaagctctgacgagatttccttgttttctgtagatacctcttgtagaggtgtcaaagttatgttgtgatcgagagagttacctgcaaggtattatgcaagcggcttgatgaggacactcaactacgtgtgagtaggagtgcgttgctcagcagttgacgcgtgagccgcttgagcagcttgataaaggtcctaatcgtcttccagctgttggcagacgacaatgtgctgtagtagtaggataggcccgtaaaagtgtagaagcaataggttgagactgactgtaatgggtacttgcgttcaaagactgaaagtccaaaggattgctcaaaggcccctgaggtctgagcctctgagcgcgctcttcgtcaaaattcgccggggccatcggtcatcctctcgagggcagcatcttcctgcttgaagactgagtccgtctcagataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatgcatcatgcatcatgcatcatgcgtcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatgggtaaggaaaagacacacgtatcgcgaccccgactcaactccaacatggacgcagacctgtacggatacaaatgggctcgagataatgtcggacagtcgggtgcgacgatctacagactctatggaaaaccggacgcccccgagctctttcttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactcgggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcg

SEQ ID NO: 77 - DNA, 4533 bp, synthetic construct
  source 1..4533: mol_type = other DNA
tgcagagccttgcaaatgatgaaagaccaatggaattgatcattgattattgatgatgcatgatgaaagagatagaaacagatgcaagatgggatggcgcgatgatcaaatgataatgaacgttcaacccttcaaaattgaaactctcaggctgagcggctgagcaagctctgagaatgtgggtcttcgaagactttcgcttgagcatggtgcatgtgcaaagagatggaatgtggaattgttgtaggtaggaacacagcccggtagagcggcgcagtggctcagcgaagctgggagaacgactgaaccggtcttgagattcatcatctcagcgtgcgtcagcgcgtcagcttcagcgtttgagaatggcacggactgacctgtcgtacatcccaaggaggcccgtgcgctatactcgccttgtggcctaatctgcgatcaacccatcctcaacgaaagcgcaagtagcgactaagcacactatctttcgggtcggtgcggtcatttagctacgatggtgtgcgtgtaactgatcagtacacccgctgtatcaaaactctatcttcaatccgctaagaatcgtcaccatcagtacatcagtacatcagtgggctaagatctaatggtgtcgttcgacggccgcgggcactagccacgagccaagagcggtacagttgctgcgaaatggtgggcagcctgacaggttcagtttgaataggatgaaaacgccatgaagaacagagaagctggcaattgacgatagtggggcgagcgcacgctgcttgctggtccgagcgtaggacgaacgacaaaccatgggcggggccaccaggccccagggagggcgtcactggttggatgtcgtctgttaggctagagctcaggcgctcagggcctctggcccaagcctcggacgcaagcctctgacgagcgagcgcgcgctggggcgggtctgcgcaggaacggcaggactgccgagttggttgaatattgaatggttggatgattggtttgctgggtttggttttgacacgcacggcacaggacaagtgagtttggaggttggatttgcgcaggtagaaagggcgctcggcctcagggcctcagcgtcagcagacgagctcgccagtccaggtcagtacccagtgttcgttacttcccaccgtccagagtttgctgctgcctctcagcttgattcgtgtcggagaagtagaaatggtcacagcgtaccacacactgtccccatgcctgcgtcctgctccacgagtgtttagccacaaaaatttatggacaaccttactgagcctgtgctggggggtgctttagcaccagcactggcgccatggcgtcaaggttaattccactgtcattcgacaaccttcaactgatgggggcaactgggcacaaaaccttggatatataaccaactccaactcccacggttttttaccttttgctcttctccccaggactcactacttctactcctctattcatcttcaccaaaatgattaatcaggtttaccagctggtggcgccgcgccagttcgacgtcacgtataataacgtggacatttacggcaaccacgttatcgtccgaccgctgtatctctcgatctgcgcggccgatcaacgatactataccggacgtcgtgatgagaatgtgctgcgaaaaaaattgccaatgtctctggtccatgaagccgttggtgaggttgtttttgatagcaagggagtgtttgagaagggcacaaaggttgttatggtcccaaatacgcctacggagcaacaccatattatcgccgagaactacctggcgtcttcgtatttccgctcgagcggttacgatggtttcatgcaggattatgttgttatggcgcatgaccgtattgtcccgctgcccaacgatattgacctgagcactatttcctatacagaattggtttcggttagctaccacgccattcaacgtttcgagcgtaagtccattccgctcaagaatagcttcggtatttggggagacggtaatttgggctacatcactgcaatccttcttcgcaagctctacccggaggcaaaaatttatgtcttcggaaaaacggattataaactttctcatttcagcttcgttgatgacatcttcacggtgaaccaaatccccgatgatttgaagattgaccacgccttcgagtgcgttggcggaaagggctcgcaagttgcgttgcagcaaatcgttgagcatatctcccctgaaggttctattgctctcttgggcgtgtccgaactccccgtcgaagttaacacccgactcgtgttggaaaagggcttgaccctgattggatcctctcgttccggttccaaggactttgagcaggttgtcgacctctatcgaaaatatcccgatattgtcgagaaactcgcacttttgaaaggccacgaaattaatgtttgtacacttcaggatatcgtgcaggcatttgaaatggacctctctacgtcttggggcaagacagttttgaagtggacgatctaacacggatcaacaaaaatgtgcgccagctttgttgtgtgcccttttcctctagcggatcctatccacatttcgtgaatgaaccgattaagctctgacgagatttccttgttttctgtagatacctcttgtagaggtgtcaaagttatgttgtgatcgagagagttacctgcaaggtattatgcaagcggcttgatgaggacactcaactacgtgtgagtaggagtgcgttgctcagcagttgacgcgtgagccgcttgagcagcttgataaaggtcctaatcgtcttccagctgttggcagacgacaatgtgctgtagtagtaggataggcccgtaaaagtgtagaagcaataggttgagactgactgtaatgggtacttgcgttcaaagactgaaagtccaaaggattgctcaaaggcccctgaggtctgagcctctgagcgcgctcttcgtcaaaattcgccggggccatcggtcatcctctcgagggcagcatcttcctgcttgaagactgagtccgtctcagataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatgcatcatgcatcatgcatcatgcgtcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatgggtaaggaaaagacacacgtatcgcgaccccgactcaactccaacatggacgcagacctgtacggatacaaatgggctcgagataatgtcggacagtcgggtgcgacgatctacagactctatggaaaaccggacgcccccgagctctttcttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactcgggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcg

SEQ ID NO: 78 - DNA, 4633 bp, synthetic construct
  source 1..4633: mol_type = other DNA
tgcagagccttgcaaatgatgaaagaccaatggaattgatcattgattattgatgatgcatgatgaaagagatagaaacagatgcaagatgggatggcgcgatgatcaaatgataatgaacgttcaacccttcaaaattgaaactctcaggctgagcggctgagcaagctctgagaatgtgggtcttcgaagactttcgcttgagcatggtgcatgtgcaaagagatggaatgtggaattgttgtaggtaggaacacagcccggtagagcggcgcagtggctcagcgaagctgggagaacgactgaaccggtcttgagattcatcatctcagcgtgcgtcagcgcgtcagcttcagcgtttgagaatggcacggactgacctgtcgtacatcccaaggaggcccgtgcgctatactcgccttgtggcctaatctgcgatcaacccatcctcaacgaaagcgcaagtagcgactaagcacactatctttcgggtcggtgcggtcacagtatcggcatctctgtctttagctacgatggtgtgcgtgtaactgatcagtacacccgctgtatcaaaactctatcttcaatccgctaagaatcgtcaccatcagtacatcagtacatcagtgggctaagatctaatggtgtcgttcgacggccgcgggcactagccacgagccaagagcggtacagttgctgcgaaatggtgggcagcctgacaggttcagtttgaataggatgaaaacgccatgaagaacagagaagctggcaattgacgatagtggggcgagcgcacgctgcttgctggtccgagcgtaggacgaacgacaaaccatgggcggggccaccaggccccagggagggcgtcactggttggatgtcgtctgttaggctagagctcaggcgctcagggcctctggcccaagcctcggacgcaagcctctgacgagcgagcgcgcgctggggcgggtctgcgcaggaacggcaggactgccgagttggttgaatattgaatggttggatgattggtttgctgggtttggttttgacacgcacggcacaggacaagtgagtttggaggttggatttgcgcaggtagaaagggcgctcggcctcagggcctcagcgtcagcagacgagctcgccagtccaggtcagtacccagtgttcgttacttcccaccgtccagagtttgctgctgcctctcagcttgattcgtgtcggagaagtagaaatggtcacagcgtaccacacactgtccccatgcctgcgtcctgctccacgagtgtttagccacaaaaatttatggacaaccttactgagcctgtgctggggggtgctttagcaccagcactggcgccatggcgtcaaggttaattccactgtcattcgacaaccttcaactgatgggggcaactgggcacaaaaccttggatatataaccaactccaactcccacggttttttaccttttgctcttctccccaggactcactacttctactcctctattcatcttcaccaaaatgtgagacctaatatcactgctgtacagacaagactgtgtatattaggtctcaatgttgttgtttgaagttgttctcccaatgatcaaccaagtttatcaacttgtcgcaccccgccaatttgaggtgacgtacaataacgtggacatttacagcgactatgttattgtccgacctctttacatgagcatttgcgctgccgaccagcgatactacaccggatcgcgtgacgagaatgtcctttcccaaaaactgcctatgtctctcattcacgaaggagtgggtgaggttgttttcgattccaagggagtctttaataagggcactaaggtggttatggtccccaatacaccgaccgagaaagacgacgtgattgcagagaattatcttaaaagctcgtattttcgttcctccggccatgacggtttcatgcaggacttcgttctccttaatcacgatcgagctgtcccgctgccggacgacatcgatctctccatcatttcctacacagagttggtgacagtttctctccatgccatccgccgatttgaaaaaaaatccatcagcaataaaaacaccttcggcatttggggagacggaaatcttggttatattaccgcaattctcctgcgcaagctctacccagaatctaaaatttacgtgtttggtaaaactgattataaactgagccatttctccttcgtggacgacgttttcttcatcaataagattcctgaaggacttacctttgatcacgcctttgagtgcgtcggtggacgaggctctcaatccgcaattaaccaaatgattgactatatttcgcccgaaggtagcatcgccttgctcggtgtttccgagttcccggttgaggtcaatacgcgcctcgtcctcgagaagggactgaccttgatcggctcttctcgctctggttctaaagactttcaagacgtggttgacctctatattcaatacccggatattgtcgacaaattggcgctccttaaaggacaggagttcgaaattgccacaattaatgacctgacagaggcgtttgaggctgacctctccacctcctggggaaagacggttttgaagtggattatgtaacacggatcaacaaaaatgtgcgccagctttgttgtgtgcccttttcctctagcggatcctatccacatttcgtgaatgaaccgattaagctctgacgagatttccttgttttctgtagatacctcttgtagaggtgtcaaagttatgttgtgatcgagagagttacctgcaaggtattatgcaagcggcttgatgaggacactcaactacgtgtgagtaggagtgcgttgctcagcagttgacgcgtgagccgcttgagcagcttgataaaggtcctaatcgtcttccagctgttggcagacgacaatgtgctgtagtagtaggataggcccgtaaaagtgtagaagcaataggttgagactgactgtaatgggtacttgcgttcaaagactgaaagtccaaaggattgctcaaaggcccctgaggtctgagcctctgagcgcgctcttcgtcaaaattcgccggggccatcggtcatcctctcgagggcagcatcttcctgcttgaagactgagtccgtctcagataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatgcatcatgcatcatgcatcatgcgtcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatgggtaaggaaaagacacacgtatcgcgaccccgactcaactccaacatggacgcagacctgtacggatacaaatgggctcgagataatgtcggacagtcgggtgcgacgatctacagactctatggaaaaccggacgcccccgagctctttcttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactcgggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcg

SEQ ID NO: 79 - DNA, 4552 bp, synthetic construct
  source 1..4552: mol_type = other DNA
tgcagagccttgcaaatgatgaaagaccaatggaattgatcattgattattgatgatgcatgatgaaagagatagaaacagatgcaagatgggatggcgcgatgatcaaatgataatgaacgttcaacccttcaaaattgaaactctcaggctgagcggctgagcaagctctgagaatgtgggtcttcgaagactttcgcttgagcatggtgcatgtgcaaagagatggaatgtggaattgttgtaggtaggaacacagcccggtagagcggcgcagtggctcagcgaagctgggagaacgactgaaccggtcttgagattcatcatctcagcgtgcgtcagcgcgtcagcttcagcgtttgagaatggcacggactgacctgtcgtacatcccaaggaggcccgtgcgctatactcgccttgtggcctaatctgcgatcaacccatcctcaacgaaagcgcaagtagcgactaagcacactatctttcgggtcggtgcggtcacagtatcggcatctctgtctttagctacgatggtgtgcgtgtaactgatcagtacacccgctgtatcaaaactctatcttcaatccgctaagaatcgtcaccatcagtacatcagtacatcagtgggctaagatctaatggtgtcgttcgacggccgcgggcactagccacgagccaagagcggtacagttgctgcgaaatggtgggcagcctgacaggttcagtttgaataggatgaaaacgccatgaagaacagagaagctggcaattgacgatagtggggcgagcgcacgctgcttgctggtccgagcgtaggacgaacgacaaaccatgggcggggccaccaggccccagggagggcgtcactggttggatgtcgtctgttaggctagagctcaggcgctcagggcctctggcccaagcctcggacgcaagcctctgacgagcgagcgcgcgctggggcgggtctgcgcaggaacggcaggactgccgagttggttgaatattgaatggttggatgattggtttgctgggtttggttttgacacgcacggcacaggacaagtgagtttggaggttggatttgcgcaggtagaaagggcgctcggcctcagggcctcagcgtcagcagacgagctcgccagtccaggtcagtacccagtgttcgttacttcccaccgtccagagtttgctgctgcctctcagcttgattcgtgtcggagaagtagaaatggtcacagcgtaccacacactgtccccatgcctgcgtcctgctccacgagtgtttagccacaaaaatttatggacaaccttactgagcctgtgctggggggtgctttagcaccagcactggcgccatggcgtcaaggttaattccactgtcattcgacaaccttcaactgatgggggcaactgggcacaaaaccttggatatataaccaactccaactcccacggttttttaccttttgctcttctccccaggactcactacttctactcctctattcatcttcaccaaaatgattaataccgtctaccaactggtggcacctcgacgattcgagattaagtattccgacattgatctcaacaataataaggttatcgtccgtcccacctacctctctatctgcaacgcagaccaacgatactaccaaggactccgcgatgccgaaattctgcgtaagaaacttccgatggcgttgatccatgaagccattggagaggtcgtgcgagatccatctggaaactttaaaaccggcgagttggtcgttatggtgccgaacacacctgttgaagaagatgacatcatcgctgagaattatttgcgatcctcgaaatttcgtgcatcgggttttgacggcttcatgcaagaatatgtggaaatcacaccggatcgcctcgttcgtcttccccagaatatcgaccgtactgttgcagcttttacggaaattgtttctgtcagcgtgcatgcaatcggtcgttttgacaagatcgcgcacaaacgacgaaacgttgtcggcatctggggagacggcaacctgggatatattactgcggttttttttaagactatgtttccagagacgaagctgtatatttttggagtcaacccagataaacttcaagattttacgtttgccgacgcgacgtttacggtggagcacattcctgaagacgttaagattgaccatgcttttgagtgcgtgggtggcgcaggctcgggcaaagccattaatcagattatcgactatatcaatcccgaaggtaccatttccattttgggcgtttctgaatacccagtgccaattaacacccgcatggtcctcgagaaaggactgcgcatctttggatcgagccgtagcggtgtcgccgattttgagaagaccgtcgcgctctacaaatccaatcctgagattatcaattatctttcgaatatcgtcggtgcagtcgtcccagtgcgaacgatcgaagatatgaaaaaggcgttcgaaatggacatccaaaaggcttttggtaagacgattatgatgtgggataaataacacggatcaacaaaaatgtgcgccagctttgttgtgtgcccttttcctctagcggatcctatccacatttcgtgaatgaaccgattaagctctgacgagatttccttgttttctgtagatacctcttgtagaggtgtcaaagttatgttgtgatcgagagagttacctgcaaggtattatgcaagcggcttgatgaggacactcaactacgtgtgagtaggagtgcgttgctcagcagttgacgcgtgagccgcttgagcagcttgataaaggtcctaatcgtcttccagctgttggcagacgacaatgtgctgtagtagtaggataggcccgtaaaagtgtagaagcaataggttgagactgactgtaatgggtacttgcgttcaaagactgaaagtccaaaggattgctcaaaggcccctgaggtctgagcctctgagcgcgctcttcgtcaaaattcgccggggccatcggtcatcctctcgagggcagcatcttcctgcttgaagactgagtccgtctcagataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatgcatcatgcatcatgcatcatgcgtcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatgggtaaggaaaagacacacgtatcgcgaccccgactcaactccaacatggacgcagacctgtacggatacaaatgggctcgagataatgtcggacagtcgggtgcgacgatctacagactctatggaaaaccggacgcccccgagctctttcttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactcgggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcg

SEQ ID NO: 80 - DNA, 4558 bp, synthetic construct
  source 1..4558: mol_type = other DNA
tgcagagccttgcaaatgatgaaagaccaatggaattgatcattgattattgatgatgcatgatgaaagagatagaaacagatgcaagatgggatggcgcgatgatcaaatgataatgaacgttcaacccttcaaaattgaaactctcaggctgagcggctgagcaagctctgagaatgtgggtcttcgaagactttcgcttgagcatggtgcatgtgcaaagagatggaatgtggaattgttgtaggtaggaacacagcccggtagagcggcgcagtggctcagcgaagctgggagaacgactgaaccggtcttgagattcatcatctcagcgtgcgtcagcgcgtcagcttcagcgtttgagaatggcacggactgacctgtcgtacatcccaaggaggcccgtgcgctatactcgccttgtggcctaatctgcgatcaacccatcctcaacgaaagcgcaagtagcgactaagcacactatctttcgggtcggtgcggtcacagtatcggcatctctgtctttagctacgatggtgtgcgtgtaactgatcagtacacccgctgtatcaaaactctatcttcaatccgctaagaatcgtcaccatcagtacatcagtacatcagtgggctaagatctaatggtgtcgttcgacggccgcgggcactagccacgagccaagagcggtacagttgctgcgaaatggtgggcagcctgacaggttcagtttgaataggatgaaaacgccatgaagaacagagaagctggcaattgacgatagtggggcgagcgcacgctgcttgctggtccgagcgtaggacgaacgacaaaccatgggcggggccaccaggccccagggagggcgtcactggttggatgtcgtctgttaggctagagctcaggcgctcagggcctctggcccaagcctcggacgcaagcctctgacgagcgagcgcgcgctggggcgggtctgcgcaggaacggcaggactgccgagttggttgaatattgaatggttggatgattggtttgctgggtttggttttgacacgcacggcacaggacaagtgagtttggaggttggatttgcgcaggtagaaagggcgctcggcctcagggcctcagcgtcagcagacgagctcgccagtccaggtcagtacccagtgttcgttacttcccaccgtccagagtttgctgctgcctctcagcttgattcgtgtcggagaagtagaaatggtcacagcgtaccacacactgtccccatgcctgcgtcctgctccacgagtgtttagccacaaaaatttatggacaaccttactgagcctgtgctggggggtgctttagcaccagcactggcgccatggcgtcaaggttaattccactgtcattcgacaaccttcaactgatgggggcaactgggcacaaaaccttggatatataaccaactccaactcccacggttttttaccttttgctcttctccccaggactcactacttctactcctctattcatcttcaccaaaatgattaatcaagtctatcgtctcgtctctccacgccaattcgaggtcgcgtataacgatcgcagcctccagtatcacgaggactttgttgtggttcgacctacgcatctgagcatctgcgctgcggatcaacgatattacactggctcgcgtggaaaagaagctatggcaaaaaagcttcccatggctctgatccacgaggccattggtaaagtggtctacgaccccaagggtgagatcaaaaatggcactatggtggtcatgattccgaacacgcctactgaaaaggatgaagtcattggtgagaattatctccgatctagcaaattccgctcgtccggatatgatggattcatgcaagattacgtcttcatgggtcgcgaccgtattgtggaactcccggaaggaattgacccgtatgttgttgcttttaccgagttgatgaccatcacaacccatgtgttgagccgatttgaatctcgttcccataaacgacgtaacgtttttggcgtttggggagacggcaaccttggtttcatcacgacactcttcctgaagaagatgtataaagattgtaaagttatcatcttcggaaaaactggatataaactcgacaatttctctttcgttgatgaggtgtatacgattgacgaaatccctgaggacctgcgcatcgaccatgcctttgagtgcgttggaggtaagggttctcagtatgcaatcgaccaaatgattgaccatctccaccctgaaggttccattgctcttatgggtgttagcgaatttccagttgaagttaatactcgtatgatcctggaaaaaggaatcacactgatcggaagctctcgatccggtcgagaggacttcgtgcgcacggtcaactttctgaaggaaaatcccgatgttgttgaatacctgggtacccttgtgggcgaggtccaagaagtcaagacgatctccgatattttgtctgcatttgagaaagaccttacatcctcttggggtaaaaccgtcatggagtggaagatctaacacggatcaacaaaaatgtgcgccagctttgttgtgtgcccttttcctctagcggatcctatccacatttcgtgaatgaaccgattaagctctgacgagatttccttgttttctgtagatacctcttgtagaggtgtcaaagttatgttgtgatcgagagagttacctgcaaggtattatgcaagcggcttgatgaggacactcaactacgtgtgagtaggagtgcgttgctcagcagttgacgcgtgagccgcttgagcagcttgataaaggtcctaatcgtcttccagctgttggcagacgacaatgtgctgtagtagtaggataggcccgtaaaagtgtagaagcaataggttgagactgactgtaatgggtacttgcgttcaaagactgaaagtccaaaggattgctcaaaggcccctgaggtctgagcctctgagcgcgctcttcgtcaaaattcgccggggccatcggtcatcctctcgagggcagcatcttcctgcttgaagactgagtccgtctcagataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatgcatcatgcatcatgcatcatgcgtcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatgggtaaggaaaagacacacgtatcgcgaccccgactcaactccaacatggacgcagacctgtacggatacaaatgggctcgagataatgtcggacagtcgggtgcgacgatctacagactctatggaaaaccggacgcccccgagctctttcttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactcgggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcg

SEQ ID NO: 81 - DNA, 4552 bp, synthetic construct
  source 1..4552: mol_type = other DNA
tgcagagccttgcaaatgatgaaagaccaatggaattgatcattgattattgatgatgcatgatgaaagagatagaaacagatgcaagatgggatggcgcgatgatcaaatgataatgaacgttcaacccttcaaaattgaaactctcaggctgagcggctgagcaagctctgagaatgtgggtcttcgaagactttcgcttgagcatggtgcatgtgcaaagagatggaatgtggaattgttgtaggtaggaacacagcccggtagagcggcgcagtggctcagcgaagctgggagaacgactgaaccggtcttgagattcatcatctcagcgtgcgtcagcgcgtcagcttcagcgtttgagaatggcacggactgacctgtcgtacatcccaaggaggcccgtgcgctatactcgccttgtggcctaatctgcgatcaacccatcctcaacgaaagcgcaagtagcgactaagcacactatctttcgggtcggtgcggtcacagtatcggcatctctgtctttagctacgatggtgtgcgtgtaactgatcagtacacccgctgtatcaaaactctatcttcaatccgctaagaatcgtcaccatcagtacatcagtacatcagtgggctaagatctaatggtgtcgttcgacggccgcgggcactagccacgagccaagagcggtacagttgctgcgaaatggtgggcagcctgacaggttcagtttgaataggatgaaaacgccatgaagaacagagaagctggcaattgacgatagtggggcgagcgcacgctgcttgctggtccgagcgtaggacgaacgacaaaccatgggcggggccaccaggccccagggagggcgtcactggttggatgtcgtctgttaggctagagctcaggcgctcagggcctctggcccaagcctcggacgcaagcctctgacgagcgagcgcgcgctggggcgggtctgcgcaggaacggcaggactgccgagttggttgaatattgaatggttggatgattggtttgctgggtttggttttgacacgcacggcacaggacaagtgagtttggaggttggatttgcgcaggtagaaagggcgctcggcctcagggcctcagcgtcagcagacgagctcgccagtccaggtcagtacccagtgttcgttacttcccaccgtccagagtttgctgctgcctctcagcttgattcgtgtcggagaagtagaaatggtcacagcgtaccacacactgtccccatgcctgcgtcctgctccacgagtgtttagccacaaaaatttatggacaaccttactgagcctgtgctggggggtgctttagcaccagcactggcgccatggcgtcaaggttaattccactgtcattcgacaaccttcaactgatgggggcaactgggcacaaaaccttggatatataaccaactccaactcccacggttttttaccttttgctcttctccccaggactcactacttctactcctctattcatcttcaccaaaatgctcaaccaagtgtatcgcctggtcgatccacgtcagattgaagttcaaactgttgcagaagagatcactgataatgacatcgtcgtccgccccaaatatctgtcggtctgtcatgcagacacgcgatactttacaggtcaacgtccccaagcaaccttgcgccagaagctcccaatggctttgatccatgaaggagttggcgaggttgttaaggaccctcaaaataaatttaagcccggtacgttggtcgctatggtccccaatacgccctttgaaaccgaccctatcattaaagaaaactacttgccaacgtctaaatttcgcagcagcggttacgatggtttcatgcaggaatacgttacacttcatcgtgatcgcgcgatcgtcgtgccagacaacttcgaccaccaaatgagcgcattcattgagatggtttccgtcggagtccacgccctcacccagctggaggccgtgatggatgcggatcgcaagatcatcggaatttggggagatggcaacttgggttttatcacagcaaccctcgtcaagcaaatctttccagactctcaactgatgatctttggacgacatcgctccaaattggactatttcagctttgcggacaagacatacctggtggacgacattcctagcgatcttcaagtttctcaggctctggaatgcaccggaggccgaggctcggagtctgctattgctcaaattatccaacacattcgaccaatgggcaccgctatccttatgggagtttccgaagatcccgttggtatcgatacacgcagcgtgttggcagagggattgacccttcgcggcgtgagccgtagcggtcgcgcggattttcagcgcgccatcgacattttgacgaaatcccccgtcacacgtgaacgtctgcaaaatcttgtgggtttcactcgaaaggtcagcacaatccaggatatcaccgatttcttcgagggtgcattgacaaactactggggcaaggccgttatggagtgggatgtttaacacggatcaacaaaaatgtgcgccagctttgttgtgtgcccttttcctctagcggatcctatccacatttcgtgaatgaaccgattaagctctgacgagatttccttgttttctgtagatacctcttgtagaggtgtcaaagttatgttgtgatcgagagagttacctgcaaggtattatgcaagcggcttgatgaggacactcaactacgtgtgagtaggagtgcgttgctcagcagttgacgcgtgagccgcttgagcagcttgataaaggtcctaatcgtcttccagctgttggcagacgacaatgtgctgtagtagtaggataggcccgtaaaagtgtagaagcaataggttgagactgactgtaatgggtacttgcgttcaaagactgaaagtccaaaggattgctcaaaggcccctgaggtctgagcctctgagcgcgctcttcgtcaaaattcgccggggccatcggtcatcctctcgagggcagcatcttcctgcttgaagactgagtccgtctcagataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatgcatcatgcatcatgcatcatgcgtcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatgggtaaggaaaagacacacgtatcgcgaccccgactcaactccaacatggacgcagacctgtacggatacaaatgggctcgagataatgtcggacagtcgggtgcgacgatctacagactctatggaaaaccggacgcccccgagctctttcttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactcgggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcg

SEQ ID NO: 82 - DNA, 4594 bp, synthetic construct
  source 1..4594: mol_type = other DNA
tgcagagccttgcaaatgatgaaagaccaatggaattgatcattgattattgatgatgcatgatgaaagagatagaaacagatgcaagatgggatggcgcgatgatcaaatgataatgaacgttcaacccttcaaaattgaaactctcaggctgagcggctgagcaagctctgagaatgtgggtcttcgaagactttcgcttgagcatggtgcatgtgcaaagagatggaatgtggaattgttgtaggtaggaacacagcccggtagagcggcgcagtggctcagcgaagctgggagaacgactgaaccggtcttgagattcatcatctcagcgtgcgtcagcgcgtcagcttcagcgtttgagaatggcacggactgacctgtcgtacatcccaaggaggcccgtgcgctatactcgccttgtggcctaatctgcgatcaacccatcctcaacgaaagcgcaagtagcgactaagcacactatctttcgggtcggtgcggtcacagtatcggcatctctgtctttagctacgatggtgtgcgtgtaactgatcagtacacccgctgtatcaaaactctatcttcaatccgctaagaatcgtcaccatcagtacatcagtacatcagtgggctaagatctaatggtgtcgttcgacggccgcgggcactagccacgagccaagagcggtacagttgctgcgaaatggtgggcagcctgacaggttcagtttgaataggatgaaaacgccatgaagaacagagaagctggcaattgacgatagtggggcgagcgcacgctgcttgctggtccgagcgtaggacgaacgacaaaccatgggcggggccaccaggccccagggagggcgtcactggttggatgtcgtctgttaggctagagctcaggcgctcagggcctctggcccaagcctcggacgcaagcctctgacgagcgagcgcgcgctggggcgggtctgcgcaggaacggcaggactgccgagttggttgaatattgaatggttggatgattggtttgctgggtttggttttgacacgcacggcacaggacaagtgagtttggaggttggatttgcgcaggtagaaagggcgctcggcctcagggcctcagcgtcagcagacgagctcgccagtccaggtcagtacccagtgttcgttacttcccaccgtccagagtttgctgctgcctctcagcttgattcgtgtcggagaagtagaaatggtcacagcgtaccacacactgtccccatgcctgcgtcctgctccacgagtgtttagccacaaaaatttatggacaaccttactgagcctgtgctggggggtgctttagcaccagcactggcgccatggcgtcaaggttaattccactgtcattcgacaaccttcaactgatgggggcaactgggcacaaaaccttggatatataaccaactccaactcccacggttttttaccttttgctcttctccccaggactcactacttctactcctctattcatcttcaccaaaatgtgagacctaatatcactgctgtacagacaagactgtgtatattaggtctcaatgatcaattacgtgtatcagctggtcagcccccacgtcttttccctcaaatatgaagaagtggacttcaagaaagaggtcatcatccgtccgaagtgtatggctgtgtgccacgcagaccagcgatattttctgggacgccgcgacgctaagatcctgcgacaaaaattgcctatggcccttatccacgaatgttgcggtcaggtggtctgggatggcacaggttccttccaacgaggccaaaaggtcgttctcattccaaatgtgccaggacgcgacgacggtgtcacatacgaaaactacgctgaaggatctcgttttcttagctcgggctgcgacggtttcatgcgcgagtatgtcaacctttccccagatcgtgtggttcccttcgaacaggttccgcttccggtggccgcgatcacagagttcgtgtccgtggcggttcacgcggccggtcgctttatgcgagcggcgcacacacgccgcgaggctatcggagtgtggggagatggctctctggcttatgtcgtctgctgcgttcttcgtcgactgctcccggagtcgcgtatttttgtggtgggtaagcatgaacgcaagttgctccagttctcgtttgccgatcgcaccttcctttttgatagcctcccggaggacttccatattgaccacgcgttcgaatgcactggaggagagggctcttactatgctatcgatgatattattaccgcggtgcgtccccaaggcgctgttatgcttatgggtgtctctgaaaaccgcgttccagttcgcacccgtgacattctggaaaaaggcttgacgttcaccggttgttctcgttctggacgcgaagactttgttaaggcagtcgagcttatggagcaaccccaattccaaaaacgccttagcgcaattatttacgaggatcgtccggtccatactgtcgccgatctgtcccgtgttttcgagacggatttgaatacgccgttcaagacagttttccgttgggagatttaacacggatcaacaaaaatgtgcgccagctttgttgtgtgcccttttcctctagcggatcctatccacatttcgtgaatgaaccgattaagctctgacgagatttccttgttttctgtagatacctcttgtagaggtgtcaaagttatgttgtgatcgagagagttacctgcaaggtattatgcaagcggcttgatgaggacactcaactacgtgtgagtaggagtgcgttgctcagcagttgacgcgtgagccgcttgagcagcttgataaaggtcctaatcgtcttccagctgttggcagacgacaatgtgctgtagtagtaggataggcccgtaaaagtgtagaagcaataggttgagactgactgtaatgggtacttgcgttcaaagactgaaagtccaaaggattgctcaaaggcccctgaggtctgagcctctgagcgcgctcttcgtcaaaattcgccggggccatcggtcatcctctcgagggcagcatcttcctgcttgaagactgagtccgtctcagataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatgcatcatgcatcatgcatcatgcgtcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatgggtaaggaaaagacacacgtatcgcgaccccgactcaactccaacatggacgcagacctgtacggatacaaatgggctcgagataatgtcggacagtcgggtgcgacgatctacagactctatggaaaaccggacgcccccgagctctttcttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactcgggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcg

SEQ ID NO: 83 - DNA, 4606 bp, synthetic construct
  source 1..4606: mol_type = other DNA
tgcagagccttgcaaatgatgaaagaccaatggaattgatcattgattattgatgatgcatgatgaaagagatagaaacagatgcaagatgggatggcgcgatgatcaaatgataatgaacgttcaacccttcaaaattgaaactctcaggctgagcggctgagcaagctctgagaatgtgggtcttcgaagactttcgcttgagcatggtgcatgtgcaaagagatggaatgtggaattgttgtaggtaggaacacagcccggtagagcggcgcagtggctcagcgaagctgggagaacgactgaaccggtcttgagattcatcatctcagcgtgcgtcagcgcgtcagcttcagcgtttgagaatggcacggactgacctgtcgtacatcccaaggaggcccgtgcgctatactcgccttgtggcctaatctgcgatcaacccatcctcaacgaaagcgcaagtagcgactaagcacactatctttcgggtcggtgcggtcacagtatcggcatctctgtctttagctacgatggtgtgcgtgtaactgatcagtacacccgctgtatcaaaactctatcttcaatccgctaagaatcgtcaccatcagtacatcagtacatcagtgggctaagatctaatggtgtcgttcgacggccgcgggcactagccacgagccaagagcggtacagttgctgcgaaatggtgggcagcctgacaggttcagtttgaataggatgaaaacgccatgaagaacagagaagctggcaattgacgatagtggggcgagcgcacgctgcttgctggtccgagcgtaggacgaacgacaaaccatgggcggggccaccaggccccagggagggcgtcactggttggatgtcgtctgttaggctagagctcaggcgctcagggcctctggcccaagcctcggacgcaagcctctgacgagcgagcgcgcgctggggcgggtctgcgcaggaacggcaggactgccgagttggttgaatattgaatggttggatgattggtttgctgggtttggttttgacacgcacggcacaggacaagtgagtttggaggttggatttgcgcaggtagaaagggcgctcggcctcagggcctcagcgtcagcagacgagctcgccagtccaggtcagtacccagtgttcgttacttcccaccgtccagagtttgctgctgcctctcagcttgattcgtgtcggagaagtagaaatggtcacagcgtaccacacactgtccccatgcctgcgtcctgctccacgagtgtttagccacaaaaatttatggacaaccttactgagcctgtgctggggggtgctttagcaccagcactggcgccatggcgtcaaggttaattccactgtcattcgacaaccttcaactgatgggggcaactgggcacaaaaccttggatatataaccaactccaactcccacggttttttaccttttgctcttctccccaggactcactacttctactcctctattcatcttcaccaaaatgtgagacctaatatcactgctgtacagacaagactgtgtatattaggtctcaatgcttaaccaagtttatcgactcgtttctccccgtcagttcgaggtggagacagtcgcagaagaagttgatgcggatgacttgatcgtgcgtcccaagtttctcagcatttgccacgcggacactcgatacttttttggacagcgctcgcaagccgcactccgagccaaacttccgatggctttgattcacgaggccgtcggtgaagtcgtttacgatccccgtaatgaatacaagcccggaaccttggtcgcaatggtgcctaacaccccggtcaagaaggatagcatcattcaagaaaattacctgcccagctcccgtttccgatctagcggttatgatggcttcatgcaagaatacgtgtgtctgcgacgtgatcgagctattactgttccttccgactttgacccgcatatggcggcgttcattgagatggtttccgttggagtgcaggcacttactaagttggaatccacaatggatgaggatcaacgcgttattggcatttggggtgacggaaatctcggttacatcaccgccaccctcgttaagcaaatctttccagactctcagctcctcgtcttcggacgccatcagtctaaactggattatttctcgtttgcagataagacgtatctggtggacgatatcccggccgacgtcaaagtttcgcaggccctcgaatgtacgggcggtcagggctccgaagccgcgatccaacagatcatccaacacattcaaccgatgggtacggcaattctcatgggtgtttctgagaatcccgtcggaatcgacactcgttcggttcttgctgaaggtctgactctccgaggagtgtcccgtagctcgcgcgaggattttaagcgtgcggttgacatcctgtcctcttcgccagttacgcgtgaacgattgcagaatctcgtcggctatgttcagaaggttagctccatccaagacattaccaacttctttgatagcgccctcactaattactggggcaaggaggtgatggagtgggacgtgtaacacggatcaacaaaaatgtgcgccagctttgttgtgtgcccttttcctctagcggatcctatccacatttcgtgaatgaaccgattaagctctgacgagatttccttgttttctgtagatacctcttgtagaggtgtcaaagttatgttgtgatcgagagagttacctgcaaggtattatgcaagcggcttgatgaggacactcaactacgtgtgagtaggagtgcgttgctcagcagttgacgcgtgagccgcttgagcagcttgataaaggtcctaatcgtcttccagctgttggcagacgacaatgtgctgtagtagtaggataggcccgtaaaagtgtagaagcaataggttgagactgactgtaatgggtacttgcgttcaaagactgaaagtccaaaggattgctcaaaggcccctgaggtctgagcctctgagcgcgctcttcgtcaaaattcgccggggccatcggtcatcctctcgagggcagcatcttcctgcttgaagactgagtccgtctcagataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatgcatcatgcatcatgcatcatgcgtcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatgggtaaggaaaagacacacgtatcgcgaccccgactcaactccaacatggacgcagacctgtacggatacaaatgggctcgagataatgtcggacagtcgggtgcgacgatctacagactctatggaaaaccggacgcccccgagctctttcttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactcgggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcg

SEQ ID NO: 84 - DNA, 4555 bp, synthetic construct
  source 1..4555: mol_type = other DNA
tgcagagccttgcaaatgatgaaagaccaatggaattgatcattgattattgatgatgcatgatgaaagagatagaaacagatgcaagatgggatggcgcgatgatcaaatgataatgaacgttcaacccttcaaaattgaaactctcaggctgagcggctgagcaagctctgagaatgtgggtcttcgaagactttcgcttgagcatggtgcatgtgcaaagagatggaatgtggaattgttgtaggtaggaacacagcccggtagagcggcgcagtggctcagcgaagctgggagaacgactgaaccggtcttgagattcatcatctcagcgtgcgtcagcgcgtcagcttcagcgtttgagaatggcacggactgacctgtcgtacatcccaaggaggcccgtgcgctatactcgccttgtggcctaatctgcgatcaacccatcctcaacgaaagcgcaagtagcgactaagcacactatctttcgggtcggtgcggtcacagtatcggcatctctgtctttagctacgatggtgtgcgtgtaactgatcagtacacccgctgtatcaaaactctatcttcaatccgctaagaatcgtcaccatcagtacatcagtacatcagtgggctaagatctaatggtgtcgttcgacggccgcgggcactagccacgagccaagagcggtacagttgctgcgaaatggtgggcagcctgacaggttcagtttgaataggatgaaaacgccatgaagaacagagaagctggcaattgacgatagtggggcgagcgcacgctgcttgctggtccgagcgtaggacgaacgacaaaccatgggcggggccaccaggccccagggagggcgtcactggttggatgtcgtctgttaggctagagctcaggcgctcagggcctctggcccaagcctcggacgcaagcctctgacgagcgagcgcgcgctggggcgggtctgcgcaggaacggcaggactgccgagttggttgaatattgaatggttggatgattggtttgctgggtttggttttgacacgcacggcacaggacaagtgagtttggaggttggatttgcgcaggtagaaagggcgctcggcctcagggcctcagcgtcagcagacgagctcgccagtccaggtcagtacccagtgttcgttacttcccaccgtccagagtttgctgctgcctctcagcttgattcgtgtcggagaagtagaaatggtcacagcgtaccacacactgtccccatgcctgcgtcctgctccacgagtgtttagccacaaaaatttatggacaaccttactgagcctgtgctggggggtgctttagcaccagcactggcgccatggcgtcaaggttaattccactgtcattcgacaaccttcaactgatgggggcaactgggcacaaaaccttggatatataaccaactccaactcccacggttttttaccttttgctcttctccccaggactcactacttctactcctctattcatcttcaccaaaatgattaatacactgtacaaacttacggccccactgattgtggaatccttttatgaaaatatcgactgtcattccgacaatattattgtgcgccccgaaaaactttctatctgcaaggctgacatccgatactacttcggaatgcgtaatgctaaagtgcttaaagagaagttgcccctcgcactcattcatgaggcatgtggctctgttgtctatgacatcaccggcaagtttaaggctggtgaccaggtcgttatgcttcctaatattccgggtattaacatgcagtatcatgagaattatcgtcgtgatagccttttccgcagctctggagcagacggattcatgcaggagttggtgtcgatccctcagtcgcaagtcatcaaatttgagaagatggatcaaaacattgccgcgttcacggagttcatctctgtcgctattcatgcagtcagctcgtatcttgatcgcagcgcgggaaagcgaaatggtatcaagcgtaaaatcggtgtgtggggtagcggcgcaatgggctacgcggtgtcgtgtgtcttgaagtacttcattccatctgcagagatcactgtcattggaggtagcaaagcaaagctggacatgttttcctttgtcaatcatacttgttccctgagcgagcttcctgacgatgcagagttcgatgattgcttcgaatgtgtcggtggagcccaatctggcaatgcaatcaatcaaattatcgatcatattctgccggaaggcctcctgctgttgttgggcgttagcgaagaggccgtgcctgtcaacactcgaatggtgctcgaaaaaggcatcactttgttgggtcgatcgcgctctgagcgttcggactttatcaaaacaatcgacatccttgatacgaacgagaatattcgacgccgcatttctaacttgatctctggcgaggtggagattaacgaggtgaacgacatccacaaggcttttaatcagtccaaaattgcagactataaaatcatcttgaattggcgtatttaacacggatcaacaaaaatgtgcgccagctttgttgtgtgcccttttcctctagcggatcctatccacatttcgtgaatgaaccgattaagctctgacgagatttccttgttttctgtagatacctcttgtagaggtgtcaaagttatgttgtgatcgagagagttacctgcaaggtattatgcaagcggcttgatgaggacactcaactacgtgtgagtaggagtgcgttgctcagcagttgacgcgtgagccgcttgagcagcttgataaaggtcctaatcgtcttccagctgttggcagacgacaatgtgctgtagtagtaggataggcccgtaaaagtgtagaagcaataggttgagactgactgtaatgggtacttgcgttcaaagactgaaagtccaaaggattgctcaaaggcccctgaggtctgagcctctgagcgcgctcttcgtcaaaattcgccggggccatcggtcatcctctcgagggcagcatcttcctgcttgaagactgagtccgtctcagataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatgcatcatgcatcatgcatcatgcgtcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatgggtaaggaaaagacacacgtatcgcgaccccgactcaactccaacatggacgcagacctgtacggatacaaatgggctcgagataatgtcggacagtcgggtgcgacgatctacagactctatggaaaaccggacgcccccgagctctttcttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactcgggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcg

SEQ ID NO: 85 - DNA, 500 bp, Moniliella pollinis
  source 1..500: mol_type = genomic DNA
tgcagagccttgcaaatgatgaaagaccaatggaattgatcattgattattgatgatgcatgatgaaagagatagaaacagatgcaagatgggatggcgcgatgatcaaatgataatgaacgttcaacccttcaaaattgaaactctcaggctgagcggctgagcaagctctgagaatgtgggtcttcgaagactttcgcttgagcatggtgcatgtgcaaagagatggaatgtggaattgttgtaggtaggaacacagcccggtagagcggcgcagtggctcagcgaagctgggagaacgactgaaccggtcttgagattcatcatctcagcgtgcgtcagcgcgtcagcttcagcgtttgagaatggcacggactgacctgtcgtacatcccaaggaggcccgtgcgctatactcgccttgtggcctaatctgcgatcaacccatcctcaacgaaagcgcaagtagcgactaagcacactatctttcgggtcggtgcg

SEQ ID NO: 86 - DNA, 1002 bp, Moniliella pollinis
  source 1..1002: mol_type = other DNA
tttagctacgatggtgtgcgtgtaactgatcagtacacccgctgtatcaaaactctatcttcaatccgctaagaatcgtcaccatcagtacatcagtacatcagtgggctaagatctaatggtgtcgttcgacggccgcgggcactagccacgagccaagagcggtacagttgctgcgaaatggtgggcagcctgacaggttcagtttgaataggatgaaaacgccatgaagaacagagaagctggcaattgacgatagtggggcgagcgcacgctgcttgctggtccgagcgtaggacgaacgacaaaccatgggcggggccaccaggccccagggagggcgtcactggttggatgtcgtctgttaggctagagctcaggcgctcagggcctctggcccaagcctcggacgcaagcctctgacgagcgagcgcgcgctggggcgggtctgcgcaggaacggcaggactgccgagttggttgaatattgaatggttggatgattggtttgctgggtttggttttgacacgcacggcacaggacaagtgagtttggaggttggatttgcgcaggtagaaagggcgctcggcctcagggcctcagcgtcagcagacgagctcgccagtccaggtcagtacccagtgttcgttacttcccaccgtccagagtttgctgctgcctctcagcttgattcgtgtcggagaagtagaaatggtcacagcgtaccacacactgtccccatgcctgcgtcctgctccacgagtgtttagccacaaaaatttatggacaaccttactgagcctgtgctggggggtgctttagcaccagcactggcgccatggcgtcaaggttaattccactgtcattcgacaaccttcaactgatgggggcaactgggcacaaaaccttggatatataaccaactccaactcccacggttttttaccttttgctcttctccccaggactcactacttctactcctctattcatcttcaccaa

SEQ ID NO: 87 - DNA, 1038 bp, synthetic construct
  source 1..1038: mol_type = other DNA
atgaaagccctcgttttggacgaatacaatcaattgaattaccgtgatttccccgagcccgaaatccactccggagaggtgctgatcgagatcaaggcgtgcggtatttgtggctctgatgtgcacggtatggacggatcgaccggacgtcgaaaaccaccgctggttatgggtcacgaagcctctggcgtcattatcgaaaccggtagcgatgttaagaactggaaaacaggtgaccgagtcacgtttgatagcaccgtgtacccacttaatgactggttcactctgaaaggccactacaacctcagcaacaatcgacaagtgttgggagtgtctcctggcgagtatcgccgtcatggtgcgtttgccgaacgacttgcggttccgcaacatatcctgcaccgaatcccagagaacgttagcttcgtggaggctgcgatggttgagcccatcgctgtggcagctcacgcggtgaatgtttctggattgaagccgggcaattcggctatggttgcgggcgcgggcatggtcggagtgtttgttgtccaactgctgaagctttttggagctaaccctgttgtggttattgacatcgatcacaagaaactgaagcttgctcacgaattcggtgctgactttacattcaatcctaaagaaaaggatattgacgaacgaatcttgcaactcacaaaatcgcgcggttttgaccttacatttgaagtcgtcggcatcaccgagacggtccagacctgcattaccaacacacgtaagggtggcacggtggttcttgttggaaacttgtcccccgaaatccaatttcccctgcaaaaagtcgttacttctgaaatcactgtgcagggctcttgtgccatcaagggtgaatacgagctggcactggacctcttggccgcaaagaaggttgacgtctcgaaactcgtttcggctgttgcaccgctgtctgagggagcggaatggtttaagcgactttacgagaaggaaccgggattgaataaagtgatcctcattccagataagtaa

SEQ ID NO: 88 - DNA, 978 bp, synthetic construct
  source 1..978: mol_type = other DNA; note = Candidatus Dormibacteraeota bacterium
atgaaagctatggtgtggcttggacccgaacgaatggaagtgcgagatcttcctgcgccgcgaccgggtgcaggtgaagtgctggttcagacggaagctgctggaatttgtggctctgaagtggaggcctatttgggtcgcatggctaatcgaacaccgccgttggtgatgggtcacgaatatgctggacttgtcaccgaggtcggagagggagtcgaccgcgtctggtcccgtcgacgcgttgcggttaacccgattgttggttgcggtcgttgtcgaaactgcgccgccggtgatcgaaacttgtgtcctgatcgtcaccttcttggcattgcagcgccaggaggattcgcagcgttctcggtggttcccgaacgatgcctcttttctctgcctgaaggaatggatgcgcgactgggcgcgctcaccaagccattggccaacggtgtgcatcttattcgcaaaagcggcgcgggacgaggccgagttgtggttatcggcgcgggaactattggactcggatgtttgcaagctgctttgttgcacggtgctgagcaagttgacgtcgttgaacgccatccgggacgacgccgtcacgcacttgccctgggagcacgccaggcggtggcggaattggcagatgtcgaacctggtgctgatcttgtggttgatgccgcaggatccgaagcgacacgccgcgcggccgtggaattggttggcccggcgggaacggcctcttttgttggactccactccgacgaaactgtcctgccttggcatcgcattatccgagccaatattcgagttcaaggaacatttggctacgcagatcgtgatttccagcaagctcttgactggttggcagcgggaaaggcagccattccattgtctgaactccgccccttggacgatggcccagcggccttcgagcgccttgctcgtggaccgatcgacgaaatcaaagtctttttggcttaa

SEQ ID NO: 89 - DNA, 1014 bp, synthetic construct
  source 1..1014: mol_type = other DNA; note = Candidatus Falkowbacteria bacterium
atgaagtcgttgatttactatggcccgcacgatctgcgatatggagattctcctaagccaaagatccgatctggagaagtcctcctccgaatcaaaagcgtcggtatctgcggcactgacctccacatctataacggaggcatgaaaaatctgccgttgcctctgattatgggtcacgagttcagcggagttattgaagagcttgcccgagacgtgaagaactttaaggttggagatcgagtgatcgctgaacacgttatcaattgtggaaaatgttcgtattgtcttagcggcaagccgaatctgtgtgctaactctcgcattattggagttcaccgccccggagcgctcgccgaatacttggctgttcccgcggacctggtgtacaagatcccggcgaaactgtcttttgaagagggcgcaatgatcgaacccctttctatcgcctattatgccgtgaagcaagctcgtagcatcctgggtaaaaaggtggcggtcatcggccaaggacccatcggcctgctgcttgaccaaatcctccgcgacgcaggcgctgaagtcattggcattgacatcgcagattggcgattgaaactcgcgaagaaaaagaagtgggcagatcatacaatcaactccagccagaaaacctggctcaagaagatccagaagatcgcaccagaaggcgtggacacagtgtttgaggttgttggccgtgaggaaactgccgagatggctattgaaatcgcgcgtcgagatggtgacgtttttatcctcggtgtctttgaaagcgccgcatctttgaacatgatgaatattgttaagaaggaactcaacgtgttcggaagctggacctgtgcgttttctttccccccttcgattgctctcgccgttgccaagaaagttgatttgaaatccttgatcacgaatcgatatgacgtgaaggacggtgccctggcatttaaggagtctgcctcttataaaggtaagcgaatcaagactgttatcaacttttaa

SEQ ID NO: 90 - DNA, 1044 bp, synthetic construct
  source 1..1044: mol_type = other DNA; note = Acidobacteria bacterium
atgaaggcaatgttgcttagccagtatcgacgactggaaattgccgagttgccgacaccgatccctggtcgtggcgaactcttgatccatgtcgcggcatgtggtatttgcggaagcgatgtgcacggatacgatggcagcagcggacgacgtatccctccaatcgtgatgggccatgaggctgcaggacgtgttgcggcactcggtccgcaggtcacgggctggtcggaaggtgatcgcgtcacattcgatagcaccatctcctgcggcacttgcccttactgctcccgcggcgagattaacctctgcgaccgtcgtcaagtgcttggagtttcgtgcggtgactatcgttgcgctggagcttttgcggagtatgttattgtgccgcaacgcattgcttataaattgccggatgccttgagcttctcggaggccgcaactttggaagcggtcgcggttgctgtgcacgctgttagcctttcccaggtttcgccaggtactcaggcactggttattggcgctggtatgattggcctcctcaccttgcaagcgctgcgcgcagccggttgcagctccgtggtgatcgcggacgttgatgcgtcccgtctgcgtttggctaaagaggttggcgcaaccgaaacgcttaatgcgacaggagatgaatttgttgccgaaatcttcaaacgcacaaacggagcgggcacagaccttgccgtcgaagctgtcggtatcgatgctaccgtccgaacggccattaactctgtccgaaaaggcggtacagttaccctggtcggcaatatcacgccggaggtcactcttccgcttcagaaggtggtgacacgccagattcgaatccaaggttcttgcgcatccgctggtgaatacccccgagctatccagctccttacgtctggcgctattaaagtcaaacctctcattaccgctgtggccccgcttgaagacggtcctcagtggtttgagcgcctttacagccgcgagcctaatcttatgaaggtggtccttaccccgggcagcgtgtcttaa

SEQ ID NO: 91 - DNA, 1035 bp, synthetic construct
  source 1..1035: mol_type = other DNA; note = Spirochaetaceae bacterium
atgaaagcgatggtgttgaaagaatataagtatctggaaatcgaagacattcctattccgcacatcgaagcagcggacgaactgttggtccgcgtgaaggctaccgccatctgcggttcggatatccatggttttgatggttccaccggacgccgtaaaccgccattggtcatgggacacgaggcatcgggtgttgtcgaaagcgtcggaagcgaagttaaatcgctgaaaatcggtgaccgtgtcaccttcgactccacagtttggtgcggcgaatgctacttttgccgacagggtcaggttaacctgtgcgacaaacgacgtgtgcttggtgtgtcgtgcgacgaataccgcaaggacggtacctttgcggaatatgtcattgtgcccgagcgtattgtgtataagttgccggataatgttgccttcgaagaagcagcattgactgagccggtttctgtcgcgatgcacgcatttgatatcaccggaatgaagccaggagaaaatgcggccgttatcggtagcggtcttatcggactcctcctgattcaaattatccgtgcttattcgccccacttgatcatcgcgttcgatacagacgaaaaacgccgaaaagcagctatcgaagctggagccgatattgcattggacccccaagatcctgagtcgcataaaactgtcctgagcatgactaataaccgaggtgtcgaccgagcatttgaggctgtgggcgcctcggcagcgatcggtacagctattgaatccgtccgaaaaggctcgtccgtcactctgatcggcaatattagcaataaagctgagatcccgctccaatctgtggttacgcgccaaattgcccttttgggttcgtgcgctatttctggagagtatcctgtctctttggctttgatggcgaatcgcactattgacgtgcgtcaagttatctccgcaatcgcccctttggaggagggtcctctgtggttcgaacgcttgtataaacgcgaacctggtcttttgaaggttgttctcacgccgtaa

SEQ ID NO: 92 - DNA, 1047 bp, synthetic construct
  source 1..1047: mol_type = other DNA; note = Candidatus Hydrogenedentes bacterium
atgaaggcactccgcctgtacgctccaggtgacttgcgtttggaggaaatcccaattcctttgccacgtcccgatgaggtgttggttcgcgtgcttgcgtgcggtatctgcggttcggatatcccccgtgtttttgaacacggtacgtataaatacccattggtgccgggccatgagttttccggtattgttgaaaaagtcggctccgaaacgggaaaagattgggttggtcgtcacgttgtcgtctatcccctcatcccgtgtcgacaatgctcctcttgctactctggtctttatacccaatgcctcaactacgactacttgggatctcgccgtgacggaggatttgcagagtatgtgactgtccctgtgaataatattatcccggtccctaatggaatctctggagagatctcggcgatcactgagccatgtgcggtcgcccttcatgccgttcgcaaggcccaagcctgcgtgggtgacacggtccttattacgggagctggaaccattggtatcttgatcgccctctgggcgcgagcagcaggtgccgaagtctttatgactgacattgtggtgggaaagttgaaactggctcgttcgttgggttttcagcatctgctggacgccaaacaaacaaatgtttctgaatgggttaagcaaaagtgtaaacacggagttgatatcgtgttcgaaaccacgggacatcccgcgggactcgaaacgtcgattgactcttgttctattacaggcaaaattattctcgtgggcaatcccagcttcgaagagatgaaagttccggctaaacaatatggaatgattctgcgcaaggagatccagattactggaatttggaattcgctcatcaacgaatatccaaaaaacgagtggcaagttgtgctgcaagagttcaagaaggagtcgtccttgtactcgaagattatcacgcataaggttcccattgagggccttaaggaattgtttgtgacgatttacaacaaacaggtctttccggtgaaggctatgtttatcaataaggagatctaa

SEQ ID NO: 93 - DNA, 1014 bp, synthetic construct
  source 1..1014: mol_type = other DNA
atgaaggcagtcgtgtacacgcaaccagacgcagtggccgtccttgagcgtgaaatgcctgcactggtgccaggagaggtgcttcttcgaattgatgccagcggaatttgcggttccgatttgcacgcatatcatggacatgacccacgtcgtaaacctggacttgtgttgggtcacgagtttgcaggcaccgtggttgcttctagcgatgaaagccgcctccctgtgggtcgacgcgtcacggctaatcccctcgttacatgcggacactgtgattactgccttcagggccgtgacaatctgtgtgcgaatcgtggcatggtgggtatgtctcgccctggtgccctttctgagtatcttgccattcccatgcgatgcgctattgctattcccgaagatctgccgagcgtgtctgctgcgcttaccgaacctgctgcgacggcactgcacgcagtcaatcttacaatgcgagcgatgagccgacctctcccggaggcgcgtgttttgattatcggaggtggagcgatcggcatgttgaccggagcacttgtcggtgcgtacggctgtcgctcccttgcggtcgcggaaaccaacgccctgcgccgtgcgtccgtccgacgtcatcttgcatgcgaggttttggaccctacagcgaacgccccggctgagtcttcgttcgacgtggtcattgacgcagtgggtgcggcaccaacccgccatctggcactggcggcaacgcgaccaggaggtgttatcatgcacattggactccaggcatggtccagcgaaatcgatatgcgtaagctcactcttgctgaaatcactctcctcggtacgtatacatacacgatggcggatatgcatgcgaccgtgcgagcacttgcctctggaagcttcggatctctggaatgggtcgagacgcgagccctgacagatgcgccatctgcttttgctgaccttgctgcgggtcgagtcgcggcgggcaaggttgtgcttctcccgcaacaggactaa

SEQ ID NO: 94 - DNA, 1044 bp, synthetic construct
  source 1..1044: mol_type = other DNA
atgaagtccgtcgttattcacgccgaaggagatgtccgtgtggaagagcgacccattccccatttgcaaacggatgaagatgttttggtgaaagtcgtgtcttccggactttgtggcagcgacattccccgcatcttcaatcgtggcgctcattattacccaatcacacttggccatgaattctcgggctatgtcgagtcctatggagcgggtgtcactgacttgcaaccaggtgatgctgtcgcttgtgtgccgctcctcccgtgttttcactgcccccagtgcgagcgaggttacttctctctgtgtaagcagtatcaatttgtcggttctcgcagcgagggaggaaacgcagagtatgtcgttatgaaacgagcgaatcttttccgactcccgtccgatatgccaattgaagacggagctttcattgagcccatcaccgtcggtctgcatgccttccaccttgcgcagggttgcgagggaaagaatgtcattatcgtgggtgcaggcaccattggcctccttgccctgcaatgtgcacgagaactcggagccaaatcgattaccgcaatcgacatcaacccacagaagcttgcattggcaaaggagctgggcgcgacttacgtgtttaattcccgagaaatgtcggcgaccgaaattcactctgatttggaaaatgtgcagttcgaccaacttattctcgaaacggctggaacgcctcagactgtcacgctctccatcgaaattgcgggtccccgagcccaattggcgcttgttggtacgttgcaccacgatttggcgcttgcgtcgggtgtctttggtcaaatcttgcgcaaagagcttactatcctcggatcttggatgaactacagctctccctggccaggcgaagaatgggtcactgcggcacgtctgctgacggagaaacgtttgcaattggaaccacttattgcgcactccggagatgccgaatcctttgcgtctgcagtgaagtctctcaatggcgcacctatgcaaggtaaaattctgctcaagctttcgtaa

SEQ ID NO: 95 - DNA, 1041 bp, synthetic construct
  source 1..1041: mol_type = other DNA
atgaaatctgttgtgaacgatacggacggtattgttcgtattgcagagtcggtgattcccgaaattaaacaccaagatcaagtgcgtgttaagatcgcctcttccggcctttgtggttctgacctgccgcgcatttttaagaacggagcacactattatcccatcactctcggacacgaattttcgggatacatcgatgcagttggctccggtgtcgatgatcttcatcctggtgacgccgtggcatgtgttccccttctgccctgttttacctgtccagaatgtctgaaaggcttttacagccaatgtgccaagtacgattttattggatcccgtcgtgatggtggtttcgcggagtatatcgtggttaagcgaaaaaacgtgtttgctttgcccactgacatgtctattgaagatggagcctttatcgaaccaattacggttggccttcacgcgttccacctcgcgcaaggttgcgaaaataagaatgtcattattattggcgccggtactatcggcctgcttgcaatccagtgtgccgtcgctctcggtgccaagtctgttaccgccatcgacatctcgtctgaaaaactcgccctggccaagtcttttggagcgatgttgacgttcaattccttggagatgtccgcccctcagatgcaaggagtcctccgagaattgcgattcaatcagttgattctggaaaccgctggcgtgcctaagaccgtggaactggcggtcgagattgctggaccccacgctcaactggccttggttggcactttgcaccaagacttgcatcttacgagcaccacttttggaaaaatcctgcgcaaagagcttactgtcatcggatcttggatgaattattcgtccccttggcctggtcaagaatgggagactgcatctcgtctcctcaccgagcgaaagttgtctctcgaaccgttgattgctcatcgtggcagctttgaatcgttcgctcaggctgtccgagacattgctcgcaatgccatgccaggaaaagtgcttctgattccttaa

SEQ ID NO: 96 - DNA, 1020 bp, synthetic construct
  source 1..1020: mol_type = other DNA; strain = K12
atgaaatctattctgattgagaaaccaaaccaattggccattgttgaacgtgaaatcccaactccatccgccggtgaagttcgtgtgaaagtgaagctggcgggaatctgcggatccgattcccatatttaccgtggtcataacccatttgcaaaatatcctcgtgtgatcggacacgaattttttggtgtcattgacgcagtcggagaaggtgttgaaagcgcccgcgttggtgaacgagttgcagtggatcccgtggtgtcgtgcggccattgctatccctgctctatcggaaagcccaatgtttgtactaccttggcggtgctgggcgtgcatgcggacggaggcttctctgagtacgcggtggttccggccaagaacgcgtggaagattcccgaggcagttgccgatcaatatgccgtgatgattgagccgtttacaatcgccgcgaacgtgacgggacacggtcagccaactgaaaatgacactgttctggtgtatggcgctggacccattggcctcacaattgttcaagtcctgaaaggagtgtataatgtcaaaaacgtgattgtcgccgatcgaattgacgagcgacttgagaaggccaaggaatccggagctgactgggctattaataatagccagacaccgctgggcgagattttcactgaaaaaggcattaagccgacgctgattattgatgccgcttgtcacccctcgattctgaaggaggcggtgacgctggcttctccagcggcacgtattgttctgatgggattctcgtctgaaccatctgaagttattcagcaaggaattacgggcaaggagctctctattttctccagccgccttaacgcaaacaagttccccatcgtgatcgattggctctccaagggcctgattaaaccagaaaagctcattacgcacacgtttgattttcaacatgttgcggacgctatcagcctttttgagcaagaccagaaacactgctgtaaggtgctgctcactttctccgagtaa

SEQ ID NO: 97 - DNA, 1074 bp, synthetic construct
  source 1..1074: mol_type = other DNA; strain = cp400
atgaacattccgaccaagatgaaggcagtcgttaaatcggaggcaggttatgacaatatgtccctgcaagagattcctgtcccagaagttaagggaaaacaggtgttgatgaaagtcgcctatacgggtatctgcggtacggacattcatacctttaaaggcgagtacgccaatgccgtgaccccacttgtcctcggccatgaattttctggcaaggtcgttgccgtcggagatgaagttaccaaggtcaaaccgggtgaccgagttacctccgaaacaacatttgatacatgcggagagtgtgtgtactgtaaaaataaggaatataacctgtgcgaaaaccgttcgggtattggtacgaaagccaatggatcgatggctaattatgtgcttacgcgagaggagtctgtgcatgtccttcctgacaatgtttcctacaaacttgccgcgatgtctgagccgctggcttcctgtgtgcacgcaatgtaccagaaaaccccgttcacccttcatgatacagttctcattatcggccccggacctatgggtttgctctctctgcagattgcaaaggaaattggagcctttaccatcgtctccggtattacgaaagacagcgaccgcctggaaatcgcaaaagaacttggagccgacatcgttgttgacacgcagaaggaagacttggcgaagatcgtcatggaggctactaacggagtgggagttaccaaagtctatgattgtagcggcgctacaccagcggtgaatgctgccctcccccttctgaagaagggaggtacttttcagcaggttggcctgtttgcaaagcctctgaacgaattggatgaacgtacaattattcagcacgaaattacatacgtgggatcccgttctcagaatccgtatgattggcctatcgccattcacctggaggccaaaggtgctattaatgaagataaaatggttacgaaagtcttcgatttggatcattggcgtgaggcattcgaggctatgatggctggaaaagaactcaaggttcttattgccagcaatccagacgacgaaactcttaattaa

SEQ ID NO: 98 - DNA, 1053 bp, synthetic construct
  source 1..1053: mol_type = other DNA
atgaaagccgcggtcctccatggaacgaacgacatgcgcttcgaggatattgaaatcaagccgtgcgaaagcgacgaggtcaagatcaaggtcatggccgctggcatttgtggatctgatcccccccgagtgctgaaacattggaagtatcctgttccggctattcctggacatgagttctctggtgttatcgcggaggtcggaaaagatgtgaaaaatgtcaaggtgggtgaccgagttgtggcgatcccctttattccgtgtaatgaatgtgagtactgtaaacgaggtctttttagcctttgtgacgatcacggaatgctgggtgccaaatcctttggagcatttgccgagtatgtgaacattaaggcgactaatgtccttcccatcggcgatatggatttcgaagacgcagcgatgattgagccccttgccgtcgccatgcacggagtgttgaacatcggagttcaagtgggagatacggtcgccgtcatgggctcgggcacaatgggtcaacttgtgattcagggtcttaaaattgctggtgctggcactatcattgctgttgacatttctgataacaaactgcgagagtccaaggaattgggcgctgacattatcattaacgcaaaggacattaaccctgttgaaaaaatcaaagagcttacaggcggaaaaggagtggacatcgctctggaatgtgcgggttccaaaattacccaagagcaatgcctcttgatcactaaaaaaaagtcgaagatcggtttcctgggaattgcgtactctgacattaccttgtcggaggaggcgttcgagaacatctttcgcaaagagctcgaacttaaaggattctggaacagctattcggcaccctttcctggccaagagtggaccaagggcattaatcttgttaacgagggaaaaattaagctgaaggaaatggtttcgcaccgcttttctcttgaggatacgtataaagcgttcgagatgatccgagatcgcaaggaagagtttaacaaaatcctgatccttccccagggcgtggaaaaataa

SEQ ID NO: 99 - DNA, 1053 bp, synthetic construct
  source 1..1053: mol_type = other DNA
atgaagtcggtccgattttatggaattcgtgatacacgagtcgaagacgttgacgtgcccaagattctggaaaaggatgacgtcattatcaaggtcaaggtggcgggcatctgtggttctgacattagcaaatactcgaaaacaggcccgcacatggttggcgagattttgggacacgagttctccggcgaggtcgctcaggtcggcaaggaagttcgctccttcaagatcggagatcgagtcgcagtttgccctgcgatgccctgcttcgagtgtgatgagtgcaaaaaaggtttgtattcccgttgcaataatgtcgctatcatcggaaataaggagctcggaggatgttttgcggagtacacgaaagttaaagagcgcaatctgattaaaattccagatgagattagctacgaaaccgctgcggcccttgaacccgtctgtatcgcaggacatggtcttttccgctctgaagccaaggtcggagatacggtcgttgtgctgggaacgggtcctatcggattgttcagcattcagtgggccaaaatctttggttccacgaagattatcgcggttgacgtgtttgacgaaaaactggacttggcgaaagagctcggtgcggatatttgtattaatgctaaggagaaaaatatcgttgaagagatcaaacgactgacagacggagatggtgcagatattgtcattgagtctgccggtaccccgcttacatgtggccaggtgcttctgctggcgaaaaaaggtggtactgtcttgtatgcaggcgtcccatacggcgacgtcgcccttactcgagaacaatttgagaaaatcgttcgaagcgagcttacggtgaaaggaacttggtttggcaattctttcccatttccgggaaaggaatggtctgctggcctgtaccacatgcagaaaggagacatgaacgtggaaaagcttgtcactcaccgtatcaatctggaagaagctccagcatacttcgagaaggtttataaacgtgatatcttctttggtaagattatgattaatattgacaattaa

SEQ ID NO: 100 - DNA, 1050 bp, synthetic construct
  source 1..1050: mol_type = other DNA
atgaaagcttccatgctggaggacttgaacaaattcagcgtcaaagagatcgacatcccgtcccccaaaaaggatgaggtcgtcgtgaaggtgatggcggcaggcacgtgtggatccgattcgcataaaatgattagcggatggaaatatggctatcccgcggtgatgggacacgaattttctggaattgtgacacaactgggagaaaacgtgtccaacgtcagcgtcggtcaacacgtcgccgtggctcccttcattccctgcttcaagtgtcattattgtcaaatcggactcttccagatgtgtgaaaattattccatgctcggtcagcaaaaatttggaggtttcgagcaatacgtttccgtcccggcacgtaacgttctggacatcggcaagatgtcgttcgaggaaggtgcccttattgaaccgatggcagttgcggcccacgctgttatgggaattaaaccggagctgggtgatacagttgcggtcttcggcctcggtacagtcggagatcttgttgtccgtcttcttatttcctcgggagcgacaaacgtcattggcattgacattgacgaccagaagcttgaaaagggactggatgagggttgcacacacgtgatcaactctgccaaagagtctcttgaagaaaagatcatggaatacaccgacggtctgggtgtcgatatttccatggaatgcgccggcagcaaaatcactgaggagcaaacactgctcgttacgaaacgccgtggaaaggttggatttgtcggtattgcttatagcgacgttctgctccatcagaaagcatttgagaacatcttccgccacgagcttacggtgactggattttggaattcctattccgcaccatttcctggacgcgagtggaccaactcgatccaattggttaatcgtggtcgtatcaagatcaaagatctcattactcatcgatttgagcttgaagacatgcaaaaagcattcaatatgatcaccactcgttcggaatcgtttaataaggtcatgtttttccccaacggcatcaattaa

SEQ ID NO: 101 - DNA, 1065 bp, synthetic construct
  source 1..1065: mol_type = other DNA
atgaaggctctcaatttgtacggtatccaggacctccgctttgaggaaacacccgctccctctatcgaacatgatgacgatatcattatcaaagtcaaggctgtcggaatctgcggatccgatttgtcgcgttataagaaactgggaccttatgtccctggaatgacttttggacacgaattcgcgggagaggtcgtcaaaattggtcgttccgtgacgggattttctatcggtgatcgtgtcgctgcttgccctacttatacttgcggccagtgccgttactgtcaactcggcgagccgacacgatgtgagcgactgtctgtgatcggcgcacgccacccaggtgcatacgcagaatacgtcaaacttcccgcgaagcatgtcatccccttgcccaacgttgttaactatgacgaggccgccctcattgaaccggcttcggttgttgcgcacggcttttatcgtactaacattaagccaggtgcaagcgtcgctattatgggcgttggctcgatcggtctgctcgccgtccaatgggcaaaaatcttcggtgcgacaacggtctttgccattgatattgacgaacagaaacttaacgtcgctaatcaacttggagcagatgtcttgatctccagcctccaacgacccgcacacaagcaaatccttgaatataccaacggaatcggtgttgacgtggctgtggagtctgctggtacaccttctacctccgcccaggtgtttgcgctgccgaaaaaaggtggtgaagttgtctttcttggtattccttatgccgatgtccagattgaacgattttacttcgaaaaaattgttcgtaatgaactgcacgtttacggatcttggaatgcactctcctccccattcccgggtaaagagtgggccacgactatccattatatgtcgtccggacaattgaatgtcgcgcccatgatttcctaccgactccctctcgctaagggccccgagacgttccagcagatcgcaaagggtgaactcaagcccacaaaagtccttttctatcctgagaagttgtctgagcgcaagtaa

SEQ ID NO: 102 - DNA, 1056 bp, synthetic construct
  source 1..1056: mol_type = other DNA; note = Lachnospiraceae bacterium
atgaaagtccttagcctgcataagcctggcgatattcgatttgaggacgctccaatgcctgagccaggagagggcgaagtgctgctgaaagttatggcatgcggcgtctgttcgtcggacgaggctcgaattttcaaggacggtgcccatattatgccaattgttcctggccacgagttctgtggccaagtggtgaaaacgggagcaggagtggacgaaagcctcatcggccgaaaagcgagcgtcttcccaatgcttccctgtttcgaatgcccttcttgtaaacgaggtgactacgcaacctgttctaactataaatactatggttcccgcaacgatggtggaatgggagaataccgagcaatcaaggcgtggaacctgaaccttctggatgattccgtcgattattgcgtcggcgcactgtccgagccatccgcggttggccatcacgccactgaagtgggaggagtgaaagccggagacaaggttttcgtcattggtagcggaaccatcggacttttgatcggcttcttcgctcaggctaagggtgcagaggtttggatcggtgcgcgtcgtaaagagagcaacgatttcgcggcgagcctgggttttaaaacgatcgacaccaataataccgaagacgaggttgcaaaggccacggatggtaacggcatggatgttgtcttcgaagcagttggtagcaataagtgtcttattgaagctattggatctgtcacggcgggaggcacacttgtcaccgtgggcaatcctgtgggagacatgaatcttccccgagatgtgtactggcgcatcctccgaaagcaactcacggtccgaggtacttggaactctagctactctgacaaggccaatgactggcacgaagtggccgcgttgatgaaagaaaaaaaaatcccgttcgaaaaactcatcacccgtaagtacaaactttctcaatgcgatgaggccttcgattttttccgtgaccactctattcagaagtcccgcgtgatgtttgttatgcacgaaaaagaatccgaagcttacaacggataa

SEQ ID NO: 103 - DNA, 1011 bp, synthetic construct
  source 1..1011: mol_type = other DNA; note = Faecalibacterium sp. OF03-6AC
atggtcaacatgatgaaagccctcgtgtttactgcgccccatacgttcgagtatcgtgacgttccccgacccgaacccgcccccgacgaggtcctggttgaggtgaaagcatgcgcaatttgtggttctgatgttcacggtagctcgggcgaaaacggtcgacgcattcctcccttggtgatgggccatgaggcaagcggcactatcgcacaattgggcgaagctgtgcagtcgtgggcagtgggagatcgcgttacatttgactctactgagtactgcggctactgttggttttgcacgcacggtatgcacaatttgtgcgcccagcgacgtatcgtgggcgttgcgtgcacagagtaccgtaaaaatggtgcaatggctcaatacattgcagtgaaagcccgtaccctttatgctttgccgccggcggtctcctttgaacagggttgccttgtcgaaccctttgctgtgggtatgcatgctgtcttgcgatctggactggcagcgggacagacggccgctgtgattggagatggaacaatcggtctgatgactcttattgcggccaagagccagggagcacaagtctgtatcagcggacaccacacccgtcccatgcagctcgcagcccagctcggagccctggattgtgcccaactgccccagctgacagctgagcgaggtgcggacgttgtttacgacgctgttggctctcaatccagctttacgcaagcgatggagcttgtccgtaagggaggccgaattatggttatcggcaatcatgcacgcacgatcgattttcctttgcaacgttgtatcacggaacagatcgacgttcagttttcgtacagctctgccggtgagtatcctgtttgtcttcaagccattgctgagggacgcgcagatttgacaggactgtggcaatctttcccactggctgagggcgctcaagttttcgaccgcctggttcaccgagatgagtccctgacgaaagccgttttgatcccataa

SEQ ID NO: 104 - DNA, 1074 bp, synthetic construct
  source 1..1074: mol_type = other DNA
atgaaggcctatgttttggatggtattggccagctgaatcaacatgagtatgtcctccgagacttgcagcctggagaagccctcgttcaagtggctgcggctggtatctgcggtagcgacctgccccgcatctttgagactggaacttatcattttccaactatcccaggccatgaattcgcaggtgttgtcaaagaactttatgctgatgccgacgaagaaatgctccgacaatggatgggtaagcgcgttgcggtgtttccactgattccctgtcgaacctgtcgaagctgcgcgaatcatcagtacgagatgtgttctaactataattacctcggctcgcgtacggacggaggcttcgctgaatactgtatcgtgcctgtttggaacttgttgcagttgccggacgaggttagcttcgcacaggccgctatgatggaaccggcagctgtggcattgcatgcaatccgacgtggcggcctgcaccccgaccaccccgagaccggtatcggcaagcgtgttctggttatgggacttggcactattggcggattgatcgcccagtggcttagccaatgtggcgttacggagctcaccgccactggtcaccacgccccccaggaagagcttatgcaccgatatgctaacgagaactaccgattcattaatactaatcaaactgcttggaccgatgacgaaaacacctacgatcttatttttgactgcattaatgacagcgaatccgttcagcaggcgttgcgctacgtcaaacccggtggtcagattgttgctgttggcaacccgaaaaaagatcttactcttcaaaaggacatctattggaaactcctgcgaaaacaagtgtgtatgaccggaacatggaactcgagcttcagccatgattcgaacgatgactggcagatcctccttcgataccttcaggagcaaaagctgcatccgcaggcacttatcacacatcgccttccgtttgcccaacttcccaagggcttggagatcatgcgtcttcgtgaagagttttacaacaaaattatgatcgacttgactgtctgttcttaa

SEQ ID NO: 105 - DNA, 1029 bp, synthetic construct
  source 1..1029: mol_type = other DNA
atgaaggccgtgacttacactcccgaccagggttttgcattggtcgaggttgagacaccccagcttggacctgacgaagttcttctccaggtggaggcttgtggcgtctgcggaagcgaccgtcaagtcgttgcaggcgaatcggttcctaccggtaccgccttccctctggtgatgggccacgagattgccggacgtatcgccgcgttgggcgaaactgtgacagattggcatgttggagactctgtgattgtccatcccttcgtggcatgtggtacgtgcgcaccctgtgttcacggccagcctaacttgtgcgtccgacaggtgtgtattggttatcaacgacctggaggatttgcagagcaagttgctgtcccggctcgtcaactggttcgacgtccagcagaattggcaccggccgccgcagcattggttgtggatgcgtttgccacgccataccacgcgatgcgagcggcgggtgttgagagcggccaaaccgttttgattattggaaccggcggacttggtctcgcggcacttatgctggccatgggcttccatgtcggccgtctgggcgcggtcacgcgacgccaagagggtattgcaatcgcggaggctcatggcgcgcagatggttgtcgtcacggaccaggatgagcgtacaattgcgcgccagcttcgccgctggtctggcgcatccggcattgacgtcgtcatcgatacggtcgctactgctgacactattacattggcagctgaaagcgttcgcccaggaggcactatcgctattgtcggaatgtcggccgacaccgccctctatcctgtcgctaagaccgttcgccgaggtctgacattcgttgcgagctacggatccgtccgagaggatgtcgagcagttggtcagctgggtggcagaaggtcgactggacccggttcgactcgtcgcgggagccctcccactccatgaagccgctcgtgcattcgccccggaccgacccgccggacgttgggttattgatccatctgaataa

SEQ ID NO: 106 - DNA, 1074 bp, synthetic construct
  source 1..1074: mol_type = other DNA; strain = KGMB03662
atgaaaagcttgattaatatcccggaaaaaatgaaagccctcgttttgtatggtgttggtgatttgcgtgtcactgaagtggacgttccgaagctcgaaaaaggaacggtcctccttaaaattaaagcttgcggcatctgttcctccgacatgcctcgttgtttcgttactggcacttaccattttccgaccattccaggacacgagttctctggacaaatcgttgcggtgggtgatgatgttgatgaatccttgttgggaaagcgttcgtgcgttttccctatgcttccctgcaaacactgcaaagcttgcaagatggaggaatatgctcagtgtagcggctattcttacttcggctctcgccaagatggtgcctgggcggaatacctcgtggtcccggtgtggaacctggtgccctttgacgatacactcgattataaaactgcagccctgtgcgaaccagctgcggtctctatgcacgccgttaatattggagacattaaagaaggtcagaatgttgtgatcatcggaacgggtaccatcggattcatgattgctctctttgccaaagaacgtacgaaaacaggaaaggttgtgatcgcgggacgctcgaaaaataagctggaatacgcgaaaaagttgggcttcgatgtcgttgacacctcggttgacgatttgcctgcccaagtgaagaaaatcatcggtattgatggagctgacgtcacattcgaagcggtcggctccaatgaggccattgcctctgctattgaatctacgggcgctttgggttcgattgttcttgtcggtaatccttccactgatctcatcttgccaaagaatatttattggtccatcctccgaaagcaaattacagttaaaggttcctggaactcgtcttataactctcgcgttaatgactggaagaaagcgttggaggtgtttgagcatagcgacgtgaattttgcagacttgatcacacacacattcactatcgaagaaaacgagaaagctttttctgccatccgtaacactggagaattcactctcaaagtcatgtttaccttcgatgattaa

SEQ ID NO: 107 - DNA, 1047 bp, synthetic construct
  source 1..1047: mol_type = other DNA; note = Clostridiaceae bacterium
atgaaagctgtcgtcctgcatggtaaaaacgacatttgctatgaagaatggcctgaacccatcctgaaaacaggaacagttaagattcatgtcaaggcttgtggcatttgcggctctgacattcctcgcgttctcggagattctgcccattactacccgatcatcctcggccatgagttctccggttgtgtcactgaggtggctgatgacgttgacaagatccaggttggcgatcatgttgcaggagtcccccttctcccgtgcatgaaatgcgatgattgtcaacagggaaactattctcagtgtaaacattactcttttatcggcagccgcgagcaaggtggatttgcagattatgttgtgatcccagccgataacgtggttaaagtcgacccaactcttccttacgaacaggtcgcactcttcgagccttgtactgttgctttgcatgcactcaaagtctcctcgtataaaggaggaaaacgagtggcgatcctgggtggcggcacgattggtctttttgcactccagtgggctaaaatctatggcgcgtcccaagtcacagtcttcggtcgcagcaaggaacacttgaagatcgctgctgctctgggtgccgacacaactatttccactctggacgataattttatgacagaggctatgcgagtcacagagaaaaagggttttgactacatttacgagtccgccggttccacaatcaccatgaagcttgcttttgaactggcagccaaccacgcgaaaatctgtttcattggcacgcccacggaaaacctgactttcagccctaagctgtgggagaacatgaatcgaaaggagttccagcttatgggttcctggatgtcgtgttcggcgccgttccccggcgatgaatggaaggagacagcccatttctttaacaccggacaacttcgatatgtgcctgagatgtttttcgcacagtatcccatggacgatgttaaaaaagcgttcgagcagttccataacaagaataaagttaagggacgtatcctgctcaaaaacaagtaa

SEQ ID NO: 108 - DNA, 1014 bp, synthetic construct
  source 1..1014: mol_type = other DNA; note = Thermoplasmata archaeon
atgaaagtggcggtttggtatggtgacaaaaatattgtcttggaggaccgtccgatcccttctatcggtgaaaaggacgtcttggtccgtgtcaagtcgtcttccatctgcggagctgaccttcatgcctacaaaggcctctctaagcgccgcaagcctccattggtgatgggacatgaattttccggtatggttgaacgcctcggcgatgagacaaagggccttaaggtcggcgatcgtgtcgtggtggagcccattattagctgcggaaagtgcaaggcatgcaaaaagggaaaaacgaatgtctgcgaaaatcgtaagctgatcggtctgaacctcgacggtgctttcgctgaatatgtttctgtcccttatgaaaagtgccatgaactcccccgtgacctttcgttcgatgagggtgcgattatcgagccgttggccgtcgccgttcatgcggtcaatattactcccatctcgtcgaacgataacgtgcttatcattggtagcggtgtgattggcctcctcatcacacaattgcttcgaagccgcacttcgggcgacattttggttaccggacgtcgcgattacctccttgaaaaggcccgtgaatttggtgccacagatgcaatcaacgtgatcgactgcgatgctgtgaaaacgctgctcaaaaaacacggtcgcgtcgatgttgtcttcgaagcagtgggctctaacgagactgtgaaccagtccatttccctggtcgaatctggaggttctatcactctcgttggcatgctcgatcgcgagatgaccgttgatatgatggaggttgttgcccgtgaaatcactctgcgaggagattatggttatacaactgacgactttaaggaggcgatcgagctcgcttcttcgggtaaagttgatctcaaaggcctcatcacgcatgttctcccgctcgatgagatcaaaaagggcttcgatatcctggcagaacgccgagaaaatgtgatcaaagttgttattaacccataa

SEQ ID NO: 109 - DNA, 1032 bp, synthetic construct
  source 1..1032: mol_type = other DNA; note = Lachnospiraceae bacterium
atgaaagcactggtgttggaggaaatcggtaaattgacccttaaagaggttgagacgcccgtgccaggacgcggagaggttttgttgcgcatccatgcctgtggtatttgctcttcggacaaagcccgtctcttcaagaacggtactcacaccatgccgcttatcccaggtcacgagatctcgggtgagattgttgcaggaggtcccggcattcgagaagagcttatcggccgacgcgctgcggtcttcccgttgattccttgtcgccaatgcgactcctgccgaatcggcaagtacgctcaatgcgagcattacgactatttcggatcgcgtcgagacggaggctttgctgagtacctcgctgtccccgtgtttaatatcaatcttcttcccgatgatatggattatcatacggctgcattgtgcgaaccagccacggttgcatctcatgcacttaagcgcgccaaaatgaaatctggtgatatcgttacgatcattggcactggcacggtcggcctcctggctgctggcatcgctcgtattcgacgagcatctaaaatcattatcgtcggacgaagcaaagaaaaaatcatgaatgcagctcatttcgctgatcatgtgatcaatgcagagggatgggacgtgaacgaggccgtggcttcgctgacggagggtcacatgagcgatgtggtcttggagactggcggcaacagcgatgcttgcacgtacgcgcttatggctgccggcaaaggtacgactgtgatgtttattggcaatcccgaagaaccgattgtttttaaccgtgatatttattggaagatcctccgaaacgaactgacactcaagggtacttggaattctggatatgctcctgaggataatgactggaatgacgtcatccgttatatggcagatggacagattccggcagataaaattatcaccgatagctttccattggaagattataataaggcattcgagcttcttctctcggatcgcttttacaccaaaatcatgtttgacatctaa

SEQ ID NO: 110 - DNA, 1047 bp, synthetic construct
  source 1..1047: mol_type = other DNA; strain = DSM 16973 = JCM 13450
atgaaagccaatgttctgtaccacattggtgacctgcgatacactgacgttgccttgccggaactccacgacggtgaggccctcgtgcgagttcgtgcatgcggtatttgtggtagcgatgttgcccgagtcttcaccactggtacgtatcatttccccaccattattggccatgagttctcgggcgaagtcgttgaaacatattctgcggctgacgcggagtgggtgggtcgccgcgtgagcgtttttcccctcatcccttgtcgccattgtgatgcctgtcatcatggtcagtacgaaatgtgcacatgttacaactatctcggcagccgtaccgatggaggcttcgcagagtacgtggctgtcccggtctggaatctctttcgtctgccagattccgtgtcttacgaggagggcgcaatgatggaaccggccgctgtcgctatgcacgcgctcaagattgcccgtgtgggtatgggtgacactgtggcaattacaggtcccggaactattggtatgattttgacccaattgatccaacgtattggcggtacaaaagcggttctgatcggacgcacccaagcaaaattggattacgcacgacgcatcggcatccgtcatgtgtgcaattcgggaaccgagcacgtcgcggaccgcgttggacaacttacaggcggacgtggagccgacatcgcaattgaaggaaccggtgcgtcggaacccatgaatctgtgcttgaatttggtgcgctctgaaggacgtattgtcgctatgggaaacccccaaggtgacatgctgttccgaaaggatgcgtattggaagcttctgcgcaagcagctgacccttcgtggaacctggaattcgacatatggattgggccgagatgattggcaggatatttccgcccttcttgctgagggagcattgaacttgcgcggccttatcacacaccgactcccactggccgagctgcagcagggactcagcctcatgcgagacgcagcagtctacactaacaaagttatgatcgtcgacgagcaaggttaa

SEQ ID NO: 111 - DNA, 1014 bp, synthetic construct
  source 1..1014: mol_type = other DNA; strain = OM05-6
atgcgcgcctatgagcttcacggagtcaacgacttgcgtcgcgaggacatcgaaaaacctgagattccctctggatgggttcttgtgcaagtcaaagcgagcggcatttgcagctccgacatccctcgtatcttcacgaacggcacttatcactttcctaccattccgggccacgaattctctggagtcgtggccgcctatggcgaaggtgtcccggaagaacgcgttggcaaacgagtgggtatttttccgctgatcccgtgtcgtacatgtccccaatgccgccagaagaaatacgaaatgtgcgagcactatgattaccttggctcgcgtcgtgacggcggattcgccgaatacgttgcagtcccggactggaacctcctggaattgccggagaacgtttcgttccgcgaggcggcgatgctcgaacctctgtcggtggcacttcatgccgtgaaacgcagcggcgtgaaacctggtgatacagccgctgtgatcggcacaggaatgatcggttttgccgcagctgcttgggccaaagcactgggagcagaaacggtgttcgtgattggacgcggcgaggccaaacgcgctattgctgaacaaattcccggaatttcctatgtgactgaagaggaatccaaggccattcaggcggacgtggtcgtcgaagcagttggtacaccacaggctgtttcctccgcggtgttgcttgcacgaccgggaggttccattgttctgatgggcaacccatccggtgatcttgcgatggaaaaaaacgtctactggcgtatcctccgtaagcaacttaacttgatgggaacgtggaatagctcgtatgaaaaagacgaagcctgtgactggaccgaagcgctcgacgcattgtccaagaaaaagattcctgcccaagcacttatcactcattgttttcccagcgaaaaacttatggatggactggaattgatgaagaatcataaggaaccatactgtaaagttatggtcagctgggagtaa

SEQ ID NO: 112 - DNA, 1065 bp, synthetic construct
  source 1..1065: mol_type = other DNA; note = Candidatus Bathyarchaeota archaeon
atgaagcgtatgcgagctgccatcttgtatggaccaaaagacatccgaattgaaaacattgagatgcccaaggttaagccggagtgggtgcttattaaggttcgcgcaacaggtatctgcggttctgatctgcacttttacaaagagaagagctttattcctattattagcgacgtgggcaaaggacgctacgtccccggtcatgaattttcgggcgaggtctacgagttgggttcgaaggtcaaaggcctgaagaaaggagatcgcgtcggcgtcgaacccatcattggttgcggcaaatgtcagtggtgcaaggtcggatgttacaacttgtgtgagcactacaaattgattggctttcactatattggaggtatggctgagtattgtgttgctcctgctgagaattgttttaagctcccaaacaatgtttcgttcgaaagcgcggcaatgcttgactgtatcgccgtggctattcacgcagtgaataaagcagaaattcgcggcggtgacagcgttctcattgtcggcgccggtgccatcggactcttcgccctccaggcagcgctcatccagggagcgagcgacgtgtacgtcgttggaaaatacgaattccagttggagaaagctcgcaagttgggagcgacagctgtgattaacgctaaaaacgaatctttctccgaaaaaatcatggaaattacttcgggtaaaggagtcgataaggtcattgaggccgtcggcggagcctccaatgttatttctgacgtccttgacgtgactcgaaaaaagggtactatcgtgaccactggtatctttacacgaccactctcggtcgattttttcaaaattctcacaaaagagttgacgatcacttcgtcgtggggatactgttacttcgaccgtcgcaaggagtttggcacggcgctcgactttctggcaaaagaaaagatcaacactcaggaacttattacacataagtatcctttggagaaggtttccgaagcctttgagacggcactcaacaaaaataaggagaagagcatcaaggtggagattatcttctaa

SEQ ID NO: 113 - DNA, 1053 bp, synthetic construct
  source 1..1053: mol_type = other DNA; note = Lachnospiraceae bacterium
atgaaagcctgggtgctgcacggtatcggagatatccaatacgaggaagttaaggagccgcagtcggctggcgacgaggttattatgaaagtgatgagcgtgggaatctgtggatctgacatccctcgcatttataaaaccggcacctatcatttcccaacaatccccggccacgaattctccggtattgtggccgatgttggtgccggagtcaacccgaaatggaagaataagcgcgtcggcgtgtttccattgatcccatgtcacgtttgtgcttcgtgtaaaaaaggtcaatatgaaatgtgtcgcaactacggatatctgggtagccgccaaaatggtggttttgcagagtatgcggcggttcctgtcaagaatctcatcgaactcccggactctgtctctatgcaggaagctgccatgcttgagccgatggccgtggcagtccatgctatgcgacgagcccgtcccatgaccggagatcgtattgtcgtgtgcggacttggaactatcggcatgcttctcgtgtcctttttgcgagagacgggaattcgagacatctttgtcatcggcaataaggcattccagcgaatcaccatcctcaaaactggcctgcaggaaaactgttattgtgattctcgcgtgcaggatgtcgagaagtggctcatggagcgaactgaaggaaagggcgcagacgtgttctttgagtgtgttggccgaaacgaaaccatctcccaagcagtcaacctgacggcacccggaggttcggtctgtctggttggtaatccttgttcggatatgacgcttgagcgtgaggtctactggaaaattctccgtaaccagcttactattactggcacctggaattcctcgttcacccacgagatcgaggacgactggcattacgtgttggatcgcctggaacaaggtaagatctcgcctggtgatctcatttctcacaaattcccgctcaagaacttggaatctggtctgcatattatgcgtgataagactcaagattatatcaaaatcatggccgagattaatgtgtaa

SEQ ID NO: 114 - DNA, 1047 bp, synthetic construct
  source 1..1047: mol_type = other DNA
atgaaggccgcagtgctccattccgaaaatctgtttacgtacgaagaagtggaaatcgatccctgcaaagacgacgaggttaagatcaaggtgatggctgcgggtatctgtggttccgatacacataagatgacgagcaaatggaaatatccccttccgaccatcatgggccatgagttttcgggctttatcgtcgagattggtgaaaaggtcactaacgcgtcgattggcgatcgagttgtggcgatccctttcttgccgtgcaatgactgcgaatactgtaagcgtggacaattctcgttgtgcgaggatcacggcatggttggatccaaaagctttggtggtttcgcggagtacgtcaatatcaaaagcactaatgttcttccgattggtgacatggattttgaggaagcagccatgattgaacctctcgcagttgcgcttcacggcgtgttgaatattcatccgcgtattggagatattgcagccgtctttggtattggcacaatcggaatcctcacaatccaatggcttaaaattgcaggagtgaaagaggttattgcgatcgacatttccgataacaagctcgcagacgcccatgctatgggctgtgagcgtactatcaatccatttaaagagaacctccttgagaaaatcgacgaatatactaacggtcgcggtgttgacatcgcctttgagtgtgctggatctaaagtcacacaagaacagtgcctgctcgtgacagccaagcgaggaaaagtcggctatttgggtatcgcttactctgatgtccatttgtcggagcaagcttttgaaaacattttccgccgcgagctcacacttaaaggattttggaactcttactccgcaccattcccgggagaggaatggaccaaaagcattgacttcattaataagggtcgtattaaactgaaagagctgattagccaccgatttaagctccaagatactcagaaagcgtttgacatgatgatcaaccgagaagagacatacggcaaggtgcttattttgcccaacaaagagtaa

SEQ ID NO: 115 - DNA, 1047 bp, synthetic construct
  source 1..1047: mol_type = other DNA
atgaaggcggcggtgcttcattctgagaatcacttcatttatgaagaaattgatattgctccttgtcaagatgatgaggttaaaatcaaagtcattgcgactggaatttgtggatcggacacgcacaagatgacaggcaaatggaaatatcctctccccgccgtgatgggacacgagttctccggatatatcgttgagaaaggaaagaacgtcactaatgcagccatcggtgaccgcgtcgtggcgatccctttcctcccgtgtaacgactgtgagtattgtaaacgtggccaatttagcctttgcgaagaccacggtatggtcggctcgaagtccttcggaggttttgctgagtacgttaatattgagtcttcgaatgtccttcccatcggagatatggaatttgaagaggcggccatgattgagccgctcgcggtggcccttcatggagtcttgaatattcatccgcgtgttggcgacgtcgcggccgtgttcggtgtcggtaccattggtatgctgaccatccagtggctgaaaattgcgggtgccaaggagatcatcgcaatcgacttgtcggatgaaaaacttgctgacgcgcgtgctatgggttgtgaacgtacgatcaatcccctgaacgaagaccttcttgaaaaaatcaacgaatacaccaacggaatgggagttgacatcgccttcgagtgtgctggctcgaagattacacaagaacagtgccttcttatcacccgcaagcaaggcaaaattggatacctgggcattgcttattccgatgtccacctgtctgagaaggctttcgagaacatttttcgacgcgagcttacgttgaaaggtttctggaattcgtattccgctccatttccgggtgaagaatggacaaaatcgattgaattcattaaaaacggccgcattaagttgaaggaactgatctctcaccgatttaagctggaggatacacaaaaagcatttgatatgatgatcaaccgtgaagagaaatatggtaaggttttgattcttccgaacgaggagtaa

SEQ ID NO: 116 - DNA, 1053 bp, synthetic construct
  source 1..1053: mol_type = other DNA
atgaaggccgcggtcctgcactccgaaaatgagattatttaccaggacattgaaatttccgagtgtcaaccaaacgaagtgaaagttaaggtcatggcggctggaatctgtggttccgatacccataaaatggtgagcaaatggaaataccccctccctgcgatcatgggtcacgaatttgccggctacgtcgttgaaaaaggcaaagacgtgacgaacgtcgaaattggcgatcgtgttgccgctatcccgttcatcccatgcaacgagtgtgagtattgcctgcgaggtcagttcagcctttgcgaaaatcacggaatgatcggcagcaagtcctttggcgcatttgccgagtttgttaatattccagctaagaacgttctcccgattggcgaaatgtctttcgaagatgcagctatgatcgaaccgtctgcggtcagcctgcatggagtgctcaacattcacccacagctcggcgatgtcgttgctgtcttcggtgtgggaactattggaatcctcgcaattcagtggttgcgtgtcgccggcgtgaaagagattattgctattgacatttccgacaagaagctccaggacgctatggatctgggttgtgagaagtcgattaaccctttgaaggaagacttgattaagaaaatttatgaatacacgaatggcctcggcgttgacatcgcatttgaatgcgctggatctaaaattacgcaagaacagtgcctgctcattacccgtaaacaaggaaaggttggttatttgggaattgcgtacagcgatgttactctctctgaaaaagcctttgaaaatattttccgccgagagctcactctcaagggtttctggaattcgtattccgcgccgtttccaggaaaggagtggatgaacacgatcgagttcgtccaacagggcaaaattaagcttaaagaattggtgtcgcatcgctacaagctcgaagacacccagaaggcgtttaacatgatgatcaaccgagaagaagaatacaataaggtgatgatcatccccaacgaggaactggagtaa

SEQ ID NO: 117 - DNA, 1041 bp, synthetic construct
  source 1..1041: mol_type = other DNA
atgaaggcagcggtgcttcattcggtcggagacatccgctacgaaaacatcgagatcaagccttacggagacgatgaagtgaaaatcaaagttaaagccgccggtatctgtggatctgacccgccacgtgcactcaaaaagtggaaatatcccgtgccagccattttgggtcacgagttctcgggttatatcgttgaagtgggaaagaacgtgaagggttttaaagtgggagatcgtgtcgttgccgtccctctggaaccgtgtcaccaatgtgaatactgcaagaaaggccaattttcgctctgcgaaagctacgatatgttgggtgctacttcctttggcggttttgcagagtacgctaacgtgaaggcaacaaatgttttgaaaattgacgatatcgactatgaagaggcggccatgattgaacctctcgcagtcgcactgcatggtgtgcttaatatcaatccgcaactcggtgatactgttgctgttttgggagctggaacgatcggtcaactcacgattcaatggctcaaggtttcgggagtcgagaagattatcgcagtcgacatttcggacaaaaaaattagcgaagctatgtccttgggagcagatattggtattaatgctcttaaagagaatccggtcgaaaagatcatggaacttacaaacggtttcggcgtggatatctgcattgaatgcgccggctctaagatcactcaggaacaatgtctgttggtcacgaagaaaaaaggtaagattggatatcagggtatcggtcacgccggaattgagttgtctgaagttgctttcgagggtattttccgtaaagaattgaacatccaaggtttttggaatagctacagcgccccgttccccggacaggaatggttcaaaagcatcgagtatgttaagcagaaaaaaatcaagctcaaacagttgatcagccatcgcttcagccttgaagacacagcgaaagcttttgagatgattggtgaacgtaaggaggagtataacaagatcatgattgtccaggactaa

SEQ ID NO: 118 - DNA, 1047 bp, synthetic construct
  source 1..1047: mol_type = other DNA
atgaaagcagcagttttgcattccgagaatcacttcatttatcaggatatggagagcttcgagtgccagaatgacgaagtcaagatcaaggttatggctgccggcatctgcggaagcgatacgcataaaatggtttcgaaatggaaatatcccttgcctgcaattatgggacatgagttctccggttacgtcgtcgaaaagggtagcgatgtggcaaacgtcgagatcggcgaccgagtggttgccattccattcttgccctgtcacacttgtgagtattgcaagtccggtgtttattctttgtgtgaagatcacggtatgatcggttccaagagctacggtggcttcgcggagtatgtcaatctcaaggcaaccaacgtccttccaattggaaacatggattttgaggaggccgctatgatcgaaccgcttgcagtcgccctccacggcgttctccatattcagccgcgtctcggtgacgtggccgccgtttttggagtcggcaccattggcatgttggccatccaatggctgaagattgcgggagtgaaagaaattatcgctattgatatttcggatgagaagttggcggaagcgaaagccatgggatgcgaacgaacgattaatcccctccaagacaatctcatcgaaaaggtccacgagtataccaacggcttgggcgtggatgtcgccctcgagtgcgcgggatctaagattacacaggaacaatgtcttttgatcactcgcaaaaagggtgaaattggttacttgggaatcgcgtattctaatgtggttttgagcgaacaagcttttgagaacatctttcgtaaagagcttacgttgaagggtttttggaattgttatagcgctcccttccctggcgaggagtggacgaaatctatcgacttcattctgaagggccgcatcaagctgaaggagcttatcagccaccgctttcccctggaagaaacgcagaaggcattcgacctgatgattaatcgaaaagaaacctatggtaaggtgatgatcatccccaataaggaataa

SEQ ID NO: 119 - DNA, 1047 bp, synthetic construct
  source 1..1047: mol_type = other DNA
atgaaagcagcagtgctccattctgagaatcagatcgtgtttcaagaatttgagatcggcgaacttaagccagacgaagttaaggtcaaagtgatggcagcgggcatctgtggctcggatacccacaagatggtgtcgaagtggaagtatcccttgccagcaatcatgggtcatgagtttgccggctatattgtcgaaaagggaaaagatgtggtgcatgtggaagtcggtgatcgtgtggcaggcatcccttttgtgccttgtaatgactgtgagtactgttttcgcggtcagttttccctttgcgagaatcacggtatggtgggttctaagtctttcggagcctttgcggaatatgttaacattccagccaaaaacgtgctgccgattggagacatggattttgaggacgcggcgatgattgagccttcggccgttgccctccatggagtcttgaacattcacccccaaatcggtgatgttgtggcggtttttggagttggcaccattggaatccttgcactccagtggttgaaagttgccggtgttaaagaaattattgccattgacatctcggacaagaagctccaggatgccatcgccctcggatgtgagcgcacgattaatccattgaaagagaatttgctggaaaaaattcatgagtatacgaatggtcttggagtggacatcgccattgaatgtgcgggcagcaagattacacaagaacagtgtcttcttattacgcgcaagcagggcaaagtcggctatcttggcatcgcttactccgatattaatctgtcggaaaaagcatttgaaaacatcttccgccgcgaactcaaaattaatggtttctggaattcttattcggctccgttcccaggtaaagagtggacaaacaccgtggagtatgtgaagcaaggcaagattaagctgaaggagcttatttcccaccgattcggactggaagacacgcagcaagcatttaatatgattatcaaccgagaggaggaatataataaagttatgattgtcccaaacggcgagtaa

SEQ ID NO: 120 - DNA, 1026 bp, synthetic construct
  source 1..1026: mol_type = other DNA
atgattaatcaggtttaccagctggtggcgccgcgccagttcgacgtcacgtataataacgtggacatttacggcaaccacgttatcgtccgaccgctgtatctctcgatctgcgcggccgatcaacgatactataccggacgtcgtgatgagaatgtgctgcgaaaaaaattgccaatgtctctggtccatgaagccgttggtgaggttgtttttgatagcaagggagtgtttgagaagggcacaaaggttgttatggtcccaaatacgcctacggagcaacaccatattatcgccgagaactacctggcgtcttcgtatttccgctcgagcggttacgatggtttcatgcaggattatgttgttatggcgcatgaccgtattgtcccgctgcccaacgatattgacctgagcactatttcctatacagaattggtttcggttagctaccacgccattcaacgtttcgagcgtaagtccattccgctcaagaatagcttcggtatttggggagacggtaatttgggctacatcactgcaatccttcttcgcaagctctacccggaggcaaaaatttatgtcttcggaaaaacggattataaactttctcatttcagcttcgttgatgacatcttcacggtgaaccaaatccccgatgatttgaagattgaccacgccttcgagtgcgttggcggaaagggctcgcaagttgcgttgcagcaaatcgttgagcatatctcccctgaaggttctattgctctcttgggcgtgtccgaactccccgtcgaagttaacacccgactcgtgttggaaaagggcttgaccctgattggatcctctcgttccggttccaaggactttgagcaggttgtcgacctctatcgaaaatatcccgatattgtcgagaaactcgcacttttgaaaggccacgaaattaatgtttgtacacttcaggatatcgtgcaggcatttgaaatggacctctctacgtcttggggcaagacagttttgaagtggacgatctaa

SEQ ID NO: 121 - DNA, 1053 bp, synthetic construct
  source 1..1053: mol_type = other DNA; strain = 71193; sub_species = aureus
atgttgttgtttgaagttgttctcccaatgatcaaccaagtttatcaacttgtcgcaccccgccaatttgaggtgacgtacaataacgtggacatttacagcgactatgttattgtccgacctctttacatgagcatttgcgctgccgaccagcgatactacaccggatcgcgtgacgagaatgtcctttcccaaaaactgcctatgtctctcattcacgaaggagtgggtgaggttgttttcgattccaagggagtctttaataagggcactaaggtggttatggtccccaatacaccgaccgagaaagacgacgtgattgcagagaattatcttaaaagctcgtattttcgttcctccggccatgacggtttcatgcaggacttcgttctccttaatcacgatcgagctgtcccgctgccggacgacatcgatctctccatcatttcctacacagagttggtgacagtttctctccatgccatccgccgatttgaaaaaaaatccatcagcaataaaaacaccttcggcatttggggagacggaaatcttggttatattaccgcaattctcctgcgcaagctctacccagaatctaaaatttacgtgtttggtaaaactgattataaactgagccatttctccttcgtggacgacgttttcttcatcaataagattcctgaaggacttacctttgatcacgcctttgagtgcgtcggtggacgaggctctcaatccgcaattaaccaaatgattgactatatttcgcccgaaggtagcatcgccttgctcggtgtttccgagttcccggttgaggtcaatacgcgcctcgtcctcgagaagggactgaccttgatcggctcttctcgctctggttctaaagactttcaagacgtggttgacctctatattcaatacccggatattgtcgacaaattggcgctccttaaaggacaggagttcgaaattgccacaattaatgacctgacagaggcgtttgaggctgacctctccacctcctggggaaagacggttttgaagtggattatgtaa

SEQ ID NO: 122 - DNA, 1026 bp, synthetic construct
  source 1..1026: mol_type = other DNA
atgattaataccgtctaccaactggtggcacctcgacgattcgagattaagtattccgacattgatctcaacaataataaggttatcgtccgtcccacctacctctctatctgcaacgcagaccaacgatactaccaaggactccgcgatgccgaaattctgcgtaagaaacttccgatggcgttgatccatgaagccattggagaggtcgtgcgagatccatctggaaactttaaaaccggcgagttggtcgttatggtgccgaacacacctgttgaagaagatgacatcatcgctgagaattatttgcgatcctcgaaatttcgtgcatcgggttttgacggcttcatgcaagaatatgtggaaatcacaccggatcgcctcgttcgtcttccccagaatatcgaccgtactgttgcagcttttacggaaattgtttctgtcagcgtgcatgcaatcggtcgttttgacaagatcgcgcacaaacgacgaaacgttgtcggcatctggggagacggcaacctgggatatattactgcggttttttttaagactatgtttccagagacgaagctgtatatttttggagtcaacccagataaacttcaagattttacgtttgccgacgcgacgtttacggtggagcacattcctgaagacgttaagattgaccatgcttttgagtgcgtgggtggcgcaggctcgggcaaagccattaatcagattatcgactatatcaatcccgaaggtaccatttccattttgggcgtttctgaatacccagtgccaattaacacccgcatggtcctcgagaaaggactgcgcatctttggatcgagccgtagcggtgtcgccgattttgagaagaccgtcgcgctctacaaatccaatcctgagattatcaattatctttcgaatatcgtcggtgcagtcgtcccagtgcgaacgatcgaagatatgaaaaaggcgttcgaaatggacatccaaaaggcttttggtaagacgattatgatgtgggataaataa

SEQ ID NO: 123 - DNA, 1032 bp, synthetic construct
  source 1..1032: mol_type = other DNA; strain = D12
atgattaatcaagtctatcgtctcgtctctccacgccaattcgaggtcgcgtataacgatcgcagcctccagtatcacgaggactttgttgtggttcgacctacgcatctgagcatctgcgctgcggatcaacgatattacactggctcgcgtggaaaagaagctatggcaaaaaagcttcccatggctctgatccacgaggccattggtaaagtggtctacgaccccaagggtgagatcaaaaatggcactatggtggtcatgattccgaacacgcctactgaaaaggatgaagtcattggtgagaattatctccgatctagcaaattccgctcgtccggatatgatggattcatgcaagattacgtcttcatgggtcgcgaccgtattgtggaactcccggaaggaattgacccgtatgttgttgcttttaccgagttgatgaccatcacaacccatgtgttgagccgatttgaatctcgttcccataaacgacgtaacgtttttggcgtttggggagacggcaaccttggtttcatcacgacactcttcctgaagaagatgtataaagattgtaaagttatcatcttcggaaaaactggatataaactcgacaatttctctttcgttgatgaggtgtatacgattgacgaaatccctgaggacctgcgcatcgaccatgcctttgagtgcgttggaggtaagggttctcagtatgcaatcgaccaaatgattgaccatctccaccctgaaggttccattgctcttatgggtgttagcgaatttccagttgaagttaatactcgtatgatcctggaaaaaggaatcacactgatcggaagctctcgatccggtcgagaggacttcgtgcgcacggtcaactttctgaaggaaaatcccgatgttgttgaatacctgggtacccttgtgggcgaggtccaagaagtcaagacgatctccgatattttgtctgcatttgagaaagaccttacatcctcttggggtaaaaccgtcatggagtggaagatctaa

SEQ ID NO: 124 - DNA, 1026 bp, synthetic construct
  source 1..1026: mol_type = other DNA; strain = EGD-AQ4
atgctcaaccaagtgtatcgcctggtcgatccacgtcagattgaagttcaaactgttgcagaagagatcactgataatgacatcgtcgtccgccccaaatatctgtcggtctgtcatgcagacacgcgatactttacaggtcaacgtccccaagcaaccttgcgccagaagctcccaatggctttgatccatgaaggagttggcgaggttgttaaggaccctcaaaataaatttaagcccggtacgttggtcgctatggtccccaatacgccctttgaaaccgaccctatcattaaagaaaactacttgccaacgtctaaatttcgcagcagcggttacgatggtttcatgcaggaatacgttacacttcatcgtgatcgcgcgatcgtcgtgccagacaacttcgaccaccaaatgagcgcattcattgagatggtttccgtcggagtccacgccctcacccagctggaggccgtgatggatgcggatcgcaagatcatcggaatttggggagatggcaacttgggttttatcacagcaaccctcgtcaagcaaatctttccagactctcaactgatgatctttggacgacatcgctccaaattggactatttcagctttgcggacaagacatacctggtggacgacattcctagcgatcttcaagtttctcaggctctggaatgcaccggaggccgaggctcggagtctgctattgctcaaattatccaacacattcgaccaatgggcaccgctatccttatgggagtttccgaagatcccgttggtatcgatacacgcagcgtgttggcagagggattgacccttcgcggcgtgagccgtagcggtcgcgcggattttcagcgcgccatcgacattttgacgaaatcccccgtcacacgtgaacgtctgcaaaatcttgtgggtttcactcgaaaggtcagcacaatccaggatatcaccgatttcttcgagggtgcattgacaaactactggggcaaggccgttatggagtgggatgtttaa

SEQ ID NO: 125 - DNA, 1014 bp, synthetic construct
  source 1..1014: mol_type = other DNA; note = uncultured Ruminococcus sp.
atgatcaattacgtgtatcagctggtcagcccccacgtcttttccctcaaatatgaagaagtggacttcaagaaagaggtcatcatccgtccgaagtgtatggctgtgtgccacgcagaccagcgatattttctgggacgccgcgacgctaagatcctgcgacaaaaattgcctatggcccttatccacgaatgttgcggtcaggtggtctgggatggcacaggttccttccaacgaggccaaaaggtcgttctcattccaaatgtgccaggacgcgacgacggtgtcacatacgaaaactacgctgaaggatctcgttttcttagctcgggctgcgacggtttcatgcgcgagtatgtcaacctttccccagatcgtgtggttcccttcgaacaggttccgcttccggtggccgcgatcacagagttcgtgtccgtggcggttcacgcggccggtcgctttatgcgagcggcgcacacacgccgcgaggctatcggagtgtggggagatggctctctggcttatgtcgtctgctgcgttcttcgtcgactgctcccggagtcgcgtatttttgtggtgggtaagcatgaacgcaagttgctccagttctcgtttgccgatcgcaccttcctttttgatagcctcccggaggacttccatattgaccacgcgttcgaatgcactggaggagagggctcttactatgctatcgatgatattattaccgcggtgcgtccccaaggcgctgttatgcttatgggtgtctctgaaaaccgcgttccagttcgcacccgtgacattctggaaaaaggcttgacgttcaccggttgttctcgttctggacgcgaagactttgttaaggcagtcgagcttatggagcaaccccaattccaaaaacgccttagcgcaattatttacgaggatcgtccggtccatactgtcgccgatctgtcccgtgttttcgagacggatttgaatacgccgttcaagacagttttccgttgggagatttaa

SEQ ID NO: 126 - DNA, 1017 bp, synthetic construct
  source 1..1017: mol_type = other DNA
atgattaacacggtgtatcgccttatcgccccaaagtccattcgagctgactttgtggatctgaaattggatgaagaacgcattatcatccgccctacatatttgtcgatctgtgcagcagatcaacgttattacaccggatcccgtggtcgcgtcgtgatgaagcaaaaacttccaatggcacttatccatgaggctgtcggagaggttgtttatgaccccaaaggtgagctcccagtgggaacgcacgttgtcatgattcctaatactcccaatgagaaagatgatattattaaagaaaattacctgcgtagctcccagttccgtgcgtccggatttgacggcttcatgcagaatgtcgtgtctatgcgtcgagatcttgtcatcccatttgattacgagccggaagtcggtgtgctgcttgaactcatgagcgtcggaatgaatgcggtgagcactttcaaaaaacatgcccatgaccgcaaagacgctattggaatctggggaaatggcaacgtgggctttgtcactgcactcattctgcgaaaagagtttcctgatgccaagatttatgtttttggtgtggaagagcataaaaatcgatacttttcttttgctgatgatacatttatcatcaatgatattcccgaggacttgcaattggaccacgcctttgagtgcgtcggaggccgaggctctgaagcggccatttctcagatcattgactacatcaaaccgcaaggaacaatcaatttgatgggcgtgagcgagaatccagttcccattaatacacgaatggtcctggagaaaggcctccaacttctcggaaattcccgaagcaactacgatgattttaaaaagtctgttgacatgatggcgaacaacaaagacgttcgtgaatacctttccactattgtctctgagatggttgtggtgaacacaattgatgatatgatccgcgctttcgaagatgatacttacaatgatttcaagaccgtcatgaagtgggaaatttaa

SEQ ID NO: 127 - DNA, 1026 bp, synthetic construct
  source 1..1026: mol_type = other DNA
atgcttaaccaagtttatcgactcgtttctccccgtcagttcgaggtggagacagtcgcagaagaagttgatgcggatgacttgatcgtgcgtcccaagtttctcagcatttgccacgcggacactcgatacttttttggacagcgctcgcaagccgcactccgagccaaacttccgatggctttgattcacgaggccgtcggtgaagtcgtttacgatccccgtaatgaatacaagcccggaaccttggtcgcaatggtgcctaacaccccggtcaagaaggatagcatcattcaagaaaattacctgcccagctcccgtttccgatctagcggttatgatggcttcatgcaagaatacgtgtgtctgcgacgtgatcgagctattactgttccttccgactttgacccgcatatggcggcgttcattgagatggtttccgttggagtgcaggcacttactaagttggaatccacaatggatgaggatcaacgcgttattggcatttggggtgacggaaatctcggttacatcaccgccaccctcgttaagcaaatctttccagactctcagctcctcgtcttcggacgccatcagtctaaactggattatttctcgtttgcagataagacgtatctggtggacgatatcccggccgacgtcaaagtttcgcaggccctcgaatgtacgggcggtcagggctccgaagccgcgatccaacagatcatccaacacattcaaccgatgggtacggcaattctcatgggtgtttctgagaatcccgtcggaatcgacactcgttcggttcttgctgaaggtctgactctccgaggagtgtcccgtagctcgcgcgaggattttaagcgtgcggttgacatcctgtcctcttcgccagttacgcgtgaacgattgcagaatctcgtcggctatgttcagaaggttagctccatccaagacattaccaacttctttgatagcgccctcactaattactggggcaaggaggtgatggagtgggacgtgtaa

SEQ ID NO: 128 - DNA, 1029 bp, synthetic construct
  source 1..1029: mol_type = other DNA; note = Lachnospiraceae bacterium
atgattaatacactgtacaaacttacggccccactgattgtggaatccttttatgaaaatatcgactgtcattccgacaatattattgtgcgccccgaaaaactttctatctgcaaggctgacatccgatactacttcggaatgcgtaatgctaaagtgcttaaagagaagttgcccctcgcactcattcatgaggcatgtggctctgttgtctatgacatcaccggcaagtttaaggctggtgaccaggtcgttatgcttcctaatattccgggtattaacatgcagtatcatgagaattatcgtcgtgatagccttttccgcagctctggagcagacggattcatgcaggagttggtgtcgatccctcagtcgcaagtcatcaaatttgagaagatggatcaaaacattgccgcgttcacggagttcatctctgtcgctattcatgcagtcagctcgtatcttgatcgcagcgcgggaaagcgaaatggtatcaagcgtaaaatcggtgtgtggggtagcggcgcaatgggctacgcggtgtcgtgtgtcttgaagtacttcattccatctgcagagatcactgtcattggaggtagcaaagcaaagctggacatgttttcctttgtcaatcatacttgttccctgagcgagcttcctgacgatgcagagttcgatgattgcttcgaatgtgtcggtggagcccaatctggcaatgcaatcaatcaaattatcgatcatattctgccggaaggcctcctgctgttgttgggcgttagcgaagaggccgtgcctgtcaacactcgaatggtgctcgaaaaaggcatcactttgttgggtcgatcgcgctctgagcgttcggactttatcaaaacaatcgacatccttgatacgaacgagaatattcgacgccgcatttctaacttgatctctggcgaggtggagattaacgaggtgaacgacatccacaaggcttttaatcagtccaaaattgcagactataaaatcatcttgaattggcgtatttaa

SEQ ID NO: 129 - skipped

SEQ ID NO: 130 - DNA, 1002 bp, Moniliella pollinis
  source 1..1002: mol_type = other DNA
aacacgtagaacacagggatcgccatttgtccgaaatgcaatctttgctctcgaagcttgaagcttgaatagcttgaagcatgagtgcatgaggggctgctatgatcttccaaccaaacttgcttgggcattgcactttggctggatagctatcatttagctgacccccaaagagactgccaatgatatgatatttgtgtaggtgcacgagtggatgagtggcgcgactacctgtcacaagatagatgctcgatgcacttcatgtgaagtggctagtggatgtccggagactggagtacagtaaagagtttggagttgggaacagcgcgaatgaaagaggctcagagagaggtaggtttgatgtgacgtcaagcgggctagtcagttagcaagggagtatggaatgcagcgcgcaacgcgcagcgcggagcccagtgcgcctgcgctcaggtactcatactcagtactcagtactcaggcgccatgccggagcttacatgcaatcaattaaccctgacgtctggcgcggcctgacgcctgaggcctgaggcctgacgcggccaagctccaagcgcaaccccaggctggcttgcgctggcgggcgcgagacgcagcagggggcaagtgtcgcagagtcgccgatcggatctgtggaacagcacaggaggcccgtgcggtcggctgcgcagtgcggtggagaagctggaagctggaagctggaaggtggaacctggaacttggaacttggaaaggaagaagagcgggaaggcgatgcacgatgcacgatgcacgatggggttggattgcgtacgagtagccgcacagcgcacagcgcacaacactcaccccatgcccccacgccccagcccgtgctcgggccatgagctgctggcaaccccccgaggcagccggcctttaactcaagtagcattcgccgccggtcggttcttcttagcctcccaccaccaccacttgcccgtgcttggacgctaattacaataa

SEQ ID NO: 131 - DNA, 679 bp, synthetic construct
  source 1..679: mol_type = other DNA
agctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatccatcatgcatcatgcatcatgcatcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcacc

SEQ ID NO: 132 - DNA, 1002 bp, Moniliella pollinis
  source 1..1002: mol_type = other DNA
catcgttccagcttgccttgggtgctagccagcgcaaaagtccgacgaacagacgaattgaccgtagtcgatgacagtcgatgacagtcgatgacagtcgatgacagccgacgaccgacgaacaacaaccaaccgcacgcagcatgaggccgagaggctcaggctgagccgctaaggcgctgaagcttgaagctcggtggtccagtcagatctcatgatctcacaccgccgcgctgctgcggtcgtatggttcaatttgctgtgcctagcctagcctgggtacgtcggtaactagccactagccactggccattatttggctactgggcacactaggaggcgcactagtccatcgcaagcggtaacttacctaccggccactacctgatgatttccctcttgcctggcggcctggcggcctggcggcgcactctttgcgctcggacccgcaaattgcaaaccaacaaacgactagtgtccggtacgtgtgcccaagtaacctgggcagacatagaaccacattgatcgtagggaaccgcaatggaccagtacgctattctcagtacattctgtacacttttgcctagattggaatctggtgaggatcaagatcaaatttcaaaccaactccacggcactggttcccgacacttggactgtagtactgaggtactgaggtactgaggtactgaggctcaactgagtctcaactgaccgtgaccgcgcttgtgtggtggcgtggattctagtgttggatgtgcgcagtggtgtgtgcggtggtgcgttcagattcgtcggtagaacttgactattcacgtcttgtgctgcgctgacgcttggaacctggaatgttaaaccttgggtcctgagggtctccaccccccaagtcgactgggttctatataaagtcgggtaggcaattggctagggagaatctctcttttctctttccccagaacacctgttcactcacttctcacctctttacttacctacttcaca

SEQ ID NO: 133 - DNA, 1000 bp, Moniliella pollinis
  source 1..1000: mol_type = other DNA
cctcgttcttgattgctttcaaaagtaatttaaggagagaacctggtcgtttgttgctctactgaatgatcttacctataccagcatacgacaccttgctgcccctccccttcaccaccaccatcaccactttcctctttttctttgggtttttctagtcgttgcatttatggttgattcgctctttgagaaccgtatggctgttattccttcccaactcttttgtctgttttatcgttgcatccaaacgtttgggctcttcgtacctcttttttctctgtttgccttttatttttggtttttctatccgtttgatggttgtgactcttgttttcctttgggaaggtttgagctcaacttctcttcggtccttgttggagtttgacttccttttctttttcagctgtggccgatcattacgatcaataatttgtgaatatgatatcgactacatcctgaatcttttagcgaaccatagcagtttagcgtcatagatatgcatgcatgatgtacatcttacacgcaccacggatgatgctcagcgctcagccacgcagccgctcagccgctcgctcggtacgctcggtcatgatgtgcatgatgcaagcctaatgattgacgggtacttgatctgtgtcccaattcgatcgagatcgatctcactatagtttgtcgaacttcgatgcacatcgagggtgcggaaaaaagctgacggcagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaaccctgccggccctatataactagtgcttcatcttcttgtcattcttgtctctttaccaacacaccggtactctttcacc

SEQ ID NO: 134 - DNA, 1007 bp, Moniliella pollinis
  source 1..1007: mol_type = other DNA
cttgacgggttcaatattcaatactcagtgttcaagcagtggtaggttgctccgtggatggaacgacgggtttaatttcttccgccatgcaccatggtccatgatcggttcagatggacagttggtatgagaaagtggtaacagataaatagaatttcgtacaagttggcaagagcctggccgatggtccatggctcgatggctcgatggctcgatggctcgatggccacacaccatgtcatctactttctgtgcttctatgcggacgagtcggaacagctgtcaaactgcgtttggggtatttgagcggaatttgaaaccttgttgaaactacattgtctcctcgttacttctctggcatctgtttgatcgaacggcgtcatgcaatagacacgcgatgaggcccaacgacggtgacgttgaacgcctggcccacctgctacaacaccgtagaacgtgttgaagttgaacgtcggacgttggacgttgagttgacgcgtgatgctggaaccgtggatgtagcagcgacagacaagccctctaagcaatctagtcgattttcaaaccaactaaacgagccttagttcggacaaccctagtccagtgcgggggcagcgtggggtcagttgccagcgaaggtcaccccagctagctgggttggctagcttgactggctcagaagtatcgatgctggagcttcaggccagtacgaaaatacactgtcgtagtgctccttccggagttctgggcttctgagcccggcggttgtgcatcggtgcacagtgcacggaccggcgggtgggacaacaaactgtctgggcgtgaaatcgtgcgagtgttgatgcaaaccggacgtacccagcacgcagttcccagtactcagacatactcagtacctcagtggttgagcctcagcatcgcctccgtatttctacaaagctgttgagtcgccttttgactttttactaccaccatcccctcttcttctaaaccccttacattccaaa

SEQ ID NO: 135 - DNA, 1000 bp, Moniliella pollinis
  source 1..1000: mol_type = other DNA
tcgacgacagtgagtatagtcaactatgctatctagtagccaaagtaagtctacattccgggctttgcaggggttagctatttcattagacaatacttacaaccttcgaagggcttccccagaattcaattctatattgattcaatatccacccatcggacccgctctccgtaccccctgttcgcttcgccactacaaccatgtcaagacctgagaatcgtagcaaattgccccactacggcccgagtgctgtcggctggttaaagtagaatgacttgggtagccagatcgagacgcctgacattgatacgcagacagcgcagtcagtttcaaactgacaactgacgcgtactggattgacaggactccttggccggtgccattcgaatcgcttgtagacctcttgccagcatcttgccagagaagcttgaaggctggatcgctcagcctgagctcacacttagtagctccactagtcactcaggtgctcagcctctcctcagcgcctcaagctcaagcctcagggcctcagctgcatcgagctctggtgccagtgccagtgccagtgccagtgccagtgccagtgccagtgccagtgaagagagtggtatgagccaagctatacccaaaagcaaatgatggttattgccagcttccatgcacccctggggcgaaggacaacgaacaacagacagagtttgaattattcactggcctagtactaccaatttaaatttggtggttcagcgcaggaacggcgcgagtgagcgtaagggcaaactccgcatggggcgtgacgtcagtgtccctctggctgaggcactccagcaggctctggtcaacgcatcaacgtgtgtgtgtgtctaccaagcacattgtgcgctcgctgtccgtgcgccgaaatcgcaccagtcagaacgtctataaaacccctcttgcttggagggttaccgggccgtcttttcccaacgctccggctcactcactactctacacaaca

SEQ ID NO: 136 - DNA, 997 bp, Moniliella pollinis
  source 1..997: mol_type = other DNA
tcggaccccaagctctctagcttcaagctttcaagctagcttccagccagggtcatggaaccgtaggagctgggaaacgtgacgttgtcgctatgcctatcccatataagaattaacgtcgaatcatgaggcgcatgggaggctgcattggtggtatgatacataacgttatgattctatatgagcgatggagcgcggccgtctgccgctagtgtggaagttcgatacgcccagtccatttcgttcagatgacgtttggtccagaagtacggtacataccgagcgcaaacgtcgaacttatggccttggtcgctcgtggtccggcgtcgtcgacactttcatcaacggacgaaaggtagagccgaacgccagcggccaatggcgatggcgatagtagaatgaatagtcgtcagatctaggtcttaggctacaggccaccctgcctgaggcctgggggtcttacagcgagtctaaaaaacgaaaagccaacgtgccaacttgggcctgcccatgcaccctgccagagagccccaagctggtctgatgcaagcggagcaaaatgttggaagaatagtttcaacgatggtttggttccagtacgcacagtcgagagatgaggtactgcgcaacgagttgttggcgacggaaccgaagatggattgaactatttgatctacaggtgatggcgtggagtcgggcgtggggccgctgtgtgatatgtagaagtggcgctattcgagggctgcgcccgtctggatggatgcagaatgaagcttgcaggccgcagctggcaggatgggcgcagaacccggtgaaacatagttaaagcgcggggccagtgtcatatttcgcttgagccattctactcgttggcagcccaggcgccgctaatttacgccaactcttggtactcgagagctcgggcaatgtggtatataagcttgaggttccctacagcaaagcctggcttcttcaccacaaccgcactctagcaaac

SEQ ID NO: 137 - DNA, 1002 bp, Moniliella pollinis
  source 1..1002: mol_type = other DNA
gagcgtagagagctccatcgatggcctggttacaagttgcctgaagaccaccaatacggcccgcacgccaacggcaggccaagaaaggataattgacaaccgtgaatctgaaagcttcggcgctcaggagctcatgtgttttgttgtagatataaaggtttctgaataccagttgggtattgacacggggagttggactgaatctctcagatctcccagatctcaagtactgagtcccaagcgtgccataattaactctaggttgcgcgggacacgccacctgagatcagggtgtacgcgcatactaatccattccaccaccaaagcaagactgagggtaccgcctgaggttctggctattccaacagtcacaagtcaccgcgattgccgtcaaattccaaagcgtagtaggcgctcgacggagggatgatggccagtacccaatttcgagcactcagttagatctgttcgtgtttagttgctcctgcagatctatcttaaatgtatactgtgccaggtaggatctctcggcacctatagatgatataaaagatatgatgatatgccaagtgccaagccgtcaaactgacaaaatgaaagaaacagcaggcaagctaggcaagcgtctatcgggggattaaacggatgagtcagcagcacaggagcagtcagtcgacaattccggctttttcccgcccgcaaggccccagaccccagaccccagccgccaccatgtccgtatccgccggggttagcatgaccacggcagatttctccgctcagcttgctcggacgactatatataaagaatcctttcctcgcagtccgtccgagcccttcctccaccccatcggttaccaccaacgacatcacttggtcttgacgccgcgctgcgttttcccgcttccgcgtctccctagtgatttagctggtgttttcctctacccgccactctactgtttcatcgcttgttgccaagttttctcagttcattctcatcaa

SEQ ID NO: 138 - DNA, 1002 bp, Moniliella pollinis
  source 1..1002: mol_type = other DNA
tggacgtggacaagcaccctgaagtcgctcagggcttccagatcaaggctttgcctaccgtctacggcttgtatgagggcaagcctgttcgtcagtgtaagttttaaaatatattgttcttgtctgttcgggttttacatcagtggtgtactgacacggctttttgttttattgctgcagtccaaggtgtacttcctgaggatatcctgcgccaatttgtccaagatcttcccagcttggccgcccaagatgcccagaacccgcctgctgaaagtagcgaggctgccaactaagtatagtccatcctatgagctgtcctaccccaaacaactttttcacgcccaacgtccaaacgtcacttcttattgcggcaaagaacagctgcttccccactcattcattacccctttctgatccacgaggtgtacccatggcctttgtacattccatgaaacaggtatccttttccccaccattacatctgaacttacaaagactttgcttcctcttgtacacagctcgtcgtccatcgtccatcatcgatgacgatcatcctgcattcggtatcatgcaccatcatgcagcgatggcaacgagggccatgcaccatgctgcatatactatgtgtatagccaatagccaatcttcaatctccaacttcctacagacacagcctgtggggaatcgcttaatcgctgtccatgcgtccatgctccgcgttcctcgcgctcgtcgcggcctcgcgacgattaagtcacagtcacgcccacacagcgcgcgagcgcacgagccaccatcatcacgtgatgagacaaagagtccacttccgtaaaagcaatcttaataatacatgtaaccctagcaatgtagccctaataatttgtttggcctttattttattgcgcccccgcagcttttgcaccccggagagtaaaagtattccggttgctcgtcagagcatccctttctttctccatcagcaaagcgtgcgaccgccaagaa

SEQ ID NO: 139 - DNA, 921 bp, synthetic construct
  source 1..921: mol_type = other DNA
tttggatgaggcattcaatgtctccatgtttgaagaccagagaaagtgtaggtaacacaatagccatggacaattagcgaatccgaatatgtgtactaacattgtgcctccaactgacagctggtcgaataatgattgaaatcatcaaacatatcagagatatatcccagaaccatttaacaactgttattgtatgtatgcattactgttttacattgtgtctttccaaatactaacattgccattatccaatccatacatatatatatatatagattacacaacgcaaacctcgacagtcgattggtttacatcgtgtaagcagccttttacggaatatgatggacaaccagatcctcatagatggagacaatgtgcagatagattccatttatgatgacattgatgtgtttttaaatgcaaaataatctcctatataagtgagttccgagtctccccatgcttatcttatcgtgttttttctgtaaattaggtcaaagtattcaaacaattccctcaatctttcccattttttgctgagcccccatatcatcaaattaacgtatcaaatttcctaattagtagccccgtatgttaaattagacatgtgtgactgccatcccggacagcctacccaatgacacccgcgcaccgcacatattgtgtttagtgcgcgccgtctgctgaagcgactccctgtttgggaggaaccgagggcgggttgcccgatcccttgcccctcgctcctcctcctgggctcccccttgcagagggacaccgaggggatccctcgtgtgagagcttggaggtggatggtggtcaattttctcatttgattgaaagatttgttatattgaaaattcagtttgtggaagttgtgattaaaaggttttactgtttgttctgtagacacattcaata

SEQ ID NO: 140 - DNA, 500 bp, Moniliella pollinis
  source 1..500: mol_type = other DNA
acggatcaacaaaaatgtgcgccagctttgttgtgtgcccttttcctctagcggatcctatccacatttcgtgaatgaaccgattaagctctgacgagatttccttgttttctgtagatacctcttgtagaggtgtcaaagttatgttgtgatcgagagagttacctgcaaggtattatgcaagcggcttgatgaggacactcaactacgtgtgagtaggagtgcgttgctcagcagttgacgcgtgagccgcttgagcagcttgataaaggtcctaatcgtcttccagctgttggcagacgacaatgtgctgtagtagtaggataggcccgtaaaagtgtagaagcaataggttgagactgactgtaatgggtacttgcgttcaaagactgaaagtccaaaggattgctcaaaggcccctgaggtctgagcctctgagcgcgctcttcgtcaaaattcgccggggccatcggtcatcctctcgagggcagcatcttcct

SEQ ID NO: 141 - DNA, 500 bp, Moniliella pollinis
  source 1..500: mol_type = other DNA
gtactaaccaagtctattgtaaagctggtgtctgctgctgcttcagcaagtgccattgctaccatcgcaagcaaattgcaaaaaaattcgttcaatggtctaatttaagtaaaatactcataatttataaaatgccgcttgtccttaccttttgtctttctccttattatagtagctgtagttatcgtagtcctcaggacttcgtcaacgctcgttgatgatcatatatacttcacctcattcattcataaagcattcattaggattgtatgatccggcagtgcatcatgcatcatgcatcatgcatcaggacatcatgcatcatgccgtatcggtcgatcattgcatttgattggataatacgatcgtgtttaggcattggcagtcgaaacgaggataaagaagaggtcagtaccagtgcacagcacgcgctcgcgctctatgcgcactttgacgcttgaaaaaaacttttcagccaatcggatatagacagcggaccc

SEQ ID NO: 142 - DNA, 493 bp, Moniliella pollinis
  source 1..493: mol_type = other DNA
atggtctgagccgcccacgattccttccttcatgagcgtatgacgacccttttacttagcatgtgcctctaaagaatacgttttgcatacccacatattcgtaaccctgtcctctatagcttctacaggtcatagtcactggctgccagtataaacgtgtcaatatctaatcctcataagatgcctcgctctgtcgtccacaataaaattcagtaacgcaaggactgcgcgactgagtgattgccgatgatatgagaaaactaatttcaagccatttgaacttcaataaatgcttccgctaacatcacggatcatcgatcatcaaccagcctccattaataggcctgccaccactcgcgcaatcatgaatctgatcatacctggctagcgattctcataacaccagaattacctgcccattaaagaaaccctattctgcctgtaacccggcaccagccggtccgaattcacgacagactaaggctctcgcgca

SEQ ID NO: 143 - DNA, 500 bp, Moniliella pollinis
  source 1..500: mol_type = other DNA
atcagttggaatcatcaaccttttttttttgtatcgatttcttcctaccctcacctcccttttcccccttaactcctcgctttgaacctccttacccgagtgcctacctttacctttttctttttctctctcccctgcctaatattctaggcttgctactatactgctctctctaacacgatttttgcgcttgtaagcatttttctctttttaaatctaggttttatcatttgttgcatttatccaagtttttttttttttttgtttgtagtggttttactgtttttgtgttttgtagcttgactaggttcttattctattgtagatgtatatgatcggatgcattcccgatctaaaaggaaaatgttctcgactgatatttatttttaccttattgtcttcctaaatggtttgactcttgccttcttccttgggacgctttgcaacgttttgtacgtacgtaagggttcatcgtttggataagcgacacagttgtcaag

SEQ ID NO: 144 - DNA, 324 bp, Moniliella pollinis
  source 1..324: mol_type = other DNA
attcaccctggagtaactgcgcttcatcgtttctttgttacgacaataataccacgctgcatgctttgaattgagtatatattttgatgactggttcaaatagatctttcctccatagccttcgagtactcgtgatctcagcattatcagatttgtgtccaggcccccgaggacgatgcggttttcctggagtcactagttgggaagatttgtactggttgggtgtaggtagagagaaccattgattttggtgtagctgttcgttttataaggccgagattacgtaatgcacatgctgccctagcaaatcgcgtcgcgtcacgc

SEQ ID NO: 145 - DNA, 496 bp, Moniliella pollinis
  source 1..496: mol_type = other DNA
atgtgcttagattaacatacacttgacttccttacgcgttgatatatacttggatctccataacttctattgtatagtttaatcaatggactccatgttgacatcccattatgcgatctatagtagctggaaatagcaaaattgaagttcggcagacgactgcaccaggaggggtagattctgtcccataactccctaccacaggccgtgctctggttcgatttaccttagcctgatgcgctcaatgtaacgcgtaattctgagtggggttcggtcgtccaaggtttcagtatgcgctgtgcagatagtactgtatcgtgaaactcgaccgttgcacatgcactttatcaagtgtcgtaggcccagagtcgtaggccaagagtcgtaggccaagatggaaggcctgaagaaaaagaaagctttgatcgttctgatctatgcaatggattcatgagctaagtattagctatacaaatgattcgagcatagcagaagg

SEQ ID NO: 146 - DNA, 500 bp, Moniliella pollinis
  source 1..500: mol_type = other DNA
gttgctccctttgctgttctatgatccaaaagatgcttagtgctccactctctactcccgactaccttttacgcattctgacatgactagtagtgcaatgttacccctggtgttttctcgcaatcactttctgtctgctatagcttttgatcgtcagtagttttttggtatagctaaagtagacttttgtacctggcgttcaatgtaattttactccttttttgtgcgatgcaacagatggtgtaaagatcgtcgttaaagttttggcttgatgcttgattcggtgacggttgcttgggtgatccttatatcgtctcctggtgttctcataagcccaggctcttcttagctatgtacaacaggtccaccactgactatgttatgtagaggcatgcatttaacttacggaaagatttaatggagaagaacgtacctagtcagatgcttgacatgaaaacgtattcctgcaattcacaaatcagtatcctgcctgtccactc

SEQ ID NO: 147 - DNA, 500 bp, Moniliella pollinis
  source 1..500: mol_type = other DNA
gcggttcgcacttttatcccaggcatgaaaaatgacgcacggctgtcgcacgctacacccagtagtcgccttttcaggcagtctatgaggggagcttttcattgcccaaaattactagcctgcaattcatgatatcaggttcactttctcccgatgtctatagatttctcatagcgctttgaaaagacaatttagctactcagtttataagcaaatgatgtggaggtatttattaggtacaagatacgataacgtttacacgcggggaagcaagtggcaaatcgtcgacaacaccgcatggtggtggtggaatcgtactccatcaaagcttgcttgtcactttgggaggcaccaggttgaggccatcgcccgaaggtggttgtggccagggtttcgatatttcatgaatcaacgtgttgatggtattggattgggtaagaggtcgatccggctgttggtcagagtgctcatcacattcgtcgttgtcaagcccgagttgc

SEQ ID NO: 148 - DNA, 500 bp, Moniliella pollinis
  source 1..500: mol_type = other DNA
gtggcgttgattcgtatcttattattgatctgtcctgttccccacgttgccaagtgattgttcatgctatcctctctccattctgtttgtgttgcacagtcggtagttgtacgtagtagctcgtcacaaaatatgaagtgagccttgcaatgtaggcattcacaagctgctaggcaggctatgatctaagctaagtggcaaaaattctcttgattcaatgccaaactatactattgttcctcaggcaaaagcagctatataagttgcattcaacgcaaccgccatacaagcaaattagagagaaacgggatataaggtgctttatccctgaaggagtagccagcgtctacttggttgctacacaatacagcaaacagtgtacttaatgagccattggctgaactggagctcgcagaggttcgagaagggcgggtcataccagctgccctccatggaagctggggtgtcgatgatgaaaagtgtcggaacgtacgctagta

SEQ ID NO: 149 - DNA, 500 bp, Moniliella pollinis
  source 1..500: mol_type = other DNA
acgaagggtttcccatctgtgtgacctgatccctttttacgtttttttgttttgttttttcttttgttcatgatcaacttgggagtgattcctcttggtgatgtcgaaaattgaactgttataccaatattctgttgcctttgtttcctgtttgtagtttactactttttacagtagtcgttgttcctgccttgcttgcgtttgtcagggtttccctttagcttagtaccaatatctagccttaattttgcttattctttctcaccgcccatgattgatttcaatcatgattaatgatttatgcatgaaaactcaaaatactcaattgtcttgtacaaggcaatcacaagctggtcagccattctcagcgcctataagagctttcagccaggccggccccaaaacgatctaccctgtggacctggctaggctaaactttggaaagtcatgcaattatgaacgtttgaagctcaagcgctcctaccctctcctggagcagt

SEQ ID NO: 150 - DNA, 500 bp, Moniliella pollinis
  source 1..500: mol_type = other DNA
tggtgttcaccactgttgcttcccctttgttggagcgtccgtcgttctgatggcgctctgagcccaaaatatcccttcttcgcccgagagcaagcctcggagtgtgcttgcaatctacttttcctactaaagttatcttcttgtggcttctgaatttgaatagaacgtgttcagacttgttagctaaatttgttgatgcgagatacttgacagaatcccgttgatgaactcatctgtgtgaccaccgcctggtgatggaatctcgcgcggaagccgtggtgttactgactctcaagcaattcagtagataaatttgtataacatcattgcaaggggtgctagcaatcaattgtccaataccaaacggcggcaagtgcaaaggggcgatagagagtttctgcgaggaagggaaggaatccgtatgcctgaaaacttttgtacaagtgtctttgtaagggcgaggtctgctctgacatctctctgagatctgagatctgata

SEQ ID NO: 151 - DNA, 500 bp, Moniliella pollinis
  source 1..500: mol_type = other DNA
gtttcttggctgttgacgaaatcttatgaaatgcacgggtctaatatttcctgccaattgtgtacccatctcgtttgtgaatgtcatagctagctctgtagtttctaaacgaccgtcttaggccggggtttgctgattggggattaaggataggatacttggccagtttgggccgatcaatatcaatctgctaggcgttccttaggtcaacttgaattatgatatatgaggtatgaggaaagtgggctaaatctgtggattaggatcagaaaaaacgtggcagaccctcgatctaggcctacgtcagcaatttcagcaatttcagcgccttgccccttagataggtttcagccgctcagcgcgcgcaatttcagcatggatgagatcaaaccaaggggcagcagttggcgaccctgtagttccaatcgcatgcttagtgcataacgcgcacatcaacgtgtcagattctcagagatctcaacaagcccactagaccctag

SEQ ID NO: 152 - DNA, 504 bp, Moniliella pollinis
  source 1..504: mol_type = genomic DNA
aatcgcctgtgtagcgaacggggccagctgggtaaggccagcggccggtagctaggactatgaggctaccaggctgctgggtaactaggcagctaggcacctaggcaactgagcaactgagcaactgggcaactgagcggtataggtttggcttgggcacagttgatattgatatcaaccagtcgctcgttcggttcaccagaacccagtgtgtggtagcgagcgtagaatagccaaaaggttttgagctgaggcgctgaggcgccacgggaggacatcaccgcctgagcacacaggatttcaaactgtcagacgggcgtggtgccagtgttggaagtgggcgaagcacctcgcatctaagacatgtaattaaactctcgacagccatcgtccattgacaccaacgtgcaccaacacacgcgagagctgcttcgttgtgcctgtcgacgacattgcctcgttccccagccaagtcgccccaccgctacaccaagcgcactcgca

SEQ ID NO: 153 - DNA, 499 bp, Moniliella pollinis
  source 1..499: mol_type = genomic DNA
catccgaaattggtggtttgacaaatgcaccttcttctccaggggctcgtgcaaacgcagtagccgagaagaggcgtaccggagaggaagacccaacaaccactgatcggtttgaagttggatgagaaagttgtgccaatcgcggagccgtagcaacgctacgagcgctacctacaacgactccacgcgcaatcggtacgcgcaaaggaacggatgagcgaccgagcaggttgcgcgaagccatgacgattgctctacccccaacgagctggatcaagttgggcagaacataatttatctcgcttccccgcgccacaagtagctccaccattcttctcaaaatagcccgattccgctttcgcggggcaaaacttccgcctataccgatccgattgaaacgccaaaaaaattaagttgtagtacgcctgttggtcgagcccgactggagcaccatttgagacactaggagataatttattcttttccttcttggccccca

SEQ ID NO: 154 - DNA, 300 bp, Moniliella pollinis
  source 1..300: mol_type = other DNA
cgggtaagaccaagtcggttggtgtttcgaacttcagcactcgtctggtcgacttggttgaggaagcttcgggcgaacgtcctgcggtaaaccagatcgaagctcaccccttgttgcaacaagacgagttggttgctcaccataagagcaagaacattgtcatcactgcttacagtcccttgggcaacaatgtcgctggtaaaccacctctgactgagaaccccggtattgtggatgctgctaagcgtttgaaccatactcctgctgctgtgctcattgcttggggtattcaacgcgggt

SEQ ID NO: 155 - DNA, 453 bp, Moniliella pollinis
  source 1..453: mol_type = genomic DNA
gcgaggagtagttactaggtagcggtatagcaattccgcccataatgcataaccgttctccctaccaaaacaaaacaatggctacgtggccggcgtgcgtggccgacatggtgcgggtgcggggggcggggtacgagtgtggggagttcactagttcgctggctagctgactagctgaccggcgtgtggttgtggcagtacgcagtgggaaagattaaatatgaaatattgaatatcaaaatgacgtttggggtggcacgtgcgcgcgccaacaacaacaagagcagccaccaagccaaagcaccaagcaccaaccccgcacaccattttgttacacccgcggcagaaatggcgtcagccagcagtcactattactcaatattcaataggggttccacttgactcacttcctcatgtatatatatatccatctaatcttcccaatacataacc

SEQ ID NO: 156 - DNA, 500 bp, Moniliella pollinis
  source 1..500: mol_type = genomic DNA
gcggcctatgcagacgcttggagcttgggcacattggatattggatattggctggctgccgttcgagatcccgttgctgtcgccaccgccaccggtcacgtggtcacttggtcacgtggaccacctgcactgcgcttagctacaagagtgcatgatgcatgatgtcatgcatgatatgcatgatacaagcatgaatattgatttcaatattgaacatttagtattgatatctcgatatgcgtaacacgtgacacgcaccatatcaatcagccgcaccggcactaaatgtttcaatttcaacatcccacacgctcgaatgctccctcgagctaatcggatttcggagctccgacatggccggcctcgagcggagtttgaaactccgttccgcccgaccccgctggtcgccgcgatgaaatcccgagtcaagtcatagcaagccaagtggctggctgactcaaaagctaataaaagtcgtagcaagtaatactccgcacgct

SEQ ID NO: 157 - DNA, 500 bp, Moniliella pollinis
  source 1..500: mol_type = genomic DNA
tcgcgtcttcaaaccgtggcgcgcgacacgtgccgcgaccaaaatttgttggtgtatgggaatagggttttctaaaattctgttgaggatgtgagtatcactggcatggcaccagtgtactgctcatcgataatcataaacgtatcaacccttcggctctgcgatctgtatctgctactggagggcacagcaagccttctgcttggttgcgctctgcgctctctgtggtgctttgccgctcgcggccaaggtccgcagcgggcattcaccaccatacgggttaagccaccagacgagtgctgctcgccgggccagctcggcgcctgtcacgcccccacattggcttgatgctcggctccttctttcatcttggaaccggcgggcaatggcgcttatcaccactcctgggccgggccccgcgtatactctcataactagtgcacgctttcaaataaatacctcgcctcctggctggccgaccgacgtttacccatccgccc

SEQ ID NO: 158 - DNA, 499 bp, Moniliella pollinis
  source 1..499: mol_type = genomic DNA
tggcatgtaggcctgtaggggctatgcctatatgctcatgcactcatgtcgcgatgttgcgatatcgcgatggaccaaccaagcaagcgtgcagcgtgggcgcttgggccaagggtgctaagctaagtgtaagcactgaagtgctgaagtgctgaagtgctgaacgtggaacgtcaggatgacacatcctctcccttcggcttcgtccaagaaaggttggtcagccatctgagatctgagatctgaaatgaatataatgatgaattgagtcgatgagatctgactatcttgggagattgcaccaatgcgccacggtgaccagatggtcttggtcttagctggtatagtggcacctgatctgccttgacagatctcatgttcatacgttcatgcattcaatcttatcgtctagccgtctgcgatctgcccaactagacgacgacaaatgtaattgatccttgtaaccctgttgctggtcgcagcagcgcgatacgatgcc

SEQ ID NO: 159 - DNA, 500 bp, Moniliella pollinis
  source 1..500: mol_type = genomic DNA
aaatcttcatccagcgtagcaagcatatcgcgcatgtccgagttcctcgagtgtgcataccgaacccaaaaagccatggatgtcattgacctgctgtattcatcaatcgagatatctttactccggagcgtacagtggcagatattatgtaggagatgatgggacaggatgcagagatacataatccatttccagttaatagggccgctagatacttgatattagcctggcagcgagcgagcgagggtagagtagaatagagtccagtatagatcccaccccatggtgcgggaagtggatgacgtaatagcgcgatagctccccgctcccttgtcacctaggccacgggaccctacgtgattgcatcgtgcgtgtcgtcgcctgtcgacgcctggtgcaggcatacgctgcgtgcttcgacgatcaacaacaccaccaaccacgactccagttccggccagccagtaccatggcgtcgttctttgggtttagtgcaccgg

SEQ ID NO: 160 - DNA, 400 bp, Moniliella pollinis
  source 1..400: mol_type = other DNA
aaccagatcgaagctcaccccttgttgcaacaagacgagttggttgctcaccataagagcaagaacattgtcatcactgcttacagtcccttgggcaacaatgtcgctggtaaaccacctctgactgagaaccccggtattgtggatgctgctaagcgtttgaaccatactcctgctgctgtgctcattgcttggggtattcaacgcgggtacagcgtcttggtcaagtcagttacaccctcacggatcaagagtaactttgaacagatcactctgtctgatgaggaattccaacgggttaccaacctcatcaaggagtacggtgagagccgtaacaacgttcctttcaattacaagccttcgtggtccattgacgtctttggtacccaggacgaggcta

SEQ ID NO: 161 - DNA, 500 bp, Moniliella pollinis
  source 1..500: mol_type = genomic DNA
atcctgtgctcgtctcttacattcatcttcctgcccataaaactttcaagctatttcctccaacgtactcaacaacctatttttttgggccccgctcaaaacgcaccttattgcgtctaagtgtattctaaccacgcgacctgaattgcaaagcctgttacccccatcgcaccttcgtcttaagatagttgtttaacgagccttggccagttttagccttgaccatgactacagatcataatggttagatgcttcttgatcgataatgatatattgcatgtcgacgattgaagatttattggatgtcgatgacgactgtcgagctcttattcttagcaacaacttaaatattcactatcgaatgcacgcgacatcatgcatcgtgctcatgaatatcactggatcactctcgatcgcagcccactaggtcagcataacacgcttgaagctgacttgaagctttggaacaaatggcagcaacagtggcggtcacggtgacc

SEQ ID NO: 162 - DNA, 486 bp, Moniliella pollinis
  source 1..486: mol_type = genomic DNA
attgttgcatcgtctcatcagatcgtccttcattgtagtgataattggtgcaaccttgtattagactgtatagcagaatccctcgctataaaaagtattatgcatacgcagttccaagcaggaattagtatggtagtgggacttggctgaagggctgacgacatggcgagaaaaacgtatcaattcggaacacactgtgcaatgtcgatcacgttttcggtggttcacggtaggcttcctagtccatgggaaagtggtaacatctatcccttgagcgcttgagcttaatgttaccttgtgcaaaagtcatttgaagcttcacttgaagcttgaagctttgaagttttgaagcttgaagcgcaaacagcagcggtacatgtatactggcgtaacctgcctacagtggtgcatcagtacgtcaagcgggtagcccgaaatctcattaaggtcatttgccctctaagcttagtacctagtgatctgttc

SEQ ID NO: 163 - DNA, 501 bp, Moniliella pollinis
  source 1..501: mol_type = genomic DNA
cctgaacgtcctgaactctgtcttattacataccattaatttccctgccttccctctctacgtcatcggaattttatctcttcctgagcagcaaagatttggtccttgtccgggatacaacggagagtcatgattgggtttatggaagagaggcgatagcacttgcattttgaccattcggggattaagcgaacccccaagtggcacaaagagctcgtatgagggtcagtaagtatagagggacatgcaaaggagagaaaaggaaacacaatattgacaaatagagacaagtacaggtgatcgcaatagcatgatgtaggggtatgcgcaagtcacaatcgttcagggcgggcataacatcagcgtacccgagcacaatggttaatgattaatgagcagaggaaaaaataatgattatgccggggatggcaggactaactggcgcagctcttcagcttcggcaacgcgtgcacatagtggtgtagcaccacgagattgagc

SEQ ID NO: 164 - DNA, 493 bp, Moniliella pollinis
  source 1..493: mol_type = genomic DNA
cctttgcctcacgcctctcgtttgcacgcttcttcacggcttatcaccgcttattatatccatggaatcccctccaaagttttcttcaacatagcttcatatcttctaccataacatctatcccaccaagtagcacatgaattcaacgtagcttgagaacttttcctcgtcagctgattgcgttctttaggcagtttgcttctaccccaattgcttctaccccaattgcttctgcttatttcaccagtccgctcggagatctctgacaactctgagatctgatgatcaacctcttgtgcaacttgtaaaatttgatcgatgagacgattagggacttgaagctttaagttagacgcgatgggcggtaatctatatgcttagactttgttgctatgtttgtcctataagatctgatgcatgatgtatcattacccagacgggctatcgcacaaggtgcagatgcatcttatcatgacagtgccacactgttggc

SEQ ID NO: 165 - DNA, 567 bp, Moniliella pollinis
  source 1..567: mol_type = genomic DNA
gcttgctggtcacattattgtcagtacctaccatgaagaattcaatattattttacattgtcaaccattacatggatcccatgttgtgcatagcctctatgtcatcctttggtcttttatgtctgtagcgtgctagtagtattcaagttgtagcactgctgtgaaacagtacgttcaagtagtgtgcagcagtacgttcagttagtggactggggcagcctcgccttcagcaaaggttaggtacatgagccagttgctcactcttgctttctacgaatcccataggctactttgccccctaaccgtgacgaccccctgtgtttgagaagtttgagtctttttacgttaccgtgaccaagcgagcagagtagaaactcttgaagttgtgtatgtaagaatacaaattgagaacacaaggttacctacctttatagttggtaaacagcaagcaataccagagattttcatcgcaacgtgactctatcattcactatattaagttgcacacggatgttgtacaacactacacgtaaaagagagaaaaatactaatgtcgatcgctcaggt

SEQ ID NO: 166 - DNA, 504 bp, Moniliella pollinis
  source 1..504: mol_type = genomic DNA
ttgctgctacgacctcattgatcgatgatctccgacactttgagtagctttccatcctctctttactgtactttgtattcttgctactttgtgttctttcttaatggtcccgaaccggtgattttgctcatgatctttttttttttttttcgtcaactgcaagaagatgaaaaatctactgttcgatagcacctcataaccgttccagagtcccgacaaccacaaggatgtttcttagataggtgtcagcaagatccacaaaaaagtgtaggtattagatggtttcaagcaaggtacaatattctaattcaatactcatttgaataatcaatattcaacagcttctagcctacaagcttcaatgttctccgtcggttaactcagtatacttagtactcagttcaagtcagtgttgttaaaataaaatcacgtgatccaaaaacttgaaacttttcatttttctcctcagatgttggtgattcgtcccatagcggttaccacaac

SEQ ID NO: 167 - DNA, 1679 bp, synthetic construct
  source 1..1679: mol_type = other DNA
aagacctctgtcccacaaaccttgatggtctgtgaaaccgtcaacaacatcattggtagaactgtcaatccaagaaacaaaaattggtcctgtggtggttcttctggtggtgaaggtgctattgttggtattagaggtggtgttattggtgtcggtactgacattggtggttccattagagtcccagctgctttcaactttttatacggtttgagaccatctcacggtagattgccatatgctaaaatggctaactctatggaaggtcaagaaaccgttcactccgtcgttggtcctatcactcactccgtcgaagacttgagattgttcaccaaatctgtcttgggtcaagaaccttggaagtacgactctaaggtcatccccatgccatggagacaatctgaatctgacatcattgcctctaagattaagaatggtggtttgaacattggttattacaatttcgacggtaacgtcttgccacacccaccaattttacgtggtgtcgaaactaccgttgccgctttggccaaggctggtcacaccgttactccatggactccatacaagcatgatttctgtcatgacttgatttcccacatctatgctgctgatggttctgccgacgtcatgagagacatttctgcctctggtgagccagccatccctaacattaaggacttgttgaacccaaatattaaggctgttaacatgaacgaattgtgggacactcatttacaaaagtggaactatcaaatggaatacttggaaaagtggcgtgaagctgaagaaaaagctggtaaggaattggacgctattatcgctccaattactcctaccgtcgctgtcagacacgatcaattcagatactacggttacgcctccgttattagcttattggatttcacctctgttgtcgtcccagtcactttcgctgataagaatattgataagaagaacgaatcttttaaagctgtttccgaattggatgctttggttcaagaagaatacgacccagaggcttatcacggtgctcctgttgctgttcaagttattggtagaagattgtccgaagagagaactttggctatcgccgaagaagtcggtaaattgttgggtaacgtcgtcactccataatggtgttcaccactgttgcttcccctttgttggagcgtccgtcgttctgatggcgctctgagcccaaaatatcccttcttcgcccgagagcaagcctcggagtgtgcttgcaatctacttttcctactaaagttatcttcttgtggcttctgaatttgaatagaacgtgttcagacttgttagctaaatttgttgatgcgagatacttgacagaatcccgttgatgaactcatctgtgtgaccaccgcctggtgatggaatctcgcgcggaagccgtggtgttactgactctcaagcaattcagtagataaatttgtataacatcattgcaaggggtgctagcaatcaattgtccaataccaaacggcggcaagtgcaaaggggcgatagagagtttctgcgaggaagggaaggaatccgtatgcctgaaaacttttgtacaagtgtctttgtaagggcgaggtctgctctgacatctctctgagatctgagatctgatacctgcaggataacttcgtataatgtatgctatacgaagttat

SEQ ID NO: 168 - DNA, 876 bp, synthetic construct
  source 1..876: mol_type = other DNA
agatgtagccggcgctgtagagttttggacggataggttgggtttcagtcgagacttcgttgaagatgactttgcaggcgtcgtgcgtgatgatgtgaccctgtttatctcggccgttcaagatcaagttgtccctgacaatacacttgcatgggtctgggtacgcggattagatgaattgtacgcggaatggtctgaagttgtatccactaactttcgtgacgcttcaggtcctgctatgactgagattggtgaacaaccctgggggcgtgagttcgcactccgagatccagctggaaattgtgttcatttcgtagctgaggaacaggattaatggtgttcaccactgttgcttcccctttgttggagcgtccgtcgttctgatggcgctctgagcccaaaatatcccttcttcgcccgagagcaagcctcggagtgtgcttgcaatctacttttcctactaaagttatcttcttgtggcttctgaatttgaatagaacgtgttcagacttgttagctaaatttgttgatgcgagatacttgacagaatcccgttgatgaactcatctgtgtgaccaccgcctggtgatggaatctcgcgcggaagccgtggtgttactgactctcaagcaattcagtagataaatttgtataacatcattgcaaggggtgctagcaatcaattgtccaataccaaacggcggcaagtgcaaaggggcgatagagagtttctgcgaggaagggaaggaatccgtatgcctgaaaacttttgtacaagtgtctttgtaagggcgaggtctgctctgacatctctctgagatctgagatctgatacctgcaggataacttcgtataatgtatgctatacgaagttat

SEQ ID NO: 169 - DNA, 1087 bp, synthetic construct
  source 1..1087: mol_type = other DNA
gaagactgagtccgtctcagataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatgcatcatgcatcatgcatcatgcgtcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatggcaaagttgacgagcgccgtgccggtgctaaccgcgagagatgtagccggcgctgtagagttttggacggataggttgggtttcagtcgagacttcgttgaagatgactttgcaggcgtcgtgcgtgatgatgtgaccctgtttatctcggccgttcaagatcaagttgtccctgacaatacacttgcatgggtctgggtacgcggattagatgaattgtacgcggaatggtctgaagttgtatccactaactttcgtgacgcttcaggtcctgctatgactgagattggtgaacaaccctgggggcgtgagttcgcactccgagatccagctggaaattgtg

SEQ ID NO: 170 - DNA, 1257 bp, synthetic construct
  source 1..1257: mol_type = other DNA
gaagactgagtccgtctcagataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatgcatcatgcatcatgcatcatgcgtcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatgggaacgactttagatgataccgcctaccgttataggacatcagtaccgggcgatgcagaagcaattgaagcccttgatggatcattcaccacagacacagtttttcgcgtgacagctactggtgacggatttactctccgtgaagtgcctgtagatccgccattgacgaaagtatttccagatgatgagagtgatgacgagagcgatgatggcgaagatggtgatcctgactcgcgcactttcgtcgcatacggtgatgatggtgacctggccggatttgttgttgtgtcctactcggggtggaaccgtagattgaccgtcgaagatatcgaagtcgcgccagaacatcgtggtcatggcgtgggacgagcactgatgggtctagctacggaattcgctcgagagcgtggggcgggtcacttatggcttgaagttaccaatgtaaatgctcctgctattcacgcttatcgccgaatgggtttcaccttgtgtggcctcgacacggcattgtat

SEQ ID NO: 171 - DNA, 1049 bp, synthetic construct
  source 1..1049: mol_type = other DNA
attgaagcccttgatggatcattcaccacagacacagtttttcgcgtgacagctactggtgacggatttactctccgtgaagtgcctgtagatccgccattgacgaaagtatttccagatgatgagagtgatgacgagagcgatgatggcgaagatggtgatcctgactcgcgcactttcgtcgcatacggtgatgatggtgacctggccggatttgttgttgtgtcctactcggggtggaaccgtagattgaccgtcgaagatatcgaagtcgcgccagaacatcgtggtcatggcgtgggacgagcactgatgggtctagctacggaattcgctcgagagcgtggggcgggtcacttatggcttgaagttaccaatgtaaatgctcctgctattcacgcttatcgccgaatgggtttcaccttgtgtggcctcgacacggcattgtatgacggtactgcttctgacggggagcaagcgctatacatgtctatgccctgcccctaatggtgttcaccactgttgcttcccctttgttggagcgtccgtcgttctgatggcgctctgagcccaaaatatcccttcttcgcccgagagcaagcctcggagtgtgcttgcaatctacttttcctactaaagttatcttcttgtggcttctgaatttgaatagaacgtgttcagacttgttagctaaatttgttgatgcgagatacttgacagaatcccgttgatgaactcatctgtgtgaccaccgcctggtgatggaatctcgcgcggaagccgtggtgttactgactctcaagcaattcagtagataaatttgtataacatcattgcaaggggtgctagcaatcaattgtccaataccaaacggcggcaagtgcaaaggggcgatagagagtttctgcgaggaagggaaggaatccgtatgcctgaaaacttttgtacaagtgtctttgtaagggcgaggtctgctctgacatctctctgagatctgagatctgatacctgcaggataacttcgtataatgtatgctatacgaagttat

SEQ ID NO: 172 - DNA, 1199 bp, synthetic construct
  source 1..1199: mol_type = other DNA
cttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactcgggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcgataacccagacatgaacaagctccagttccatttgatgctggatgaatttttctaatggtgttcaccactgttgcttcccctttgttggagcgtccgtcgttctgatggcgctctgagcccaaaatatcccttcttcgcccgagagcaagcctcggagtgtgcttgcaatctacttttcctactaaagttatcttcttgtggcttctgaatttgaatagaacgtgttcagacttgttagctaaatttgttgatgcgagatacttgacagaatcccgttgatgaactcatctgtgtgaccaccgcctggtgatggaatctcgcgcggaagccgtggtgttactgactctcaagcaattcagtagataaatttgtataacatcattgcaaggggtgctagcaatcaattgtccaataccaaacggcggcaagtgcaaaggggcgatagagagtttctgcgaggaagggaaggaatccgtatgcctgaaaacttttgtacaagtgtctttgtaagggcgaggtctgctctgacatctctctgagatctgagatctgatacctgcaggataacttcgtataatgtatgctatacgaagttat

SEQ ID NO: 173 - DNA, 1621 bp, synthetic construct
  source 1..1621: mol_type = other DNA
tattctggtacgagccatcccaaaaatggatcatgacagctgccaagagccaggactataagattgagatctacagcagcgatgacctcaagtcctggaagctcgaaagcgcctttgcgaacgaaggctttcttggctatcagtatgagtgccccggcctcatcgaggtgcctaccgagcaagatcctagcaagagctactgggtgatgttcatctctatcaatccgggagcacccgcgggcggctccttcaaccagtattttgtgggttcgttcaacggaacgcactttgaagcttttgataatcagtcaagagtcgtcgactttggcaaagactactacgccctccagaccttctttaacacggatccaacgtacggttcggcactgggcatcgcgtgggcgtcgaactgggagtatagcgcctttgtccccactaacccttggcgctcgtcgatgtcgttagtgcgtaagttctcgctgaacaccgagtaccaggcaaatccggagacagaactaatcaaccttaaggcagagcctatcttgaacatctctaacgccggaccctggtctaggttcgccaccaacaccacgctcactaaggccaatagttacaatgtcgatctaagcaattcaacgggcacactggaattcgagctcgtttacgcagtcaacaccacccagacgatctcgaaatccgtctttgcagatttgtcgctgtggttcaagggccttgaggacccggaagagtacctccgcatgggttttgaagtctcagcgtcatcgttcttcctcgaccgcgggaattctaaggtaaagttcgtgaaggaaaacccctactttaccaatcgtatgtctgtgaacaatcagccattcaaatcggagaacgacctgtcctactacaaggtttacggccttctcgaccaaaacatcctcgagttgtactttaacgacggtgatgttgtgtcgacaaacacttatttcatgacaacgggaaacgctctcggatccgtcaatatgactacgggtgtcgacaatctgttttacattgacaaattccaggtgcgagaagtcaagtaatggtgttcaccactgttgcttcccctttgttggagcgtccgtcgttctgatggcgctctgagcccaaaatatcccttcttcgcccgagagcaagcctcggagtgtgcttgcaatctacttttcctactaaagttatcttcttgtggcttctgaatttgaatagaacgtgttcagacttgttagctaaatttgttgatgcgagatacttgacagaatcccgttgatgaactcatctgtgtgaccaccgcctggtgatggaatctcgcgcggaagccgtggtgttactgactctcaagcaattcagtagataaatttgtataacatcattgcaaggggtgctagcaatcaattgtccaataccaaacggcggcaagtgcaaaggggcgatagagagtttctgcgaggaagggaaggaatccgtatgcctgaaaacttttgtacaagtgtctttgtaagggcgaggtctgctctgacatctctctgagatctgagatctgatacctgcaggataacttcgtataatgtatgctatacgaagttat

SEQ ID NO: 174 - DNA, 1903 bp, synthetic construct
  source 1..1903: mol_type = other DNA
ataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatgcatcatgcatcatgcatcatgcgtcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatgccacaatcctgggaagaattggccgccgacaaacgtgcccgtttggctaaaaccattcctgacgaatggaaggttcaaactttgcctgccgaagattccgttattgatttcccaaagaagtccggtattttgtctgaggctgaattgaagattaccgaagcctctgctgctgatttggtttccaagttggccgctggtgagttgacttctgttgaagtcactttggctttttgtaagagagctgctattgctcaacaattaaccaactgtgctcacgaattcttcccagatgctgctttagctcaagctagagaattagatgaatactacgctaagcataagcgtccagttggtccattacacggtttaccaatctctttaaaggaccaattgcgtgttaagggttacgaaacctccatgggttacatttcctggttaaacaaatacgatgaaggtgattccgtcttaaccaccatgttgagaaaagctggtgctgttttctacgttaagacctctgtcccacaaaccttgatggtctgtgaaaccgtcaacaacatcattggtagaactgtcaatccaagaaacaaaaattggtcctgtggtggttcttctggtggtgaaggtgctattgttggtattagaggtggtgttattggtgtcggtactgacattggtggttccattagagtcccagctgctttcaactttttatacggtttaagaccatctcacggtagattgccatatgctaaaatggctaactctatggaaggtcaagaaaccgttcactccgtcgttggtcctatcactcactccgtcgaagacttgagattgttcaccaaatctgtcttgggtcaagaaccttggaagtacgactctaaggtcatccccatgccatggagacaatctgaatctgacatcattgcctctaagattaagaatggtggtttgaacattggttattacaatttcgacggtaacgtcttgccacacccaccaattttacgtggtgtcgaaactaccgttgccgctttggccaaggctggtcacaccgttactccatggactccatacaagcatgatttctgtcatgacttgatttcccacatctatgctgctgatggttctgccgacgtcatgagagacatttctgcctctggtgagccagccatccctaac

SEQ ID NO: 175 - DNA, 1475 bp, synthetic construct
  source 1..1475: mol_type = other DNA
ataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatccatcatgcatcatgcatcatgcatcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatgggtaaggaaaagacacacgtatcgcgaccccgactcaactccaacatggacgcagacctgtacggatacaaatgggctcgagataatgtcggacagtcgggtgcgacgatctacagactctatggaaaaccggacgcccccgagctctttcttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactcgggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcg

SEQ ID NO: 176 - DNA, 1982 bp, synthetic construct
  source 1..1982: mol_type = other DNA
ataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatgcatcatgcatcatgcatcatgcgtcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatgttgcttcaagctttcctttttctgctcgctggtttcgccgctaagattagcgctagcatgacgaacgaaacctctgaccggccgttggtccacttcacccccaacaagggttggatgaacgacccaaacggcctctggtatgatgagaaagacgctaagtggcacctgtacttccagtataaccctaacgatacagtttggggtacgcccttgttttggggccatgcgacctccgacgacctcaccaactgggaagatcagcctatcgcgattgctccgaagcgtaacgattcaggtgcctttagtggcagtatggtcgtagactacaacaacaccagcggtttcttcaacgacactatcgatccacgacagcgctgtgttgccatatggacctacaacacgccggagtccgaggaacaatacattagttactcgctggacgggggatacacctttacagagtaccagaaaaatcccgtcctcgccgcaaactcgactcaattccgcgatcctaaggtattctggtacgagccatcccaaaaatggatcatgacagctgccaagagccaggactataagattgagatctacagcagcgatgacctcaagtcctggaagctcgaaagcgcctttgcgaacgaaggctttcttggctatcagtatgagtgccccggcctcatcgaggtgcctaccgagcaagatcctagcaagagctactgggtgatgttcatctctatcaatccgggagcacccgcgggcggctccttcaaccagtattttgtgggttcgttcaacggaacgcactttgaagcttttgataatcagtcaagagtcgtcgactttggcaaagactactacgccctccagaccttctttaacacggatccaacgtacggttcggcactgggcatcgcgtgggcgtcgaactgggagtatagcgcctttgtccccactaacccttggcgctcgtcgatgtcgttagtgcgtaagttctcgctgaacaccgagtaccaggcaaatccggagacagaactaatcaaccttaaggcagagcctatcttgaacatctctaacgccggaccctggtctaggttcgccaccaacaccacgctcactaaggccaatagttacaatgtcgatctaagcaattcaacgggcacactggaattcgagctcgtttacgcagtcaacaccacccagacgatctcgaaatccgtctttgcagatttgtcgctgtggttcaagggccttgaggacc

SEQ ID NO: 177 - DNA, 500 bp, Moniliella pollinis
  source 1..500: mol_type = other DNA
tatcagatctcagatctcagagagatgtcagagcagacctcgcccttacaaagacacttgtacaaaagttttcaggcatacggattccttcccttcctcgcagaaactctctatcgcccctttgcacttgccgccgtttggtattggacaattgattgctagcaccccttgcaatgatgttatacaaatttatctactgaattgcttgagagtcagtaacaccacggcttccgcgcgagattccatcaccaggcggtggtcacacagatgagttcatcaacgggattctgtcaagtatctcgcatcaacaaatttagctaacaagtctgaacacgttctattcaaattcagaagccacaagaagataactttagtaggaaaagtagattgcaagcacactccgaggcttgctctcgggcgaagaagggatattttgggctcagagcgccatcagaacgacggacgctccaacaaaggggaagcaacagtggtgaacacca

(19) United States
(12) Patent Application Publication
(10) Pub. No.: US 2025/0277243 A1
(43) Pub. Date: Sep. 4, 2025

(54) GENETICALLY MODIFIED YEAST AND FERMENTATION PROCESSES FOR THE PRODUCTION OF XYLITOL

(71) Applicant: Cargill, Incorporated, Wayzata, MN (US)

(72) Inventors: Peter Alan Jauert, Minneapolis, MN (US); Christopher Kenneth Miller, Andover, MN (US); Catherine Bradshaw Poor, Minneapolis, MN (US)

(73) Assignee: Cargill, Incorporated, Wayzata, MN (US)

(21) Appl. No.: 18/863,702

(22) PCT Filed: May 5, 2023
(86) PCT No.: PCT/US2023/066629

§ 371 Date: Nov. 7, 2024

Related U.S. Application Data
(60) Provisional application No. 63/364,375, filed on May 9, 2022.

(51) Int. Cl. C12P 7/18; C12N 1/16; C12N 9/04; C12R 1/645

(52) U.S. Cl. CPC C12P 7/18; C12N 1/16; C12N 9/0006; C12Y 101/01009; C12R 2001/645

(57) ABSTRACT

Disclosed herein are genetically engineered yeast cells capable of producing xylitol. The engineered yeast cell may comprise an exogenous polynucleotide sequence encoding a xylitol-phosphate dehydrogenase (XPDH) enzyme comprising a sequence at least 60%, at least 65%, at least 70%, at least 75%, at least 80%, at least 85%, at least 90%, at least 95%, at least 98%, at least 99%, or 100% identical to at least one of SEQ ID NOs: 12-15, 28-31, or 33.

### CROSS-REFERENCE TO RELATED APPLICATIONS

This application claims the benefit of U.S. Provisional Application No. 63/364,375, filed May 9, 2022, which is incorporated by reference herein in its entirety.

### REFERENCE TO A SEQUENCE LISTING SUBMITTED VIA PATENT CENTER

The content of the Sequence Listing XML file of the sequence listing named “PT-1349-WO-PCT.xml” which is 448,927 bytes in size created on May 4, 2023 and electronically submitted via Patent Center herewith the application is incorporated by reference in its entirety.

### BACKGROUND

Xylitol is a low-calorie sweetener used as a food additive and sugar substitute. Commonly used in drug, dietary supplement, confectionary, and toothpaste compositions, xylitol has also been associated with anticariogenic properties when used in chewing gums. Traditional methods of xylitol production, including chemically catalyzed hydrogenation of xylose hydrolyzed from biomass extracted xylan, are both monetarily and environmentally costly. These methods require high temperatures and pressures, large amounts of water, and metal catalysts that must be mined. In contrast, fermentation processes have been used commercially at large scale to produce other organic molecules, such as ethanol, citric acid, lactic acid, and the like, and may offer a cost effective and sustainable alternative to traditional xylitol processing methods.

Accordingly, provided herein are genetically modified yeast and fermentation methods for the production of xylitol while reducing production of erythritol.

### SUMMARY

The present disclosure provides a genetically engineered yeast cell capable of producing xylitol, the engineered yeast cell comprising an exogenous polynucleotide sequence encoding a xylitol-phosphate dehydrogenase (XPDH) enzyme. The XPDH enzyme may comprise a sequence at least 60%, at least 65%, at least 70%, at least 75%, at least 80%, at least 85%, at least 90%, at least 95%, at least 98%, at least 99%, or 100% identical to at least one of SEQ ID NOs: 12-15, 28-31, and 33. The yeast cell may be an osmotolerant yeast cell. The yeast cell may be a cell of the subphylum Ustilaginomycotina. The yeast cell may be selected form the group consisting of Trichosporonoides megachiliensis, Trychosporonoides oedocephalis, Trychosporonoides nigrescens, Pseudozyma tsukubaensis, Trigonopsis variabilis, Moniliella, Ustilaginomycetes, Trichosporon, Yarrowia lipolytica, Penicillium, Torula, Pichia, Candida, Candida magnoliae, and Aureobasidium. The yeast cell may be a yeast cell of the genus.

The disclosure also provides a genetically engineered Moniliella cell capable of producing xylitol, the engineered Moniliella cell comprising an exogenous polynucleotide sequence encoding a xylitol-phosphate dehydrogenase (XPDH) enzyme comprising a sequence at least 60%, at least 65%, at least 70%, at least 75%, at least 80%, at least 85%, at least 90%, at least 95%, at least 98%, at least 99%, or 100% identical to at least one of SEQ ID NOs: 12-15, 28-31,and 33.

The XPDH enzyme may have a sequence at least 85% identical to at least one of SEQ ID NOs: 12-15, 28-31, and 33 or to at least one of SEQ ID NOs: 14, 15, 28, or 31. The XPDH enzyme may have a sequence at least 90% identical to at least one of SEQ ID NOs: 12-15, 28-31,and 33 or to at least one of SEQ ID NOs: 14, 15, 28, or 31.

The engineered cell described herein may be a Moniliella pollinis cell. The yeast cell may be capable of producing xylitol at a titer of at least 20, 30, 50, 75, or 100 g/L when used in a fermentation process in the presence of dextrose at 35° for 96 hours. Erythritol production by the yeast cell may be reduced relative to erythritol production in an equivalent yeast cell lacking the exogenous polynucleotide sequence. The exogenous polynucleotide sequence may be integrated into the genome of the yeast cell at a loci selected from the ER1 locus, the ER3 locus, the PDC1 locus, the pyrF locus, the TRP3 locus, the gpdIIA locus, and the gpdIIB locus. The exogenous polynucleotide sequence may be operably linked to a heterologous or artificial promoter. The promoter may be a constitutive promoter. The promoter may be selected from the group consisting of pyruvate kinase 1 promoter (PYKIp; SEQ ID NO:86), 6-phosphogluconate dehydrogenase promoter (6PGDp; SEQ ID NO:130), glyceraldehyde-3-phosphate dehydrogenase promoter (TDH3p; SEQ ID NO:132), translational elongation factor 1 promoter (TEFp; SEQ ID NO:133), modified TEFp (SEQ ID NO:131), phosphoglucomutase 1 promoter (PGMlp; SEQ ID NO:134), 3-phosphoglycerate kinase promoter (PGKIp; SEQ ID NO:135), enolase promoter (ENOlp; SEQ ID NO: 136), asparagine synthetase promoter (ASNSp; SEQ ID NO:137), 50S ribosomal protein L1 promoter (RPLAp; SEQ ID NO:138), and RPL16B (SEQ ID NO:139).

The disclosure provides a method for producing xylitol, the method comprising contacting a substrate comprising dextrose with an engineered yeast cell described herein, wherein fermentation of the substrate by the engineered yeast produces xylitol. The disclosure also provides a method for producing xylitol, the method comprising contacting a substrate comprising dextrose with an engineered yeast cell comprising an exogenous polynucleotide sequence encoding a xylitol-phosphate dehydrogenase (XPDH) enzyme comprising a sequence at least 60%, at least 65%, at least 70%, at least 75%, at least 80%, at least 85%, at least 90%, at least 95%, at least 98%, at least 99%, or 100% identical to at least one of SEQ ID NOs: 12-15, 28-31, and 33, wherein fermentation of the substrate by the engineered yeast produces xylitol. The fermentation temperature may be at or between 25° C. to 45° C., 30° C. to 40° C., or 32° C. to 37° C. The volumetric oxygen uptake rate (OUR) may be between 0.5 to 40, 1 to 35, 2 to 30, 3 to 25, 4 to 20,or 5 to 15 mmol O₂/(L·h). Xylitol may be produced at a rate of at least 0.1, 0.2, 0.3, 0.4, 0.5, 0.75,or at least 1.0 g L⁻¹ h⁻¹. Xylitol production may be at least at least 10, 20, 30, 40, 50, 75, or 100 g/L when the fermentation is run at 35° C. for 96 hours. Erythritol production may be reduced relative to an equivalent fermentation run with an equivalent yeast cell lacking the exogenous polynucleotide sequence. Erythritol production may be less than 50, 40, 30, or less than 20 g/L when the fermentation is run at 35° C. for 96 hours. Glycerol production may be reduced relative to an equivalent fermentation run with an equivalent yeast cell lacking the exogenous polynucleotide sequence. Ethanol production may be reduced relative to an equivalent fermentation run with an equivalent yeast cell lacking the exogenous polynucleotide sequence.

### BRIEF DESCRIPTION OF THE FIGURES

This patent or application contains at least one drawing executed in color. Copies of this patent or patent application publication with color drawings will be provided by the Office upon request and the payment of the necessary fee.

The drawings illustrate generally, by way of example, but not by way of limitation, various aspects discussed herein.

FIG. 1 shows the native pentose phosphate pathway (dotted lines and arrows) and the native glycolysis pathways (solid lines and arrows) in Moniliella pollinis.

FIG. 2 shows diversity in the galactitol-1-phosphate-5-dehydrogenase (G1PDH)/xylitol-phosphate dehydrogenase (XPDH) sequence space.

FIG. 3 shows the structural characteristics of the NAD or NADP binding pocket located +23 amino acids from the characteristic GXGXXG motif (SEQ ID NO:133) of XPDH enzymes.

FIG. 4 shows diversity in the ribulose-5-phosphate reductase sequence space.

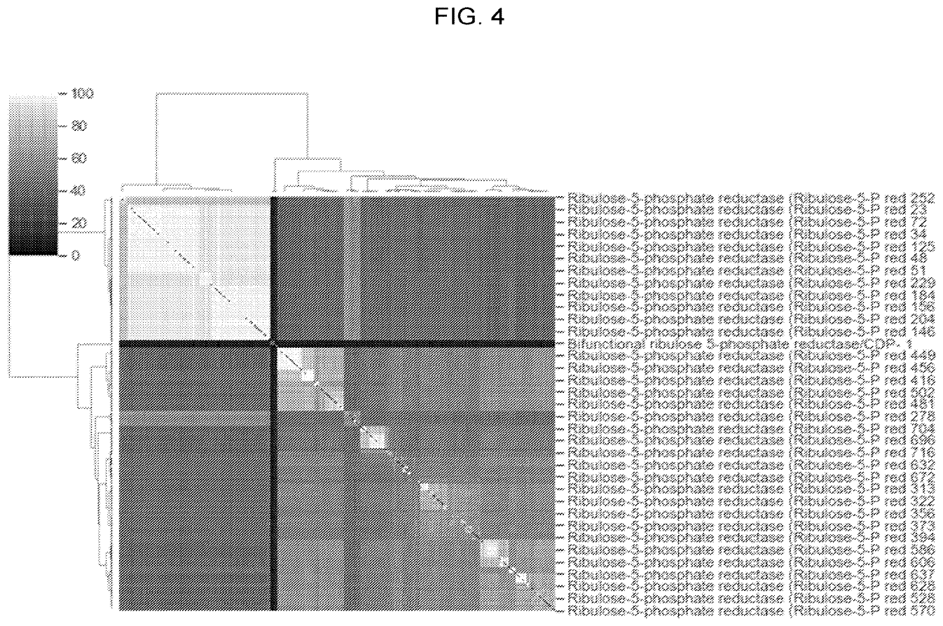

FIG. 5 shows in vitro activity of TarJ′ and XPDH enzymes as outlined in Example 3.

FIG. 6 shows erythritol, ribitol, and xylitol metabolite concentrations (g/L) at 96 hours of shake flask fermentations of strains 1-1, 1-13a-f, and 1-15a-f as outlined in Example 5. Data labels report the concentration (g/L) of xylitol.

FIG. 7 shows erythritol, ribitol, and xylitol metabolite concentrations (g/L) at 96 hours of shake flask fermentations of strains 1-1, 1-35a-d, 1-37a-d, 1-38a-f, and 1-39a-f as outlined in Example 5. Data labels report the concentration (g/L) of xylitol.

FIG. 8 shows erythritol, ribitol, and xylitol metabolite concentrations (g/L) at 96 hours of shake flask fermentations of strains 1-13c, 1-29a-e, 1-33a-e, and 1-34a-e as outlined in Example 6. Data labels report the concentration (g/L) of xylitol.

FIG. 9 shows erythritol, ribitol, arabitol, and xylitol metabolite concentrations (g/L) at 96 hours of shake flask fermentations of strains 1-13c, 1-12a-e, 1-14a-e, and 1-16a-e as outlined in Example 8. Data labels report the concentration (g/L) of xylitol (strains 1-13c, 1-14a-e, and 1-16a-e) or arabitol (strains 12a-e).

FIG. 10 shows erythritol, ribitol, and xylitol metabolite concentrations (g/L) at 96 hours of shake flask fermentations of strains-13c, 1-36a-e, and 1-40a-e as outlined in Example 9. Data labels report the concentration (g/L) of xylitol.

FIG. 11 shows erythritol, ribitol, and xylitol metabolite concentrations (g/L) at 96 hours of shake flask fermentations of strains 1-30a-e, 1-31a-e, 1-32a-e, and 1-13c as outlined in Example 10. Data labels report the concentration (g/L) of xylitol.

### DETAILED DESCRIPTION

Reference will now be made in detail to certain aspects of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

In this document, the terms “a,” “an,” or “the” are used to include one or more than one unless the context clearly dictates otherwise. The term “or” is used to refer to a nonexclusive “or” unless otherwise indicated. All publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

Values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

Unless expressly stated, ppm (parts per million), percentage, and ratios are on a by weight basis. Percentage on a by weight basis is also referred to as wt % or % (wt) below.

This disclosure relates to various recombinant cells engineered to produce xyltiol. In general, the recombinant cells described herein have an active pentose phosphate pathway and are characterized by expression of an exogenous xylitol-phosphate dehydrogenase (XPDH) enzyme. The disclosure further provides fermentation methods for the production of xylitol from dextrose using the genetically engineered cells described herein.

In general, recombinant cells described herein are yeast cells. As used herein, “yeast” refers to eukaryotic single celled microorganisms classified as members of the fungus kingdom. Yeast are unicellular organisms which evolved from multicellular ancestors with some species retaining multicellular characteristics such as forming strings of connected budding cells known as pseudo hyphae or false hyphae. Yeast cells may also be referred to in the art as yeast-like cells, and as used herein “yeast cell” encompasses both yeast and yeast-like cells. Suitable yeast and yeast-like host cells for modification may include, but are not limited to, Saccharomyces cerevisiae, Komagataella sp., Kluyveromyces (e.g., Kluyveromyces lactis, Kluveromyces marxianus), Yarrowia lipolytica, Issatchenkia orientalis, Pichia galeiformis, Pichia sp. YB-4149 (NRRL designation), Pichia pastoris, Candida (e.g., Candida magnoliae, Candida ethanolica), Pichia deserticola, Pichia membranifadens, Pichia fermentans, Aspergillus, Trichoderma, Myceliphthora thermophila, Moniliella (e.g., Moniliella pollinis), Pfaffia, Yamadazyma, Hansenula, Pichia kudriavzevvi, Trichosporonoides (e.g., Trichosporonoides megachiliensis, Trychosporonoides oedocephalis, Trychosporonoides nigrescens), Pseudozyma tsukubaensis, Trigonopsis variabilis, Penicillium, and Torula. An ordinarily skilled artisan would understand the requirements for selection of a suitable yeast cell, and recombinant yeast cells of the present disclosure are not limited to those expressly recited herein. Methods for genetic engineering of yeast cells are known and described in the art and a skilled artisan would understand the methods necessary to transform and engineer a suitable yeast cell.

A suitable yeast cell may be a cell of the phylum Basidiomycota and the subphylum Ustilaginomycotina. Suitable yeast of the subphylum Ustilaginomycotina include, but are not limited to, Ustilago (e.g., U. cynodontis, U. maydis, U. sphaerogena, U. cordal, U. scitaminea, U. coicis, U. syntherismae, U. esculenta, U. neglecta, U. crus-galli, Ustilago avenae), Sporisorium (e.g., Sporisorium exsertum), Moniliella (e.g., M. pollinis, M. tomentosa, M. acetoabutans, M. fonsecae, M. madida, M. megachiliensis, M. ocedocephalis, M. nigrescens), and Pseudozyma (e.g., Pseudozyma tsukubaensis), and Trichosporonoides (e.g., Trichosporonoides megachiliensis, Trychosporonoides oedocephalis, Trychosporonoides nigrescens). Yeast of the subphylum Ustilaginomycotina have been known and described in the art as potential production organisms for valuable chemicals such as itaconate, malate, succinate, mannitol, and erythritol and other valuable biotechnological applications. See, for example, Geiser et al. (Prospecting the biodiversity of the fungal family Ustilaginacceae for the production of value-added chemicals,” Fungal Biol Biotechnol, 2014, 1:2), Feldbrugge et al., (“The biotechnological use and potential of plant pathogenic smut fungi,” Appl Microbiol Biotechnol, 2013, 97 (8): 3253-65), Guevarra et al., (“Accumulation of itaconic, 2-hydroxyparaconic, itatartaric, and malic acids by strains of the genus Ustilago, Agric. Biol. Chem., 1990, 54 (9), 2353-2358), and Moon et al., (“Biotechnological production of erythritol and its applications,” Appl Microbiol Biotechnol, 2010, 86:1017-1025).

A suitable yeast cell will have an active pentose phosphate pathway that produces ribulose-5-phosphate. As used herein “active pentose phosphate pathway” refers to expression of one or more functional enzymes which, together, convert glucose-6-phosphate, NADP⁺ or NAD+,and water to NADPH or NADH, CO2, and ribulose-5-phosphate. Continuing in a non-oxidative phase, the pathway may also produce other pentose (i.e., 5-carbon) sugars. For example, the pentose phosphate pathway may produce ribulose-5-phosphate, ribose-5-phosphate, xylulose-5-phosphate, fructose 6-phosphate, combinations thereof, and the like, depending on the enzymatic activities present. The active pentose phosphate pathway may be native to the yeast cell or it may be introduced into the yeast cell by genetic engineering.

The yeast cell may be an osmotolerant yeast cell. As used herein, “osmotolerant” refers to a yeast capable of growth and reproduction under conditions of high osmolarity, such as at least 10% (w/v), at least 20% (w/v), at least 30% (w/v), at least 40% (w/v), at least 50% (w/v), or at least 60% (w/v) glucose and/or at least 6% (w/v), at least 10% (w/v), at least 12% (w/v), at least 13% (w/v), at least 15% (w/v) sodium chloride. Species and strains of osmotolerant yeast are known and described in the art, including many species of yeast used in industrial fermentation processes. Likewise, methods for assaying yeast osmotolerance are known and described in the art. See, for example, Tiwari, S., et al., (“Nectar yeast community of tropical flowering plants and assessment of their osmotolerance and xylitol-producing potential,” Current Microbiology, 2022, 79:28).

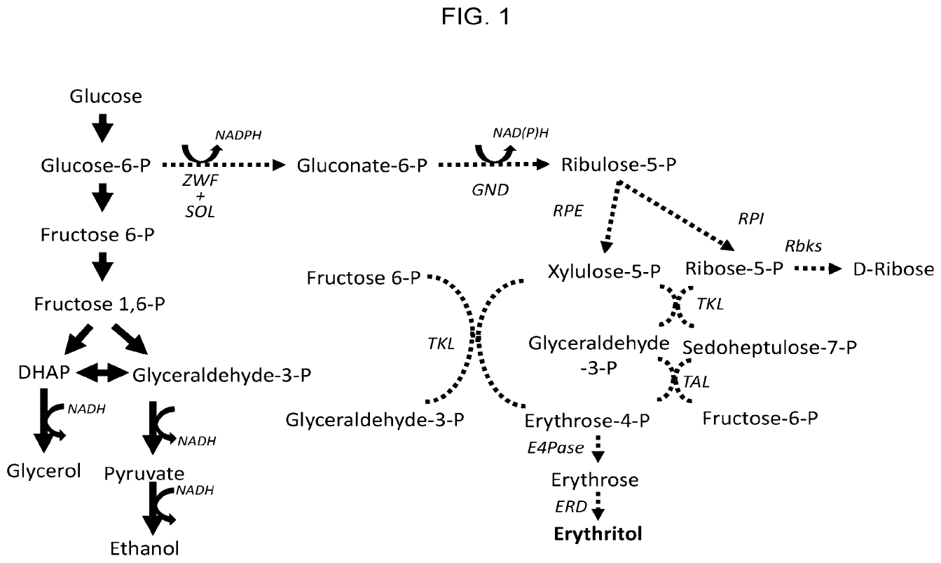

The recombinant yeast cell may be a recombinant Moniliella cell, for example, a Moniliella pollinis cell. FIG. 1 shows the predicted native pentose phosphate and glycolysis pathways in Moniliella pollinis. Moniliella has previously been used in the fermentation production of erythritol and methods for genetically modifying and fermenting Moniliella are known and described in the art. See, for example, Li et al. (“Methods for genetic transformation of filamentous fungi,” 2017, Microb Cell Fact, 16:168).

Various plasmids and methods for transformation of Moniliella are also described in the Examples below. For example, Moniliella may be transformed using a bipartite polynucleotide sequence(s) in which, following recombination, the exogenous polynucleotide of interest is integrated at the specified locus and the selection marker is expressible within the cell. Suitable selection markers are known and used in the art. The selectable marker may include, but is not limited to, amdS (for example broken into a 3′ portion, SEQ ID NO: 167, and a 5′ portion, SEQ ID NO: 174), G418 resistance gene (for example broken into a 3′ portion, SEQ ID NO:172, and a 5′ portion, SEQ ID NO:175), zeocin resistance gene (for example broken into a 3′ portion, SEQ ID NO: 168, and a 5′ portion, SEQ ID NO:169), nourseothricin N-acetyl transferase (NAT) (for example broken into a 3′ portion, SEQ ID NO:171, and a 5′ portion, SEQ ID NO:170), and invertase gene (SUC2) (for example a 3′ portion of SEQ ID NO: 173 and a 5′ portion of SEQ ID NO: 176).

The recombinant cells described herein include one or more exogenous polynucleotide sequences encoding one or more exogenous polypeptides that, when expressed improve the fermentation of glucose to ribitol by the recombinant cells.

The terms “glucose” and “dextrose” are used interchangeably herein and refer to D-glucose except where expressly indicated otherwise.

As used herein, “exogenous” refers to genetic material or an expression product thereof that originates from outside of the host organism. For example, the exogenous genetic material or expression product thereof can be a modified form of genetic material native to the host organism, it can be derived from another organism, it can be a modified form of a component derived from another organism, or it can be a synthetically derived component. For example, a K. lactis invertase gene is exogenous when introduced into S. cerevisiae.

As used herein, “native” refers to genetic material or an expression product thereof that is found, apart from individual-to-individual mutations which do not affect function or expression, within the genome of wild-type cells of the host cell. For the purposes of this application, the Moniliella pollinis cell “Moniliella tomentosa var pollinis TCV364” described in U.S. Pat. No. 6,440,712,which is incorporated herein by reference in its entirety, and deposited under the Budapest Treaty at BCCM/MUCL (Belgian Coordinated Collections of Micro-organisms/Mycothèque de l′Université Catholique de Louvain by Eridania Béghin Say, Vilvoorde R&D Centre, Havenstraat 84, B-1800 Vilvoorde) on Mar. 28, 1997 under number MUCL40385, is considered the wild-type Moniliella pollinis cell.

As used herein, the terms “polypeptide” and “peptide” are used interchangeably and refer to the collective primary, secondary, tertiary, and quaternary amino acid sequences and structure necessary to give the recited macromolecule its function and properties. As used herein, “enzyme” or “biosynthetic pathway enzyme” refer to a protein that catalyzes a chemical reaction. The recitation of any particular enzyme, either independently or as part of a biosynthetic pathway is understood to include the co-factors, co-enzymes, and metals necessary for the enzyme to properly function. A summary of the amino acids and their three and one letter symbols as understood in the art is presented in Table 1. The amino acid name, three letter symbol, and one letter symbol are used interchangeably herein.

TABLE 1
Amino Acid three and one letter symbols

| Amino Acid | Three letter symbol | One letter symbol |
|---|---|---|
| Alanine | Ala | A |
| Arginine | Arg | R |
| Asparagine | Asn | N |
| Aspartic acid | Asp | D |
| Cysteine | Cys | C |
| Glutamic acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

Variants or sequences having substantial identity or homology with the polypeptides described herein can be utilized in the practice of the disclosed recombinant cells, compositions, and methods. Such sequences can be referred to as variants or modified sequences. That is, a polypeptide sequence can be modified yet still retain the ability to exhibit the desired activity. Generally, the variant or modified sequence may include greater than about 45%, 45%, 50%, 55%, 60%, 65%, 70%, 75%, 80%, 85%, 90%, or 95% sequence identity with the wild-type, naturally occurring polypeptide sequence, or with a variant polypeptide as described herein.

As used herein, the phrases “% sequence identity,” “% identity,” and “percent identity,” are used interchangeably and refer to the percentage of residue matches between at least two amino acid sequences or at least two nucleic acid sequences aligned using a standardized algorithm. Methods of amino acid and nucleic acid sequence alignment are well-known. Sequence alignment and generation of sequence identity include global alignments and local alignments which are carried out using computational approaches. An alignment can be performed using BLAST (National Center for Biological Information (NCBI) Basic Local Alignment Search Tool) version 2.2.31 software with default parameters. Amino acid % sequence identity between amino acid sequences can be determined using standard protein BLAST with the following default parameters: Max target sequences: 100; Short queries: Automatically adjust parameters for short input sequences: Expect threshold: 10; Word size: 6; Max matches in a query range: 0; Matrix: BLOSUM62; Gap Costs: (Existence: 11, Extension: 1); Compositional adjustments: Conditional compositional score matrix adjustment; Filter: none selected; Mask: none selected. Nucleic acid % sequence identity between nucleic acid sequences can be determined using standard nucleotide BLAST with the following default parameters: Max target sequences: 100; Short queries: Automatically adjust parameters for short input sequences; Expect threshold: 10; Word size: 28; Max matches in a query range: 0; Match/Mismatch Scores: 1, −2; Gap costs: Linear; Filter: Low complexity regions; Mask: Mask for lookup table only. A sequence having an identity score of XX % (for example, 80%) with regard to a reference sequence using the NCBI BLAST version 2.2.31 algorithm with default parameters is considered to be at least XX % identical or, equivalently, have XX % sequence identity to the reference sequence.

Polypeptide or polynucleotide sequence identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30,at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

The polypeptides disclosed herein may include “variant” polypeptides, “mutants,” and “derivatives thereof.” As used herein the term “wild-type” is a term of the art understood by skilled persons and means the typical form of a polypeptide as it occurs in nature as distinguished from variant or mutant forms. As used herein, a “variant,” “mutant,” or “derivative” refers to a polypeptide molecule having an amino acid sequence that differs from a reference protein or polypeptide molecule. A variant or mutant may have one or more insertions, deletions, or substitutions of an amino acid residue relative to a reference molecule.

The amino acid sequences of the polypeptide variants, mutants, derivatives, or fragments as contemplated herein may include conservative amino acid substitutions relative to a reference amino acid sequence. For example, a variant, mutant, derivative, or fragment polypeptide may include conservative amino acid substitutions relative to a reference molecule. “Conservative amino acid substitutions” are those substitutions that are a substitution of an amino acid for a different amino acid where the substitution is predicted to interfere least with the properties of the reference polypeptide. In other words, conservative amino acid substitutions substantially conserve the structure and the function of the reference polypeptide. Conservative amino acid substitutions generally maintain (a) the structure of the polypeptide backbone in the area of the substitution, for example, as a beta sheet or alpha helical conformation, (b) the charge and/or hydrophobicity of the molecule at the site of the substitution, and/or (c) the bulk of the side chain.

As used herein, terms “polynucleotide,” “polynucleotide sequence,” and “nucleic acid sequence,” and “nucleic acid,” are used interchangeably and refer to a sequence of nucleotides or any fragment thereof. These phrases also refer to DNA or RNA of natural or synthetic origin, which may be single-stranded or double-stranded and may represent the sense or the antisense strand. The DNA polynucleotides may be a cDNA (e.g., coding DNA) or a genomic DNA sequence (e.g., including both introns and exons).

A polynucleotide is said to encode a polypeptide if, in its native state or when manipulated by methods known to those skilled in the art, it can be transcribed and/or translated to produce the polypeptide or a fragment thereof. The anti-sense strand of such a polynucleotide is also said to encode the sequence.

Those of skill in the art understand the degeneracy of the genetic code and that a variety of polynucleotides can encode the same polypeptide. In some aspects, the polynucleotides (e.g., polynucleotides encoding an XPDH polypeptide) may be codon-optimized for expression in a particular cell including, without limitation, a plant cell, bacterial cell, fungal cell, or animal cell. While polypeptides encoded by polynucleotide sequences found in various species are disclosed herein any polynucleotide sequences may be used which encodes a desired form of the polypeptides described herein. Thus, non-naturally occurring sequences may be used. These may be desirable, for example, to enhance expression in heterologous expression systems of polypeptides or proteins. Computer programs for generating degenerate coding sequences are available and can be used for this purpose. Pencil, paper, the genetic code, and a human hand can also be used to generate degenerate coding sequences.

The recombinant cells described herein may include deletions or disruptions in one or more native genes. The phase “deletion or disruption” refers to the status of a native gene in the recombinant cell that has either a completely eliminated coding region (deletion) or a modification of the gene, its promoter, or its terminator (such as by a deletion, insertion, or mutation) so that the gene no longer produces an active expression product, produces severely reduced quantities of the expression product (e.g., at least a 75% reduction or at least a 90% reduction) or produces an expression product with severely reduced activity (e.g., at least 75% reduced or at least 90% reduced). The deletion or disruption can be achieved by genetic engineering methods, forced evolution, mutagenesis, RNA interference (RNAi), and/or selection and screening. The native gene to be deleted or disrupted may be replaced with an exogenous nucleic acid of interest for the expression of an exogenous gene product (e.g., polypeptide, enzyme, and the like).

The recombinant cells described herein may include one or more genetic modifications in which an exogenous nucleic acid is integrated into the genome of the host cell. One of skill in the art know how to select suitable loci in a yeast genome for integration of the exogenous nucleic acid. Suitable integration loci may include, but are not limited to, the PDC1, GPDI, CYB2A, CYB2B, g4240, YMR226, MDHB, ATO2, Adh9091, Adh1202, ADE2, ADH2556, GAL6, MDH1, SCW11, ER1, ER3, pyrF, TRP3, gpdIIA, and gpdIIB loci. For example, in a M. pollinis host cells, suitable interaction loci may include, but are not limited to, the ER1 locus (defined as the locus flanked by SEQ ID NO:85 and SEQ ID NO:162), the ER3 locus (defined as the locus flanked by SEQ ID NO: 155 and SEQ ID NO:165), the PDC1 locus (defined as the locus flanked by SEQ ID NO: 152 and SEQ ID NO:164), the pyrF locus (defined as the locus flanked by SEQ ID NO: 153 and SEQ ID NO: 163), the TRP3 locus (defined as the locus flanked by SEQ ID NO:156 and SEQ ID NO: 159), the gpdIIA locus (defined as the locus flanked by SEQ ID NO:157 and SEQ ID NO: 161); and the gpdIIB locus (defined as the locus flanked by SEQ ID NO: 158 and SEQ ID NO: 166). The exogenous nucleic acid may also be integrated in an intergenic region or other location in the host cell genome not specifically specified herein. Other suitable integration loci may be determined by one of skill in the art. Furthermore, one of skill in the art would recognize how to use sequences to design primers to verify correct gene integration at the chosen locus.

The recombinant cell may have one or more copies of a given exogenous nucleic acid sequence integrated in a host chromosome(s) and replicated together with the chromosome(s) into which it has been integrated. For example, the yeast cell may be transformed with nucleic acid construct including a polynucleotide sequence encoding for a polypeptide described herein and the polynucleotide sequence encoding for the polypeptide may be integrated in one or more copies in a host chromosome(s). The recombinant cell may include multiple copies (two or more) of a given polynucleotide sequence encoding a polypeptide described herein. The recombinant cell may have one, two, three, four, five, six, seven, eight, nine, ten, or more copies of a polynucleotide sequence encoding a polypeptide described herein integrated into the genome. The multiple copies of said polynucleotide sequence may all be incorporated at a single locus or may be incorporated at multiple loci.

The recombinant cells described herein are capable of producing xylitol and include an exogenous polynucleotide sequence encoding a xylitol-phosphate dehydrogenase (XPDH) enzyme. The exogenous polynucleotide sequence may be an exogenous xylitol-phosphate dehydrogenase (XPDH) gene. Following conversion of xylulose 5-phosphate to xylitol 5-phosphate, it is believed a native phosphatase enzyme removes the phosphate to produce xylitol.

A “xylitol-phosphate dehydrogenase gene” and an “XPDH gene” are used interchangeably herein and refer to any gene or polynucleotide that encodes a polypeptide with xylitol-phosphate dehydrogenase activity. As used herein, “xylitol-phosphate dehydrogenase activity” refer to the ability to catalyze the conversion of xylulose-5-phosphate and NADPH or NADH to xylitol 5-phosphate and NADP⁺ or NAD⁺. The XPDH gene may be derived from any suitable source. For example, the XPDH gene may be derived from Clostridium difficile, Lactobacillus rhamnosus, Bacillus halodurans, Alkalihalobacillus ligniniphilus, Jeotgalibacillus soli, Heyndrickxia sporothermodurans, Clostridium fungisolvens, or Neobacillus cucumis. The XPDH gene may encode an amino acid at least 50%, at least 60%, at least 70%, at least 80%, at least 85%, at least 90%, at least 95%, at least 97%, at least 99%, or 100% sequence identity to the amino acid sequence of at least one of SEQ ID NOs: 12-15, 28-32, or 33. The XPDH gene may encode an amino acid at least 50%, at least 60%, at least 70%, at least 80%, at least 85%, at least 90%, at least 95%, at least 97%, at least 99%, or 100% sequence identity to the amino acid sequence of at least one of SEQ ID NOs: 14, 15, 28, or 31.

The recombinant cell may comprise an exogenous polynucleotide that is or may be derived from a Clostridium difficile gene encoding the amino acid of SEQ ID NO:12. The exogenous polynucleotide may encode an amino acid sequence with at least 50%, at least 60%, at least 70%, at least 80%, at least 85%, at least 90%, at least 95%, at least 97%, or at least 99% sequence identity to the amino acid sequence of SEQ ID NO: 12.

The recombinant cell may comprise an exogenous polynucleotide that is or may be derived from a Clostridium difficile gene encoding the amino acid of SEQ ID NO:13. The exogenous polynucleotide may encode an amino acid sequence with at least 50%, at least 60%, at least 70%, at least 80%, at least 85%, at least 90%, at least 95%, at least 97%, or at least 99% sequence identity to the amino acid sequence of SEQ ID NO: 13.

The recombinant cell may comprise an exogenous polynucleotide that is or may be derived from a Lactobacillus rhamnosus gene encoding the amino acid of SEQ ID NO:14. The exogenous polynucleotide may encode an amino acid sequence with at least 50%, at least 60%, at least 70%, at least 80%, at least 85%, at least 90%, at least 95%, at least 97%, or at least 99% sequence identity to the amino acid sequence of SEQ ID NO: 14.

The recombinant cell may comprise an exogenous polynucleotide that is or may be derived from a Bacillus halodurans gene encoding the amino acid of SEQ ID NO:15. The exogenous polynucleotide may encode an amino acid sequence with at least 50%, at least 60%, at least 70%, at least 80%, at least 85%, at least 90%, at least 95%, at least 97%, or at least 99% sequence identity to the amino acid sequence of SEQ ID NO:15.

The recombinant cell may comprise an exogenous polynucleotide that is or may be derived from a Alkalihalobacillus ligniniphilus gene encoding the amino acid of SEQ ID NO:28. The exogenous polynucleotide may encode an amino acid sequence with at least 50%, at least 60%, at least 70%, at least 80%, at least 85%, at least 90%, at least 95%, at least 97%, or at least 99% sequence identity to the amino acid sequence of SEQ ID NO:28.

The recombinant cell may comprise an exogenous polynucleotide that is or may be derived from a Jeotgalibacillus soli gene encoding the amino acid of SEQ ID NO:29. The exogenous polynucleotide may encode an amino acid sequence with at least 50%, at least 60%, at least 70%, at least 80%, at least 85%, at least 90%, at least 95%, at least 97%, or at least 99% sequence identity to the amino acid sequence of SEQ ID NO:29.

The recombinant cell may comprise an exogenous polynucleotide that is or may be derived from a Heyndrickxia sporothermodurans gene encoding the amino acid of SEQ ID NO:30. The exogenous polynucleotide may encode an amino acid sequence with at least 50%, at least 60%, at least 70%, at least 80%, at least 85%, at least 90%, at least 95%, at least 97%, or at least 99% sequence identity to the amino acid sequence of SEQ ID NO:30.

The recombinant cell may comprise an exogenous polynucleotide that is or may be derived from a Clostridium fungisolvens gene encoding the amino acid of SEQ ID NO:31. The exogenous polynucleotide may encode an amino acid sequence with at least 50%, at least 60%, at least 70%, at least 80%, at least 85%, at least 90%, at least 95%, at least 97%, or at least 99% sequence identity to the amino acid sequence of SEQ ID NO:31.

The recombinant cell may comprise an exogenous polynucleotide that is or may be derived from a Neobacillus cucumis gene encoding the amino acid of SEQ ID NO:33. The exogenous polynucleotide may encode an amino acid sequence with at least 50%, at least 60%, at least 70%, at least 80%, at least 85%, at least 90%, at least 95%, at least 97%, or at least 99% sequence identity to the amino acid sequence of SEQ ID NO:33.

The exogenous polynucleotides in the recombinant cells described herein may be under the control of a promoter. For example, the exogenous nucleic acid may be operably linked to a heterologous or artificial promoter. Suitable promoters are known and described in the art. Promoters may include, but are not limited to, pyruvate decarboxylase promoter (PDC), translation elongation factor 2 promoter (TEF2), SED1, alcohol dehydrogenase 1A promoter (ADH1), hexokinase 2 promoter (HXK2), FLO5 promoter, pyruvate kinase 1 promoter (PYKIp; SEQ ID NO: 86); 6-phosphogluconate dehydrogenase promoter (6PGDp; SEQ ID NO:130); glyceraldehyde-3-phosphate dehydrogenase promoter (TDH3p; SEQ ID NO:132); translational elongation factor 1 promoter (TEFp; SEQ ID NO:133); modified TEFp (SEQ ID NO:131); phosphoglucomutase 1 promoter (PGM1p; SEQ ID NO:134); 3-phosphoglycerate kinase promoter (PGK1p; SEQ ID NO:135); enolase promoter (ENOIp; SEQ ID NO:136); asparagine synthetase promoter (ASNSp; SEQ ID NO:137); 50S ribosomal protein L1 promoter (RPLAp; SEQ ID NO: 138); and RPL16B (SEQ ID NO:139).

The exogenous nucleic acids in the recombinant cells described herein may be under the control of a terminator. For example, the exogenous nucleic acid may be operably linked to a heterologous or artificial terminator. Suitable terminators are known and described in the art. Terminators may include, but are not limited to, GAL10 terminator, PDC terminator, transaldolase terminator (TAL) 6PGD terminator (6PGDt; SEQ ID NO:140); ASNS terminator (ASNSt; SEQ ID NO: 141); ENO1 terminator (ENO1t; SEQ ID NO:142); hexokinase 1 terminator (HXK1t; SEQ ID NO: 143); PGK1 terminator (PGK1t; SEQ ID NO:144); PGM1 terminator (PGM1t; SEQ ID NO: 145); PYK1 terminator (PYK1t; SEQ ID NO:146); RPLA terminator (RPLAt: SEQ ID NO: 147); transaldolase 1 terminator (TALIt; SEQ ID NO:148); TDH3 terminator (TDH3t; SEQ ID NO: 149); translation elongation factor 2 terminator (TEF2t; SEQ ID NO:150); and triosephosphate isomerase 1 terminator (TPIIt; SEQ ID NO:151).

A promoter or terminator is “operably linked” to a given polynucleotide (e.g., a gene) if its position in the genome or expression cassette relative to said polynucleotide is such that the promoter or terminator, as the case may be, performs its transcriptional control function.

The polypeptides described herein may be provided as part of a construct. As used herein, the term “construct” refers to recombinant polynucleotides including, without limitation, DNA and RNA, which may be single-stranded or double-stranded and may represent the sense or the antisense strand. Recombinant polynucleotides are polynucleotides formed by laboratory methods that include polynucleotide sequences derived from at least two different natural sources or they may be synthetic. Constructs thus may include new modifications to endogenous genes introduced by, for example, genome editing technologies. Constructs may also include recombinant polynucleotides created using, for example, recombinant DNA methodologies. The construct may be a vector including a promoter operably linked to the polynucleotide encoding a polypeptide as described herein. As used herein, the term “vector” refers to a polynucleotide capable of transporting another polynucleotide to which it has been linked. The vector may be a plasmid, which refers to a circular double-stranded DNA loop into which additional DNA segments may be integrated.

The disclosure also provides fermentation methods for the production of xylitol using the recombinant cells described herein. The fermentation methods include the step of fermenting a substrate using the genetically engineered yeasts described herein to product xyltiol. The fermentation method can include additional steps, as would be understood by a person skilled in the art. Non-limiting examples of additional process steps include maintaining the temperature of the fermentation broth within a predetermined range, adjusting the pH during fermentation, and isolating the xyltiol from the fermentation broth. The fermentation process may be a fully aerobic process.

The fermentation method can be run using a suitable fermentation substrate. The substrate of the fermentation method can include glucose, sucrose, galactose, mannose, molasses, xylose, fructose, hydrolysates of starch, lignocellulosic hydrolysates, or a combination thereof. One skilled in the art will recognize what fermentation substrate is suitable for a given fermentation organism and system.

The fermentation process can be run under various conditions. The fermentation temperature, i.e., the temperature of the fermentation broth during processing, may be ambient temperature. Alternatively, or additionally, the fermentation temperature may be maintained within a predetermined range. For example, the fermentation temperature can be maintained in the range of 25° C. to 45° C., 30° C. to 40° C., or 32° C. to 37° C., preferably about 35° C. However, a skilled artisan will recognize that the fermentation temperature is not limited to any specific range or temperature recited herein and may be modified as appropriate.

The fermentation process can be run within certain oxygen uptake rate (OUR) ranges. The volumetric OUR of the fermentation process can be in the range of 0.5 to 40, 1 to 35, 2 to 30, 3 to 25, 4 to 20, or 5 to 15 mmol O₂/(L·h). In some embodiments, the specific OUR can be in the range of 0.05 to 10, 0.1 to 8, 0.15 to 5, 0.2 to 1, or 0.3 to 0.75 mmol O₂/(g cell dry weight· h). However, the volumetric or specific OURs of the fermentation process are not limited to any specific rates or ranges recited herein.

The fermentation process can be run at various cell concentrations. In some embodiments, the cell dry weight at the end of fermentation can be 5 to 40, 8 to 30, or 10 to 20 g cell dry weight/L. Further, the pitch density or pitching rate of the fermentation process can vary. In some embodiments, the pitch density can be 0.05 to 11, 0.1 to 10, or 0.25 to 8 g cell dry weight/L.

The initial dextrose concentration of the fermentation may be at least 100, 200, 250, 300, 350, or at least 400 g/L dextrose. The initial dextrose concentration may be between 100 to 400, 150 to 350, or 250 to 325 g/L.

The fermentation process can be associated with various characteristics, such as, but not limited to, fermentation production rate, pathway fermentation yield, final titer, and peak fermentation rate. These characteristics can be affected by the selection of the yeast and/or genetic modification of the yeast used in the fermentation process. These characteristics can be affected by adjusting the fermentation process conditions. These characteristics can be adjusted via a combination of yeast selection or modification and the selection of fermentation process conditions.

The xylitol production rate of the process may be at least at least 0.2, 0.3, 0.5, 0.75, or at least 1.0 g L⁻¹ h⁻¹. The xylitol mass yield of the process may be at least 55 percent, at least 65 percent, at least 70 percent, at least 75 percent, at least 80 percent, or at least 85 percent. The final xylitol titer of the process may be at least 20, 30, 50, 75, or 100 g/L.

The fermentation process can be run as a dextrose-fed batch. Further, the fermentation process can be a batch process, continuous process, or semi-continuous process, as would be understood by a person skilled in the art.

### EXAMPLES

The invention is further described in detail by reference to the following experimental examples. These examples are provided for purposes of illustration only and are not intended to be limiting unless otherwise specified. Thus, the invention should in no way be construed as being limited to the following examples, but rather should be construed to encompass any and all variations which become evident as a result of the teaching provided herein.

### Example 1-Xylitol-Phosphate Dehydrogenase Diversity

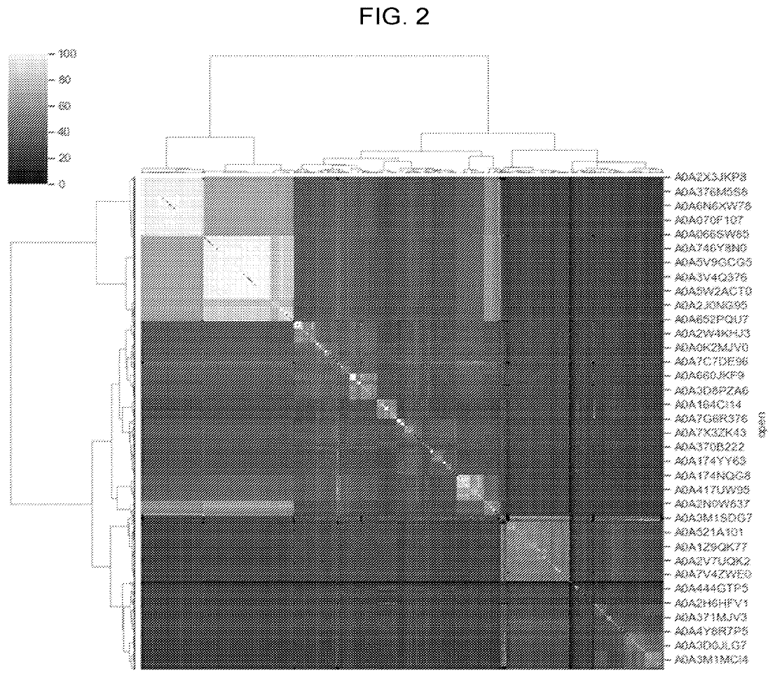

Roughly three thousand galactitol-1-phosphate-5-dehydrogenase (G1PDH)/xylitol-phosphate dehydrogenase (XPDH) enzyme sequences were obtained from Uniprot and analyzed. FIG. 2 illustrates the natural sequence diversity for this set of sequences. This set is diverse, with ˜25% of the enzymes having no homologue more than 75% identical. As these enzymes tend to prefer NAD to NADP as a cofactor, the cofactor binding preferences of the homologs were assessed in a manner similar to that described by Duax et al., (“Rational proteomics I. Fingerprinting identification and cofactor specificity in the short-chain oxidoreductase (SCOR) enzyme family,” Proteins, 2003, 53 (4): 931-943). Cofactor binding pockets were identified by proximity to the Rossman fold (+23 to +30 amino acids from the GXGXXG motif (SEQ ID NO: 129)) and scored on the basis of total charge in an 8-residue window. The top 8 candidates that were predicted to use NADP were selected for further characterization, along with 4 candidates predicted to use NAD, and 3 controls.

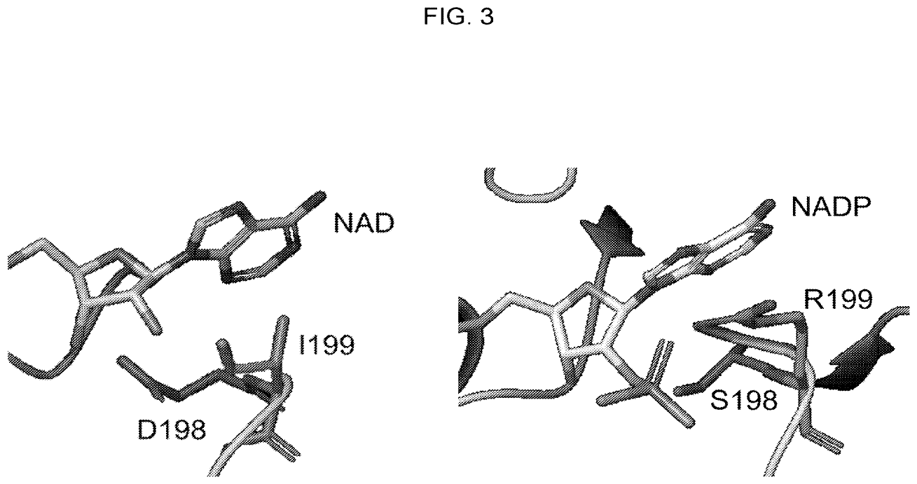

Upon further review of the structural characteristics of the predicted binding pocket for factors that may influence cofactor preference, an important aspartate residue was identified. See FIG. 3. The polypeptide of SEQ ID NO:34 and substitutions thereof were used to construct a structural homology model to predict cofactor binding pocket confirmations FIG. 3 shows the C-terminal end of the penultimate β-strand on the outside of the Rossman Fold domain. Without wishing to be bound by any particular theory, it is predicted that enzymes in which the first residue in this region (residue 198 relative to SEQ ID NO:34) is an aspartate and the second residue (residue 199 relative to SEQ ID NO:34) is a large hydrophobic amino acid (for example, isoleucine) will prefer an NAD cofactor due to the hydrogen bonding of the aspartate to the hydroxyl groups of the NAD ribose. However, enzymes in which that first residue (residue 198 relative to SEQ ID NO:34) is an alanine, glycine, or serine and the second residue (residue 199 relative to SEQ ID NO:34) is lysine or arginine will prefer an NADP cofactor as the positive charge on the lysine or arginine residue will interact with the negative charge of the phosphate of the NADP and the smaller residue in the first position allows space in the binding pocket for said phosphate. Based on this analysis, 12 additional enzymes were selected for their predicted preference for NADP. Finally, 6 additional enzymes with sequence similarity to active XPDH enzymes were selected for testing.

### Example 2-TarJ′ Diversity

Roughly eight hundred ribulose 5-phosphate reductase sequences were obtained from Uniprot and analyzed. FIG. 3 illustrates the natural sequence diversity for this set of sequences. Overall, the diversity in this set is low, as only 10% of the enzymes have no sequence similarity more than 75% identical. As these enzymes tend to prefer NADP to NAD as a cofactor, no scoring was performed, and the sequences were simply aligned in Geneious (ClustalW, default settings). Eight enzymes were selected for further analysis based on sequence similarity.

### Example 3-In Vitro Enzyme Assays

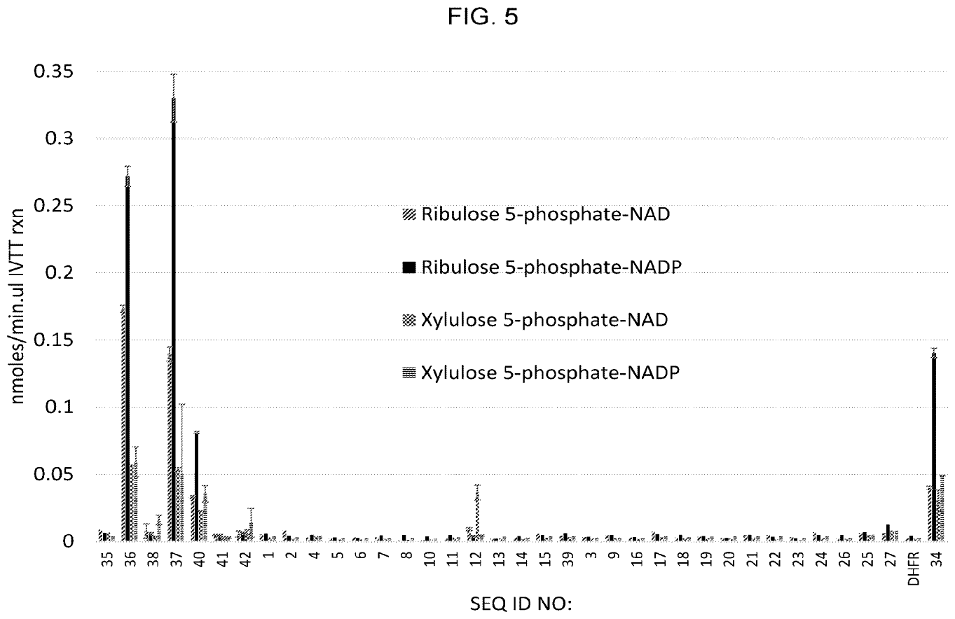

Polynucleotides encoding suspected XPDH homologs (Table 2) or TarJ′ homologs (Table 3) were cloned into a vector containing a T7 promoter and terminator for cell-free protein expression (New England Biolabs, PURExpress® In Vitro Protein Synthesis). Cell-free synthesized proteins were analyzed for activity on four substrates (ribulose 5-phosphate, xylulose 5-phosphate, ribulose, and xylulose) with either NADP or NAD cofactors. Seven enzymes (XPDH of SEQ ID NOs: 12 and 34, TarJ′ of SEQ ID NOs: 36, 37, 38, 40, and 42), were able to catalyze the reduction of either ribulose 5-phosphate or xylulose 5-phosphate (FIG. 5) but not the reduction of xylulose or ribulose (data not shown).

TABLE 2
XPDH Homologs

| Source | Gene Name/ | Polypeptide |
|---|---|---|
| Organism | Accession Numbers | SEQ ID NO: |
| Mariniphaga sediminis | D1164_16045 | 1 |
| Candidatus Dormibacteraeota bacterium | DLM67_26945 | 2 |
| Candidatus Falkowbacteria bacterium | COT93_01110 | 3 |
| Acidobacteria bacterium | DMG39_01315 | 4 |
| Spirochaetaceae bacterium | DCP56_03760 | 5 |
| Candidatus Hydrogenedentes bacterium | ENW01_11985 | 6 |
| Rhizobiumsp. | E6Q76_20070 | 7 |
| Citrobacter freundii | C2U38_22630 | 8 |
| Escherichia coli | gatD | 9 |
| Escherichia coli (strain K12) | rspB | 10 |
| Lactobacillus salivarius cp400 | LSCP400_11731 | 11 |
| Clostridium difficile | WP_004454628.1 | 12 |
| Clostridium difficile | WP_011860836.1 | 13 |
| Lactobacillus rhamnosus | AAT02414.1 | 14 |
| Bacillus halodurans | WP_010896369.1 | 15 |
| Lachnospiraceae bacterium | A0A3C1WRV0_9FIRM | 16 |
| Faecalibacterium sp. OF03-6AC | DXA66_03105 | 17 |
| Agathobacter ruminis | CSX02_08830 | 18 |
| Alicyclobacillus acidoterrestris | AAC03nite_18160 | 19 |
| Ruminococcus sp. KGMB03662 | FFK04_01895 | 20 |
| Clostridiaceae bacterium | D3Z58_08610 | 21 |
| Thermoplasmata archaeon | A0A497HL05 | 22 |
| Lachnospiraceae bacterium | A0A3B918G1 | 23 |
| Prevotella marshii DSM 16973 = JCM | E0NTD1 | 24 |
| 13450 | | |
| Blautia sp. OM05-6 | A0A417S5N4 | 25 |
| Candidatus Bathyarchaeota archaeon | A0A7C4RGE8 | 26 |
| Lachnospiraceae bacterium | A0A7U9REN4 | 27 |

TABLE 3
TarJ' Homologs

| Source | Gene Name/ | Polypeptide |
|---|---|---|
| Organism | Accession Numbers | SEQ ID NO: |
| Staphylococcus aureus | AMV81374.1 | 34 |
| Staphylococcus aureus subsp. aureus 71193 | ST398NM01_0269 | 35 |
| Eubacterium ventriosum | DW918_{—} | 36 |
| Pradoshia sp. D12 | F7984_17520 | 37 |
| Lactobacillus plantarum EGD-AQ4 | N692_02285 | 38 |
| uncultured Ruminococcus sp. | DPQ25_03780 | 39 |
| Kandleria vitulina | SAMN04487759_1396 | 40 |
| Lactiplantibacillus plantarum | C7M34_02724 C7M35_00347 | 41 |
| | C7M36_02765 C7M47_01038 | |
| | FET70_01115 LPJSA22_01709 | |
| | Nizo2802_1951 | |
| Lachnospiraceae bacterium | IMSAGC019_01831 | 42 |

### Example 4-Genetically Modified Moniliella pollinis Strains

Strain 1-1 is the Moniliella pollinis host strain “Moniliella tomentosa var pollinis TCV364” described in U.S. Pat. No. 6,440,712, which is incorporated herein by reference in its entirety, and deposited under the Budapest Treaty at BCCM/MUCL (Belgian Coordinated Collections of Micro-organisms/Mycothèque de l′Université Catholique de Louvain by Eridania Béghin Say, Vilvoorde R&D Centre, Havenstraat 84, B-1800 Vilvoorde) on Mar. 28, 1997 under number MUCL40385. Table 4 below lists various Moniliella pollinis strains, including information on the parent strain, the sequence with which the parent strain was transformed, and characterizations of the expression cassette(s) contained on the transformed sequence. Each “XPDH/TarJ′ Homolog Expression Cassette” contained, in order, a 5′ ER1 flanking sequence (SEQ ID NO:85), a MpPYK1 promoter (SEQ ID NO:86), a gene encoding the indicated XPDH or TarJ′ homolog (one of SEQ ID NOs: 87-128), a Mp6PGD terminator (SEQ ID NO:140), and a 5′ portion of a G418 resistance gene expression cassette (SEQ ID NO:175). Each “Selectable Marker Cassette” contained, in order, a 3′ portion of a G418 resistance gene expression cassette (SEQ ID NO:172), an MpTEF2 terminator (SEQ ID NO:150), and a 3′ ER1 flanking sequence (SEQ ID NO:160). Upon bipartite transformation with both the XPDH/TarJ′ Homolog Expression Cassette and the Selectable Marker Cassette, the two cassettes recombine for integration of both the nucleotide sequence encoding the XPDH or TarJ′ homolog and the G418 resistance marker at the ER1 locus.

The indicated Moniliella pollinis parent strain was transformed with the indicated sequence(s) by first protoplasting the parent strain by adding an enzyme mixture containing 0.6M MgSO₄, 7.5 g/L driselase, and 12.5 g/L Trichoderma harzianum lysing enzyme to a mycelial pellet of the parent strain. Protoplasts were then pelleted, washed with 0.6M MgSO₄, and resuspended in STC medium (0.6M sucrose, 50 mM CaCl2, 10 mM Tris-HCl, pH 7.5). Bipartite transformations were prepared by adding 100 μg single stranded salmon sperm DNA and 1.5 to 5 μg each of the 5′ and 3′ DNA transformation fragments (3-10 μg total; see Table 4 for list of fragments) to approximately 200 μL protoplast mixture (10⁸ cells/mL). 1 mL 50% PEG in STC medium was then added to the salmon sperm DNA, transformation DNA, and protoplast mixture and the resulting combination was incubated for 15 minutes at room temperature. Following incubation, recovery broth (0.4M sucrose, 1 g/L yeast extract, 1 g/L malt extract, 10 g/L glucose, pH 4.5) was added to the mixture and incubated at 27° C., 100 rpm, for 16 to 24 hours. Following the incubation, protoplasts were pelleted by centrifugation and resuspended in 1 mL PBS.

The resuspended protoplasts were plated on PDA+250 mg/L geneticin (G418)selection plates and incubated at 30-35° C. for at least 2-4 days until transformants grow. Resulting transformants were evaluated by colony PCR for integration of the indicated sequence. A PCR verified isolate was then designated as the indicated strain number. In some instances, more than one PCR verified isolate, e.g., “sister” isolates, are indicated by letters following the strain number. For example, strain 1-2 has 5 sister isolates, strains 1-2a, 1-2b, 1-2c, 1-2d, and 1-2e.

For example, Strain 1-1 was transformed with SEQ ID NO:43 and SEQ ID NO:44. SEQ ID NO: 43 contains (i) 3′ flanking DNA for targeted chromosomal integration into the ER1 locus (SEQ ID NO:162), and (ii) a 3′ portion of the G418 resistance gene selectable marker (SEQ ID NO: 172). SEQ ID NO:44 contains (i) an expression cassette for the XPDH homolog from M. sediminis, SEQ ID NO:87 encoding the amino acid sequence of SEQ ID NO:1, under the control of the PYK1 promoter of SEQ ID NO:86 and the PGD terminator of SEQ ID NO:140; (ii) 5′ flanking DNA for targeted chromosomal integration into the ER1 locus (SEQ ID NO:85); and (iii) a 5′ portion of the G418 resistance gene selectable marker (SEQ ID NO: 175). Transformants were selected on PDA+250 mg/L geneticin (G418) selection plates and incubated at 30-35° C. for at least 2 days until transformants grow. Resulting transformants were streaked for single colony isolation on PDA+geneticin (G418) plates and single colonies were selected. Selected colonies were evaluated by colony PCR for integration of the indicated sequence. PCR verified isolates were designated strains 1-2a, 1-2b, 1-2c, 1-2d, and 1-2e.

TABLE 4

| | | | | | Selectable Marker |
|---|---|---|---|---|---|
| | | XPDH/TarJ' Homolog Expression Cassette | | | Cassette |
| | Parent | Transformation | Nucleotide | Encoded Polypeptide | Transformation |
| Strain | Strain | SEQ ID NO: | SEQ ID NO: | SEQ ID NO: | SEQ ID NO: |
| 1-2a-e | 1-1 | 44 | 87 | 1 | 43 |
| 1-3a-e | 1-1 | 45 | 88 | 2 | 43 |
| 1-4a-e | 1-1 | 46 | 89 | 3 | 43 |
| 1-5a-e | 1-1 | 47 | 90 | 4 | 43 |
| 1-6a-e | 1-1 | 48 | 91 | 5 | 43 |
| 1-7a-e | 1-1 | 49 | 92 | 6 | 43 |
| 1-8a-d | 1-1 | 50 | 93 | 7 | 43 |
| 1-9a-e | 1-1 | 51 | 94 | 8 | 43 |
| 1-10a-e | 1-1 | 52 | 95 | 9 | 43 |
| 1-11a-e | 1-1 | 53 | 96 | 10 | 43 |
| 1-12a-e | 1-1 | 54 | 97 | 11 | 43 |
| 1-13a-f | 1-1 | 55 | 98 | 12 | 43 |
| 1-14a-e | 1-1 | 56 | 99 | 13 | 43 |
| 1-15a-f | 1-1 | 57 | 100 | 14 | 43 |
| 1-16a-e | 1-1 | 58 | 101 | 15 | 43 |
| 1-17a-e | 1-1 | 59 | 102 | 16 | 43 |
| 1-18a-e | 1-1 | 60 | 103 | 17 | 43 |
| 1-19a-e | 1-1 | 61 | 104 | 18 | 43 |
| 1-20a-e | 1-1 | 62 | 105 | 19 | 43 |
| 1-21a-e | 1-1 | 63 | 106 | 20 | 43 |
| 1-22a-e | 1-1 | 64 | 107 | 21 | 43 |
| 1-23a-e | 1-1 | 65 | 108 | 22 | 43 |
| 1-24a-e | 1-1 | 66 | 109 | 23 | 43 |
| 1-25a-e | 1-1 | 67 | 110 | 24 | 43 |
| 1-26a-e | 1-1 | 68 | 111 | 25 | 43 |
| 1-27a-e | 1-1 | 69 | 112 | 26 | 43 |
| 1-28a-e | 1-1 | 70 | 113 | 27 | 43 |
| 1-29a-e | 1-1 | 71 | 114 | 28 (Alkalihalobacillus | 43 |
| | | | | ligniniphilus | |
| | | | | XPDH WP_017729640.1) | |
| 1-30a-e | 1-1 | 72 | 115 | 29 (Jeotgalibacillus soli | 43 |
| | | | | XPDH WP_041086875.1) | |
| 1-31a-e | 1-1 | 73 | 116 | 30 (Heyndrickxia | 43 |
| | | | | sporothermodurans | |
| | | | | XPDH WP_066235909.1) | |
| 1-32a-e | 1-1 | 74 | 117 | 31 (Clostridium | 43 |
| | | | | fungisolvens | |
| | | | | XPDH WP_183276691.1) | |
| 1-33a-e | 1-1 | 75 | 118 | 32 (Alkalihalobacillus | 43 |
| | | | | nanhaiisediminis | |
| | | | | XPDH WP144449381.1) | |
| 1-34a-e | 1-1 | 76 | 119 | 33 (Neobacillus cucumis | 43 |
| | | | | XHPD WP_198313751.1) | |
| 1-35a-d | 1-1 | 77 | 120 | 34 | 43 |
| 1-36a-e | 1-1 | 78 | 121 | 35 | 43 |
| 1-37a-d | 1-1 | 79 | 122 | 36 | 43 |
| 1-38a-f | 1-1 | 80 | 123 | 37 | 43 |
| 1-39a-f | 1-1 | 81 | 124 | 38 | 43 |
| 1-40a-e | 1-1 | 82 | 125 | 39 | 43 |
| 1-41a-e | 1-1 | 83 | 127 | 41 | 43 |
| 1-42a-f | 1-1 | 84 | 128 | 42 | 43 |

### Example 5-Shake Flask Fermentation Assay

Strains 1-1, 1-35a-d, 1-37a-d, 1-38a-f, 1-39a-f, 1-42a-f, 1-13a-f, and 1-15a-f (outlined in Table 4 above), were run in shake flasks to assess glucose consumption as well as ribitol, xylitol, glycerol, and ethanol production.

Strains were streaked out for biomass growth on YPD plates (bacteriological peptone 20 g/L, yeast extract 10 g/L, glucose 20 g/L, and agar 15 g/L) and incubated at 30° C. for 48-72 hours. Cells from the incubated YPD plates were scraped into 40 mL rich medium (170 g/L glucose, 10 g/L yeast extract) in a 250 mL non-baffled flask. Cells were incubated at 30° C. and 250 rpm until the optical density (OD600) reached 15-20 to form the seed culture. Optical density is measured at a wavelength of 600 nm with a 1 cm path length cuvette using a model Genesys20 spectrophotometer (Thermo Scientific). The seed culture reached an OD600 between 15-20 in about 32-50 hours.

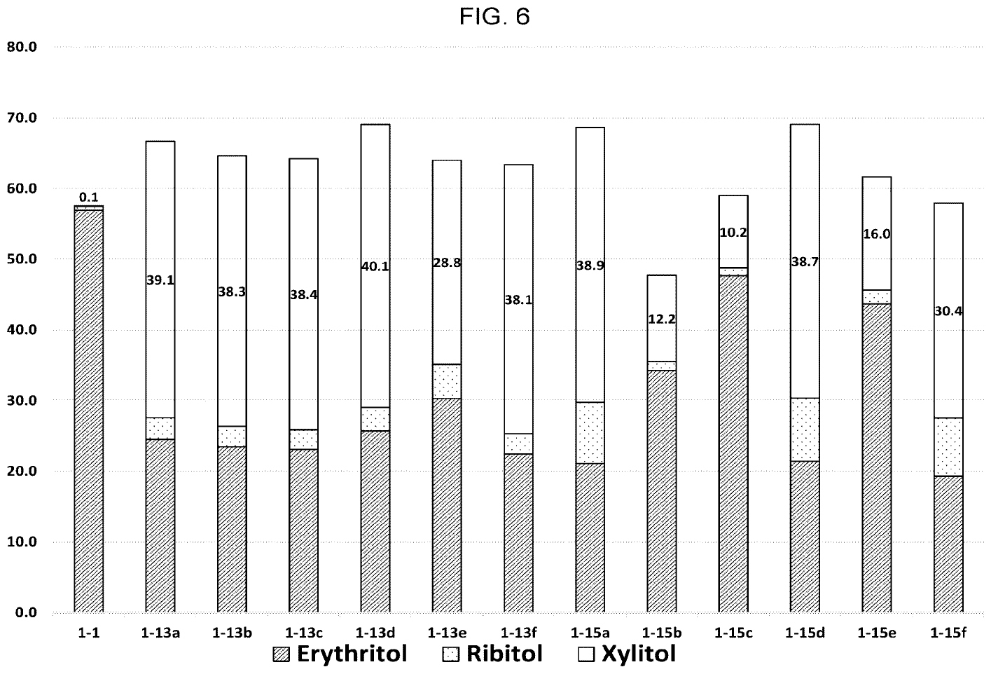

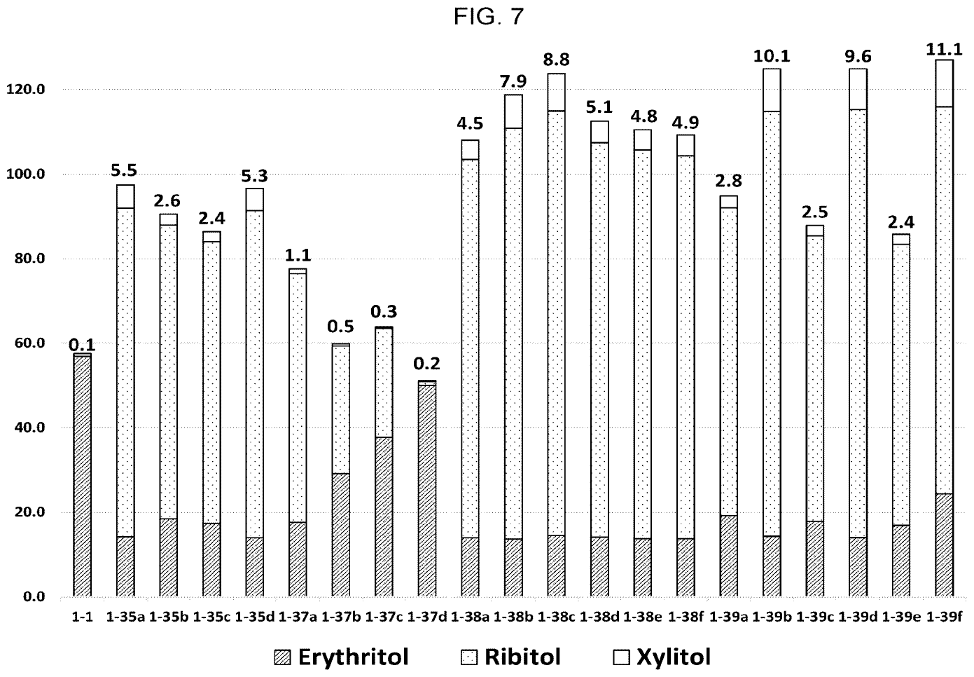

A 250 ml non-baffled flask containing production medium (Table 5) was inoculated with 0.8 mL of the seed culture to form the production culture. The production culture was incubated at 35° C. and 250 rpm. Samples were taken from the production culture after 72 and 96 hours of incubation. Samples were analyzed for glucose, ribitol, xylitol, erythritol, glycerol, and ethanol by high performance liquid chromatography with refractive index detector. Fermentation results are reported in Table 6 and FIGS. 6 and 7.

TABLE 5
Production Medium

| Component | Concentration (units) | |
|---|---|---|
| Glucose | 300 | (g/L) |
| KH2PO4 | 1.27 | (g/L) |
| (NH4)2HPO4 | 0.13 | (g/L) |
| (NH4)2SO4 | 1.80 | (g/L) |
| Urea | 2.85 | (g/L) |
| Citric Acid | 200.0 | (mg/L) |
| MgSO4—7H2O | 690.2 | (mg/L) |
| FeSO4—7H2O | 32.35 | (mg/L) |
| ZnSO4—7H2O | 4.40 | (mg/L) |
| MnSO4—H2O | 0.53 | (mg/L) |
| CuSO4—5H2O | 0.45 | (mg/L) |
| CaCl2—2H2O | 91.60 | (mg/L) |
| Na2SO4 | 154.50 | (mg/L) |
| Thiamine-HCl | 10 | (mg/L) |
| Choline-Cl | 100 | (mg/L) |
| Antifoam CF-32 | 1.00 | (g/L) |

While PCR verification indicated that the transformed polynucleotide sequence was present in the indicated strains, further analysis indicated that in some strains, the sequence was not correctly integrated at the ER1 locus. Further analysis indicated that strains 1-35a, 1-37a-d, 1-38a-c, 1-39d-f, 1-42a-b, 1-42d, 1-13a-b, 1-13d-e, 1-15b-c, and 1-15e-f include the transformed polynucleotide sequence, but it is not integrated at the ER1 locus.

TABLE 6
96-hour Shake Flask Results

| | Fermentation Broth Analyte (g/L) | | | | | |
|---|---|---|---|---|---|---|
| Strain | Glucose | Ribitol | Xylitol | Erythritol | Glycerol | Ethanol |
| 1-1 | 54.2270 | 0.5672 | 0.1156 | 56.9108 | 14.4101 | 45.2717 |
| 1-35a | 19.0690 | 77.8018 | 5.5078 | 14.1262 | 11.6922 | 45.6143 |
| 1-35b | 22.4343 | 69.5363 | 2.6051 | 18.4159 | 6.7667 | 51.1619 |
| 1-35c | 31.4030 | 66.5826 | 2.4193 | 17.3705 | 7.5255 | 49.2545 |
| 1-35d | 28.2827 | 77.4169 | 5.2674 | 13.9265 | 7.1954 | 49.1733 |
| 1-37a | 42.3156 | 58.8415 | 1.1308 | 17.6218 | 8.1394 | 44.6216 |
| 1-37b | 92.0525 | 30.1794 | 0.5249 | 29.1345 | 17.4800 | 28.4251 |
| 1-37c | 77.9520 | 25.8442 | 0.3365 | 37.6779 | 28.5369 | 33.4608 |
| 1-37d | 62.2726 | 0.9251 | 0.2062 | 50.0038 | 18.7245 | 45.7655 |
| 1-38a | 19.5123 | 89.5643 | 4.5079 | 13.9258 | 6.0135 | 46.2436 |
| 1-38b | 15.4952 | 97.2570 | 7.8962 | 13.6124 | 6.7302 | 35.1221 |
| 1-38c | 9.1530 | 100.4624 | 8.8382 | 14.4767 | 6.4634 | 41.8789 |
| 1-38d | 3.5262 | 93.3559 | 5.1135 | 14.0524 | 7.8424 | 46.9099 |
| 1-38e | 5.8345 | 91.9564 | 4.7706 | 13.7265 | 7.3560 | 48.8018 |
| 1-38f | 5.6278 | 90.6644 | 4.9442 | 13.6917 | 7.7348 | 47.5447 |
| 1-39a | 18.6244 | 72.8649 | 2.8000 | 19.1466 | 8.1946 | 40.2287 |
| 1-39b | 2.3933 | 100.5639 | 10.0874 | 14.2583 | 6.9797 | 44.4668 |
| 1-39c | 31.7163 | 67.5513 | 2.4932 | 17.8548 | 7.1724 | 47.5427 |
| 1-39d | 3.3290 | 101.3044 | 9.6496 | 13.9511 | 6.9558 | 46.4375 |
| 1-39e | 38.9049 | 66.5485 | 2.4033 | 16.8345 | 7.1407 | 48.7270 |
| 1-39f | 0.8569 | 91.5653 | 11.1004 | 24.3341 | 6.1712 | 37.6166 |
| 1-42a | 53.2303 | 1.3617 | 0.1586 | 62.1601 | 16.1305 | 44.7835 |
| 1-42b | 70.3640 | 0.5390 | 0.1319 | 52.0362 | 13.1695 | 44.2371 |
| 1-42c | 54.3271 | 0.6740 | 0.1317 | 61.0264 | 17.1397 | 44.9797 |
| 1-42d | 55.3052 | 0.7063 | 0.1484 | 63.2909 | 12.3513 | 43.1762 |
| 1-42e | 61.0003 | 0.5738 | 0.1502 | 56.8237 | 13.3096 | 39.6208 |
| 1-42f | 64.0496 | 0.5451 | 0.1352 | 55.2579 | 13.6874 | 42.7263 |
| 1-13a | 58.2322 | 3.0713 | 39.0606 | 24.5223 | 9.9512 | 43.5850 |
| 1-13b | 64.5326 | 2.8987 | 38.2994 | 23.4455 | 9.2364 | 42.1528 |
| 1-13c | 62.8811 | 2.8168 | 38.3638 | 23.0771 | 9.2614 | 43.8806 |
| 1-13d | 47.8236 | 3.3178 | 40.0543 | 25.7124 | 9.9847 | 40.8596 |
| 1-13e | 59.3667 | 4.8671 | 28.8414 | 30.2839 | 9.5968 | 42.2960 |
| 1-13f | 66.1336 | 2.8202 | 38.0642 | 22.4811 | 8.8765 | 42.3954 |
| 1-15a | 58.6182 | 8.6323 | 38.8703 | 21.1152 | 9.3006 | 43.9871 |
| 1-15b | 78.7837 | 1.2617 | 12.2069 | 34.2608 | 11.5806 | 43.9409 |
| 1-15c | 55.9848 | 1.1212 | 10.2043 | 47.6605 | 12.0438 | 44.2130 |
| 1-15d | 54.0847 | 8.9671 | 38.7047 | 21.4173 | 9.3022 | 44.1645 |
| 1-15e | 60.7735 | 1.9279 | 16.0149 | 43.7036 | 11.1713 | 46.0607 |
| 1-15f | 102.6556 | 8.2515 | 30.3843 | 19.3150 | 13.0177 | 32.7172 |

### Example 6-Shake Flask Fermentation Assay

Strains 1-13c, 1-29a-e, 1-33a-e, and 1-34a-e (outlined in Table 4 above), were run in shake flasks to assess glucose consumption as well as ribitol, xylitol, glycerol, and ethanol production.

Strains were streaked out for biomass growth on YPD plates (bacteriological peptone 20 g/L, yeast extract 10 g/L, glucose 20 g/L, and agar 15 g/L) and incubated at 30° C. for 48-72 hours. Cells from the incubated YPD plates were scraped into 40 mL rich medium (170 g/L glucose, 10 g/L yeast extract) in a 250 mL non-baffled flask. Cells were incubated at 30° C. and 250 rpm until the optical density (OD600) reached 15-20 to form the seed culture. Optical density is measured at a wavelength of 600 nm with a 1 cm path length cuvette using a model Genesys20 spectrophotometer (Thermo Scientific). The seed culture reached an OD600 between 15-20 in about 32-50 hours.

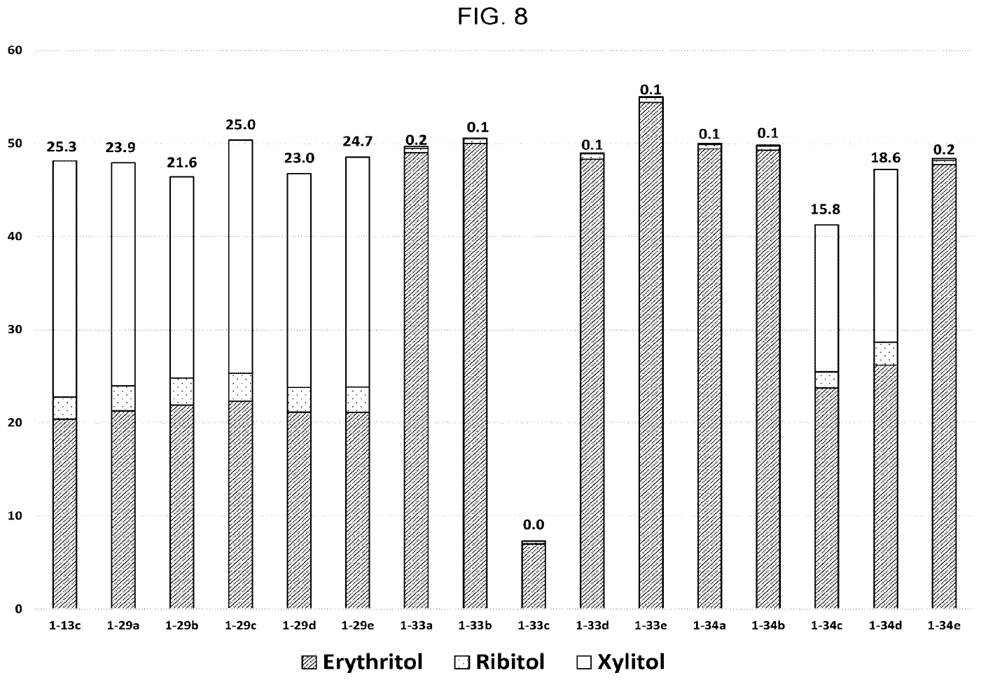

A 250 ml non-baffled flask containing production medium (Table 5) was inoculated with 0.8 mL of the seed culture to form the production culture. The production culture was incubated at 35° C. and 250 rpm. Samples were taken from the production culture after 96 hours of incubation. Samples were analyzed for glucose, ribitol, xylitol, erythritol, glycerol, and ethanol by high performance liquid chromatography with refractive index detector. Fermentation results are reported in Table 7 and FIG. 8.

As seen in FIG. 8, while sister strains 1-34c and 1-34d produced 15.8 and 18.6 g/L xylitol, respectively, strains 1-34a, 1-34b, and 1-34e did not produce significantly more xylitol than wild-type (strain 1-1, FIG. 6). While strains 1-34a, 1-34b, and 1-34e were initially PCR verified, it was later determined that the integrated polynucleotide, which should encode the N. cucumis XPDH homolog, contained a frameshift mutation and no functional XPDH was expressed. Therefore, while the results appear varied, they are in fact consistent given that strains 1-34a, 1-34b, and 1-34e did not contain a polynucleotide that encoded a functional XPDH.

TABLE 7
96-hour Shake Flask Results

| | Fermentation Broth Analyte (g/L) | | | | | |
|---|---|---|---|---|---|---|
| Strain | Glucose | Ribitol | Xylitol | Erythritol | Glycerol | Ethanol |
| 1-13c | 104.7609 | 2.4041 | 25.3431 | 20.3872 | 8.3483 | 28.8597 |
| 1-29a | 107.59 | 2.7375 | 23.9236 | 21.2592 | 8.327 | 29.7606 |
| 1-29b | 117.0722 | 2.8833 | 21.624 | 21.9028 | 7.5941 | 30.1983 |
| 1-29c | 92.8027 | 3.0122 | 25.0203 | 22.3197 | 8.607 | 34.1865 |
| 1-29d | 113.7418 | 2.654 | 22.973 | 21.1195 | 7.992 | 30.2967 |
| 1-29e | 106.1983 | 2.7505 | 24.6828 | 21.1105 | 8.3573 | 30.0138 |
| 1-33a | 102.5508 | 0.4847 | 0.2175 | 48.9982 | 9.7203 | 31.4812 |
| 1-33b | 101.4479 | 0.507 | 0.0751 | 50.0129 | 9.8179 | 31.3012 |
| 1-33c | 231.4215 | 0.2896 | 0.0353 | 6.9913 | 16.2002 | 6.2899 |
| 1-33d | 107.2532 | 0.5626 | 0.0542 | 48.3365 | 10.36 | 31.982 |
| 1-33e | 89.76 | 0.5258 | 0.0847 | 54.4229 | 10.6124 | 27.6685 |
| 1-34a | 97.4677 | 0.487 | 0.0849 | 49.4197 | 10.4031 | 31.8369 |
| 1-34b | 99.6607 | 0.4724 | 0.0682 | 49.2765 | 9.7575 | 31.2798 |
| 1-34c | 125.3303 | 1.7402 | 15.7865 | 23.7361 | 8.5658 | 28.3016 |
| 1-34d | 109.6517 | 2.4549 | 18.5615 | 26.2014 | 8.1791 | 30.7102 |
| 1-34e | 92.4856 | 0.434 | 0.22 | 47.7303 | 9.2374 | 32.5853 |

### Example 7-Shake Flask Fermentation Assay

Strains 1-13c, 1-17a-e, 1-18a-e, 19a-e, 1-21a-e, 1-22a-e, 1-23a-e, 1-24a-e, 1-25a-e, and 1-27a-d (outlined in Table 4 above), were run in shake flasks to assess glucose consumption as well as ribitol, xylitol, glycerol, and ethanol production.

Strains were streaked out for biomass growth on YPD plates (bacteriological peptone 20 g/L, yeast extract 10 g/L, glucose 20 g/L, and agar 15 g/L) and incubated at 30° C. for 48-72 hours. Cells from the incubated YPD plates were scraped into 40 mL rich medium (170 g/L glucose, 10 g/L yeast extract) in a 250 mL non-baffled flask. Cells were incubated at 30° C. and 250 rpm until the optical density (OD600) reached 15-20 to form the seed culture. Optical density is measured at a wavelength of 600 nm with a 1 cm path length cuvette using a model Genesys20 spectrophotometer (Thermo Scientific). The seed culture reached an OD600 between 15-20 in about 32-50 hours.

A 250 ml non-baffled flask containing production medium (Table 5) was inoculated with 0.8 mL of the seed culture to form the production culture. The production culture was incubated at 35° C. and 250 rpm. Samples were taken from the production culture after 96 hours of incubation. Samples were analyzed for glucose, ribitol, xylitol, erythritol, glycerol, and ethanol by high performance liquid chromatography with refractive index detector. Fermentation results are reported in Table 8.

TABLE 8
96-hour Shake Flask Results

| | Fermentation Broth Analyte (g/L) | | | | | |
|---|---|---|---|---|---|---|
| Strain | Glucose | Ribitol | Xylitol | Erythritol | Glycerol | Ethanol |
| 1-13a | 98.6422 | 2.6387 | 24.7712 | 25.2307 | 13.8005 | 28.8903 |
| 1-17a | 74.915 | 0.5991 | 0.2543 | 60.8569 | 13.0048 | 33.3547 |
| 1-17b | 79.2438 | 0.577 | 0.0725 | 58.9302 | 12.8216 | 35.3409 |
| 1-17c | 151.0953 | 0.4237 | 0.0975 | 24.7655 | 21.0532 | 20.2202 |
| 1-17d | 90.0013 | 1.4266 | 0.0645 | 55.808 | 18.8406 | 27.8706 |
| 1-17e | 84.2381 | 0.7091 | 0.0428 | 59.7786 | 15.6082 | 29.9387 |
| 1-18a | 187.0194 | 0.3163 | 0.1069 | 21.6446 | 15.8366 | 10.9522 |
| 1-18b | 83.2939 | 0.6768 | 0.0524 | 63.3983 | 14.7387 | 31.6461 |
| 1-18c | 88.21 | 0.6902 | 0.0625 | 59.74 | 14.7915 | 31.5115 |
| 1-18d | 76.4267 | 0.8183 | 0.0692 | 57.1505 | 16.2282 | 34.0999 |
| 1-18e | 89.0621 | 0.7336 | 0.0841 | 42.5495 | 26.7038 | 25.7627 |
| 1-19a | 94.518 | 0.6166 | 0.0801 | 54.4468 | 18.8522 | 28.9871 |
| 1-19b | 83.559 | 0.6632 | 0.0659 | 60.2352 | 14.9726 | 31.2065 |
| 1-19c | 93.3168 | 0.7412 | 0.0646 | 60.6521 | 14.3042 | 29.9464 |
| 1-19d | 165.946 | 0.4311 | 0.1236 | 24.1998 | 15.9581 | 19.0694 |
| 1-19e | 78.3357 | 0.675 | 0.0637 | 61.3653 | 15.847 | 29.361 |
| 1-21a | 77.8724 | 0.882 | 0.0789 | 65.2592 | 16.0186 | 31.302 |
| 1-21b | 85.2124 | 0.6777 | 0.0619 | 56.1455 | 15.4518 | 33.3606 |
| 1-21c | 85.008 | 1.0769 | 0.0802 | 58.6289 | 14.8934 | 32.8785 |
| 1-21d | 83.4408 | 1.9855 | 0.0373 | 56.0976 | 14.2115 | 34.3856 |
| 1-21e | 60.5121 | 2.039 | 0.0418 | 63.3641 | 15.5567 | 31.3166 |
| 1-22a | 96.2241 | 0.8385 | 0.0644 | 62.4003 | 13.1106 | 29.8987 |
| 1-22b | 90.5783 | 0.5506 | 0.0722 | 54.306 | 13.9584 | 34.1293 |
| 1-22c | 85.3285 | 0.6442 | 0.0595 | 60.2244 | 15.0443 | 31.2101 |
| 1-22d | 87.0015 | 0.6859 | 0.0824 | 59.9578 | 14.9889 | 31.3812 |
| 1-22e | 78.5983 | 0.5432 | 0.0718 | 58.3192 | 14.9655 | 30.6604 |
| 1-23a | 84.0794 | 0.4999 | 0.109 | 52.3855 | 13.872 | 35.266 |
| 1-23b | 84.7042 | 0.5799 | 0.0696 | 58.9566 | 15.0869 | 33.9501 |
| 1-23c | 87.9261 | 0.7983 | 0.0538 | 55.5429 | 17.3164 | 30.1523 |
| 1-23d | 87.2081 | 0.6745 | 0.0623 | 59.4328 | 14.8259 | 31.3395 |
| 1-23e | 82.9585 | 1.0568 | 0.0795 | 62.242 | 18.8816 | 23.5949 |
| 1-24a | 86.7546 | 0.534 | 0.0725 | 53.0638 | 13.0757 | 34.4192 |
| 1-24b | 104.4802 | 0.583 | 0.0821 | 48.7903 | 14.9732 | 29.8021 |
| 1-24c | 104.5435 | 0.563 | 0.0872 | 48.1204 | 14.5086 | 31.0036 |
| 1-24d | 169.2148 | 0.425 | 0.1078 | 25.2327 | 14.0784 | 19.6567 |
| 1-24e | 78.1939 | 0.5172 | 0.082 | 55.4618 | 15.2743 | 36.423 |
| 1-25a | 85.5913 | 0.8201 | 0.0711 | 57.2974 | 18.9442 | 29.1298 |
| 1-25b | 83.3265 | 0.6891 | 0.0801 | 60.4083 | 15.034 | 31.4877 |
| 1-25c | 139.0213 | 0.6194 | 0.1079 | 28.2606 | 22.9621 | 22.0197 |
| 1-25d | 137.1956 | 0.6387 | 0.1254 | 28.3686 | 22.7144 | 22.2391 |
| 1-25e | 78.0893 | 0.6945 | 0.0792 | 61.3866 | 15.5101 | 31.7923 |
| 1-27a | 54.0049 | 0.8002 | 0.0653 | 73.3789 | 15.1766 | 36.3078 |
| 1-27b | 88.2258 | 0.5067 | 0.0543 | 53.4474 | 13.5761 | 34.0372 |
| 1-27c | 114.4683 | 0.7334 | 0.0729 | 43.7274 | 17.9455 | 26.3899 |
| 1-27d | 76.682 | 0.5437 | 0.0552 | 58.8882 | 12.8684 | 38.8414 |

### Example 8-Shake Flask Fermentation Assay

Strains 1-13c, 1-3a-e, 1-10a-e, 1-11a-e, 1-12a-e, 1-14a-e, 1-16a-e, 1-28a-e, and 1-2a-e (outlined in Table 4 above), were run in shake flasks to assess glucose consumption as well as ribitol, xylitol, glycerol, and ethanol production.

Strains were streaked out for biomass growth on YPD plates (bacteriological peptone 20 g/L, yeast extract 10 g/L, glucose 20 g/L, and agar 15 g/L) and incubated at 30° C. for 48-72 hours. Cells from the incubated YPD plates were scraped into 40 mL rich medium (170 g/L glucose, 10 g/L yeast extract) in a 250 mL non-baffled flask. Cells were incubated at 30° C. and 250 rpm until the optical density (OD600) reached 15-20 to form the seed culture. Optical density is measured at a wavelength of 600 nm with a 1 cm path length cuvette using a model Genesys20 spectrophotometer (Thermo Scientific). The seed culture reached an OD600 between 15-20 in about 32-50 hours.

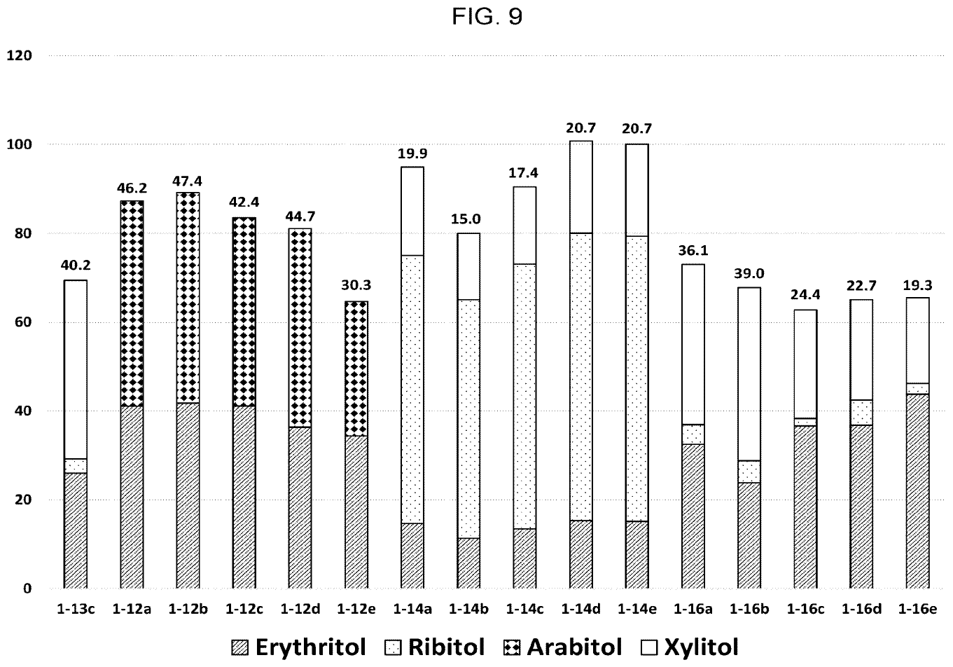

A 250 ml non-baffled flask containing production medium (Table 5) was inoculated with 0.8 mL of the seed culture to form the production culture. The production culture was incubated at 35° C. and 250 rpm. Samples were taken from the production culture after 72 and 96 hours of incubation. Samples were analyzed for glucose, ribitol, xylitol, erythritol, glycerol, and ethanol by high performance liquid chromatography with refractive index detector. Fermentation results are reported in Table 9 and FIG. 9.

While PCR verification indicated that the transformed polynucleotide sequence was present in the indicated strains, further analysis indicated that in some strains, the sequence was not correctly integrated at the ER1 locus. Further analysis indicated that strain 1-16b-e includes the transformed polynucleotide sequence, but it is not at the ER1 locus. Further analysis was inconclusive on the integration location in strains 1-2c and 1-2d.

TABLE 9
96-hour Shake Flask Results

| | Fermentation Broth Analyte (g/L) | | | | | | |
|---|---|---|---|---|---|---|---|
| Strain | Glucose | Ribitol | Xylitol | Erythritol | Glycerol | Ethanol | Arabitol |
| 1-13c | 52.365 | 3.1859 | 40.2435 | 26.0209 | 11.1145 | 37.6398 | |
| 1-3a | 58.7394 | 0.6129 | 0.3235 | 59.7362 | 15.12 | 40.891 | |
| 1-3b | 40.836 | 0.4677 | 0.1051 | 49.0415 | 15.6583 | 51.2491 | |
| 1-3c | 55.8652 | 0.5887 | 0.1253 | 59.2426 | 14.1511 | 42.761 | |
| 1-3d | 47.4122 | 0.5038 | 0.1279 | 50.8589 | 16.8177 | 41.1038 | |
| 1-3e | 45.8606 | 0.5127 | 0.1267 | 50.1914 | 16.3743 | 48.2382 | |
| 1-10a | 58.5369 | 0.5891 | 0.1201 | 57.9712 | 15.3885 | 40.4663 | |
| 1-10b | 58.9318 | 0.6075 | 0.1492 | 57.9368 | 14.8489 | 41.9797 | |
| 1-10c | 67.992 | 0.4588 | 0.142 | 49.0939 | 14.307 | 40.394 | |
| 1-10d | 46.0003 | 0.6906 | 0.0995 | 62.4692 | 19.849 | 32.9163 | |
| 1-10e | 55.0568 | 0.655 | 0.136 | 61.0313 | 15.9185 | 38.2694 | |
| 1-11a | 51.9376 | 0.429 | 0.0526 | 60.4417 | 14.8425 | 40.9479 | |
| 1-11b | 45.6503 | 0.554 | 0.1326 | 61.3534 | 18.1726 | 44.3344 | |
| 1-11c | 5.6815 | 1.5471 | 0.0801 | 86.3607 | 27.719 | 41.9823 | |
| 1-11d | 40.3772 | 0.5489 | 0.1304 | 61.8324 | 18.171 | 41.9493 | |
| 1-11e | 53.0558 | 0.6124 | 0.115 | 60.6504 | 15.489 | 39.7766 | |
| 1-12a | 30.456 | n.a. | n.a. | 41.1173 | 9.6187 | 44.7618 | 46.2065 |
| 1-12b | 28.2127 | n.a. | n.a. | 41.7725 | 9.3555 | 45.1461 | 47.4416 |
| 1-12c | 45.2228 | n.a. | n.a. | 41.1422 | 9.0341 | 39.2179 | 42.4228 |
| 1-12d | 30.6334 | n.a. | n.a. | 36.381 | 10.0025 | 39.6166 | 44.7009 |
| 1-12e | 81.9896 | n.a. | n.a. | 34.348 | 12.8173 | 30.9024 | 30.2901 |
| 1-14a | 36.1578 | 60.3639 | 19.8883 | 14.677 | 6.9789 | 39.9443 | |
| 1-14b | 29.7316 | 53.6709 | 14.9948 | 11.3645 | 12.08 | 43.9787 | |
| 1-14c | 27.9273 | 59.5839 | 17.4099 | 13.471 | 7.8201 | 45.0742 | |
| 1-14d | 13.2601 | 64.6881 | 20.7494 | 15.3596 | 7.6729 | 41.5184 | |
| 1-14e | 24.8568 | 64.1917 | 20.7387 | 15.176 | 7.2937 | 39.5752 | |
| 1-16a | 53.8198 | 4.4367 | 36.058 | 32.5055 | 10.8652 | 39.1211 | |
| 1-16b | 52.0735 | 4.9579 | 38.9526 | 23.8416 | 10.1467 | 41.0993 | |
| 1-16c | 55.1727 | 1.6863 | 24.425 | 36.6549 | 11.7111 | 41.8186 | |
| 1-16d | 45.5429 | 5.6345 | 22.6578 | 36.7881 | 11.2977 | 41.0565 | |
| 1-16e | 41.996 | 2.3924 | 19.2866 | 43.8223 | 17.3556 | 40.5198 | |
| 1-28a | 57.3853 | 0.6449 | 0.2041 | 58.1115 | 15.5452 | 41.2408 | |
| 1-28b | 58.7477 | 0.5753 | 0.1016 | 56.9917 | 14.4135 | 41.1243 | |
| 1-28c | 59.1807 | 0.5957 | 0.1202 | 57.6647 | 14.5907 | 42.3829 | |
| 1-28d | 32.0943 | 0.6578 | 0.1353 | 62.5783 | 18.1666 | 40.946 | |
| 1-28e | 56.5966 | 0.5859 | 0.1193 | 57.5414 | 14.0459 | 41.5865 | |
| 1-2a | 50.0384 | 0.7556 | 0.1162 | 61.3047 | 15.3111 | 41.1167 | |
| 1-2b | 34.3286 | 0.79 | 0.1848 | 58.4062 | 19.6802 | 48.8752 | |
| 1-2c | 43.167 | 0.7455 | 0.0948 | 70.8234 | 11.4465 | 42.3114 | |
| 1-2d | 41.842 | 0.6423 | 0.1082 | 63.7359 | 15.0666 | 39.6665 | |
| 1-2e | 47.5807 | 0.6122 | 0.1415 | 60.543 | 13.8641 | 43.2135 | |

### Example 9-Shake Flask Fermentation Assay

Strains 1-13c, 1-8a-d, 1-26a-e, 1-36a-e, 1-41a-e, 1-40a-e, and 1-20a-e (outlined in Table 4 above), were run in shake flasks to assess glucose consumption as well as ribitol, xylitol, glycerol, and ethanol production.

Strains were streaked out for biomass growth on YPD plates (bacteriological peptone 20 g/L, yeast extract 10 g/L, glucose 20 g/L, and agar 15 g/L) and incubated at 30° C. for 48-72 hours. Cells from the incubated YPD plates were scraped into 40 mL rich medium (170 g/L glucose, 10 g/L yeast extract) in a 250 mL non-baffled flask. Cells were incubated at 30° C. and 250 rpm until the optical density (OD600) reached 15-20 to form the seed culture. Optical density is measured at a wavelength of 600 nm with a 1 cm path length cuvette using a model Genesys20 spectrophotometer (Thermo Scientific). The seed culture reached an OD600 between 15-20 in about 32-50 hours.

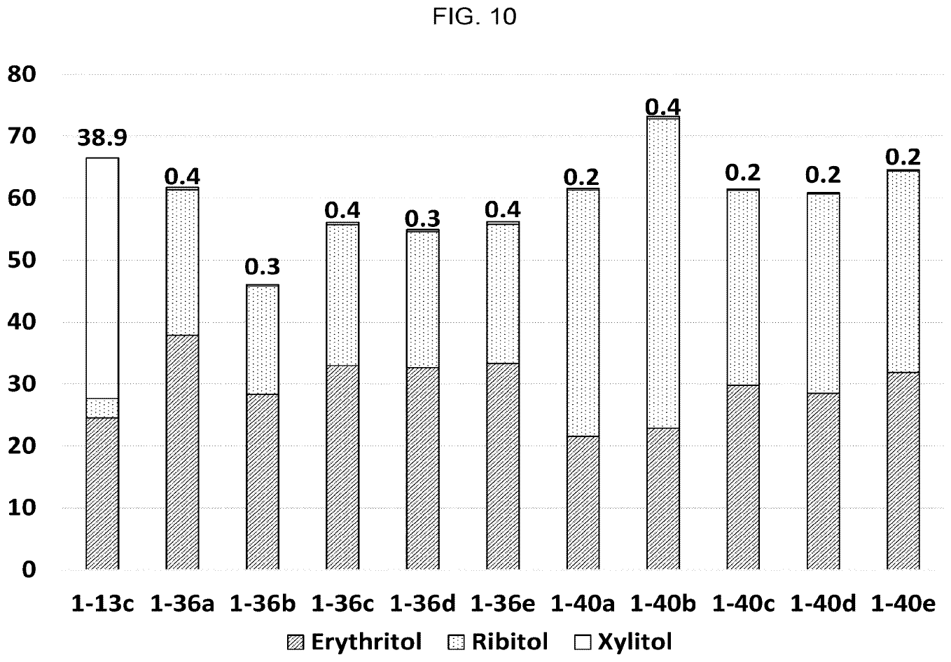

A 250 ml non-baffled flask containing production medium (Table 5) was inoculated with 0.8 mL of the seed culture to form the production culture. The production culture was incubated at 35° C. and 250 rpm. Samples were taken from the production culture after 96 hours of incubation. Samples were analyzed for glucose, ribitol, xylitol, erythritol, glycerol, and ethanol by high performance liquid chromatography with refractive index detector. Fermentation results are reported in Table 10 and FIG. 10.

While PCR verification indicated that the transformed polynucleotide sequence was present in the indicated strains, further analysis indicated that in some strains, the sequence was not correctly integrated at the ER1 locus. Further analysis indicated that strains 1-8c, 1-8d and 1-41c include the transformed polynucleotide sequence, but it is not integrated at the ER1 locus. Further analysis was inconclusive on integration locus in strains 1-36a, 1-41b, 1-41e, and 1-20a-e.

TABLE 10
96-hour Shake Flask Results

| | Fermentation Broth Analyte (g/L) | | | | | |
|---|---|---|---|---|---|---|
| Strain | Glucose | Ribitol | Xylitol | Erythritol | Glycerol | Ethanol |
| 1-13c | 43.7126 | 3.1176 | 38.8613 | 24.5197 | 9.666 | 42.859 |
| 1-8a | 46.3518 | 0.537 | 0.3381 | 57.0411 | 14.4597 | 46.2915 |
| 1-8b | 47.5195 | 0.5334 | 0.1431 | 56.9461 | 13.7069 | 47.0977 |
| 1-8c | 48.0894 | 0.5594 | 0.1063 | 55.9984 | 13.6091 | 47.8657 |
| 1-8d | 37.4186 | 0.6121 | 0.1105 | 56.5403 | 13.8863 | 51.6109 |
| 1-26a | 44.7559 | 0.6392 | 0.1211 | 58.7843 | 13.0302 | 45.1907 |
| 1-26b | 26.8789 | 0.7454 | 0.1365 | 65.6702 | 14.7237 | 48.8557 |
| 1-26c | 42.4506 | 0.7303 | 0.1018 | 65.269 | 12.7456 | 45.1127 |
| 1-26d | 54.3955 | 0.5664 | 0.0934 | 50.543 | 16.7051 | 47.0098 |
| 1-26e | 36.6369 | 0.7275 | 0.1162 | 70.0149 | 10.7288 | 48.273 |
| 1-36a | 43.9038 | 23.4607 | 0.4003 | 37.8737 | 12.3842 | 43.023 |
| 1-36b | 46.2296 | 17.4734 | 0.2509 | 28.3498 | 17.1992 | 51.7303 |
| 1-36c | 33.5473 | 22.7172 | 0.384 | 32.9889 | 10.9137 | 53.8241 |
| 1-36d | 30.3483 | 21.9108 | 0.3459 | 32.6397 | 12.4463 | 55.6903 |
| 1-36e | 34.2625 | 22.4675 | 0.4111 | 33.3163 | 10.3248 | 53.7075 |
| 1-41a | 50.84 | 0.6102 | 0.1238 | 53.2111 | 12.3563 | 47.0405 |
| 1-41b | 27.0994 | 0.9407 | 0.0998 | 67.672 | 11.9654 | 54.0057 |
| 1-41c | 54.6053 | 0.5387 | 0.1112 | 52.0424 | 14.0354 | 47.3747 |
| 1-41d | 58.9086 | 0.6276 | 0.1701 | 51.36 | 21.0093 | 44.2126 |
| 1-41e | 48.8262 | 0.5743 | 0.1143 | 57.4473 | 13.8819 | 45.8719 |
| 1-40a | 38.7776 | 39.7726 | 0.2401 | 21.5304 | 12.246 | 44.721 |
| 1-40b | 44.7092 | 49.927 | 0.3738 | 22.8583 | 10.4058 | 44.314 |
| 1-40c | 43.6888 | 31.47 | 0.2197 | 29.7888 | 10.3401 | 50.5487 |
| 1-40d | 43.0144 | 32.1802 | 0.1935 | 28.5121 | 13.9717 | 49.4035 |
| 1-40e | 38.1811 | 32.5265 | 0.2004 | 31.8564 | 10.647 | 51.5381 |
| 1-20a | 43.2545 | 0.7591 | 0.0973 | 60.0985 | 13.8425 | 44.7316 |
| 1-20b | 44.7893 | 0.7386 | 0.1007 | 69.3394 | 13.0988 | 46.5606 |
| 1-20c | 29.207 | 0.673 | 0.1105 | 63.3536 | 16.4597 | 49.4451 |
| 1-20d | 39.0349 | 0.612 | 0.1224 | 57.5853 | 14.8878 | 49.7504 |
| 1-20e | 48.061 | 0.7296 | 0.1017 | 61.7944 | 10.9017 | 47.5667 |

### Example 10-Shake Flask Fermentation Assay

Strains 1-13c, 1-30a-e, 1-31a-e, 1-32a-e, 1-4a-e, 1-5a-e, 1-6a-e, 1-7a-e, and 1-9a-e (outlined in Table 4 above), were run in shake flasks to assess glucose consumption as well as ribitol, xylitol, glycerol, and ethanol production.

Strains were streaked out for biomass growth on YPD plates (bacteriological peptone 20 g/L, yeast extract 10 g/L, glucose 20 g/L, and agar 15 g/L) and incubated at 30° C. for 48-72 hours. Cells from the incubated YPD plates were scraped into 40 mL rich medium (170 g/L glucose, 10 g/L yeast extract) in a 250 mL non-baffled flask. Cells were incubated at 30° C. and 250 rpm until the optical density (OD600) reached 15-20 to form the seed culture. Optical density is measured at a wavelength of 600 nm with a 1 cm path length cuvette using a model Genesys20 spectrophotometer (Thermo Scientific). The seed culture reached an OD600 between 15-20 in about 32-50 hours.

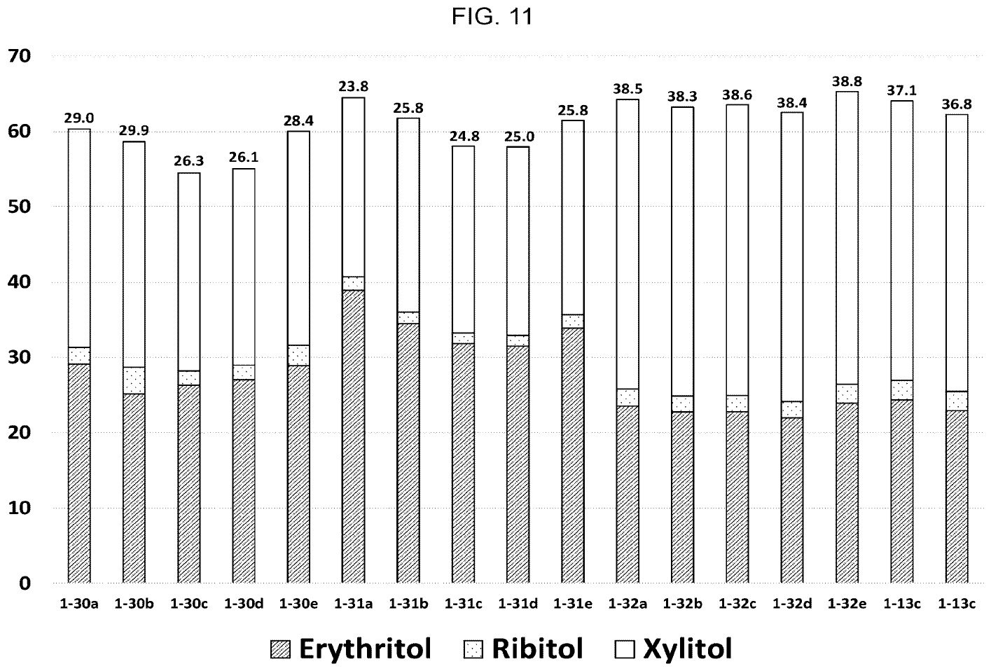

A 250 ml non-baffled flask containing production medium (Table 5) was inoculated with 0.8 mL of the seed culture to form the production culture. The production culture was incubated at 35° C. and 250 rpm. Samples were taken from the production culture after 72 and 96 hours of incubation. Samples were analyzed for glucose, ribitol, xylitol, erythritol, glycerol, and ethanol by high performance liquid chromatography with refractive index detector. Fermentation results are reported in Table 11 and FIG. 11.

While PCR verification indicated that the transformed polynucleotide sequence was present in the indicated strains, further analysis indicated that in some strains, the sequence was not correctly integrated at the ER1 locus. Further analysis indicated that strains 1-30c and 1-30d include the transformed polynucleotide sequence, but it is not integrated at the ER1 locus. Further analysis was inconclusive on the integration locus in strain 1-6c.

TABLE 11
96-hour Shake Flask Results

| | Fermentation Broth Analyte (g/L) | | | | | |
|---|---|---|---|---|---|---|
| Strain | Glucose | Ribitol | Xylitol | Erythritol | Glycerol | Ethanol |
| 1-13c | 65.2534 | 2.6255 | 37.1061 | 24.3306 | 10.3659 | 40.8187 |
| 1-13c | 63.7146 | 2.5391 | 36.7742 | 22.9396 | 9.9483 | 41.7289 |
| 1-30a | 64.53 | 2.2035 | 28.9854 | 29.1288 | 11.3525 | 39.7976 |
| 1-30b | 58.571 | 3.5146 | 29.9395 | 25.1729 | 12.5878 | 42.2656 |
| 1-30c | 61.7808 | 1.8694 | 26.2541 | 26.3438 | 15.1573 | 43.1151 |
| 1-30d | 57.4209 | 1.9473 | 26.054 | 27.0466 | 13.4411 | 44.8634 |
| 1-30e | 74.7224 | 2.7504 | 28.386 | 28.8879 | 14.2582 | 37.7887 |
| 1-31a | 65.2966 | 1.7919 | 23.8031 | 38.9002 | 11.2516 | 37.7702 |
| 1-31b | 58.0672 | 1.528 | 25.7531 | 34.4798 | 12.434 | 40.5374 |
| 1-31c | 60.9593 | 1.4068 | 24.8131 | 31.8421 | 11.6425 | 41.8413 |
| 1-31d | 60.5991 | 1.4246 | 25.033 | 31.5056 | 11.5117 | 42.3126 |
| 1-31e | 65.0496 | 1.7865 | 25.7844 | 33.8874 | 12.8272 | 40.8807 |
| 1-32a | 67.1283 | 2.236 | 38.4541 | 23.5687 | 11.1949 | 38.707 |
| 1-32b | 70.2815 | 2.1497 | 38.3141 | 22.7553 | 10.5468 | 38.8618 |
| 1-32c | 68.0248 | 2.2063 | 38.5515 | 22.7602 | 10.3843 | 38.5577 |
| 1-32d | 66.3494 | 2.1502 | 38.3931 | 21.9976 | 11.0603 | 40.7174 |
| 1-32e | 63.8648 | 2.5133 | 38.8442 | 23.9287 | 10.8515 | 39.9071 |
| 1-4a | 70.2783 | 0.4834 | 0.3673 | 46.1316 | 14.32 | 43.4293 |
| 1-4b | 72.2855 | 0.7763 | 0.1831 | 57.21 | 19.3193 | 35.128 |
| 1-4c | 56.1849 | 0.5101 | 0.1291 | 47.8537 | 15.7451 | 47.9659 |
| 1-4d | 77.2722 | 0.5838 | 0.1901 | 50.3863 | 14.1389 | 39.9826 |
| 1-4e | 70.9407 | 0.4671 | 0.1525 | 46.2662 | 15.3398 | 44.2176 |
| 1-5a | 56.7359 | 0.6505 | 0.1281 | 69.7951 | 13.2652 | 40.3786 |
| 1-5b | 66.4853 | 0.4758 | 0.1138 | 47.5548 | 17.2347 | 44.8984 |
| 1-5c | 56.2946 | 0.4561 | 0.1072 | 46.0599 | 15.9891 | 48.8712 |
| 1-5d | 63.8624 | 0.5188 | 0.1223 | 55.0207 | 15.5579 | 41.5538 |
| 1-5e | 53.5706 | 0.4438 | 0.0978 | 46.4767 | 15.7478 | 49.9892 |
| 1-6a | 53.5487 | 0.4466 | 0.0857 | 46.0621 | 14.3516 | 50.2479 |
| 1-6b | 56.3098 | 0.5689 | 0.0987 | 59.4119 | 14.2449 | 44.3129 |
| 1-6c | 62.2098 | 0.4975 | 0.1159 | 50.7104 | 13.912 | 46.5117 |
| 1-6d | 88.3493 | 0.392 | 0.0963 | 46.7061 | 15.7906 | 39.0682 |
| 1-6e | 66.3903 | 0.513 | 0.1236 | 54.7569 | 15.6941 | 42.6935 |
| 1-7a | 84.3102 | 0.7204 | 0.0924 | 52.2635 | 19.827 | 33.7597 |
| 1-7b | 69.0519 | 0.4897 | 0.1187 | 51.9057 | 14.6164 | 43.9185 |
| 1-7c | 67.7409 | 0.5193 | 0.1426 | 53.5386 | 14.7419 | 42.8362 |
| 1-7d | 61.1123 | 0.5279 | 0.1161 | 54.4933 | 14.502 | 43.378 |
| 1-7e | 58.0693 | 0.4778 | 0.1356 | 50.1206 | 18.9325 | 46.1013 |
| 1-9a | 52.6189 | 0.5986 | 0.1156 | 66.6905 | 13.1314 | 43.3405 |
| 1-9b | 59.3034 | 0.464 | 0.1102 | 58.2455 | 14.7562 | 43.3825 |
| 1-9c | 53.516 | 0.4302 | 0.1246 | 51.9753 | 15.0032 | 48.9462 |
| 1-9d | 53.9501 | 0.4221 | 0.0876 | 46.6648 | 15.398 | 49.7159 |
| 1-9e | 54.575 | 0.4136 | 0.0957 | 46.5254 | 15.3139 | 49.828 |

1. A genetically engineered yeast cell capable of producing xylitol, the engineered yeast cell comprising:
an exogenous polynucleotide sequence encoding a xylitol-phosphate dehydrogenase (XPDH) enzyme comprising a sequence at least 80% identical to at least one of SEQ ID NOs: 12-15, 28-31, and 33.

2. (canceled)

3. The yeast cell of claim 1, wherein the yeast cell is an osmotolerant yeast cell.

4. The yeast cell of claim 1, wherein the yeast cell is a cell of the subphylum Ustilaginomycotina.

5. The yeast cell of claim 1, wherein the yeast cell is selected from the group consisting of Trichosporonoides megachiliensis, Trychosporonoides oedocephalis, Trychosporonoides nigrescens, Pseudozyma tsukubaensis, Trigonopsis variabilis, Moniliella, Ustilaginomycetes, Trichosporon, Yarrowia lipolytica, Penicillium, Torula, Pichia, Candida, Candida magnoliae, and Aureobasidium

6. The yeast cell of claim 1, wherein the yeast cell is a yeast cell of the Moniliella genus.

7. (canceled)

8. The yeast cell of claim 1, wherein the cell is a Moniliella pollinis cell.

9. The yeast cell of claim 1, wherein the yeast cell is capable of producing xylitol at a titer of at least 20, 30, 50, 75, or 100 g/L when used in a fermentation process in the presence of dextrose at 35° for 96 hours.

10. The yeast cell of claim 1, wherein erythritol production by the yeast cell is reduced relative to erythritol production in an equivalent yeast cell lacking the exogenous polynucleotide sequence.

11. The yeast cell of claim 1, wherein the exogenous polynucleotide sequence is integrated into the genome of the yeast cell at a loci selected from the ER1 locus, the ER3 locus, the PDC1 locus, the pyrF locus, the TRP3 locus, the gpdIIA locus, and the gpdIIB locus.

12. The yeast cell of claim 1, wherein the exogenous polynucleotide sequence is operably linked to a heterologous or artificial promoter.

13. The yeast cell of claim 12, wherein the promoter is a constitutive promoter.

14. The yeast cell of claim 12, wherein the constitutive heterologous or artificial promoter is selected from the group consisting of pyruvate kinase 1 promoter (PYK1p; SEQ ID NO:86), 6-phosphogluconate dehydrogenase promoter (6PGDp; SEQ ID NO:130), glyceraldehyde-3-phosphate dehydrogenase promoter (TDH3p; SEQ ID NO: 132), translational elongation factor 1 promoter (TEFp; SEQ ID NO:133), modified TEFp (SEQ ID NO:131), phosphoglucomutase 1 promoter (PGMlp; SEQ ID NO:134), 3-phosphoglycerate kinase promoter (PGK1p; SEQ ID NO:135), enolase promoter (ENO1p; SEQ ID NO:136), asparagine synthetase promoter (ASNSp; SEQ ID NO:137), 50S ribosomal protein L1 promoter (RPLAp; SEQ ID NO:138), and RPL16B (SEQ ID NO:139).

15. The yeast cell of claim 1, wherein the XPDH enzyme has a sequence at least 85% identical to at least one of SEQ ID NOs: 14, 15, 28, or 31.

16. The yeast cell of claim 1, wherein the XPDH enzyme has a sequence at least 90% identical to at least one of SEQ ID NOs: 14, 15, 28, or 31.

17. A method for producing xylitol, the method comprising:
contacting a substrate comprising dextrose with the engineered yeast cell of claim 1, wherein fermentation of the substrate by the engineered yeast produces xylitol.

18. (canceled)

19. (canceled)

20. The method of claim 17, wherein the fermentation temperature is at or between 25° C. to 45° C. and the volumetric oxygen uptake rate (OUR) is between 0.5 to 40, mmol O₂/(L· h).

21. The method of claim 17, wherein xylitol is produced at a rate of at least 0.2 g L⁻¹ h⁻¹.

22. The method of claim 17, wherein xylitol production is at least 20 g/L when the fermentation is run at 35° C. for 96 hours.

23. The method of claim 17, wherein erythritol production is reduced relative to an equivalent fermentation run with an equivalent yeast cell lacking the exogenous polynucleotide sequence.

24. The method of claim 17, wherein erythritol production is less than 20 g/L when the fermentation is run at 35° C. for 96 hours.

25. (canceled)

26. (canceled)
